(19) United States
(12) Patent Application Publication
(10) Pub. No.: US 2025/0360158 A1
(43) Pub. Date: Nov. 27, 2025

(54) OLIGONUCLEOTIDE ANALOGUE FORMULATIONS

(71) Applicant: NeuBase Therapeutics, Inc., Pittsburgh, PA (US)

(72) Inventors: Dani STOLTZFUS, Pittsburgh, PA (US); Barry BADEAU, Evanston, IL (US); Nathan TAVENOR, Swissvale, PA (US)

(21) Appl. No.: 18/873,193

(22) PCT Filed: Jun. 12, 2023
(86) PCT No.: PCT/US2023/025061

§ 371 Date: Dec. 9, 2024

Related U.S. Application Data
(60) Provisional application No. 63/351,666, filed on Jun. 13, 2022.

(51) Int. Cl. A61K 31/7125; A61K 9/08; A61K 47/22; A61K 47/26

(52) U.S. Cl. CPC A61K 31/7125; A61K 9/08; A61K 47/22; A61K 47/26

(57) ABSTRACT

The present disclosure relates to formulations of peptide nucleic acids (PNAs) and derivatives thereof useful for the modulation of target nucleic acids. The present disclosure further relates to the treatment of trinucleotide repeat disorders and cancer with pharmaceutical compositions disclosed herein.

### CROSS REFERENCE

This application claims the benefit of U.S. Provisional Application No. 63/351,666, filed Jun. 13, 2022, which is incorporated herein by reference in its entirety.

### INCORPORATION BY REFERENCE

Each patent, publication, and non-patent literature cited in the application is hereby incorporated by reference in its entirety as if each was incorporated by reference individually.

### BACKGROUND

Peptide nucleic acids (PNAs) are nucleobase-bearing polymeric constructs with repeating units based on a N-(2-aminoethyl)glycine scaffold. PNAs can mimic the arrangement of nucleobases in nucleic acids and can be tailored to hybridize with DNA or RNA with high affinity and specificity. PNAs have been used as biosensors, antivirals, antiparasitics, antibacterials, and therapeutic agents for treatment of genetic disorders.

### SUMMARY

In some embodiments, the present disclosure provides a pharmaceutical composition comprising:
(a) an oligonucleotide analogue comprising a structure according to formula (II):

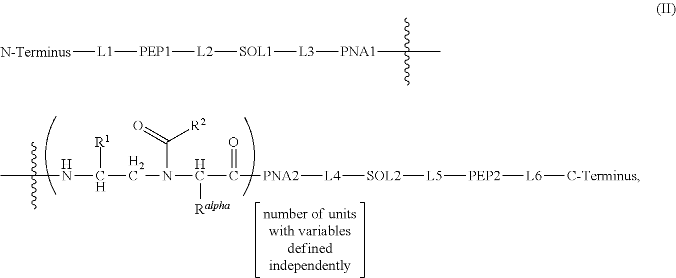

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof; and
(b) an amino acid or a pharmaceutically-acceptable salt or ionized form thereof thereof, wherein the amino acid comprises a side chain that is positively charged at physiological pH.

### DETAILED DESCRIPTION

Described herein are pharmaceutical compositions for the modulation of nucleic acids bearing mutations associated with a disease, including trinucleotide repeat disorders such as Huntington's disease and Myotonic Dystrophy, and Ras cancers.

The disclosed compositions can comprise a schedule of one or more pharmaceutically-acceptable agents, which alone or in combination can solubilize a compound herein or a pharmaceutically-acceptable salt or ionized form thereof and can provide a formulation that has a higher amount of the compound in solution relative to another formulation of the compound that lacks the schedule of agents(s) but is otherwise identical. Additives that increase solubility can decrease the dosage required to achieve an efficacious outcome and reduce the amount of time required for a therapeutically-effective concentration of a compound to be reached at the target site of therapy in a subject. Such compositions can also offer enhanced chemical and physical stability. The stability of a compound in solution, or the ability of the compound to maintain a homogenous state over time, can contribute to enhanced shelf life.

In some embodiments, the pharmaceutical composition comprises one or more pharmaceutically-acceptable additives, such as a buffer, tonicity modifying agent, or anti-aggregation agent. In some embodiments, the one or more pharmaceutically-acceptable additives comprise an amino acid. In some embodiments, the one or more pharmaceutically-acceptable additives comprise a monosaccharide and/or disaccharide.

### Compounds of the Disclosure.

Compounds provided in the pharmaceutical compositions disclosed herein can be effective for the modulation of target nucleic acids, such as RNA or DNA. Such compounds can be effective for binding to nucleic acid molecules containing mutations associated with a disease, such as, for example, a trinucleotide repeat disorder or cancer.

In some embodiments, a compound disclosed herein comprises an oligomeric structure, wherein the oligomeric structure comprises a repeating unit of formula (I):

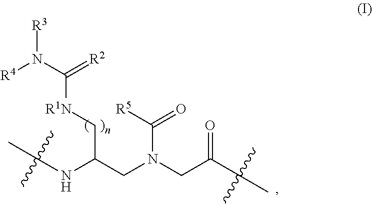

or an ionized form thereof, wherein:
R¹ is H, alkyl, or a nitrogen atom protecting group;
R² is O, NH, N(alkyl), or N(Pg^{N}), wherein Pg^{N} is a nitrogen atom protecting group;
R³ is H, alkyl, or a nitrogen atom protecting group;
R⁴ is H, alkyl, or a nitrogen atom protecting group;
R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
n is 1, 2, 3, 4, 5, 6, 7, 8, 9, or 10,
   or a pharmaceutically-acceptable salt or ionized form thereof.

In some embodiments, each of R¹, R³, and R⁴ is hydrogen; and R² is NH or N(Pg^{N}). In some embodiments, R⁵ is linear alkyl. In some embodiments, R⁵ is methyl. In some embodiments, n is 3. In some embodiments, n is 4.

In some embodiments, the compound further comprises a first chemical moiety attached to the oligomeric structure, and a second chemical moiety attached to the oligomeric structure, wherein the oligomeric structure, wherein the first chemical moiety, and the second chemical moiety form a compound according to formula (Ia):

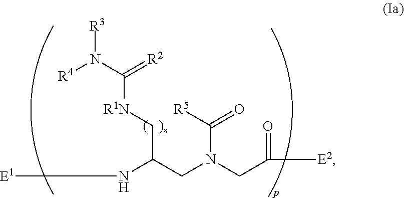

E¹ is the first chemical moiety;
E² is the second chemical moiety; and
p is an integer that is 1-100.

In some embodiments, p is 6. In some embodiments, p is 7. In some embodiments, p is 8.

In some embodiments, E¹ is hydrogen, acyl, a group that together with the nitrogen atom to which E¹ is bound forms a carbamate, a probe, a metal chelator, an imaging agent, or a biologically-active agent; and E² is OH, OMe, NH₂, a probe, a metal chelator, an imaging agent, or a biologically-active agent. In some embodiments, E¹ is hydrogen and E² is the biologically-active agent.

In some embodiments, the biologically-active agent comprises a structure that interferes with expression of a gene associated with a neuromuscular disease phenotype. In some embodiments, the neuromuscular disease phenotype is a DM1 disease phenotype. In some embodiments, the DM1 disease phenotype is associated with a non-wild-type DM1 gene that differs from a wild type DM1 gene in a repeat expansion mutation. In some embodiments, the biologically-active agent binds to a mRNA sequence at a region that is (CUG)_{z}, wherein z is an integer from 1-100 (SEQ ID NO: 324). In some embodiments, the biologically active agent is a peptide nucleic acid according to any one of SEQ ID NOs: 1-14 and 19-25. In some embodiments, the biologically active agent and the oligomeric structure form a sequence according to any one of SEQ ID NOs: 27-44 and 83. In some embodiments, the compound is a sequence according to any one of SEQ ID NOs: 27-44 and 83.

In some embodiments, the biologically-active agent comprises a structure that interferes with expression of a gene associated with a neurodegenerative disease phenotype. In some embodiments, the neurodegenerative disease phenotype is a Huntington's disease phenotype. In some embodiments, the Huntington's disease phenotype is associated with a non-wild-type HTT gene that differs from a wild type HTT gene in a repeat expansion mutation. In some embodiments, the biologically-active agent binds to a mRNA sequence at a region that is (CAG)_{z}, wherein z is an integer from 1-100 (SEQ ID NO: 325). In some embodiments, the biologically active agent is a peptide nucleic acid according to any one of SEQ ID NOs: 15-18, 26, 45, 26, 45-47, 55, 57, 59, 61, 63, 65, 67-77, 80, and 81. In some embodiments, the biologically active agent and the oligomeric structure form a sequence according to any one of SEQ ID NOs: 27-44 and 83. In some embodiments, the compound is a sequence according to any one of SEQ ID NOs: 27-44 and 83.

In some embodiments, the biologically-active agent comprises a structure that interferes with expression of a cancer-causing protein. In some embodiments, the cancer-causing protein is mutant K-ras. In some embodiments, the cancer-causing protein is G12D K-ras. In some embodiments, the cancer-causing protein is G12C K-ras. In some embodiments, the cancer-causing protein is G12V K-ras.

In some embodiments, the biologically-active agent binds to a nucleic acid sequence encoding a cancer gene. In some embodiments, the biologically-active agent binds to a mRNA sequence transcripted from a cancer gene. In some embodiments, the biologically-active agent binds to a DNA sequence encoding a cancer gene. In some embodiments, the cancer gene is non-wild type KRAS. In some embodiments, the cancer gene is G12D KRAS. In some embodiments, the cancer gene is G12C KRAS. In some embodiments, the cancer gene is G12V KRAS.

In some embodiments, the biologically-active agent binds to the nucleic acid sequence encoding the mutant K-ras by interactions between the heterocycles of the R² groups and nucleobases of the nucleic acid sequence. In some embodiments, the nucleic acid sequence is a mRNA sequence. In some embodiments, the nucleic acid sequence is a DNA sequence. In some embodiments, the biologically-active agent is a peptide nucleic acid according to any one of SEQ ID NOs: 84-123, 135-142, 163-166, 193-219, and 235-277. In some embodiments, the biologically-active agent and the water solubilizing group form a sequence according to any one of SEQ ID NOs: 124-134, 143-160, 167-192, 220-234, and 278-296. In some embodiments, the compound is a sequence according to any one of SEQ ID NOs: 124-134, 143-160, 167-192, 220-234, and 278-296.

In some embodiments, the biologically-active agent is an oligonucleotide or oligonucleotide analogue. In some embodiments, the biologically-active agent is a peptide nucleic acid.

In some embodiments, the compound has a structure according to formula (I-1):

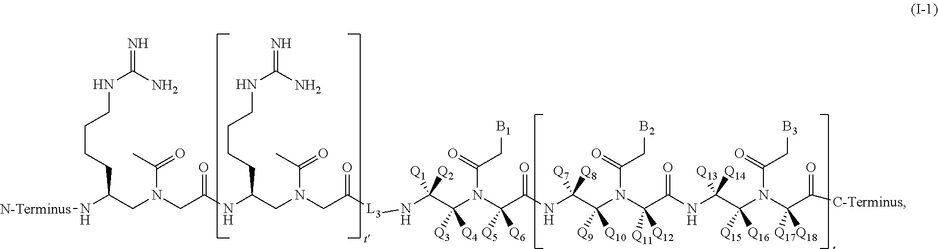

wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L3 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9, or a pharmaceutically acceptable salt or ionized form thereof.

In some embodiments, L3 is absent. In some embodiments, N-Terminus is H. In some embodiments, C-Terminus is NH₂. In some embodiments, each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl that is substituted or unsubstituted, or hydrogen.

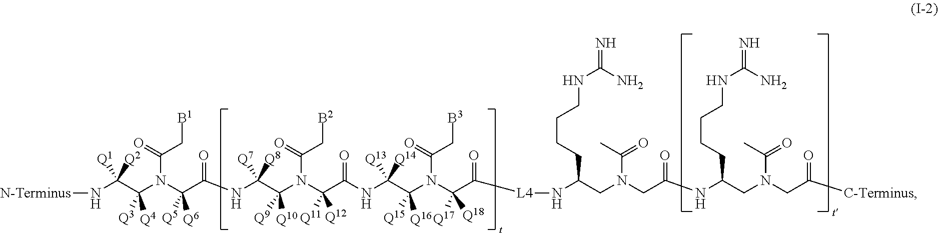

wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L4 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

In some embodiments, L4 is absent. In some embodiments, C-Terminus is NH₂. In some embodiments, N-Terminus is H. In some embodiments, each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl that is substituted or unsubstituted, or hydrogen.

In some embodiments, the present disclosure provides a compound comprising a structure according to formula (II):

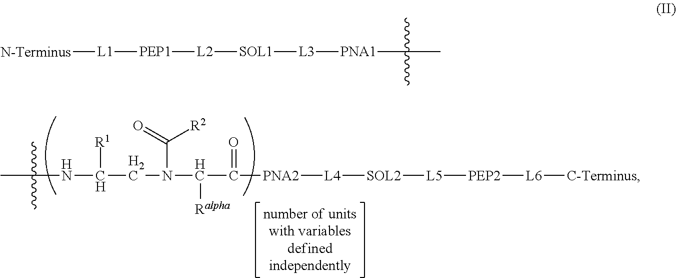

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof.

In some embodiments, the number of units with variables defined independently is 11-1,000. In some embodiments, the number of units with variables defined independently is 11-100. In some embodiments, the number of units with variables defined independently is 11-50.

In some embodiments, the number of units with variables defined independently is 11, 12, 13, 14, 15, 16, 17, 18, 19, 20, 21, 22, 23, 24, 25, 26, 27, 28, 29, or 30.

In some embodiments, each R^{alpha} is H. In some embodiments, each R^{alpha} is independently alkyl that is unsubstituted. In some embodiments, each alkyl that is unsubstituted is independently methyl, ethyl, prop-1-yl, prop-2-yl, 2-methylprop-1-yl, but-1yl, but-2-yl, or pent-1-yl. In some embodiments, each alkyl that is unsubstituted is independently methyl, prop-2-yl, 2-methylprop-1-yl, or but-2-yl. In some embodiments, each R^{alpha} is independently alkyl that is substituted.

In some embodiments, each alkyl that is substituted is independently substituted with —OH, —SH, —SMe, —NH₂, a heterocycle, an aryl group, a carboxylic acid, a guanidino group, a N-methylguanidino group, or an amido group. In some embodiments, each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

In some embodiments, each R^{alpha} is independently H, 3-guanidinoprop-1-yl, or 4-guanidinobut-1-yl. In some embodiments, at least one iteration of R^{alpha} is 3-guanidinoprop-1-yl.

In some embodiments, at least a third of the iterations of R^{alpha} are 3-guanidinoprop-1-yl. In some embodiments, at least half the iterations of R^{alpha} are 3-guanidinoprop-1-yl.

In some embodiments, the number of units with variables defined independently is at least 11; and at least one iteration of R¹ is a hydroxyalkyl group.

In some embodiments, at least a third of the R² groups in the structure are methyl substituted with a heterocycle. In some embodiments, at least half of the R² groups in the structure are methyl substituted with a heterocycle. In some embodiments, the heterocycles of the R² groups are nucleobases or analogues of nucleobases. In some embodiments, at least one of the heterocycles of the R² groups is a divalent nucleobase. In some embodiments, the heterocycles of the R² groups are divalent nucleobases.

In some embodiments, the heterocycles of the R² groups are each independently:

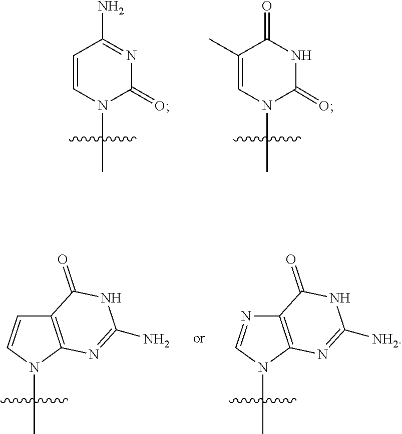

In some embodiments, each R² is independently: methyl,

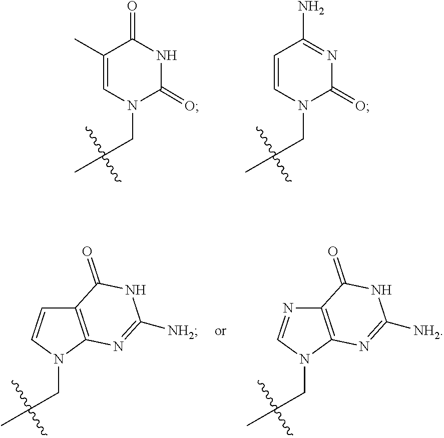 [SMILES: *Cn1cc(C)c(=O)[nH]c1=O ; *Cn1ccc(N)nc1=O ; *Cn1ccc2c(=O)[nH]c(N)nc21 ; "or" ; *Cn1cnc2c(=O)[nH]c(N)nc21]

In some embodiments, the heterocycles of the R² groups form a sequence that repeats at least twice, wherein the sequence is, from N-Terminus to C-Terminus:

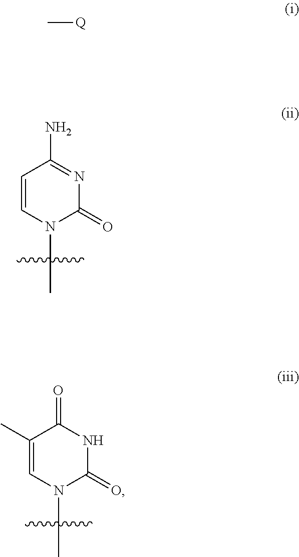

wherein Q is

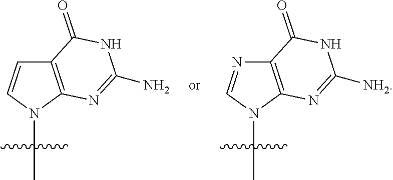 [SMILES: *n1ccc2c(=O)[nH]c(N)nc21 ; *n1cnc2c(=O)[nH]c(N)nc21]

In some embodiments, the heterocycles of the R² groups are each independently:

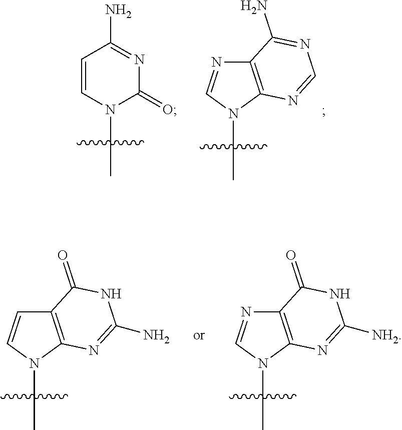

In some embodiments, each R² is independently: methyl,

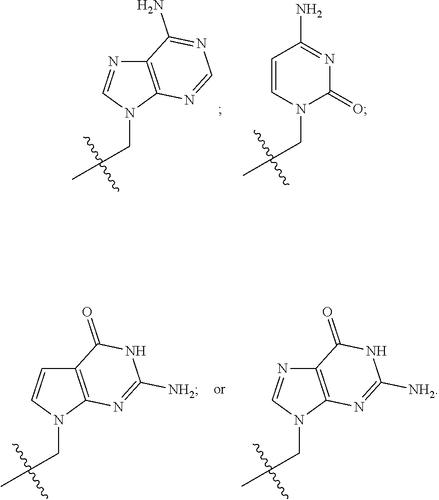 [SMILES: *Cn1cnc2c(N)ncnc21 ; *Cn1ccc(N)nc1=O ; *Cn1ccc2c(=O)[nH]c(N)nc21 ; *Cn1cnc2c(=O)[nH]c(N)nc21]

In some embodiments, the heterocycles of the R² groups form a sequence that repeats at least twice, wherein the sequence is, from N-Terminus to C-Terminus:

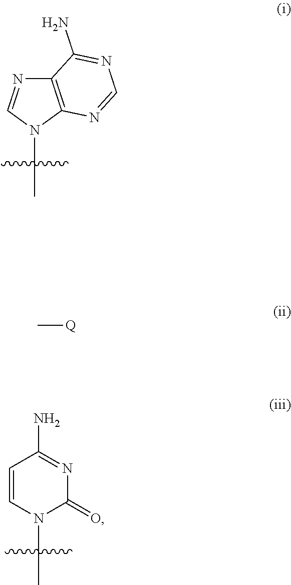

wherein Q is

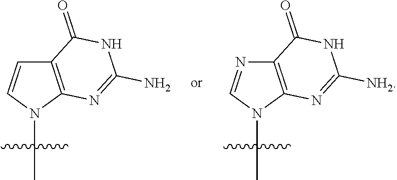 [SMILES: *n1ccc2c(=O)[nH]c(N)nc21 ; *n1cnc2c(=O)[nH]c(N)nc21]

In some embodiments, the heterocycles of the R² groups are each independently:

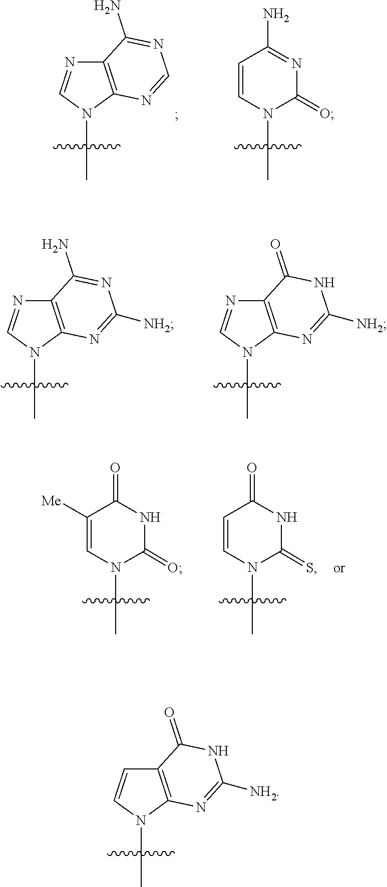

In some embodiments, each R² is independently: methyl,

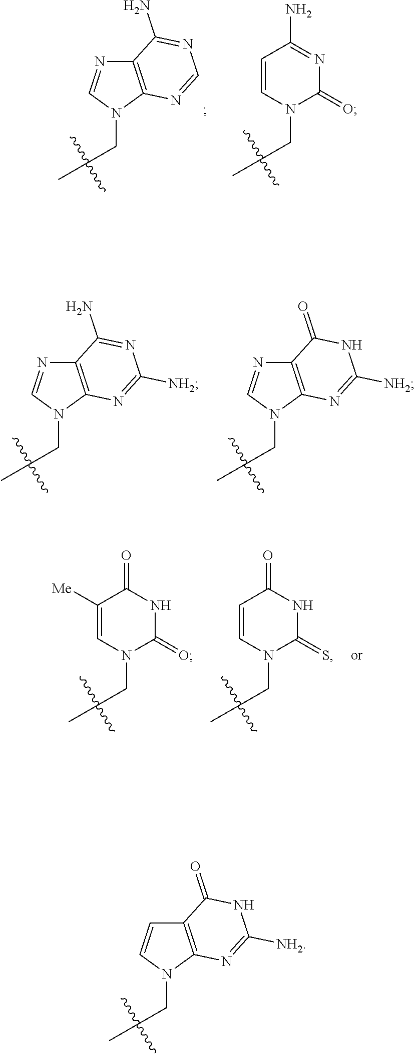

In some embodiments, of the units with variables defined independently, counting from N-Terminus, the first unit, the fourth unit, the seventh unit, the tenth unit, the thirteenth unit, the sixteenth unit, the nineteenth unit, the twenty-second unit, and the twenty-fifth unit, independently if present, each have

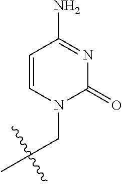 [SMILES: *Cn1ccc(N)nc1=O]

at R². In some embodiments, of the units with variables defined independently, counting from N-Terminus, the second unit, the fifth unit, the eighth unit, the eleventh unit, the fourteenth unit, the seventeenth unit, the twentieth unit, the twenty-third unit, and the twenty-sixth unit, independently if present, each have

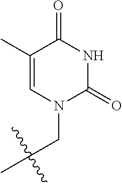 [SMILES: *Cn1cc(C)c(=O)[nH]c1=O]

### at R².

In some embodiments, of the units with variables defined independently, counting from N-Terminus, the second unit, the fifth unit, the eighth unit, the eleventh unit, the fourteenth unit, the seventeenth unit, the twentieth unit, the twenty-third unit, and the twenty-sixth unit, independently if present, each have

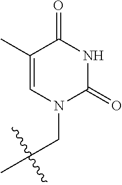 [SMILES: *Cn1cc(C)c(=O)[nH]c1=O]

### at R².

In some embodiments, of the units with variables defined independently, counting from N-Terminus, the second unit, the fifth unit, the eighth unit, the eleventh unit, the fourteenth unit, the seventeenth unit, the twentieth unit, the twenty-third unit, and the twenty-sixth unit, independently if present, each have

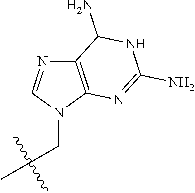 [SMILES: *Cn1cnc2c1N=C(N)NC2N]

### at R².

In some embodiments, of the units with variables defined independently, counting from N-Terminus, the third unit, the sixth unit, the ninth unit, the twelfth unit, the fifteenth unit, the eighteenth unit, the twenty-first unit, and the twenty-fourth unit, independently if present, each have

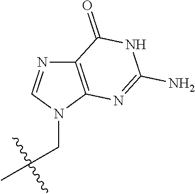 [SMILES: *Cn1cnc2c(=O)[nH]c(N)nc21]

### at R².

In some embodiments, of the units with variables defined independently, counting from N-Terminus, the third unit, the sixth unit, the ninth unit, the twelfth unit, the fifteenth unit, the eighteenth unit, the twenty-first unit, and the twenty-fourth unit, independently if present, each have

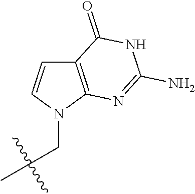 [SMILES: *Cn1ccc2c(=O)[nH]c(N)nc21]

### at R².

In some embodiments, the number of units with variables defined independently is 14, 15, 16, 17, or 18, wherein:
a first unit is present, and in the first unit:
R¹ is H or —CH₂OH; and R² is

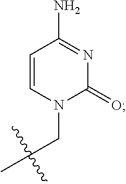 [SMILES: *Cn1ccc(N)nc1=O]

a second unit is present, and in the second unit:
R¹ is H or —CH₂OH; and R² is

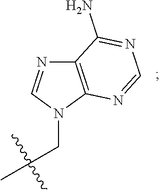 [SMILES: *Cn1cnc2c(N)ncnc21]

a third unit is present, and in the third unit:
R¹ is H or —CH₂OH; and R² is

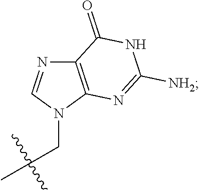 [SMILES: *Cn1cnc2c(=O)[nH]c(N)nc21]

a fourth unit is present, and in the fourth unit:
R¹ is H or —CH₂OH; and R² is

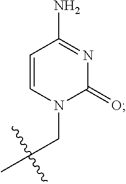 [SMILES: *Cn1ccc(N)nc1=O]

a fifth unit is present, and in the fifth unit:
R¹ is H or —CH₂OH; and R² is

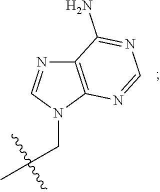 [SMILES: *Cn1cnc2c(N)ncnc21]

a sixth unit is present, and in the sixth unit:
R¹ is H or —CH₂OH; and R² is

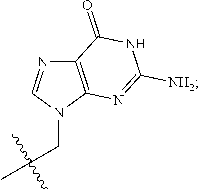 [SMILES: *Cn1cnc2c(=O)[nH]c(N)nc21]

a seventh unit is present, and in the seventh unit:
R¹ is H or —CH₂OH; and R² is

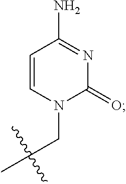 [SMILES: *Cn1ccc(N)nc1=O]

an eighth unit is present, and in the eighth unit:
R¹ is H or —CH₂OH; and R² is

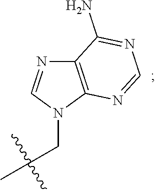 [SMILES: *Cn1cnc2c(N)ncnc21]

a ninth unit is present, and in the ninth unit:
R¹ is H or —CH₂OH; and R² is

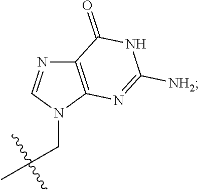 [SMILES: *Cn1cnc2c(=O)[nH]c(N)nc21]

a tenth unit is present, and in the tenth unit:
R¹ is H or —CH₂OH; and R² is

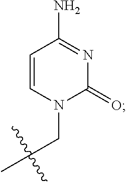 [SMILES: *Cn1ccc(N)nc1=O]

an eleventh unit is present, and in the eleventh unit:
R¹ is H or —CH₂OH; and R² is

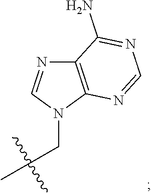 [SMILES: *Cn1cnc2c(N)ncnc21]

a twelfth unit is present, and in the twelfth unit:
R¹ is H or —CH₂OH; and R² is

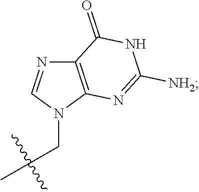 [SMILES: *Cn1cnc2c(=O)[nH]c(N)nc21]

a thirteenth unit is present, and in the thirteenth unit:
R¹ is H or —CH₂OH; and R² is

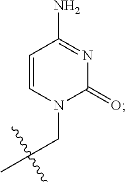 [SMILES: *Cn1ccc(N)nc1=O]

a fourteenth unit is present, and in the fourteenth unit:
R¹ is H or —CH₂OH; and R² is

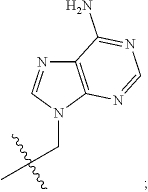 [SMILES: *Cn1cnc2c(N)ncnc21]

a fifteenth unit is present or absent, and in the fifteenth unit:
R¹ is H or —CH₂OH; and R² is

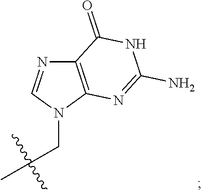 [SMILES: *Cn1cnc2c(=O)[nH]c(N)nc21]

a sixteenth unit is present or absent, and in the sixteenth unit:
R¹ is H or —CH₂OH; and R² is

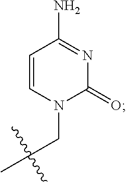 [SMILES: *Cn1ccc(N)nc1=O]

a seventeenth unit is present or absent, and in the seventeenth unit:
R¹ is H or —CH₂OH; and R² is

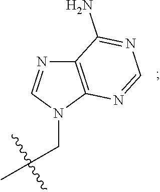 [SMILES: *Cn1cnc2c(N)ncnc21]

and
an eighteenth unit is present or absent, and in the eighteenth unit:
R¹ is H or —CH₂OH; and R² is

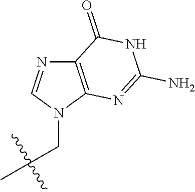 [SMILES: *Cn1cnc2c(=O)[nH]c(N)nc21]

(SEQ ID NO: 326).

In some embodiments, the number of units with variables defined independently is 12, 13, 14, 15, 16, 17, 18, 19, 20, 21, 22, 23, 24, 25, or 26, wherein:
a first unit is present or absent, and in the first unit:
R¹ is H or —CH₂OH; and R² is

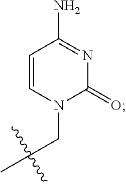 [SMILES: *Cn1ccc(N)nc1=O]

a second unit is present or absent, and in the second unit:
R¹ is H or —CH₂OH; and R² is

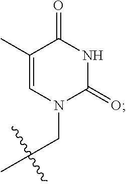 [SMILES: *Cn1cc(C)c(=O)[nH]c1=O]

a third unit is present, and in the third unit:
R¹ is H or —CH₂OH; and R² is

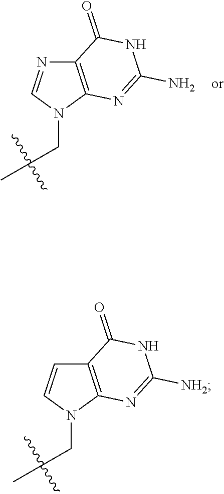 [SMILES: *Cn1cnc2c(=O)[nH]c(N)nc21 ; *Cn1ccc2c(=O)[nH]c(N)nc21]

a fourth unit is present, and in the fourth unit:
R¹ is H or —CH₂OH; and R² is

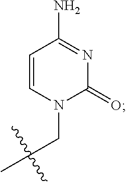 [SMILES: *Cn1ccc(N)nc1=O]

a fifth unit is present, and in the fifth unit:
R¹ is H or —CH₂OH; and R² is

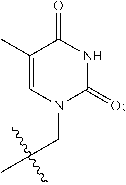 [SMILES: *Cn1cc(C)c(=O)[nH]c1=O]

a sixth unit is present, and in the sixth unit:
R¹ is H or —CH₂OH; and R² is

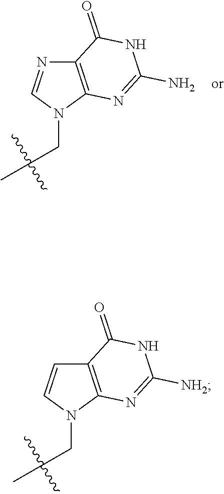 [SMILES: *Cn1cnc2c(=O)[nH]c(N)nc21 ; *Cn1ccc2c(=O)[nH]c(N)nc21]

a seventh unit is present, and in the seventh unit:
R¹ is H or —CH₂OH; and R² is

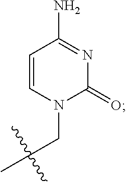 [SMILES: *Cn1ccc(N)nc1=O]

an eighth unit is present, and in the eighth unit:
R¹ is H or —CH₂OH; and R² is

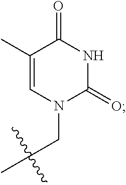 [SMILES: *Cn1cc(C)c(=O)[nH]c1=O]

a ninth unit is present, and in the ninth unit:
R¹ is H or —CH₂OH; and R² is

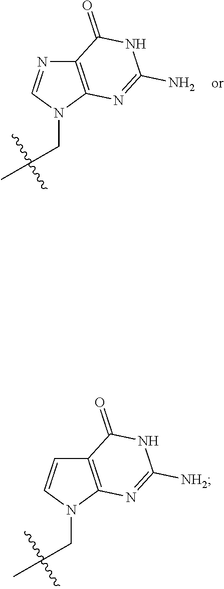 [SMILES: *Cn1cnc2c(=O)[nH]c(N)nc21 ; *Cn1ccc2c(=O)[nH]c(N)nc21]

a tenth unit is present, and in the tenth unit:
R¹ is H or —CH₂OH; and R² is

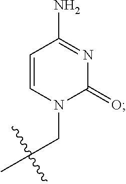 [SMILES: *Cn1ccc(N)nc1=O]

an eleventh unit is present, and in the eleventh unit:
R¹ is H or —CH₂OH; and R² is

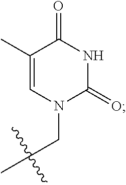 [SMILES: *Cn1cc(C)c(=O)[nH]c1=O]

a twelfth unit is present, and in the twelfth unit:
R¹ is H or —CH₂OH; and R² is

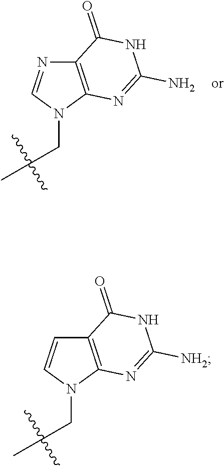 [SMILES: *Cn1cnc2c(=O)[nH]c(N)nc21 ; *Cn1ccc2c(=O)[nH]c(N)nc21]

a thirteenth unit is present, and in the thirteenth unit:
R¹ is H or —CH₂OH; and R² is

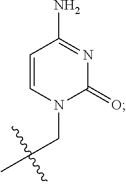 [SMILES: *Cn1ccc(N)nc1=O]

a fourteenth unit is present, and in the fourteenth unit:
R¹ is H or —CH₂OH; and R² is

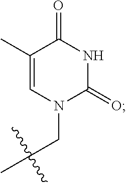 [SMILES: *Cn1cc(C)c(=O)[nH]c1=O]

a fifteenth unit is present or absent, and in the fifteenth unit:
R¹ is H or —CH₂OH; and R² is

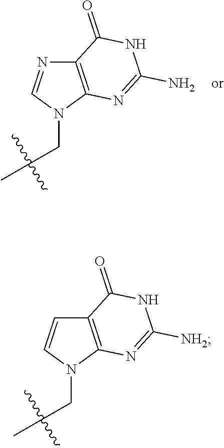 [SMILES: *Cn1cnc2c(=O)[nH]c(N)nc21 ; *Cn1ccc2c(=O)[nH]c(N)nc21]

a sixteenth unit is present or absent, and in the sixteenth unit:
R¹ is H or —CH₂OH; and R² is

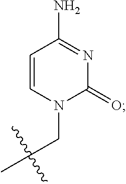 [SMILES: *Cn1ccc(N)nc1=O]

a seventeenth unit is present or absent, and in the seventeenth unit:
R¹ is H or —CH₂OH; and R² is

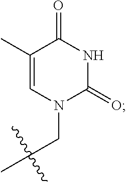 [SMILES: *Cn1cc(C)c(=O)[nH]c1=O]

and
an eighteenth unit is present or absent, and in the eighteenth unit:
R¹ is H or —CH₂OH; and R² is

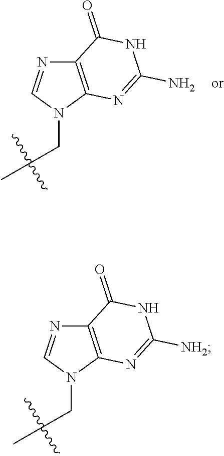 [SMILES: *Cn1cnc2c(=O)[nH]c(N)nc21 ; *Cn1cnc2c(=O)[nH]c(N)nc21]

a nineteenth unit is present or absent, and in the nineteenth unit:
R¹ is H or —CH₂OH; and R² is

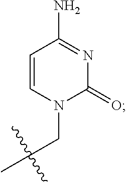 [SMILES: *Cn1ccc(N)nc1=O]

a twentieth unit is present or absent, and in the twentieth unit:
R¹ is H or —CH₂OH; and R² is

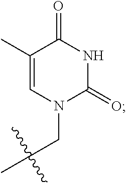 [SMILES: *Cn1cc(C)c(=O)[nH]c1=O]

a twenty-first unit is present or absent, and in the twenty-first unit:
R¹ is H or —CH₂OH; and R² is

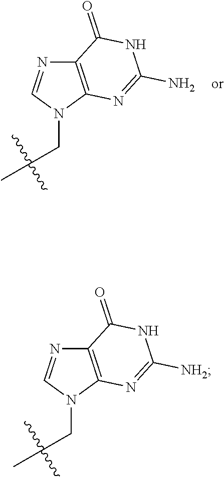 [SMILES: *Cn1cnc2c(=O)[nH]c(N)nc21 ; *Cn1cnc2c(=O)[nH]c(N)nc21]

a twenty-second unit is present or absent, and in the twenty-second unit:
R¹ is H or —CH₂OH; and R² is

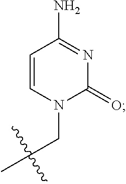 [SMILES: *Cn1ccc(N)nc1=O]

a twenty-third unit is present or absent, and in the twenty-third unit:
R¹ is H or —CH₂OH; and R² is

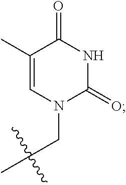 [SMILES: *Cn1cc(C)c(=O)[nH]c1=O]

a twenty-fourth unit is present or absent, and in the twenty-fourth unit:
R¹ is H or —CH₂OH; and R² is

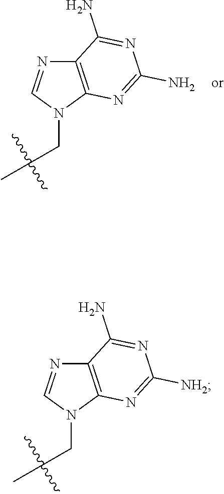 [SMILES: *Cn1cnc2c(N)nc(N)nc21 ; *Cn1cnc2c(N)nc(N)nc21]

a twenty-fifth unit is present or absent, and in the twenty-fifth unit:
R¹ is H or —CH₂OH; and R² is

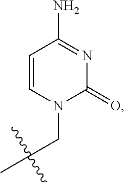 [SMILES: *Cn1ccc(N)nc1=O]

and
a twenty-sixth unit is present or absent, and in the twenty-sixth unit:
R¹ is H or —CH₂OH; and R² is

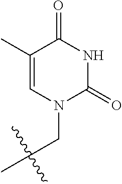 [SMILES: *Cn1cc(C)c(=O)[nH]c1=O]

(SEQ ID NO: 327).

In some embodiments, of the units with variables defined independently, counting from N-Terminus, the second unit, the fourth unit, the sixth unit, the eighth unit, the tenth unit, the twelfth unit, the fourteenth unit, the sixteenth unit, the eighteenth unit, the twentieth unit, the twenty-second unit, the twenty-fourth unit, and the twenty sixth unit, independently if present, each have hydrogen at R¹.

In some embodiments, of the units with variables defined independently, counting from N-Terminus, the fourth unit, the sixth unit, the seventh unit, the ninth unit, the tenth unit, the twelfth unit, the fourteenth unit, the sixteenth unit, the seventeenth unit, the nineteenth unit, the twentieth unit, the twenty-second unit, the twenty-third unit, the twenty-fifth unit, and the twenty-sixth unit, independently if present, each have hydrogen at R¹.

In some embodiments, of the units with variables defined independently, counting from N-Terminus, the third unit, the fifth unit, the seventh unit, the ninth unit, the eleventh unit, the thirteenth unit, the fifteenth unit, the seventeenth unit, the nineteenth unit, the twenty-first unit, the twenty-third unit, and the twenty-fifth unit, independently if present, each have —CH₂OH at R¹.

In some embodiments, of the units with variables defined independently, counting from N-Terminus, the third unit, the fifth unit, the eighth unit, the eleventh unit, the thirteenth unit, the fifteenth unit, the eighteenth unit, the twenty-first unit, and the twenty-fourth unit, independently if present, each have —CH₂OH at R¹.

In some embodiments, of the units with variables defined independently, counting from N-Terminus, the third unit, the fifth unit, the seventh unit, the ninth unit, the eleventh unit, the thirteenth unit, the fifteenth unit, the seventeenth unit, the nineteenth unit, the twenty-first unit, the twenty-third unit, and the twenty-fifth unit, independently if present, each have —CH₂OH at R^{alpha}.

In some embodiments, of the units with variables defined independently, counting from N-Terminus, the third unit, the fifth unit, the eighth unit, the eleventh unit, the thirteenth unit, the fifteenth unit, the eighteenth unit, the twenty-first unit, and the twenty-fourth unit, independently if present, each have —CH₂OH at R^{alpha}.

In some embodiments, of the units with variables defined independently, counting from N-Terminus, the third unit, the fifth unit, the seventh unit, the ninth unit, the eleventh unit, the thirteenth unit, the fifteenth unit, the seventeenth unit, the nineteenth unit, the twenty-first unit, the twenty-third unit, and the twenty-fifth unit, independently if present, each have 3-guanidino-prop-1-yl at R^{alpha}.

In some embodiments, of the units with variables defined independently, counting from N-Terminus, the third unit, the fifth unit, the eighth unit, the eleventh unit, the thirteenth unit, the fifteenth unit, the eighteenth unit, the twenty-first unit, and the twenty-fourth unit, independently if present, each have 3-guanidino-prop-1-yl at R^{alpha}.

In some embodiments, of the units with variables defined independently, counting from N-Terminus, the third unit, the fifth unit, the eighth unit, the eleventh unit, the thirteenth unit, the fifteenth unit, the eighteenth unit, the twenty-first unit, and the twenty-fourth unit, independently if present, each have 4-guanidino-but-1-yl at R¹.

In some embodiments, of the units with variables defined independently, counting from N-Terminus, the third unit, the fifth unit, the seventh unit, the ninth unit, the eleventh unit, the thirteenth unit, the fifteenth unit, the seventeenth unit, the nineteenth unit, the twenty-first unit, the twenty-third unit, and the twenty-fifth unit, independently if present, each have 4-guanidino-but-1-yl at R¹.

In some embodiments, of the units with variables defined independently, counting from N-Terminus, the first, third, sixth, ninth, eleventh, thirteenth, sixteenth, nineteenth, and twenty-second units, independently if present, each have 3-guanidinoprop-1-yl at R^{alpha}.

In some embodiments, the number of units with variables defined independently is 17, wherein:
a first unit is present or absent, and in the first unit:
R¹ is H, —CH₂OH, or 4-guanidinobut-1-yl; and R² is

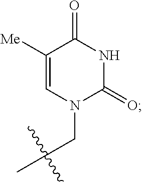 [SMILES: *Cn1cc(C)c(=O)[nH]c1=O]

a second unit is present or absent, and in the second unit:
R¹ is H, —CH₂OH, or 4-guanidinobut-1-yl; and R² is

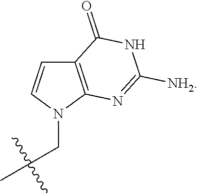 [SMILES: *Cn1ccc2c(=O)[nH]c(N)nc21]

a third unit is present, and in the third unit:
R¹ is H, —CH₂OH, or 4-guanidinobut-1-yl; and R² is

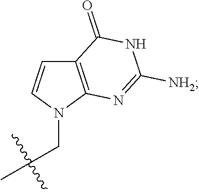 [SMILES: *Cn1ccc2c(=O)[nH]c(N)nc21]

a fourth unit is present, and in the fourth unit:
R¹ is H, —CH₂OH, or 4-guanidinobut-1-yl; and R² is

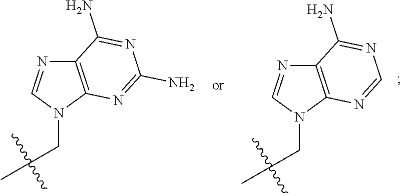 [SMILES: *Cn1cnc2c(N)nc(N)nc21 ; *Cn1cnc2c(N)ncnc21]

a fifth unit is present, and in the fifth unit:
R¹ is H, —CH₂OH, or 4-guanidinobut-1-yl; and R² is

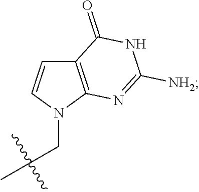 [SMILES: *Cn1ccc2c(=O)[nH]c(N)nc21]

a sixth unit is present, and in the sixth unit:
R¹ is H, —CH₂OH, or 4-guanidinobut-1-yl; and R² is

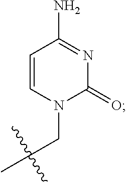 [SMILES: *Cn1ccc(N)nc1=O]

a seventh unit is present, and in the seventh unit:
R¹ is H, —CH₂OH, or 4-guanidinobut-1-yl; and R² is

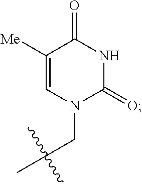 [SMILES: *Cn1cc(C)c(=O)[nH]c1=O]

an eighth unit is present, and in the eighth unit:
R¹ is H, —CH₂OH, or 4-guanidinobut-1-yl; and R² is

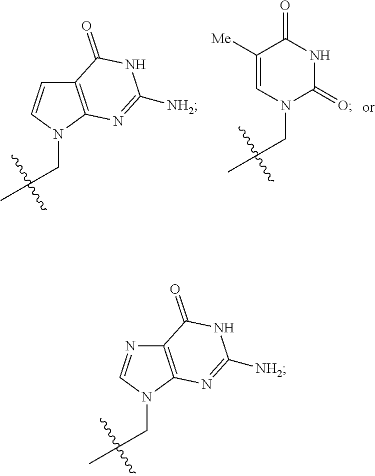 [SMILES: *Cn1ccc2c(=O)[nH]c(N)nc21 ; *Cn1cc(C)c(=O)[nH]c1=O ; *Cn1cnc2c(=O)[nH]c(N)nc21]

a ninth unit is present, and in the ninth unit:
R¹ is H, —CH₂OH, or 4-guanidinobut-1-yl; and R² is

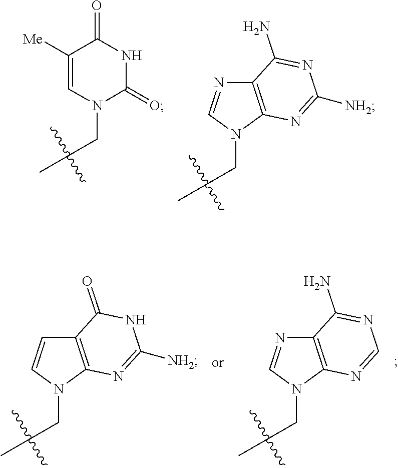 [SMILES: *Cn1cc(C)c(=O)[nH]c1=O ; *Cn1cnc2c(N)nc(N)nc21 ; *Cn1ccc2c(=O)[nH]c(N)nc21 ; *Cn1cnc2c(N)ncnc21]

a tenth unit is present, and in the tenth unit:
R¹ is H, —CH₂OH, or 4-guanidinobut-1-yl; and R² is

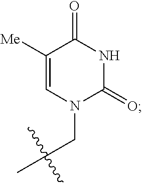 [SMILES: *Cn1cc(C)c(=O)[nH]c1=O]

an eleventh unit is present, and in the eleventh unit:
R¹ is H, —CH₂OH, or 4-guanidinobut-1-yl; and R² is

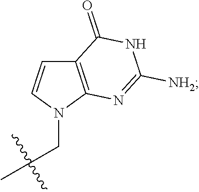 [SMILES: *Cn1ccc2c(=O)[nH]c(N)nc21]

a twelfth unit is present, and in the twelfth unit:
R¹ is H, —CH₂OH, or 4-guanidinobut-1-yl; and R² is

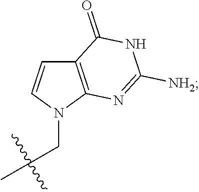 [SMILES: *Cn1ccc2c(=O)[nH]c(N)nc21]

a thirteenth unit is present, and in the thirteenth unit:
R¹ is H, —CH₂OH, or 4-guanidinobut-1-yl; and R² is

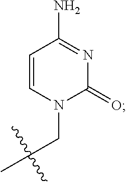 [SMILES: *Cn1ccc(N)nc1=O]

a fourteenth unit is present, and in the fourteenth unit:
R¹ is H, —CH₂OH, or 4-guanidinobut-1-yl; and R² is

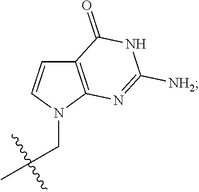 [SMILES: *Cn1ccc2c(=O)[nH]c(N)nc21]

a fifteenth unit is present, and in the fifteenth unit:
R¹ is H, —CH₂OH, or 4-guanidinobut-1-yl; and R² is

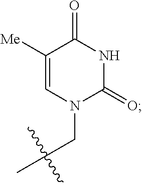 [SMILES: *Cn1cc(C)c(=O)[nH]c1=O]

a sixteenth unit is present, and in the sixteenth unit:
R¹ is H, —CH₂OH, or 4-guanidinobut-1-yl; and R² is

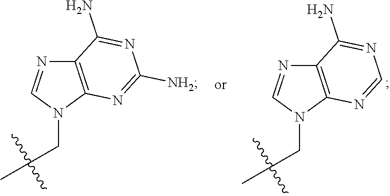 [SMILES: *Cn1cnc2c(N)nc(N)nc21 ; *Cn1cnc2c(N)ncnc21]

and
a seventeenth unit is present or absent, and in the seventeenth unit:
R¹ is H, —CH₂OH, or 4-guanidinobut-1-yl; and R² is

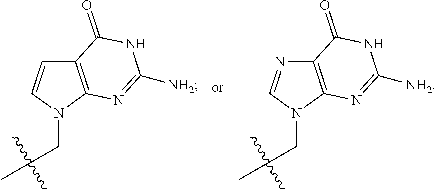 [SMILES: *Cn1ccc2c(=O)[nH]c(N)nc21 ; *Cn1cnc2c(=O)[nH]c(N)nc21]

In some embodiments, the number of units with variables defined independently is 14, 15, 16, or 17, wherein:
a first unit is present or absent, and in the first unit:
R¹ is H, —CH₂OH, or 4-guanidinobut-1-yl; and R² is

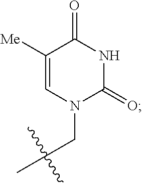 [SMILES: *Cn1cc(C)c(=O)[nH]c1=O]

a second unit is present or absent, and in the second unit:
R¹ is H, —CH₂OH, or 4-guanidinobut-1-yl; and R² is

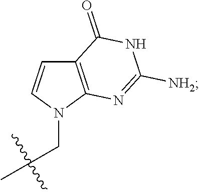 [SMILES: *Cn1ccc2c(=O)[nH]c(N)nc21]

a third unit is present, and in the third unit:
R¹ is H, —CH₂OH, or 4-guanidinobut-1-yl; and R² is

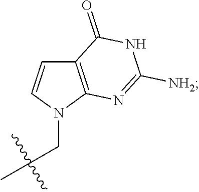 [SMILES: *Cn1ccc2c(=O)[nH]c(N)nc21]

a fourth unit is present, and in the fourth unit:
R¹ is H, —CH₂OH, or 4-guanidinobut-1-yl; and R² is

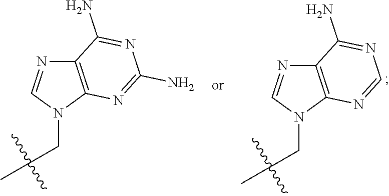 [SMILES: *Cn1cnc2c(N)nc(N)nc21 ; *Cn1cnc2c(N)ncnc21]

a fifth unit is present, and in the fifth unit:
R¹ is H, —CH₂OH, or 4-guanidinobut-1-yl; and R² is

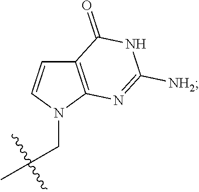 [SMILES: *Cn1ccc2c(=O)[nH]c(N)nc21]

a sixth unit is present, and in the sixth unit:
R¹ is H, —CH₂OH, or 4-guanidinobut-1-yl; and R² is

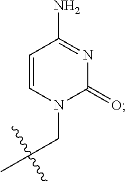 [SMILES: *Cn1ccc(N)nc1=O]

a seventh unit is present, and in the seventh unit:
R¹ is H, —CH₂OH, or 4-guanidinobut-1-yl; and R² is

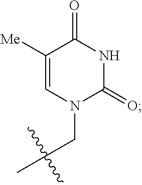 [SMILES: *Cn1cc(C)c(=O)[nH]c1=O]

an eighth unit is present, and in the eighth unit:
R¹ is H, —CH₂OH, or 4-guanidinobut-1-yl; and R² is

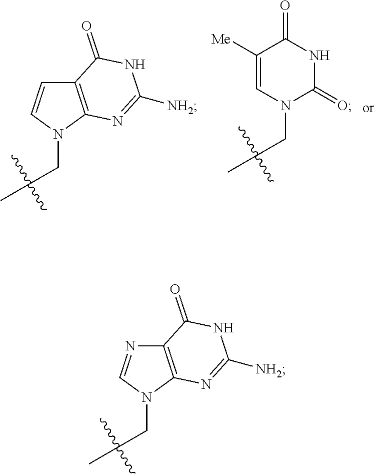 [SMILES: *Cn1ccc2c(=O)[nH]c(N)nc21 ; *Cn1cc(C)c(=O)[nH]c1=O ; *Cn1cnc2c(=O)[nH]c(N)nc21]

a ninth unit is present, and in the ninth unit:
R¹ is H, —CH₂OH, or 4-guanidinobut-1-yl; and R² is

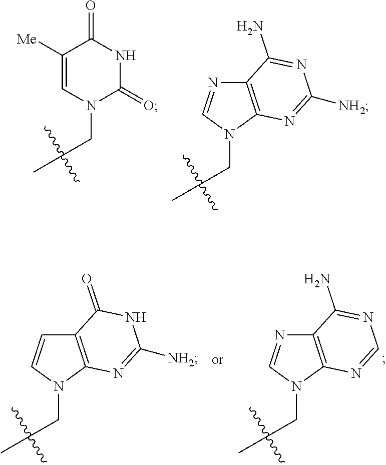 [SMILES: *Cn1cc(C)c(=O)[nH]c1=O ; *Cn1cnc2c(N)nc(N)nc21 ; *Cn1ccc2c(=O)[nH]c(N)nc21 ; *Cn1cnc2c(N)ncnc21]

a tenth unit is present, and in the tenth unit:
R¹ is H, —CH₂OH, or 4-guanidinobut-1-yl; and R² is

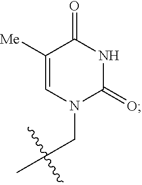 [SMILES: *Cn1cc(C)c(=O)[nH]c1=O]

an eleventh unit is present, and in the eleventh unit:
R¹ is H, —CH₂OH, or 4-guanidinobut-1-yl; and R² is

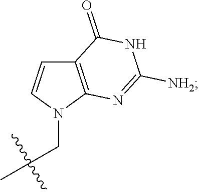 [SMILES: *Cn1ccc2c(=O)[nH]c(N)nc21]

a twelfth unit is present, and in the twelfth unit:
R¹ is H, —CH₂OH, or 4-guanidinobut-1-yl; and R² is

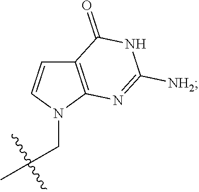 [SMILES: *Cn1ccc2c(=O)[nH]c(N)nc21]

a thirteenth unit is present, and in the thirteenth unit:
R¹ is H, —CH₂OH, or 4-guanidinobut-1-yl; and R² is

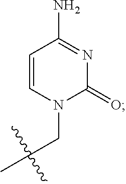 [SMILES: *Cn1ccc(N)nc1=O]

a fourteenth unit is present, and in the fourteenth unit:
R¹ is H, —CH₂OH, or 4-guanidinobut-1-yl; and R² is

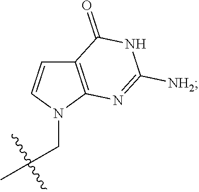 [SMILES: *Cn1ccc2c(=O)[nH]c(N)nc21]

a fifteenth unit is present, and in the fifteenth unit:
R¹ is H, —CH₂OH, or 4-guanidinobut-1-yl; and R² is

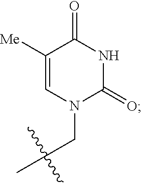 [SMILES: *Cn1cc(C)c(=O)[nH]c1=O]

a sixteenth unit is present, and in the sixteenth unit:
R¹ is H, —CH₂OH, or 4-guanidinobut-1-yl; and R² is

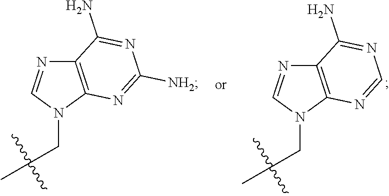 [SMILES: *Cn1cnc2c(N)nc(N)nc21 ; *Cn1cnc2c(N)ncnc21]

and
a seventeenth unit is present or absent, and in the seventeenth unit:
R¹ is H, —CH₂OH, or 4-guanidinobut-1-yl; and R² is

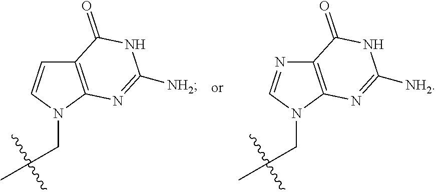 [SMILES: *Cn1ccc2c(=O)[nH]c(N)nc21 ; *Cn1cnc2c(=O)[nH]c(N)nc21]

In some embodiments, the number of units with variables defined independently is 11, 12, 13, 14, 15, 16, or 17, wherein:
a first unit is present or absent, and in the first unit:
R¹ is H, —CH₂OH, or 4-guanidinobut-1-yl; and R² is

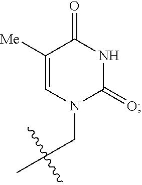 [SMILES: *Cn1cc(C)c(=O)[nH]c1=O]

a second unit is present or absent, and in the second unit:
R¹ is H, —CH₂OH, or 4-guanidinobut-1-yl; and R² is

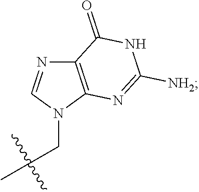 [SMILES: *Cn1cnc2c(=O)[nH]c(N)nc21]

or methyl;
a third unit is present or absent, and in the third unit:
R¹ is H, —CH₂OH, or 4-guanidinobut-1-yl; and R² is

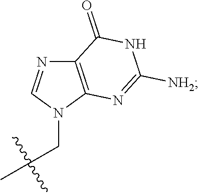 [SMILES: *Cn1cnc2c(=O)[nH]c(N)nc21]

a fourth unit is present or absent, and in the fourth unit:
R¹ is H, —CH₂OH, or 4-guanidinobut-1-yl; and R² is

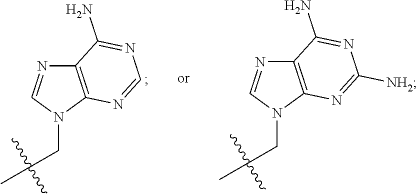 [SMILES: *Cn1cnc2c(N)ncnc21 ; *Cn1cnc2c(N)nc(N)nc21]

a fifth unit is present, and in the fifth unit:
R¹ is H, —CH₂OH, or 4-guanidinobut-1-yl; and R² is

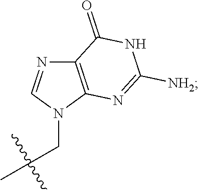 [SMILES: *Cn1cnc2c(=O)[nH]c(N)nc21]

or methyl;
a sixth unit is present, and in the sixth unit:
R¹ is H, —CH₂OH, or 4-guanidinobut-1-yl; and R² is

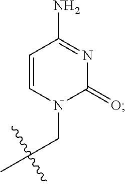 [SMILES: *Cn1ccc(N)nc1=O]

a seventh unit is present, and in the seventh unit:
R¹ is H, —CH₂OH, or 4-guanidinobut-1-yl; and R² is

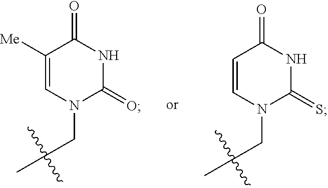 [SMILES: *Cn1cc(C)c(=O)[nH]c1=O ; "or" ; *Cn1ccc(=O)[nH]c1=S]

an eighth unit is present, and in the eighth unit:
R¹ is H, —CH₂OH, or 4-guanidinobut-1-yl; and R² is

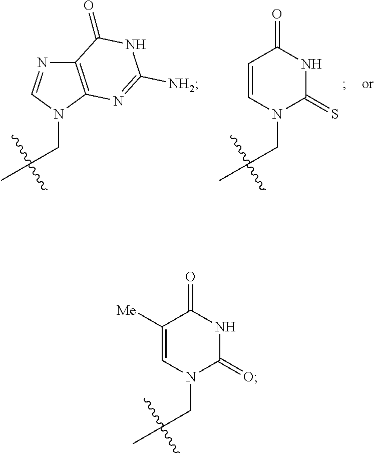 [SMILES: *Cn1cnc2c(=O)[nH]c(N)nc21 ; *Cn1ccc(=O)[nH]c1=S ; ";" ; *Cn1cc(C)c(=O)[nH]c1=O]

a ninth unit is present, and in the ninth unit:
R¹ is H, —CH₂OH, or 4-guanidinobut-1-yl; and R² is

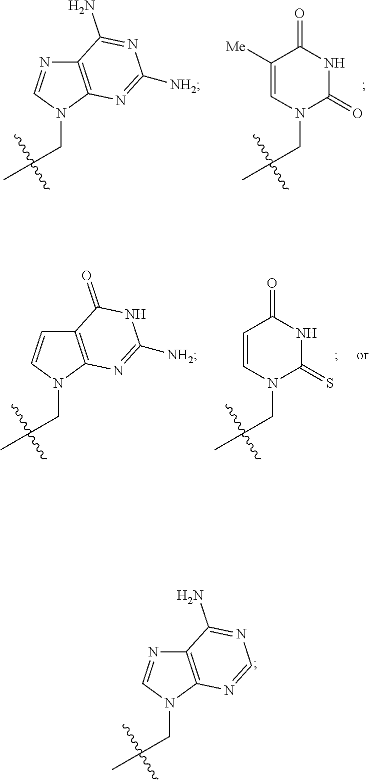

a tenth unit is present, and in the tenth unit:
R¹ is H, —CH₂OH, or 4-guanidinobut-1-yl; and R² is

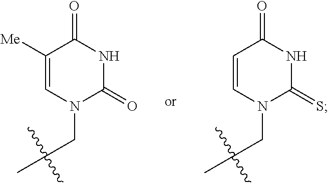 [SMILES: *Cn1cc(C)c(=O)[nH]c1=O ; "or" ; *Cn1ccc(=O)[nH]c1=S]

an eleventh unit is present, and in the eleventh unit:
R¹ is H, —CH₂OH, or 4-guanidinobut-1-yl; and R² is

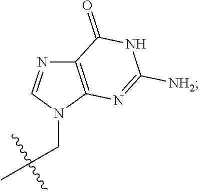 [SMILES: *Cn1cnc2c(=O)[nH]c(N)nc21]

or methyl;
a twelfth unit is present, and in the twelfth unit:
R¹ is H, —CH₂OH, or 4-guanidinobut-1-yl; and R² is

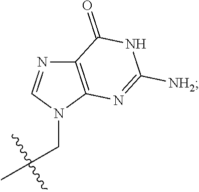 [SMILES: *Cn1cnc2c(=O)[nH]c(N)nc21]

a thirteenth unit is present, and in the thirteenth unit:
R¹ is H, —CH₂OH, or 4-guanidinobut-1-yl; and R² is

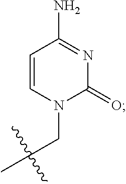 [SMILES: *Cn1ccc(N)nc1=O]

a fourteenth unit is present, and in the fourteenth unit:
R¹ is H, —CH₂OH, or 4-guanidinobut-1-yl; and R² is

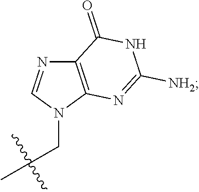 [SMILES: *Cn1cnc2c(=O)[nH]c(N)nc21]

or methyl;
a fifteenth unit is present or absent, and in the fifteenth unit:
R¹ is H, —CH₂OH, or 4-guanidinobut-1-yl; and R² is

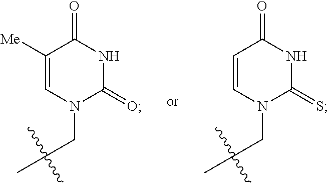 [SMILES: *Cn1cc(C)c(=O)[nH]c1=O ; "or" ; *Cn1ccc(=O)[nH]c1=S]

a sixteenth unit is present or absent, and in the sixteenth unit:
R¹ is H, —CH₂OH, or 4-guanidinobut-1-yl; and R² is

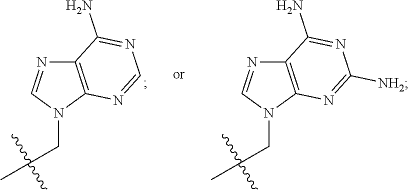 [SMILES: *Cn1cnc2c(N)ncnc21 ; *Cn1cnc2c(N)nc(N)nc21]

and
a seventeenth unit is present or absent, and in the seventeenth unit:
R¹ is H, —CH₂OH, or 4-guanidinobut-1-yl; and R² is

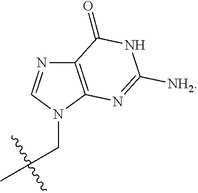 [SMILES: *Cn1cnc2c(=O)[nH]c(N)nc21]

In some embodiments, of the units with variables defined independently, counting from N-Terminus, the first unit, the second unit, the third unit, the fourth unit, the fifth unit, the sixth unit, the seventh unit, the eighth unit, the ninth unit, the tenth unit, the eleventh unit, the twelfth unit, the thirteenth unit, the fourteenth unit, the fifteenth unit, the sixteenth unit, and the seventeenth unit, independently if present, each have —CH₂OH at R¹.

In some embodiments, of the units with variables defined independently, counting from N-Terminus, the first unit, the second unit, the third unit, the fourth unit, the fifth unit, the sixth unit, the seventh unit, the eighth unit, the ninth unit, the tenth unit, the eleventh unit, the twelfth unit, the thirteenth unit, the fourteenth unit, the fifteenth unit, the sixteenth unit, and the seventeenth unit, independently if present, each have H at R¹.

In some embodiments, of the units with variables defined independently, counting from N-Terminus, the first unit, the third unit, the fifth unit, the seventh unit, the ninth unit, the eleventh unit, the thirteenth unit, the fifteenth unit, and the seventeenth unit, independently if present, each have —CH₂OH at R¹. In some embodiments, of the units with variables defined independently, counting from N-Terminus, the second unit, the fourth unit, the sixth unit, the eighth unit, the tenth unit, the twelfth unit, the fourteenth unit, and the sixteenth unit, independently if present, each have H at R¹.

In some embodiments, of the units with variables defined independently, counting from N-Terminus, the second unit, the fourth unit, the sixth unit, the eighth unit, the tenth unit, the twelfth unit, the fourteenth unit, and the sixteenth unit, independently if present, each have —CH₂OH at RU. In some embodiments, of the units with variables defined independently, counting from N-Terminus, the first unit, the third unit, the fifth unit, the seventh unit, the ninth unit, the eleventh unit, the thirteenth unit, the fifteenth unit, and the seventeenth unit, independently if present, each have H at R¹.

In some embodiments, the disclosed herein is a compound of the formula above, wherein: in the first unit: R¹ is H or —CH₂OH; in the second unit: R¹ is H or —CH₂OH; in the third unit: R¹ is H or —CH₂OH; in the fourth unit: R¹ is H or —CH₂OH; in the fifth unit: R¹ is H or —CH₂OH; in the sixth unit: R¹ is H or —CH₂OH; in the seventh unit: R¹ is H or —CH₂OH; in the eighth unit: R¹ is H or —CH₂OH; in the ninth unit: R¹ is H or —CH₂OH; in the tenth unit: R¹ is H or —CH₂OH; in the eleventh unit: R¹ is H or —CH₂OH; in the twelfth unit: R¹ is H or —CH₂OH; in the thirteenth unit: R¹ is H or —CH₂OH; in the fourteenth unit: R¹ is H or —CH₂OH; in the fifteenth unit: R¹ is H or —CH₂OH; in the sixteenth unit: R¹ is H or —CH₂OH; and in the seventeenth unit: R¹ is H or —CH₂OH.

In some embodiments, the number of units with variables defined independently is 17, wherein:
a first unit is present or absent, and in the first unit:
R¹ is H, —CH₂OH, or 4-guanidinobut-1-yl; and R² is

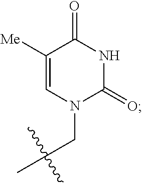 [SMILES: *Cn1cc(C)c(=O)[nH]c1=O]

a second unit is present or absent, and in the second unit:
R¹ is H, —CH₂OH, or 4-guanidinobut-1-yl; and R² is

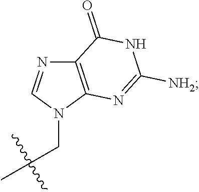 [SMILES: *Cn1cnc2c(=O)[nH]c(N)nc21]

or methyl;
a third unit is present or absent, and in the third unit:
R¹ is H, —CH₂OH, or 4-guanidinobut-1-yl; and R² is

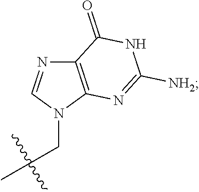 [SMILES: *Cn1cnc2c(=O)[nH]c(N)nc21]

a fourth unit is present or absent, and in the fourth unit:
R¹ is H, —CH₂OH, or 4-guanidinobut-1-yl; and R² is

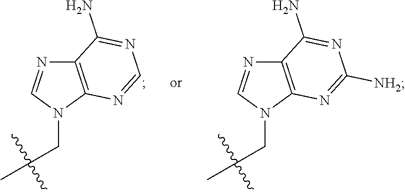 [SMILES: *Cn1cnc2c(N)ncnc21 ; *Cn1cnc2c(N)nc(N)nc21]

a fifth unit is present, and in the fifth unit:
R¹ is H, —CH₂OH, or 4-guanidinobut-1-yl; and R² is

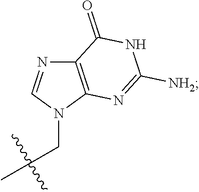 [SMILES: *Cn1cnc2c(=O)[nH]c(N)nc21]

or methyl;
a sixth unit is present, and in the sixth unit:
R¹ is H, —CH₂OH, or 4-guanidinobut-1-yl; and R² is

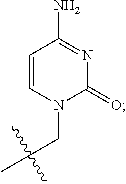 [SMILES: *Cn1ccc(N)nc1=O]

a seventh unit is present, and in the seventh unit:
R¹ is H, —CH₂OH, or 4-guanidinobut-1-yl; and R² is

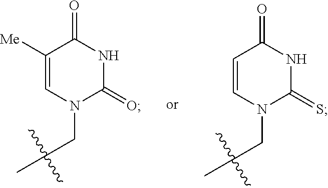 [SMILES: *Cn1cc(C)c(=O)[nH]c1=O ; "or" ; *Cn1ccc(=O)[nH]c1=S]

an eighth unit is present, and in the eighth unit:
R¹ is H, —CH₂OH, or 4-guanidinobut-1-yl; and R² is

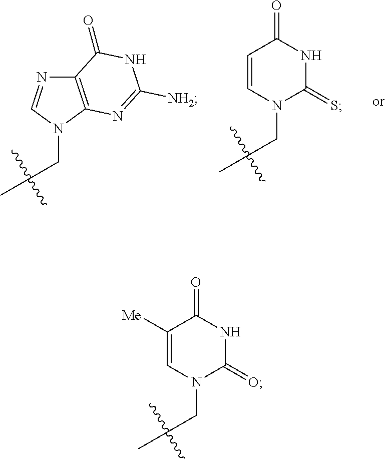 [SMILES: *Cn1cnc2c(=O)[nH]c(N)nc21 ; *Cn1ccc(=O)[nH]c1=S ; *Cn1cc(C)c(=O)[nH]c1=O]

a ninth unit is present, and in the ninth unit:
R¹ is H, —CH₂OH, or 4-guanidinobut-1-yl; and R² is

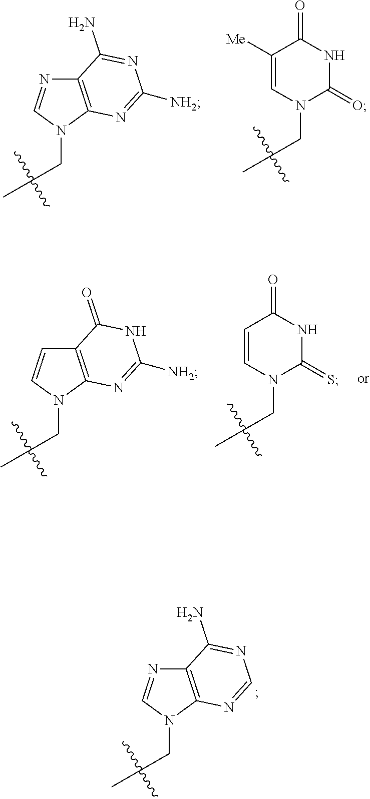 [SMILES: *Cn1cnc2c(N)nc(N)nc21 ; *Cn1cc(C)c(=O)[nH]c1=O ; *Cn1ccc2c(=O)[nH]c(N)nc21 ; *Cn1ccc(=O)[nH]c1=S ; *Cn1cnc2c(N)ncnc21]

a tenth unit is present, and in the tenth unit:
R¹ is H, —CH₂OH, or 4-guanidinobut-1-yl; and R² is

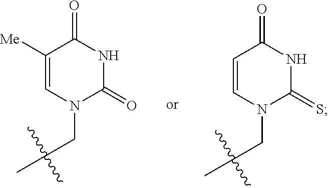 [SMILES: *Cn1cc(C)c(=O)[nH]c1=O ; "or" ; *Cn1ccc(=O)[nH]c1=S]

an eleventh unit is present, and in the eleventh unit:
R¹ is H, —CH₂OH, or 4-guanidinobut-1-yl; and R² is

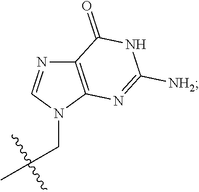 [SMILES: *Cn1cnc2c(=O)[nH]c(N)nc21]

or methyl;
a twelfth unit is present, and in the twelfth unit:
R¹ is H, —CH₂OH, or 4-guanidinobut-1-yl; and R² is

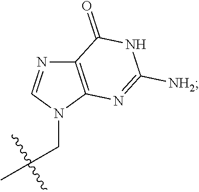 [SMILES: *Cn1cnc2c(=O)[nH]c(N)nc21]

a thirteenth unit is present, and in the thirteenth unit:
R¹ is H, —CH₂OH, or 4-guanidinobut-1-yl; and R² is

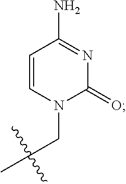 [SMILES: *Cn1ccc(N)nc1=O]

a fourteenth unit is present, and in the fourteenth unit:
R¹ is H, —CH₂OH, or 4-guanidinobut-1-yl; and R² is

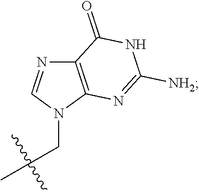 [SMILES: *Cn1cnc2c(=O)[nH]c(N)nc21]

or methyl;
a fifteenth unit is present or absent, and in the fifteenth unit:
R¹ is H, —CH₂OH, or 4-guanidinobut-1-yl; and R² is

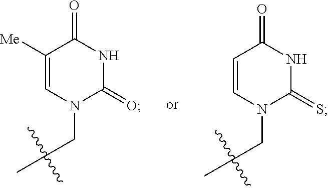 [SMILES: *Cn1cc(C)c(=O)[nH]c1=O ; "or" ; *Cn1ccc(=O)[nH]c1=S]

a sixteenth unit is present or absent, and in the sixteenth unit:
R¹ is H, —CH₂OH, or 4-guanidinobut-1-yl; and R² is

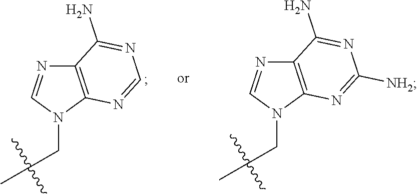 [SMILES: *Cn1cnc2c(N)ncnc21 ; *Cn1cnc2c(N)nc(N)nc21]

and
a seventeenth unit is present or absent, and in the seventeenth unit:
R¹ is H, —CH₂OH, or 4-guanidinobut-1-yl; and R² is

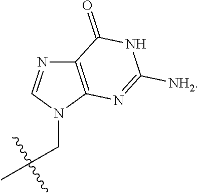 [SMILES: *Cn1cnc2c(=O)[nH]c(N)nc21]

In some embodiments, disclosed herein is a compound of the formula above, wherein: in the first unit: R¹ is H or —CH₂OH; in the second unit: R¹ is H or —CH₂OH; in the third unit: R¹ is H or —CH₂OH; in the fourth unit: R¹ is H or —CH₂OH; in the fifth unit: R¹ is H or —CH₂OH; in the sixth unit: R¹ is H or —CH₂OH; in the seventh unit: R¹ is H or —CH₂OH; in the eighth unit: R¹ is H or —CH₂OH; in the ninth unit: R¹ is H or —CH₂OH; in the tenth unit: R¹ is H or —CH₂OH; in the eleventh unit: R¹ is H or —CH₂OH; in the twelfth unit: R¹ is H or —CH₂OH; in the thirteenth unit: R¹ is H or —CH₂OH; in the fourteenth unit: R¹ is H or —CH₂OH; in the fifteenth unit: R¹ is H or —CH₂OH; in the sixteenth unit: R¹ is H or —CH₂OH; and in the seventeenth unit: R¹ is H or —CH₂OH.

In some embodiments, the number of units with variables defined independently is 27, wherein:
a first unit is present or absent, and in the first unit:
R¹ is H, —CH₂OH, or 4-guanidinobut-1-yl; and R² is

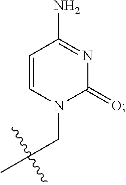 [SMILES: *Cn1ccc(N)nc1=O]

a second unit is present or absent, and in the second unit:
R¹ is H, —CH₂OH, or 4-guanidinobut-1-yl; and R² is

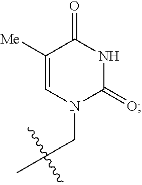 [SMILES: *Cn1cc(C)c(=O)[nH]c1=O]

a third unit is present or absent, and in the third unit:
R¹ is H, —CH₂OH, or 4-guanidinobut-1-yl; and R² is

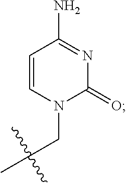 [SMILES: *Cn1ccc(N)nc1=O]

a fourth unit is present or absent, and in the fourth unit: R¹ is H, —CH₂OH, or 4-guanidinobut-1-yl; and R² is

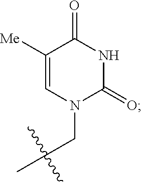 [SMILES: *Cn1cc(C)c(=O)[nH]c1=O]

a fifth unit is present or absent, and in the fifth unit:
R¹ is H, —CH₂OH, or 4-guanidinobut-1-yl; and R² is

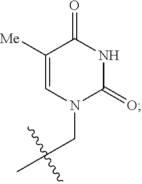 [SMILES: *Cn1cc(C)c(=O)[nH]c1=O]

a sixth unit is present or absent, and in the sixth unit:
R¹ is H, —CH₂OH, or 4-guanidinobut-1-yl; and R² is

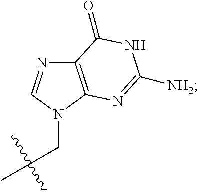 [SMILES: *Cn1cnc2c(=O)[nH]c(N)nc21]

a seventh unit is present or absent, and in the seventh unit:
R¹ is H, —CH₂OH, or 4-guanidinobut-1-yl; and R² is

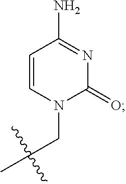 [SMILES: *Cn1ccc(N)nc1=O]

an eighth unit is present or absent, and in the eighth unit:
R¹ is H, —CH₂OH, or 4-guanidinobut-1-yl; and R² is

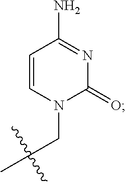 [SMILES: *Cn1ccc(N)nc1=O]

a ninth unit is present or absent, and in the ninth unit:
R¹ is H, —CH₂OH, or 4-guanidinobut-1-yl; and R² is

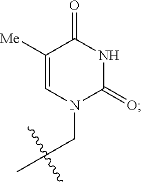 [SMILES: *Cn1cc(C)c(=O)[nH]c1=O]

a tenth unit is present or absent, and in the tenth unit:
R¹ is H, —CH₂OH, or 4-guanidinobut-1-yl; and R² is

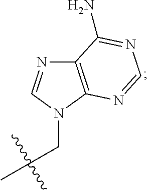 [SMILES: *Cn1cnc2c(N)ncnc21]

an eleventh unit is present or absent, and in the eleventh unit:
R¹ is H, —CH₂OH, or 4-guanidinobut-1-yl; and R² is

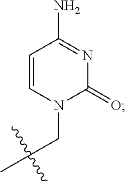 [SMILES: *Cn1ccc(N)nc1=O]

a twelfth unit is present, and in the twelfth unit:
R¹ is H, —CH₂OH, or 4-guanidinobut-1-yl; and R² is

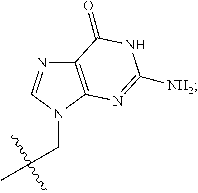 [SMILES: *Cn1cnc2c(=O)[nH]c(N)nc21]

a thirteenth unit is present, and in the thirteenth unit:
R¹ is H, —CH₂OH, or 4-guanidinobut-1-yl; and R² is

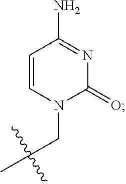 [SMILES: *Cn1ccc(N)nc1=O]

a fourteenth unit is present, and in the fourteenth unit:
R¹ is H, —CH₂OH, or 4-guanidinobut-1-yl; and R² is

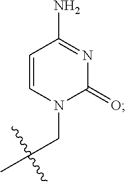 [SMILES: *Cn1ccc(N)nc1=O]

a fifteenth unit is present, and in the fifteenth unit:
R¹ is H, —CH₂OH, or 4-guanidinobut-1-yl; and R² is

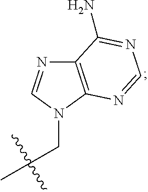 [SMILES: *Cn1cnc2c(N)ncnc21]

a sixteenth unit is present, and in the sixteenth unit:
R¹ is H, —CH₂OH, or 4-guanidinobut-1-yl; and R² is

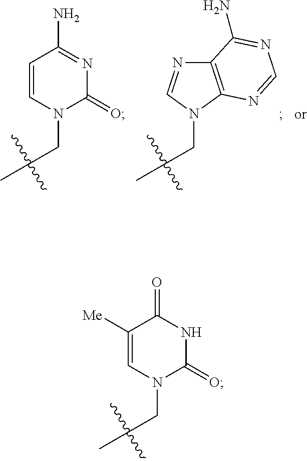 [SMILES: *Cn1ccc(N)nc1=O ; *Cn1cnc2c(N)ncnc21 ; *Cn1cc(C)c(=O)[nH]c1=O]

a seventeenth unit is present, and in the seventeenth unit:
R¹ is H, —CH₂OH, or 4-guanidinobut-1-yl; and R² is

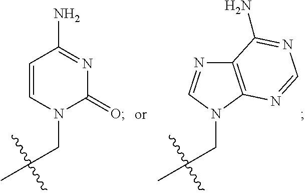 [SMILES: *Cn1ccc(N)nc1=O ; *Cn1cnc2c(N)ncnc21]

an eighteenth unit is present, and in the eighteenth unit:
R¹ is H, —CH₂OH, or 4-guanidinobut-1-yl; and R² is

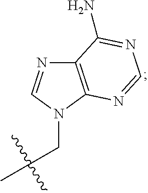 [SMILES: *Cn1cnc2c(N)ncnc21]

a nineteenth unit is present, and in the nineteenth unit:
R¹ is H, —CH₂OH, or 4-guanidinobut-1-yl; and R² is

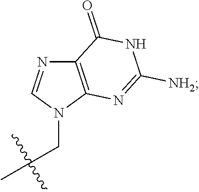 [SMILES: *Cn1cnc2c(=O)[nH]c(N)nc21]

a twentieth unit is present, and in the twentieth unit:
R¹ is H, —CH₂OH, or 4-guanidinobut-1-yl; and R² is

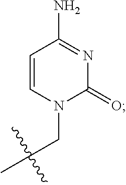 [SMILES: *Cn1ccc(N)nc1=O]

a twenty-first unit is present, and in the twenty-first unit:
R¹ is H, —CH₂OH, or 4-guanidinobut-1-yl; and R² is

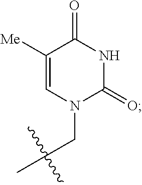 [SMILES: *Cn1cc(C)c(=O)[nH]c1=O]

a twenty-second unit is present or absent, and in the twenty-second unit:
R¹ is H, —CH₂OH, or 4-guanidinobut-1-yl; and R² is

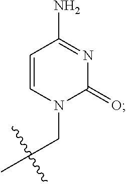 [SMILES: *Cn1ccc(N)nc1=O]

a twenty-third unit is present or absent, and in the twenty-third unit:
R¹ is H, —CH₂OH, or 4-guanidinobut-1-yl; and R² is

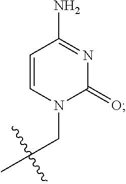 [SMILES: *Cn1ccc(N)nc1=O]

a twenty-fourth unit is present or absent, and in the twenty-fourth unit:
R¹ is H, —CH₂OH, or 4-guanidinobut-1-yl; and R² is

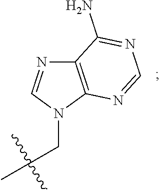 [SMILES: *Cn1cnc2c(N)ncnc21]

a twenty-fifth unit is present or absent, and in the twenty-fifth unit:
R¹ is H, —CH₂OH, or 4-guanidinobut-1-yl; and R² is

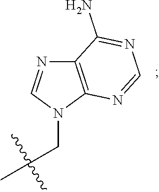 [SMILES: *Cn1cnc2c(N)ncnc21]

a twenty-sixth unit is present or absent, and in the twenty-sixth unit:
R¹ is H, —CH₂OH, or 4-guanidinobut-1-yl; and R² is

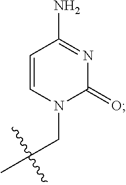 [SMILES: *Cn1ccc(N)nc1=O]

and
a twenty-seventh unit is present or absent, and in the twenty-seventh unit:
R¹ is H, —CH₂OH, or 4-guanidinobut-1-yl; and R² is

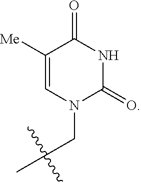 [SMILES: *Cn1cc(C)c(=O)[nH]c1=O]

In some embodiments, the number of units with variables defined independently is 11, 12, 13, 14, 15, 16, 17, 18, 19, 20, 21, 22, 23, 24, 25, 26, or 27, wherein:
a first unit is present or absent, and in the first unit:
R¹ is H, —CH₂OH, or 4-guanidinobut-1-yl; and R² is

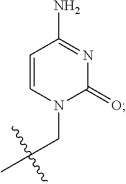 [SMILES: *Cn1ccc(N)nc1=O]

a second unit is present or absent, and in the second unit:
R¹ is H, —CH₂OH, or 4-guanidinobut-1-yl; and R² is

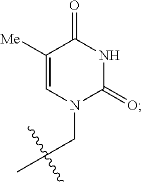 [SMILES: *Cn1cc(C)c(=O)[nH]c1=O]

a third unit is present or absent, and in the third unit:
R¹ is H, —CH₂OH, or 4-guanidinobut-1-yl; and R² is

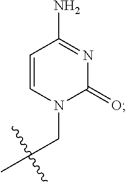 [SMILES: *Cn1ccc(N)nc1=O]

a fourth unit is present or absent, and in the fourth unit:
R¹ is H, —CH₂OH, or 4-guanidinobut-1-yl; and R² is

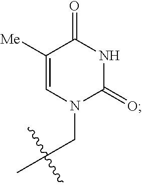 [SMILES: *Cn1cc(C)c(=O)[nH]c1=O]

a fifth unit is present or absent, and in the fifth unit:
R¹ is H, —CH₂OH, or 4-guanidinobut-1-yl; and R² is

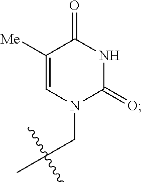 [SMILES: *Cn1cc(C)c(=O)[nH]c1=O]

a sixth unit is present or absent, and in the sixth unit:
R¹ is H, —CH₂OH, or 4-guanidinobut-1-yl; and R² is

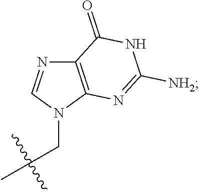 [SMILES: *Cn1cnc2c(=O)[nH]c(N)nc21]

a seventh unit is present or absent, and in the seventh unit:
R¹ is H, —CH₂OH, or 4-guanidinobut-1-yl; and R² is

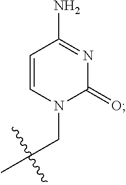 [SMILES: *Cn1ccc(N)nc1=O]

an eighth unit is present or absent, and in the eighth unit:
R¹ is H, —CH₂OH, or 4-guanidinobut-1-yl; and R² is

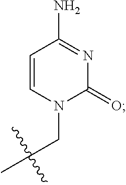 [SMILES: *Cn1ccc(N)nc1=O]

a ninth unit is present or absent, and in the ninth unit:
R¹ is H, —CH₂OH, or 4-guanidinobut-1-yl; and R² is

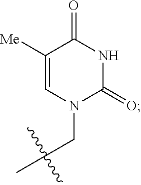 [SMILES: *Cn1cc(C)c(=O)[nH]c1=O]

a tenth unit is present or absent, and in the tenth unit:
R¹ is H, —CH₂OH, or 4-guanidinobut-1-yl; and R² is

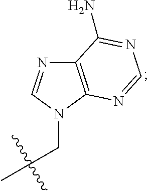 [SMILES: *Cn1cnc2c(N)ncnc21]

an eleventh unit is present or absent, and in the eleventh unit:
R¹ is H, —CH₂OH, or 4-guanidinobut-1-yl; and R² is

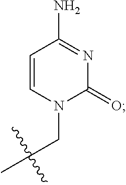 [SMILES: *Cn1ccc(N)nc1=O]

a twelfth unit is present, and in the twelfth unit:
R¹ is H, —CH₂OH, or 4-guanidinobut-1-yl; and R² is

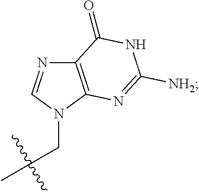 [SMILES: *Cn1cnc2c(=O)[nH]c(N)nc21]

a thirteenth unit is present, and in the thirteenth unit:
R¹ is H, —CH₂OH, or 4-guanidinobut-1-yl; and R² is

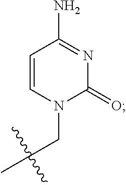 [SMILES: *Cn1ccc(N)nc1=O]

a fourteenth unit is present, and in the fourteenth unit:
R¹ is H, —CH₂OH, or 4-guanidinobut-1-yl; and R² is

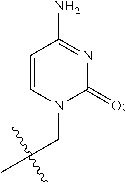 [SMILES: *Cn1ccc(N)nc1=O]

a fifteenth unit is present, and in the fifteenth unit:
R¹ is H, —CH₂OH, or 4-guanidinobut-1-yl; and R² is

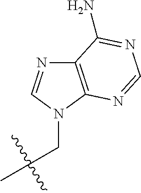 [SMILES: *Cn1cnc2c(N)ncnc21]

or methyl;
a sixteenth unit is present, and in the sixteenth unit:
R¹ is H, —CH₂OH, or 4-guanidinobut-1-yl; and R² is

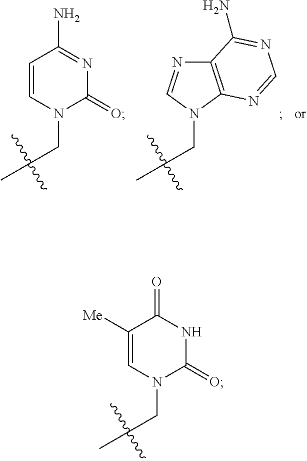 [SMILES: *Cn1ccc(N)nc1=O ; *Cn1cnc2c(N)ncnc21 ; *Cn1cc(C)c(=O)[nH]c1=O]

a seventeenth unit is present, and in the seventeenth unit:
R¹ is H, —CH₂OH, or 4-guanidinobut-1-yl; and R² is

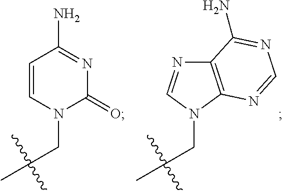 [SMILES: *Cn1ccc(N)nc1=O ; *Cn1cnc2c(N)ncnc21]

or methyl;
an eighteenth unit is present, and in the eighteenth unit:
R¹ is H, —CH₂OH, or 4-guanidinobut-1-yl; and R² is

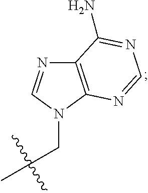 [SMILES: *Cn1cnc2c(N)ncnc21]

a nineteenth unit is present, and in the nineteenth unit:
R¹ is H, —CH₂OH, or 4-guanidinobut-1-yl; and R² is

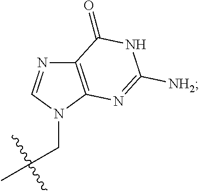 [SMILES: *Cn1cnc2c(=O)[nH]c(N)nc21]

a twentieth unit is present, and in the twentieth unit:
R¹ is H, —CH₂OH, or 4-guanidinobut-1-yl; and R² is

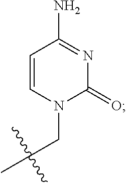 [SMILES: *Cn1ccc(N)nc1=O]

a twenty-first unit is present, and in the twenty-first unit:
R¹ is H, —CH₂OH, or 4-guanidinobut-1-yl; and R² is

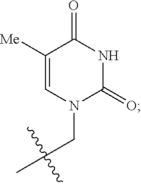 [SMILES: *Cn1cc(C)c(=O)[nH]c1=O]

a twenty-second unit is present or absent, and in the twenty-second unit:
R¹ is H, —CH₂OH, or 4-guanidinobut-1-yl; and R² is

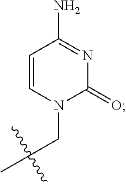 [SMILES: *Cn1ccc(N)nc1=O]

a twenty-third unit is present or absent, and in the twenty-third unit:
R¹ is H, —CH₂OH, or 4-guanidinobut-1-yl; and R² is

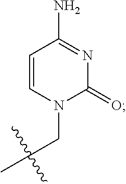 [SMILES: *Cn1ccc(N)nc1=O]

a twenty-fourth unit is present or absent, and in the twenty-fourth unit:
R¹ is H, —CH₂OH, or 4-guanidinobut-1-yl; and R² is

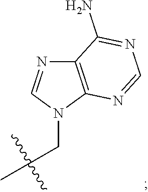 [SMILES: *Cn1cnc2c(N)ncnc21]

a twenty-fifth unit is present or absent, and in the twenty-fifth unit:
R¹ is H, —CH₂OH, or 4-guanidinobut-1-yl; and R² is

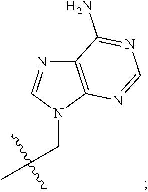 [SMILES: *Cn1cnc2c(N)ncnc21]

a twenty-sixth unit is present or absent, and in the twenty-sixth unit:
R¹ is H, —CH₂OH, or 4-guanidinobut-1-yl; and R² is; and

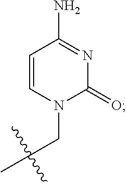 [SMILES: *Cn1ccc(N)nc1=O]

and a twenty-seventh unit is present or absent, and in the twenty-seventh unit:
R¹ is H, —CH₂OH, or 4-guanidinobut-1-yl; and R² is

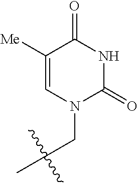 [SMILES: *Cn1cc(C)c(=O)[nH]c1=O]

(SEQ ID NO: 328).

In some embodiments, of the units with variables defined independently, counting from N-Terminus, the first unit, the second unit, the third unit, the fourth unit, the fifth unit, the sixth unit, the seventh unit, the eighth unit, the ninth unit, the tenth unit, the eleventh unit, the twelfth unit, the thirteenth unit, the fourteenth unit, the fifteenth unit, the sixteenth unit, the seventeenth unit, the eighteenth unit, the nineteenth unit, the twentieth unit, the twenty-first unit, the twenty-second unit, the twenty-third unit, the twenty-fourth unit, the twenty-fifth unit, the twenty-sixth unit, and the twenty-seventh unit, independently if present, each have —CH₂OH at R¹.

In some embodiments, of the units with variables defined independently, counting from N-Terminus, the first unit, the third unit, the fifth unit, the seventh unit, the ninth unit, the eleventh unit, the thirteenth unit, the fifteenth unit, the seventeenth unit, the nineteenth unit, the twentieth unit, the twenty-first unit, the twenty-third unit, the twenty-fifth unit, and the twenty-seventh unit, independently if present, each have —CH₂OH at R¹. In some embodiments, of the units with variables defined independently, counting from N-Terminus, the second unit, the fourth unit, the sixth unit, the eighth unit, the tenth unit, the twelfth unit, the fourteenth unit, the sixteenth unit, the eighteenth unit, the twentieth unit, the twenty-second unit, the twenty-fourth unit, and the twenty-sixth unit, independently if present, each have H at R¹.

In some embodiments, of the units with variables defined independently, counting from N-Terminus, the second unit, the fourth unit, the sixth unit, the eighth unit, the tenth unit, the twelfth unit, the fourteenth unit, the sixteenth unit, the eighteenth unit, the twentieth unit, the twenty-second unit, the twenty-fourth unit, and the twenty-sixth unit, independently if present, each have —CH₂OH at RU. In some embodiments, of the units with variables defined independently, counting from N-Terminus, the first unit, the third unit, the fifth unit, the seventh unit, the ninth unit, the eleventh unit, the thirteenth unit, the fifteenth unit, the seventeenth unit, the nineteenth unit, the twentieth unit, the twenty-first unit, the twenty-third unit, the twenty-fifth unit, and the twenty-seventh unit, independently if present, each have H at RU.

In some embodiments, of the units with variables defined independently, counting from N-Terminus, the fourth unit, the eighth unit, the twelfth unit, the sixteenth unit, the twentieth unit, and the twenty-fourth unit, independently if present, each have —CH₂OH at R¹. In some embodiments, of the units with variables defined independently, counting from N-Terminus, the first unit, the second unit, the third unit, the fifth unit, the sixth unit, the seventh unit, the ninth unit, the tenth unit, the eleventh unit, the thirteenth unit, the fourteenth unit, the fifteenth unit, the seventeenth unit, the eighteenth unit, the nineteenth unit, the twenty-first unit, the twenty-second unit, the twenty-third unit, the twenty-fifth unit, the twenty-sixth unit, and the twenty-seventh unit, independently if present, each have H at R¹.

In some embodiments, of the units with variables defined independently, counting from N-Terminus, the thirteenth unit, the fourteenth unit, the fifteenth unit, the sixteenth unit, the seventeenth unit, the eighteenth unit, and the nineteenth unit, independently if present, each have —CH₂OH at R¹. In some embodiments, of the units with variables defined independently, counting from N-Terminus, the first unit, the second unit, the third unit, the fourth unit, the fifth unit, the sixth unit, the seventh unit, the eighth unit, the ninth unit, the tenth unit, the eleventh unit, the twelfth unit, the twentieth unit, the twenty-first unit, the twenty-second unit, the twenty-third unit, the twenty-fourth unit, the twenty-fifth unit, the twenty-sixth unit, and the twenty-seventh unit, independently if present, each have H at R¹.

In some embodiments, of the units with variables defined independently, counting from N-Terminus, the first unit, the third unit, the fourth unit, the fifth unit, the seventh unit, the eighth unit, the ninth unit, the eleventh unit, the twelfth unit, the thirteenth unit, the fifteenth unit, the sixteenth unit, the seventeenth unit, the nineteenth unit, the twentieth unit, the twenty-first unit, the twenty-third unit, the twenty-fourth unit, the twenty-fifth unit, and the twenty-seventh unit, independently if present, each have —CH₂OH at RU. In some embodiments, of the units with variables defined independently, counting from N-Terminus, the second unit, the sixth unit, the tenth unit, the fourteenth unit, the eighteenth unit, the twenty-second unit, and the twenty-sixth unit, independently if present, each have H at R¹.

In some embodiments, of the units with variables defined independently, counting from N-Terminus, the fourth unit, the eighth unit, the twelfth unit, the sixteenth unit, the seventeenth unit, the twentieth unit, and the twenty-fourth unit, independently if present, each have —CH₂OH at RU. In some embodiments, of the units with variables defined independently, counting from N-Terminus, the first unit, the second unit, the third unit, the fifth unit, the sixth unit, the seventh unit, the ninth unit, the tenth unit, the eleventh unit, the thirteenth unit, the fourteenth unit, the fifteenth unit, the eighteenth unit, the nineteenth unit, the twenty-first unit, the twenty-second unit, the twenty-third unit, the twenty-fifth unit, the twenty-sixth unit, and the twenty-seventh unit, independently if present, each have H at R¹.

In some embodiments, of the units with variables defined independently, counting from N-Terminus, the thirteenth unit, the fourteenth unit, the fifteenth unit, the sixteenth unit, the seventeenth unit, the eighteenth unit, and the nineteenth unit, independently if present, each have —CH₂OH at R¹. In some embodiments, of the units with variables defined independently, counting from N-Terminus, the first unit, the second unit, the third unit, the fourth unit, the fifth unit, the sixth unit, the seventh unit, the eighth unit, the ninth unit, the tenth unit, the eleventh unit, the twelfth unit, the twentieth unit, the twenty-first unit, the twenty-second unit, the twenty-third unit, the twenty-fourth unit, the twenty-fifth unit, the twenty-sixth unit, and the twenty-seventh unit, independently if present, each have H at R¹.

In some embodiments, disclosed herein is a compound of the formula above, wherein: in the first unit: R¹ is H or —CH₂OH; in the second unit: R¹ is H or —CH₂OH; in the third unit: R¹ is H or —CH₂OH; in the fourth unit: R¹ is H or —CH₂OH; in the fifth unit: R¹ is H or —CH₂OH; in the sixth unit: R¹ is H or —CH₂OH; in the seventh unit: R¹ is H or —CH₂OH; in the eighth unit: R¹ is H or —CH₂OH; in the ninth unit: R¹ is H or —CH₂OH; in the tenth unit: R¹ is H or —CH₂OH; in the eleventh unit: R¹ is H or —CH₂OH; in the twelfth unit: R¹ is H or —CH₂OH; in the thirteenth unit: R¹ is H or —CH₂OH; in the fourteenth unit: R¹ is H or —CH₂OH; in the fifteenth unit: R¹ is H or —CH₂OH; in the sixteenth unit: R¹ is H or —CH₂OH; in the seventeenth unit: R¹ is H or —CH₂OH; in the eighteenth unit: R¹ is H or —CH₂OH; in the nineteenth unit: R¹ is H or —CH₂OH; in the twentieth unit: R¹ is H or —CH₂OH; in the twenty-first unit: R¹ is H or —CH₂OH; in the twenty-second unit: R¹ is H or —CH₂OH; in the twenty-third unit: R¹ is H or —CH₂OH; in the twenty-fourth unit: R¹ is H or —CH₂OH; in the twenty-fifth unit: R¹ is H or —CH₂OH; in the twenty-sixth unit: R¹ is H or —CH₂OH; and in the twenty-seventh unit: R¹ is H or —CH₂OH.

In some embodiments, each R¹ is independently alkyl that is unsubstituted. In some embodiments, each alkyl that is unsubstituted is independently methyl, ethyl, prop-1-yl, prop-2-yl, 2-methylprop-1-yl, but-1yl, but-2-yl, or pent-1-yl. In some embodiments, each alkyl that is unsubstituted is independently methyl, prop-2-yl, 2-methylprop-1-yl, or but-2-yl.

In some embodiments, each R¹ is independently alkyl that is substituted. In some embodiments, each alkyl that is substituted is independently substituted with —OH, —SH, —SMe, —NH₂, a heterocycle, an aryl group, a carboxylic acid, a guanidino group, a N-methylguanidino group, or an amido group. In some embodiments, each alkyl that is substituted is independently hydroxymethyl, 1-hydroxyeth-1-yl, sulfhydrylmethyl, 2-thiomethyleth-1-yl, 4-aminobut-1-yl, 3-aminoprop-1-yl, 1-H-imidazol-4-ylmethyl, 1-H-indol-3-ylmethyl, benzyl, 4-hydroxyphen-1-ylmethyl, 2-carboxylatoeth-1-yl, 3-carboxylatoprop-1-yl, 3-guanidinoprop-1-yl, 4-guanidinobut-1-yl, 2-carbamoyleth-1-yl, or 3-carbamoylprop-1-yl.

In some embodiments, each R¹ is independently H, hydroxylmethyl, or 4-guanidinobut-1-yl. In some embodiments, at least one iteration of R¹ is a hydroxyalkyl group. In some embodiments, at least one iteration of R¹ is hydroxylmethyl. In some embodiments, at least a third of the iterations of R¹ are hydroxylmethyl. In some embodiments, at least half the iterations of R¹ are hydroxylmethyl.

In some embodiments, PEP1 is absent. In some embodiments, PEP1 is the peptide sequence. In some embodiments, the peptide sequence of PEP1 is a nuclear localization sequence. In some embodiments, at least one of PEP1 and PEP2 is a peptide sequence of at least three amino acid residues.

In some embodiments, PEP1 is -Lys-. In some embodiments, PEP1 is -(D-Lys)-. In some embodiments, PEP1 is a sequence that is -(D-Arg)-(D-Arg)-(D-Arg)-(D-Arg)- (SEQ ID NO: 317). In some embodiments, PEP1 is a sequence that is -Tyr-Gly-Arg-Lys-Lys-Arg-Arg-Gln-Arg-Arg-Arg- (SEQ ID NO: 313). In some embodiments, PEP1 is a sequence that is -Arg-Arg-Arg-Arg-Arg-Arg-Arg-Arg- (SEQ ID NO: 314). In some embodiments, PEP1 is a sequence that is -Leu-Cys-Leu-Arg-Pro-Val-Gly- (SEQ ID NO: 315). In some embodiments, PEP1 is -Cys¹-Leu-Ser-Ser-Arg-Leu-Asp-Ala-Cys²-, wherein Cys¹ and Cys² are bound to one another via an intrachain disulfide bond (CEQ ID NO: 316). In some embodiments, PEP1 is a sequence that is -Cys¹-Ala-Gly-Ala-Leu-Cys²-Tyr-, wherein Cys¹ and Cys² are bound to one another via an intrachain disulfide bond (SEQ ID NO: 318). In some embodiments, PEP1 is a sequence that is -Cys¹-Leu-Glu-Val-Ser-Arg-Lys-Asn-Cys²-, wherein Cys¹ and Cys² are bound to one another via an intrachain disulfide bond (SEQ ID NO: 319). In some embodiments, PEP1 is a sequence that is -Cys¹-Arg-Thr-Ile-Gly-Pro-Ser-Val-Cys²-, wherein Cys¹ and Cys² are bound to one another via an intrachain disulfide bond SEQ ID NO: 301). In some embodiments, PEP1 is a sequence that is -Cys¹-Thr-Ser-Thr-Ser-Ala-Pro-Tyr-Cys²-, wherein Cys¹ and Cys² are bound to one another via an intrachain disulfide bond (SEQ ID NO: 320). In some embodiments, PEP1 is a sequence that is -Cys¹-Met-Pro-Arg-Leu-Arg-Gly-Cys²-, wherein Cys¹ and Cys² are bound to one another via an intrachain disulfide bond (SEQ ID NO: 321). In some embodiments, PEP1 is a sequence that is -Thr-Gly-Asn-Tyr-Lys-Ala-Leu-His-Pro-His-Asn-Gly- (SEQ ID NO: 322).

In some embodiments, PEP1 is -Arg-Arg-. In some embodiments, PEP1 is a sequence that is -Pro-Lys-Lys-Lys-Arg-Lys-Val- (SEQ ID NO: 300). In some embodiments, PEP1 is a sequence that is -Ala-Lys-Ala-Ser-Ser-Leu-Asn-Ile-Ala- (SEQ ID NO: 329). In some embodiments, PEP1 is a sequence that is -Ala-Ser-Ser-Leu-Asn-Ile-Ala-. In some embodiments, PEP1 is a sequence that is -Arg-Phe-Gln-Ile-Leu-Tyr-Arg- (SEQ ID NO: 330). In some embodiments, PEP1 is -(D-Arg)-(D-Arg)-(D-Arg)-. In some embodiments, PEP1 is a sequence that is -Arg-Gly-Arg-Gly-Arg-Gly-Arg-Gly-Arg-Gly-Arg-Gly-Arg-Gly- (SEQ ID NO: 305). In some embodiments, PEP1 is a sequence that is -Arg-Tyr-Gln-Phe-Leu-Ile-Arg- (SEQ ID NO: 331). In some embodiments, PEP1 is a sequence that is -Arg-Ile-Gln-Phe-Leu-Ile-Arg- (SEQ ID NO: 332). In some embodiments, PEP1 is a sequence that is -Arg-Arg-Trp-Trp-Arg-Arg-Trp-Arg-Arg- (SEQ ID NO: 309). In some embodiments, PEP1 is a sequence that is -Arg-Arg-Trp-Gln-Trp- (SEQ ID NO: 310). In some embodiments, PEP1 is a sequence that is (D-Thr)-(D-His)-(D-Arg)-(D-Pro)-(D-Pro)-(D-Met)-(D-Trp)-(D-Ser)-(D-Pro)-(D-Val)-(D-Trp)-(D-Pro-)- (SEQ ID NO: 302). In some embodiments, PEP1 is a sequence that is -(D-Pro)-(D-Trp)-(D-Val)-(D-Pro)-(D-Ser)-(D-Trp)-(D-Met)-(D-Pro)-(D-Pro)-(D-Arg)-(D-His)-(D-Thr)-(SEQ ID NO: 303). In some embodiments, PEP1 is a sequence that is -(D-His)-(D-Arg)-(D-Pro)-(D-Tyr)-(D-Ile)-(D-Ala)-(D-His)- (SEQ ID NO: 304).

In some embodiments, PEP2 is absent. In some embodiments, PEP2 is the peptide sequence. In some embodiments, the peptide sequence of PEP2 is a nuclear localization sequence.

In some embodiments, PEP2 is -Lys-. In some embodiments, PEP2 is -(D-Lys)-. In some embodiments, PEP2 is a sequence that is -(D-Arg)-(D-Arg)-(D-Arg)-(D-Arg)- (SEQ ID NO: 317). In some embodiments, PEP2 is a sequence that is -Tyr-Gly-Arg-Lys-Lys-Arg-Arg-Gln-Arg-Arg-Arg- (SEQ ID NO: 313). In some embodiments, PEP2 is a sequence that is -Arg-Arg-Arg-Arg-Arg-Arg-Arg-Arg- (SEQ ID NO: 314). In some embodiments, PEP2 is a sequence that is -Leu-Cys-Leu-Arg-Pro-Val-Gly- (SEQ ID NO: 315). In some embodiments, PEP2 is -Cys¹-Leu-Ser-Ser-Arg-Leu-Asp-Ala-Cys²-, wherein Cys¹ and Cys² are bound to one another via an intrachain disulfide bond (CEQ ID NO: 316). In some embodiments, PEP2 is a sequence that is -Cys¹-Ala-Gly-Ala-Leu-Cys²-Tyr-, wherein Cys¹ and Cys² are bound to one another via an intrachain disulfide bond (SEQ ID NO: 318). In some embodiments, PEP2 is a sequence that is -Cys¹-Leu-Glu-Val-Ser-Arg-Lys-Asn-Cys²-, wherein Cys¹ and Cys² are bound to one another via an intrachain disulfide bond (SEQ ID NO: 319). In some embodiments, PEP2 is a sequence that is -Cys¹-Arg-Thr-Ile-Gly-Pro-Ser-Val-Cys²-, wherein Cys¹ and Cys² are bound to one another via an intrachain disulfide bond SEQ ID NO: 301). In some embodiments, PEP2 is a sequence that is -Cys¹-Thr-Ser-Thr-Ser-Ala-Pro-Tyr-Cys²-, wherein Cys¹ and Cys² are bound to one another via an intrachain disulfide bond (SEQ ID NO: 320). In some embodiments, PEP2 is a sequence that is -Cys¹-Met-Pro-Arg-Leu-Arg-Gly-Cys²-, wherein Cys¹ and Cys² are bound to one another via an intrachain disulfide bond (SEQ ID NO: 321). In some embodiments, PEP2 is a sequence that is -Thr-Gly-Asn-Tyr-Lys-Ala-Leu-His-Pro-His-Asn-Gly- (SEQ ID NO: 322).

In some embodiments, PEP2 is -Arg-Arg-. In some embodiments, PEP2 is a sequence that is -Pro-Lys-Lys-Lys-Arg-Lys-Val- (SEQ ID NO: 300). In some embodiments, PEP2 is a sequence that is -Ala-Lys-Ala-Ser-Ser-Leu-Asn-Ile-Ala- (SEQ ID NO: 329). In some embodiments, PEP2 is a sequence that is -Ala-Ser-Ser-Leu-Asn-Ile-Ala-. In some embodiments, PEP2 is a sequence that is -Arg-Phe-Gln-Ile-Leu-Tyr-Arg- (SEQ ID NO: 330). In some embodiments, PEP2 is -(D-Arg)-(D-Arg)-(D-Arg)-. In some embodiments, PEP2 is a sequence that is -Arg-Gly-Arg-Gly-Arg-Gly-Arg-Gly-Arg-Gly-Arg-Gly-Arg-Gly- (SEQ ID NO: 305). In some embodiments, PEP2 is a sequence that is -Arg-Tyr-Gln-Phe-Leu-Ile-Arg- (SEQ ID NO: 331). In some embodiments, PEP2 is a sequence that is -Arg-Ile-Gln-Phe-Leu-Ile-Arg- (SEQ ID NO: 332). In some embodiments, PEP2 is a sequence that is -Arg-Arg-Trp-Trp-Arg-Arg-Trp-Arg-Arg- (SEQ ID NO: 309). In some embodiments, PEP2 is a sequence that is -Arg-Arg-Trp-Gln-Trp- (SEQ ID NO: 310). In some embodiments, PEP2 is a sequence that is (D-Thr)-(D-His)-(D-Arg)-(D-Pro)-(D-Pro)-(D-Met)-(D-Trp)-(D-Ser)-(D-Pro)-(D-Val)-(D-Trp)-(D-Pro-)- (SEQ ID NO: 302). In some embodiments, PEP2 is a sequence that is -(D-Pro)-(D-Trp)-(D-Val)-(D-Pro)-(D-Ser)-(D-Trp)-(D-Met)-(D-Pro)-(D-Pro)-(D-Arg)-(D-His)-(D-Thr)- (SEQ ID NO: 303). In some embodiments, PEP2 is a sequence that is -(D-His)-(D-Arg)-(D-Pro)-(D-Tyr)-(D-Ile)-(D-Ala)-(D-His)- (SEQ ID NO: 304).

In some embodiments, SOL1 is absent. In some embodiments, SOL1 is the water-solubilizing group. In some embodiments, the water-solubilizing group of SOL1 is a peptide sequence. In some embodiments, the water-solubilizing group of SOL1 is a group that contains multiple electrical charges at physiological pH. In some embodiments, the water-solubilizing group of SOL1 is a group that contains multiple positive charges at physiological pH. In some embodiments, the water-solubilizing group of SOL1 is a polyethyleneglycol group. In some embodiments, the water-solubilizing group of SOL1 is -Arg-Arg-NH(CH₂)₂C(O)-Arg-Arg-I SEQ ID NO: 333).

In some embodiments, the water-solubilizing group of SOL1 is a group of formula:

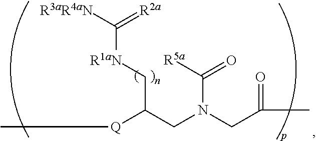

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

In some embodiments, the water-solubilizing group of SOL1 is a group of formula:

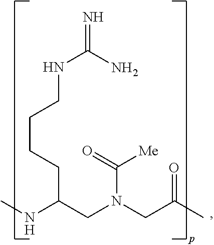

wherein p is an integer that is 1-1,000.

In some embodiments, the water-solubilizing group of SOL1 is a group of formula:

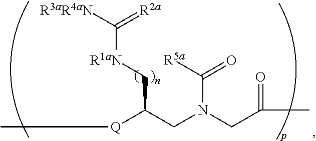

R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

In some embodiments, the water-solubilizing group of SOL1 is a group of formula:

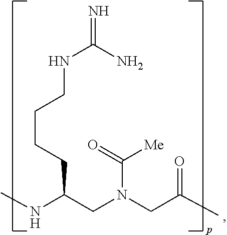

wherein p is an integer that is 1-1,000.

In some embodiments, p is an integer that is 1-100. In some embodiments, p is an integer that is 1-50.

In some embodiments, p is an integer that is 1, 2, 3, 4, 5, 6, 7, 8, 9, 10, 11, 12, 13, 14, 15, 16, 17, 18, 19, or 20. In some embodiments, p is an integer that is 1, 2, 3, 4, 5, 6, 7, 8, 9, 10, 11, or 12. In some embodiments, p is an integer that is 3, 4, 5, 6, 7, 8, 9, or 10. In some embodiments, p is an integer that is 5, 6, 7, 8, or 9. In some embodiments, p is an integer that is 6, 7, or 8. In some embodiments, p is an integer that is 7.

In some embodiments, SOL2 is absent. In some embodiments, SOL2 is the water-solubilizing group. In some embodiments, the water-solubilizing group of SOL2 is a peptide sequence. In some embodiments, the water-solubilizing group of SOL2 is a group that contains multiple electrical charges at physiological pH. In some embodiments, the water-solubilizing group of SOL2 is a group that contains multiple positive charges at physiological pH. In some embodiments, the water-solubilizing group of SOL2 is a polyethyleneglycol group. In some embodiments, the water-solubilizing group of SOL2 is -Arg-Arg-NH(CH₂)₂C(O)-Arg-Arg- (SEQ ID NO: 333).

In some embodiments, the water-solubilizing group of SOL2 is a group of formula:

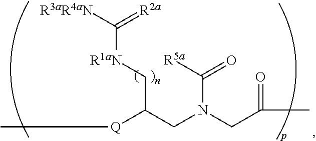

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

In some embodiments, the water-solubilizing group of SOL2 is a group of formula:

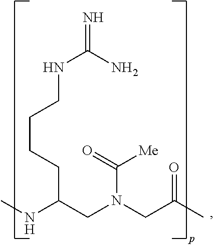

wherein p is an integer that is 1-1,000.

In some embodiments, the water-solubilizing group of SOL2 is a group of formula:

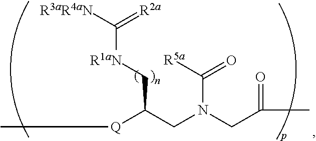

R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted;
Q is O, NH, N(alkyl), or N(Pg^{N});
n is 1, 2, 3, 4, 5, 6, 7, 8, 9, or 10; and
p is an integer that is 1-1,000.

In some embodiments, the water-solubilizing group of SOL2 is a group of formula:

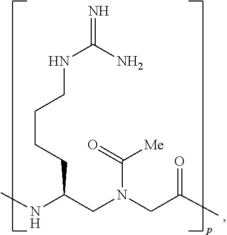

wherein p is an integer that is 1-1,000.

In some embodiments, p is an integer that is 1-100. In some embodiments, p is an integer that is 1-50.

In some embodiments, p is an integer that is 1, 2, 3, 4, 5, 6, 7, 8, 9, 10, 11, 12, 13, 14, 15, 16, 17, 18, 19, or 20. In some embodiments, p is an integer that is 1, 2, 3, 4, 5, 6, 7, 8, 9, 10, 11, or 12. In some embodiments, p is an integer that is 3, 4, 5, 6, 7, 8, 9, or 10. In some embodiments, p is an integer that is 5, 6, 7, 8, or 9. In some embodiments, p is an integer that is 6, 7, or 8. In some embodiments, p is an integer that is 7.

In some embodiments, PNA1 is the peptide nucleic acid sequence. In some embodiments, PNA2 is the peptide nucleic acid sequence.

In some embodiments, L1 is the linker group. In some embodiments, the linker group of L1 is cleavable. In some embodiments, the linker group of L1 is non-cleavable. In some embodiments, the linker group of L1 is a peptide sequence. In some embodiments, the linker group of L1 is a polyamine sequence. In some embodiments, the linker group of L1 is a polyamide sequence.

In some embodiments, the linker group of L1 is a residue of an omega-amino fatty acid. In some embodiments, the linker group of L1 is a residue of an omega-amino caproic acid. In some embodiments, the linker group of L1 is a residue of a dicarboxylic acid. In some embodiments, the linker group of L1 is a residue of oxalic acid. In some embodiments, the linker group of L1 is a residue of succinic acid.

In some embodiments, the linker group of L1 is a peptide sequence that is -Glu-Val-Citrulline-. In some embodiments, the linker group of L1 is —NHCH(COOH)C(CH₃)₂S—SC(CH₃)₂CH(NH₂)C(O)—. In some embodiments, the linker group of L1 is —NHCH(COOH)C(CH₃)₂S—SCH₂CH(NH₂)C(O)—. In some embodiments, the linker group of L1 is -Lys-NHCH(COOH)C(CH₃)₂S—SC(CH₃)₂CH(NH₂)C(O)—. In some embodiments, the linker group of L1 is -Lys-NHCH(COOH)C(CH₃)₂S—SCH₂CH(NH₂)C(O)—.

In some embodiments, the linker group of L1 is -Arg-NH(CH₂)₅C(O)—. In some embodiments, the linker group of L1 is —NH(CH₂)₅C(O)—. In some embodiments, the linker group of L1 is —NH(CH₂)₂C(O)-Arg-NH(CH₂)₅C(O)NH(CH₂)₂C(O)—. In some embodiments, the linker group of L1 is —NH(CH₂)₅C(O)-Arg-NH(CH₂)₂C(O)-Arg-NH(CH₂)₅C(O)-Arg-NH(CH₂)₂C(O)—. In some embodiments, the linker group of L1 is -Arg-NH(CH₂)₅C(O)-Arg-Arg-NH(CH₂)₂C(O)-Arg-Arg-NH(CH₂)₅C(O)— (SEQ ID NO: 334). In some embodiments, the linker group of L1 is —NH(CH₂)₅C(O)NH(CH₂)₂-(D-arginine)-(D-arginine)-(D-arginine)-. In some embodiments, the linker group of L1 is —NH(CH₂CH₂O)₂CH₂C(O)—. In some embodiments, the linker group of L1 is —NH(CH₂CH₂O)₂CH₂C(O)-Arg-. In some embodiments, the linker group of L1 is —NH(CH₂CH₂O)₂CH₂C(O)—NH(CH₂CH₂O)₂CH₂C(O)—. In some embodiments, the linker group of L1 is —NH(CH₂CH₂O)₂CH₂C(O)—NH(CH₂CH₂O)₂CH₂C(O)-Arg-. In some embodiments, the linker group of L1 is —NH(CH₂CH₂O)₂CH₂C(O)—NH(CH₂CH₂O)₂CH₂C(O)-Arg-Arg-. In some embodiments, the linker group of L1 is —NH(CH₂CH₂O)₂CH₂C(O)-Arg-NH(CH₂CH₂O)₂CH₂C(O)-Arg-. In some embodiments, the linker group of L1 is a peptide sequence that is -Lys-. In some embodiments, the linker group of L1 is a peptide sequence that is -(D-Arg)-(D-Arg)-(D-Arg)-.

In some embodiments, L2 is the linker group. In some embodiments, the linker group of L2 is cleavable. In some embodiments, the linker group of L2 is non-cleavable. In some embodiments, the linker group of L2 is a peptide sequence. In some embodiments, the linker group of L2 is a polyamine sequence. In some embodiments, the linker group of L2 is a polyamide sequence.

In some embodiments, the linker group of L2 is a residue of an omega-amino fatty acid. In some embodiments, the linker group of L2 is a residue of an omega-amino caproic acid. In some embodiments, the linker group of L2 is a residue of a dicarboxylic acid. In some embodiments, the linker group of L2 is a residue of oxalic acid. In some embodiments, the linker group of L2 is a residue of succinic acid.

In some embodiments, the linker group of L2 is a peptide sequence that is -Glu-Val-Citrulline-. In some embodiments, the linker group of L2 is —NHCH(COOH)C(CH₃)₂S—SC(CH₃)₂CH(NH₂)C(O)—. In some embodiments, the linker group of L2 is —NHCH(COOH)C(CH₃)₂S—SCH₂CH(NH₂)C(O)—. In some embodiments, the linker group of L2 is -Lys-NHCH(COOH)C(CH₃)₂S—SC(CH₃)₂CH(NH₂)C(O)—. In some embodiments, the linker group of L2 is -Lys-NHCH(COOH)C(CH₃)₂S—SCH₂CH(NH₂)C(O)—.

In some embodiments, the linker group of L2 is -Arg-NH(CH₂)₅C(O)—. In some embodiments, the linker group of L2 is —NH(CH₂)₅C(O)—. In some embodiments, the linker group of L2 is —NH(CH₂)₂C(O)-Arg-NH(CH₂)₅C(O)NH(CH₂)₂C(O)—. In some embodiments, the linker group of L2 is —NH(CH₂)₅C(O)-Arg-NH(CH₂)₂C(O)-Arg-NH(CH₂)₅C(O)-Arg-NH(CH₂)₂C(O)—. In some embodiments, the linker group of L2 is -Arg-NH(CH₂)₅C(O)-Arg-Arg-NH(CH₂)₂C(O)-Arg-Arg-NH(CH₂)₅C(O)— (SEQ ID NO: 334). In some embodiments, the linker group of L2 is —NH(CH₂)₅C(O)NH(CH₂)₂-(D-arginine)-(D-arginine)-(D-arginine)-. In some embodiments, the linker group of L2 is —NH(CH₂CH₂O)₂CH₂C(O)—. In some embodiments, the linker group of L2 is —NH(CH₂CH₂O)₂CH₂C(O)-Arg-. In some embodiments, the linker group of L2 is —NH(CH₂CH₂O)₂CH₂C(O)—NH(CH₂CH₂O)₂CH₂C(O)—. In some embodiments, the linker group of L2 is —NH(CH₂CH₂O)₂CH₂C(O)—NH(CH₂CH₂O)₂CH₂C(O)-Arg-. In some embodiments, the linker group of L2 is —NH(CH₂CH₂O)₂CH₂C(O)—NH(CH₂CH₂O)₂CH₂C(O)-Arg-Arg-. In some embodiments, the linker group of L2 is —NH(CH₂CH₂O)₂CH₂C(O)-Arg-NH(CH₂CH₂O)₂CH₂C(O)-Arg-. In some embodiments, the linker group of L2 is a peptide sequence that is -Lys-. In some embodiments, the linker group of L2 is a peptide sequence that is -(D-Arg)-(D-Arg)-(D-Arg)-.

In some embodiments, L3 is the linker group. In some embodiments, the linker group of L3 is cleavable. In some embodiments, the linker group of L3 is non-cleavable. In some embodiments, the linker group of L3 is a peptide sequence. In some embodiments, the linker group of L3 is a polyamine sequence. In some embodiments, the linker group of L3 is a polyamide sequence.

In some embodiments, the linker group of L3 is a residue of an omega-amino fatty acid. In some embodiments, the linker group of L3 is a residue of an omega-amino caproic acid. In some embodiments, the linker group of L3 is a residue of a dicarboxylic acid. In some embodiments, the linker group of L3 is a residue of oxalic acid. In some embodiments, the linker group of L3 is a residue of succinic acid.

In some embodiments, the linker group of L3 is a peptide sequence that is -Glu-Val-Citrulline-. In some embodiments, the linker group of L3 is —NHCH(COOH)C(CH₃)₂S—SC(CH₃)₂CH(NH₂)C(O)—. In some embodiments, the linker group of L3 is —NHCH(COOH)C(CH₃)₂S—SCH₂CH(NH₂)C(O)—. In some embodiments, the linker group of L3 is -Lys-NHCH(COOH)C(CH₃)₂S—SC(CH₃)₂CH(NH₂)C(O)—. In some embodiments, the linker group of L3 is -Lys-NHCH(COOH)C(CH₃)₂S—SCH₂CH(NH₂)C(O)—.

In some embodiments, the linker group of L3 is -Arg-NH(CH₂)₅C(O)—. In some embodiments, the linker group of L3 is —NH(CH₂)₅C(O)—. In some embodiments, the linker group of L3 is —NH(CH₂)₂C(O)-Arg-NH(CH₂)₅C(O)NH(CH₂)₂C(O)—. In some embodiments, the linker group of L3 is —NH(CH₂)₅C(O)-Arg-NH(CH₂)₂C(O)-Arg-NH(CH₂)₅C(O)-Arg-NH(CH₂)₂C(O)—. In some embodiments, the linker group of L3 is -Arg-NH(CH₂)₅C(O)-Arg-Arg-NH(CH₂)₂C(O)-Arg-Arg-NH(CH₂)₅C(O)— (SEQ ID NO: 334). In some embodiments, the linker group of L3 is —NH(CH₂)₅C(O)NH(CH₂)₂-(D-arginine)-(D-arginine)-(D-arginine)-. In some embodiments, the linker group of L3 is —NH(CH₂CH₂O)₂CH₂C(O)—. In some embodiments, the linker group of L3 is —NH(CH₂CH₂O)₂CH₂C(O)-Arg-. In some embodiments, the linker group of L3 is —NH(CH₂CH₂O)₂CH₂C(O)—NH(CH₂CH₂O)₂CH₂C(O)—. In some embodiments, the linker group of L3 is —NH(CH₂CH₂O)₂CH₂C(O)—NH(CH₂CH₂O)₂CH₂C(O)-Arg-. In some embodiments, the linker group of L3 is —NH(CH₂CH₂O)₂CH₂C(O)—NH(CH₂CH₂O)₂CH₂C(O)-Arg-Arg-. In some embodiments, the linker group of L3 is —NH(CH₂CH₂O)₂CH₂C(O)-Arg-NH(CH₂CH₂O)₂CH₂C(O)-Arg-. In some embodiments, the linker group of L3 is a peptide sequence that is -Lys-. In some embodiments, the linker group of L3 is a peptide sequence that is -(D-Arg)-(D-Arg)-(D-Arg)-.

In some embodiments, L4 is the linker group. In some embodiments, the linker group of L4 is cleavable. In some embodiments, the linker group of L4 is non-cleavable. In some embodiments, the linker group of L4 is a peptide sequence. In some embodiments, the linker group of L4 is a polyamine sequence. In some embodiments, the linker group of L4 is a polyamide sequence.

In some embodiments, the linker group of L4 is a residue of an omega-amino fatty acid. In some embodiments, the linker group of L4 is a residue of an omega-amino caproic acid. In some embodiments, the linker group of L4 is a residue of a dicarboxylic acid. In some embodiments, the linker group of L4 is a residue of oxalic acid. In some embodiments, the linker group of L4 is a residue of succinic acid.

In some embodiments, the linker group of L4 is a peptide sequence that is -Glu-Val-Citrulline-. In some embodiments, the linker group of L4 is —NHCH(COOH)C(CH₃)₂S—SC(CH₃)₂CH(NH₂)C(O)—. In some embodiments, the linker group of L4 is —NHCH(COOH)C(CH₃)₂S—SCH₂CH(NH₂)C(O)—. In some embodiments, the linker group of L4 is -Lys-NHCH(COOH)C(CH₃)₂S—SC(CH₃)₂CH(NH₂)C(O)—. In some embodiments, the linker group of L4 is -Lys-NHCH(COOH)C(CH₃)₂S—SCH₂CH(NH₂)C(O)—.

In some embodiments, the linker group of L4 is -Arg-NH(CH₂)₅C(O)—. In some embodiments, the linker group of L4 is —NH(CH₂)₅C(O)—. In some embodiments, the linker group of L4 is —NH(CH₂)₂C(O)-Arg-NH(CH₂)₅C(O)NH(CH₂)₂C(O)—. In some embodiments, the linker group of L4 is —NH(CH₂)₅C(O)-Arg-NH(CH₂)₂C(O)-Arg-NH(CH₂)₅C(O)-Arg-NH(CH₂)₂C(O)—. In some embodiments, the linker group of L4 is -Arg-NH(CH₂)₅C(O)-Arg-Arg-NH(CH₂)₂C(O)-Arg-Arg-NH(CH₂)₅C(O)— (SEQ ID NO: 334). In some embodiments, the linker group of L4 is —NH(CH₂)₅C(O)NH(CH₂)₂-(D-arginine)-(D-arginine)-(D-arginine)-. In some embodiments, the linker group of L4 is —NH(CH₂CH₂O)₂CH₂C(O)—. In some embodiments, the linker group of L4 is —NH(CH₂CH₂O)₂CH₂C(O)-Arg-. In some embodiments, the linker group of L4 is —NH(CH₂CH₂O)₂CH₂C(O)—NH(CH₂CH₂O)₂CH₂C(O)—. In some embodiments, the linker group of L4 is —NH(CH₂CH₂O)₂CH₂C(O)—NH(CH₂CH₂O)₂CH₂C(O)-Arg-. In some embodiments, the linker group of L4 is —NH(CH₂CH₂O)₂CH₂C(O)—NH(CH₂CH₂O)₂CH₂C(O)-Arg-Arg-. In some embodiments, the linker group of L4 is —NH(CH₂CH₂O)₂CH₂C(O)-Arg-NH(CH₂CH₂O)₂CH₂C(O)-Arg-. In some embodiments, the linker group of L4 is a peptide sequence that is -Lys-. In some embodiments, the linker group of L4 is a peptide sequence that is -(D-Arg)-(D-Arg)-(D-Arg)-.

In some embodiments, L5 is the linker group. In some embodiments, the linker group of L5 is cleavable. In some embodiments, the linker group of L5 is non-cleavable. In some embodiments, the linker group of L5 is a peptide sequence. In some embodiments, the linker group of L5 is a polyamine sequence. In some embodiments, the linker group of L5 is a polyamide sequence.

In some embodiments, the linker group of L5 is a residue of an omega-amino fatty acid. In some embodiments, the linker group of L5 is a residue of an omega-amino caproic acid. In some embodiments, the linker group of L5 is a residue of a dicarboxylic acid. In some embodiments, the linker group of L5 is a residue of oxalic acid. In some embodiments, the linker group of L5 is a residue of succinic acid.

In some embodiments, the linker group of L5 is a peptide sequence that is -Glu-Val-Citrulline-. In some embodiments, the linker group of L5 is —NHCH(COOH)C(CH₃)₂S—SC(CH₃)₂CH(NH₂)C(O)—. In some embodiments, the linker group of L5 is —NHCH(COOH)C(CH₃)₂S—SCH₂CH(NH₂)C(O)—. In some embodiments, the linker group of L5 is -Lys-NHCH(COOH)C(CH₃)₂S—SC(CH₃)₂CH(NH₂)C(O)—. In some embodiments, the linker group of L5 is -Lys-NHCH(COOH)C(CH₃)₂S—SCH₂CH(NH₂)C(O)—.

In some embodiments, the linker group of L5 is -Arg-NH(CH₂)₅C(O)—. In some embodiments, the linker group of L5 is —NH(CH₂)₅C(O)—. In some embodiments, the linker group of L5 is —NH(CH₂)₂C(O)-Arg-NH(CH₂)₅C(O)NH(CH₂)₂C(O)—. In some embodiments, the linker group of L5 is —NH(CH₂)₅C(O)-Arg-NH(CH₂)₂C(O)-Arg-NH(CH₂)₅C(O)-Arg-NH(CH₂)₂C(O)—. In some embodiments, the linker group of L5 is -Arg-NH(CH₂)₅C(O)-Arg-Arg-NH(CH₂)₂C(O)-Arg-Arg-NH(CH₂)₅C(O)— (SEQ ID NO: 334). In some embodiments, the linker group of L5 is —NH(CH₂)₅C(O)NH(CH₂)₂-(D-arginine)-(D-arginine)-(D-arginine)-. In some embodiments, the linker group of L5 is —NH(CH₂CH₂O)₂CH₂C(O)—. In some embodiments, the linker group of L5 is —NH(CH₂CH₂O)₂CH₂C(O)-Arg-. In some embodiments, the linker group of L5 is —NH(CH₂CH₂O)₂CH₂C(O)—NH(CH₂CH₂O)₂CH₂C(O)—. In some embodiments, the linker group of L5 is —NH(CH₂CH₂O)₂CH₂C(O)—NH(CH₂CH₂O)₂CH₂C(O)-Arg-. In some embodiments, the linker group of L5 is —NH(CH₂CH₂O)₂CH₂C(O)—NH(CH₂CH₂O)₂CH₂C(O)-Arg-Arg-. In some embodiments, the linker group of L5 is —NH(CH₂CH₂O)₂CH₂C(O)-Arg-NH(CH₂CH₂O)₂CH₂C(O)-Arg-. In some embodiments, the linker group of L5 is a peptide sequence that is -Lys-. In some embodiments, the linker group of L5 is a peptide sequence that is -(D-Arg)-(D-Arg)-(D-Arg)-.

In some embodiments, L6 is the linker group. In some embodiments, the linker group of L6 is cleavable. In some embodiments, the linker group of L6 is non-cleavable. In some embodiments, the linker group of L6 is a peptide sequence. In some embodiments, the linker group of L6 is a polyamine sequence. In some embodiments, the linker group of L6 is a polyamide sequence.

In some embodiments, the linker group of L6 is a residue of an omega-amino fatty acid. In some embodiments, the linker group of L6 is a residue of an omega-amino caproic acid. In some embodiments, the linker group of L6 is a residue of a dicarboxylic acid. In some embodiments, the linker group of L6 is a residue of oxalic acid. In some embodiments, the linker group of L6 is a residue of succinic acid.

In some embodiments, the linker group of L6 is a peptide sequence that is -Glu-Val-Citrulline-. In some embodiments, the linker group of L6 is —NHCH(COOH)C(CH₃)₂S—SC(CH₃)₂CH(NH₂)C(O)—. In some embodiments, the linker group of L6 is —NHCH(COOH)C(CH₃)₂S—SCH₂CH(NH₂)C(O)—. In some embodiments, the linker group of L6 is -Lys-NHCH(COOH)C(CH₃)₂S—SC(CH₃)₂CH(NH₂)C(O)—. In some embodiments, the linker group of L6 is -Lys-NHCH(COOH)C(CH₃)₂S—SCH₂CH(NH₂)C(O)—.

In some embodiments, the linker group of L6 is -Arg-NH(CH₂)₅C(O)—. In some embodiments, the linker group of L6 is —NH(CH₂)₅C(O)—. In some embodiments, the linker group of L6 is —NH(CH₂)₂C(O)-Arg-NH(CH₂)₅C(O)NH(CH₂)₂C(O)—. In some embodiments, the linker group of L6 is —NH(CH₂)₅C(O)-Arg-NH(CH₂)₂C(O)-Arg-NH(CH₂)₅C(O)-Arg-NH(CH₂)₂C(O)—. In some embodiments, the linker group of L6 is -Arg-NH(CH₂)₅C(O)-Arg-Arg-NH(CH₂)₂C(O)-Arg-Arg-NH(CH₂)₅C(O)— (SEQ ID NO: 334). In some embodiments, the linker group of L6 is —NH(CH₂)₅C(O)NH(CH₂)₂-(D-arginine)-(D-arginine)-(D-arginine)-. In some embodiments, the linker group of L6 is —NH(CH₂CH₂O)₂CH₂C(O)—. In some embodiments, the linker group of L6 is —NH(CH₂CH₂O)₂CH₂C(O)-Arg-. In some embodiments, the linker group of L6 is —NH(CH₂CH₂O)₂CH₂C(O)—NH(CH₂CH₂O)₂CH₂C(O)—. In some embodiments, the linker group of L6 is —NH(CH₂CH₂O)₂CH₂C(O)—NH(CH₂CH₂O)₂CH₂C(O)-Arg-. In some embodiments, the linker group of L6 is —NH(CH₂CH₂O)₂CH₂C(O)—NH(CH₂CH₂O)₂CH₂C(O)-Arg-Arg-. In some embodiments, the linker group of L6 is —NH(CH₂CH₂O)₂CH₂C(O)-Arg-NH(CH₂CH₂O)₂CH₂C(O)-Arg-. In some embodiments, the linker group of L6 is a peptide sequence that is -Lys-. In some embodiments, the linker group of L6 is a peptide sequence that is -(D-Arg)-(D-Arg)-(D-Arg)-.

In some embodiments, the compound binds to a nucleic acid sequence transcribed from a gene associated with a neuromuscular disease phenotype. In some embodiments, the compound binds to a nucleic acid sequence transcribed from a gene associated with a DM1 disease phenotype.

In some embodiments, the compound comprises a structure that interferes with expression of a gene associated with a neuromuscular disease phenotype. In some embodiments, the neuromuscular disease phenotype is a DM1 disease phenotype. In some embodiments, the DM1 disease phenotype is associated with a non-wild-type DM1 gene that differs from a wild type DM1 gene in a repeat expansion mutation. In some embodiments, the compound binds to a mRNA sequence at a region that is (CUG)_{z}, wherein z is an integer from 1-100 (SEQ ID NO: 324). In some embodiments, the units with variables defined independently form a sequence according to any one of SEQ ID NOs: 1-14 and 19-25. In some embodiments, the units with variables defined independently and the water solubilizing group form a sequence according to any one of SEQ ID NOs: 27-44 and 83. In some embodiments, the compound comprises a sequence according to any one of SEQ ID NOs: 1-14, 19-25, and 27-44 and 83. In some embodiments, the compound is a sequence according to any one of SEQ ID NOs: 1-14, 19-25, and 27-44 and 83.

In some embodiments, the compound binds to a nucleic acid sequence transcribed from a gene associated with a neurodegenerative disease phenotype. In some embodiments, the compound binds to a nucleic acid sequence transcribed from a gene associated with a Huntington's disease phenotype.

In some embodiments, the compound binds to a nucleic acid sequence transcribed from a gene associated with a Huntington's disease phenotype by interactions between the heterocycles of the R² groups and nucleobases of a HTT gene. In some embodiments, the HTT gene is a non-wild type HTT gene. In some embodiments, the non-wild type HTT gene differs from a wild type HTT gene in a repeat expansion mutation. In some embodiments, the compound binds to a mRNA sequence at a region that is (CAG)_{z}, wherein z is an integer from 1-100 (SEQ ID NO: 325). In some embodiments, the units with variables defined independently form a peptide nucleic acid according to any one of SEQ ID NOs: 15-18, 26, 45, 26, 45-47, 55, 57, 59, 61, 63, 65, 67-77, 80, and 81. In some embodiments, the units with variables defined independently and the water solubilizing group form a sequence according to any one of SEQ ID NOs: 56, 58, 60, 62, 64, 66, 78, 79, and 82. In some embodiments, the compound comprises a sequence according to any one of SEQ ID NOs: 15-18, 26, 45, 26, 45-47, and 55-82. In some embodiments, the compound is a sequence according to any one of SEQ ID NOs: 15-18, 26, 45, 26, 45-47, and 55-82.

In some embodiments, the compound binds to a nucleic acid sequence encoding a cancer-causing protein. In some embodiments, the cancer-causing protein is mutant K-ras. In some embodiments, the cancer-causing protein is G12D K-ras. In some embodiments, the cancer-causing protein is G12C K-ras. In some embodiments, the cancer-causing protein is G12V K-ras. In some embodiments, the compound binds to the nucleic acid sequence encoding the mutant K-ras by interactions between the heterocycles of the R² groups and nucleobases of the nucleic acid sequence. In some embodiments, the nucleic acid sequence is a mRNA sequence. In some embodiments, the nucleic acid sequence is a DNA sequence. In some embodiments, the units with variables defined independently form a peptide nucleic acid according to any one of SEQ ID NOs: 84-123, 135-142, 163-166, 193-219, and 235-277. In some embodiments, the units with variables defined independently and the water solubilizing group form a sequence according to any one of SEQ ID NOs: 124-134, 143-160, 167-192, 220-234, and 278-296. In some embodiments, the compound comprises a sequence according to any one of SEQ ID NOs: 84-296. In some embodiments, the compound is a sequence according to any one of SEQ ID NOs: 84-296.

In some embodiments, each of L1, L2, L3, L4, L5, L6, PEP1, PEP1, SOL1, and SOL2 is absent.

In some embodiments, the structure is:

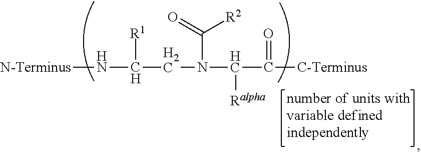

wherein the number of units with variables defined independently is at least 11;
or a pharmaceutically-acceptable salt or ionized form thereof.

In some embodiments, the structure is:

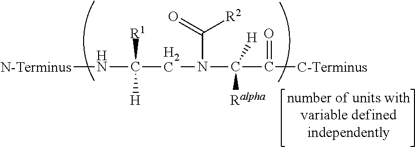

or a pharmaceutically-acceptable salt or ionized form thereof.

In some embodiments, at least one iteration of R¹ is a hydroxyalkyl group.

In some embodiments, N-Terminus is H. In some embodiments, N-Terminus is acyl. In some embodiments, N-Terminus is the biological agent. In some embodiments, the biological agent is a vitamin E group. In some embodiments, the biological agent is an O-bound tocopherol group.

In some embodiments, C-Terminus is OH, O-alkyl, or NH₂. In some embodiments, C-Terminus is OH, OMe, OEt, Ot-Bu, or NH₂. In some embodiments, C-Terminus is NH₂. In some embodiments, C-Terminus is the peptide sequence.

In some embodiments, the compound is Compound 1:

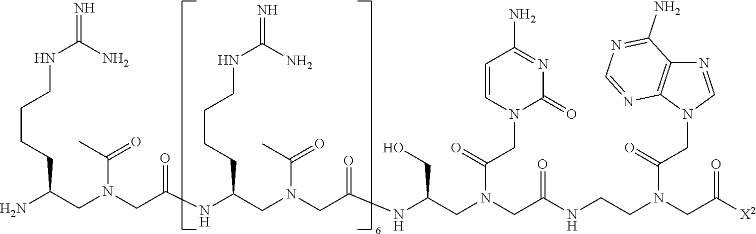

wherein X² is

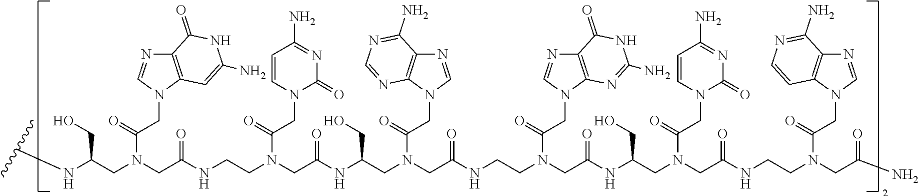

(Compound 1) (SEQ ID NO: 27) or a pharmaceutically-acceptable salt or ionized form thereof.

### Selected Compounds.

Selected compounds of the disclosure are provided in are provided in TABLE 1 and TABLE 2. Residue structures, pendant nucleobase identities (when present), and monomer chemical names associated with the symbols used in the structure codes of TABLE 1, TABLE 2, and TABLE 3 are provided in TABLE 4. Compounds can be provided as a pharmaceutically-acceptable salt, tautomer, or ionized form thereof.

TABLE 1

| | | | {SEQ |
|---|---|---|---|
| Cpd | | | ID |
| # | Structure Code (N to C terminus)^{a} | [Z]^{b} | NO}^{c} |
| 1 | [Z] | SEQ ID NO: 27 | |
| 2 | {RHxRRBxRRHxRFQILYRHxRBxRHxRBx}[Z] | SEQ ID NO: 1 | 297 |
| 3 | {ASSLNIAHxBxR*R*R*}[Z] | SEQ ID NO: 1 | 298 |
| 4 | VeSx[Z]{RHxRRBxRRHxRRBxRHxBx} | SEQ ID NO: 1 | 299 |
| 5 | VeSx[Z]{RHxRRBxRRHxRFQILYRHxRBxRHxRBx} | SEQ ID NO: 1 | 297 |
| 6 | YgYgYgYgYgYgYgPe&&Pe[Z]{PKKKRKV} | SEQ ID NO: 1 | 300 |
| 7 | YgYgYgYgYgYgYgPe&&Pe[Z] | SEQ ID NO: 1 | |
| 8 | YgYgYgYgYgYgYgEVCt[Z] | SEQ ID NO: 1 | |
| 9 | [Z] | SEQ ID NO: 43 | |
| 10 | [Z] | SEQ ID NO: 44 | |
| 11 | [Z] | SEQ ID NO: 42 | |
| 12 | KEVCtQxQx[Z]QxQxRQxRR | SEQ ID NO: 1 | |
| 13 | KEVCtQxQx[Z]QxQxRRQxR | SEQ ID NO: 1 | |
| 14 | KEVCtQxQx[Z]QxQxRQxQxRQxR | SEQ ID NO: 1 | |
| 15 | KEVCtQxQx[Z]QxQxRQxQxRR | SEQ ID NO: 1 | |
| 16 | KEVCtQxQx[Z]QxQxRRQxQxR | SEQ ID NO: 1 | |
| 17 | KEVCtQxQx[Z]QxQxRQxRQxQxR | SEQ ID NO: 1 | |
| 18 | KEVCtQxQx[Z]QxQxRRQxRR | SEQ ID NO: 1 | |
| 19 | KEVCtQxQx[Z]QxQxRRQxRQxR | SEQ ID NO: 1 | |
| 20 | KEVCtQxQx[Z]QxQxRQxRQxRQxR | SEQ ID NO: 1 | |
| 21 | QxQx[Z]QxQxYgYgYg | SEQ ID NO: 1 | |
| 22 | [Z]QxQxYgYgYg | SEQ ID NO: 1 | |
| 23 | [Z] | SEQ ID NO: 33 | |
| 24 | QxQx[Z] | SEQ ID NO: 33 | |
| 25 | KEVCtQxQx[Z]QxQxRR | SEQ ID NO: 1 | |
| 26 | KEVCtQxQx[Z]QxQxRRR | SEQ ID NO: 1 | |
| 27 | KEVCtQxQx[Z]QxQxRQxRQxR | SEQ ID NO: 1 | |
| 28 | KEVCtQxQx[Z]QxQxRQxQxRQxQxR | SEQ ID NO: 1 | |
| 29 | {C(&)RTIGPSVC(&)}[Z]R*R*R* | SEQ ID NO: 1 | 301 |
| 30 | {T*H*R*P*P*M*W*S*P*V*W*P*}[Z]R*R*R* | SEQ ID NO: 1 | 302 |
| 31 | {P*W*V*P*S*W*M*P*P*R*H*T*}[Z]R*R*R* | SEQ ID NO: 1 | 303 |
| 32 | {H*R*P*Y*I*A*H*}[Z]R*R*R* | SEQ ID NO: 1 | 304 |
| 33 | [Z] | SEQ ID NO: 34 | |
| 34 | [Z] | SEQ ID NO: 28 | |
| 35 | [Z] | SEQ ID NO: 29 | |
| 36 | [Z] | SEQ ID NO: 30 | |
| 37 | Hd[Z]R*R*R* | SEQ ID NO: | |
| 38 | LcSx[Z]{ASSLNIAHxBxR*R*R*} | SEQ ID NO: 1 | 298 |
| 39 | Lh[Z]{RHxRRBxRRHxRFQILYRHxRBxRHxRBx} | SEQ ID NO: 1 | 297 |
| 40 | Lh[Z]{RHxRRBxRRHxRRBxRHxBx} | SEQ ID NO: 1 | 299 |
| 41 | Lh[Z]{ASSLNIAHxBxR*R*R*} | SEQ ID NO: | 298 |
| 42 | VeSx[Z]{ASSLNIAHxBxR*R*R*} | SEQ ID NO: 1 | 298 |
| 43 | Hd[Z] | SEQ ID NO: 32 | |
| 44 | LcSx[Z]R*R*R* | SEQ ID NO: 1 | |
| 45 | VeSx[Z] | SEQ ID NO: 32 | |
| 46 | Lh[Z]R*R*R* | SEQ ID NO: 1 | |
| 47 | VeSx[Z]R*R*R* | SEQ ID NO: 1 | |
| 48 | Hd[Z]{RHxRRBxRRHxRFQILYRHxRBxRHxRBx} | SEQ ID NO: 1 | 297 |
| 49 | Hd[Z]{RHxRRBxRRHxRRBxRHxBx} | SEQ ID NO: 1 | 299 |
| 50 | Hd[Z]{ASSLNIAHxBxR*R*R*} | SEQ ID NO: 1 | 298 |
| 51 | LcSx[Z]{RHxRRBxRRHxRFQILYRHxRBxRHxRBx} | SEQ ID NO: | 297 |
| 52 | LcSx[Z]{RHxRRBxRRHxRRBxRHxBx} | SEQ ID NO: 1 | 299 |
| 53 | [Z] | SEQ ID NO: 35 | |
| 54 | [Z] | SEQ ID NO: 36 | |
| 55 | YgYgYgYgYgYgYgQxQx[Z] | SEQ ID NO: 3 | |
| 56 | [Z] | SEQ ID NO: 37 | |
| 57 | YgYgYgYgYgYgYgQxQx[Z] | SEQ ID NO: 5 | |
| 58 | [Z] | SEQ ID NO: 38 | |
| 59 | [Z] | SEQ ID NO: 39 | |
| 60 | [Z] | SEQ ID NO: 30 | |
| 61 | YgYgYgYgYgYgYgQxQx[Z] | SEQ ID NO: 8 | |
| 62 | [Z] | SEQ ID NO: 41 | |
| 63 | [Z] | SEQ ID NO: 10 | |
| 64 | [Z] | SEQ ID NO: 11 | |
| 65 | [Z] | SEQ ID NO: 12 | |
| 66 | [Z] | SEQ ID NO: 13 | |
| 67 | [Z] | SEQ ID NO: 14 | |
| 68 | [Z] | SEQ ID NO: 15 | |
| 69 | [Z] | SEQ ID NO: 16 | |
| 70 | [Z] | SEQ ID NO: 17 | |
| 71 | [Z] | SEQ ID NO: 18 | |
| 72 | [Z] | SEQ ID NO: 19 | |
| 73 | [Z] | SEQ ID NO: 9 | |
| 74 | [Z] | SEQ ID NO: 20 | |
| 75 | [Z] | SEQ ID NO: 21 | |
| 76 | [Z] | SEQ ID NO: 22 | |
| 77 | [Z] | SEQ ID NO: 23 | |
| 78 | [Z] | SEQ ID NO: 24 | |
| 79 | [Z] | SEQ ID NO: 25 | |
| 80 | [Z] | SEQ ID NO: 1 | |
| 81 | {RGRGRGRGRGRGRG}[Z] | SEQ ID NO: 1 | 305 |
| 82 | [Z] | SEQ ID NO: 26 | |
| 83 | {RHxRRBxRRHxRYQFLIRHxRBxRHxRBx}[Z] | SEQ ID NO: 1 | 306 |
| 84 | {RHxRRBxRRHxRIQFLIRHxRBxRHxRBx}[Z] | SEQ ID NO: 1 | 307 |
| 85 | {RHxRRBxRRHxYRFLIRHxRBxRHxRBx}[Z] | SEQ ID NO: 1 | 308 |
| 86 | {RHxRRBxRRHxRRBxRHxBx}[Z] | SEQ ID NO: 1 | 299 |
| 87 | {RRWWRRWRR}[Z] | SEQ ID NO: 1 | 309 |
| 88 | {RRWQW}[Z] | SEQ ID NO: 1 | 310 |
| 89 | {GRKKRRQRRRPQ}Px{GFWFG}[Z] | SEQ ID NO: 1 | 311, |
| | | | 312 |
| 90 | [Z] | SEQ ID NO: 31 | |
| 91 | K[Z]K | SEQ ID NO: 45 | |
| 92 | K[Z]K | SEQ ID NO: 46 | |
| 93 | K*[Z]K* | SEQ ID NO: 47 | |
| 94 | K*GpBxTpBxBx[Z]BxBxGpK* | SEQ ID NO: 48 | |
| 95 | K*GpBxTpGnBxTpGnBxTpGnCpBxGpCnBxGpCnBxG | — | |
| | pK* | | |
| 96 | [Z] | SEQ ID NO: 49 | |
| 97 | [Z] | SEQ ID NO: 50 | |
| 98 | [Z] | SEQ ID NO: 51 | |
| 99 | [Z] | SEQ ID NO: 52 | |
| 100 | [Z] | SEQ ID NO: 53 | |
| 101 | [Z] | SEQ ID NO: 54 | |
| 102 | [Z] | SEQ ID NO: 55 | |
| 103 | [Z]{YGRKKRRQRRR} | SEQ ID NO: 55 | 313 |
| 104 | [Z]{RRRRRRRR} | SEQ ID NO: 55 | 314 |
| 105 | K*[Z]{LCLRPVG} | SEQ ID NO: 55 | 315 |
| 106 | [Z] | SEQ ID NO: 56 | |
| 107 | YgYgYgYgYgYgYgPe&&Pe[Z] | SEQ ID NO: 55 | |
| 108 | [Z] | SEQ ID NO: 57 | |
| 109 | [Z]{YGRKKRRQRRR} | SEQ ID NO: 57 | 313 |
| 110 | [Z]{RRRRRRRR} | SEQ ID NO: 57 | 314 |
| 111 | K*[Z]{LCLRPVG} | SEQ ID NO: 57 | 315 |
| 112 | [Z] | SEQ ID NO: 58 | |
| 113 | CgAgGgCgAgGgKPe&&Pe[Z] | SEQ ID NO: 57 | |
| 114 | YgYgYgYgYgYgYgPe&&Pe[Z] | SEQ ID NO: 57 | |
| 115 | [Z] | SEQ ID NO: 59 | |
| 116 | [Z]{YGRKKRRQRRR} | SEQ ID NO: 59 | 313 |
| 117 | [Z]{RRRRRRRR} | SEQ ID NO: 59 | 314 |
| 118 | K*[Z]{LCLRPVG} | SEQ ID NO: 59 | 315 |
| 119 | [Z] | SEQ ID NO: 60 | |
| 120 | CgAgGgCgAgGgKPe&&Pe[Z] | SEQ ID NO: 59 | |
| 121 | YgYgYgYgYgYgYgPe&&Pe[Z] | SEQ ID NO: 59 | |
| 122 | [Z] | SEQ ID NO: 61 | |
| 123 | [Z]{YGRKKRRQRRR} | SEQ ID NO: 61 | 313 |
| 124 | [Z]{RRRRRRRR} | SEQ ID NO: 61 | 314 |
| 125 | K*[Z]{LCLRPVG} | SEQ ID NO: 61 | 315 |
| 126 | [Z] | SEQ ID NO: 62 | |
| 127 | CgAgGgCgAgGgKPe&&Pe[Z] | SEQ ID NO: 61 | |
| 128 | YgYgYgYgYgYgYgPe&&Pe[Z] | SEQ ID NO: 61 | |
| 129 | [Z] | SEQ ID NO: 63 | |
| 130 | [Z]{YGRKKRRQRRR} | SEQ ID NO: 63 | 313 |
| 131 | [Z]{RRRRRRRR} | SEQ ID NO: 63 | 314 |
| 132 | K*[Z]{LCLRPVG} | SEQ ID NO: 63 | 315 |
| 133 | [Z] | SEQ ID NO: 64 | |
| 134 | CgAgGgCgAgGgKPe&&Pe[Z] | SEQ ID NO: 63 | |
| 135 | YgYgYgYgYgYgYgPe&&Pe[Z] | SEQ ID NO: 63 | |
| 136 | [Z] | SEQ ID NO: 65 | |
| 137 | [Z]{YGRKKRRQRRR} | SEQ ID NO: 65 | 313 |
| 138 | [Z]{RRRRRRRR} | SEQ ID NO: 65 | 314 |
| 139 | K*[Z]{LCLRPVG} | SEQ ID NO: 65 | 315 |
| 140 | [Z] | SEQ ID NO: 66 | |
| 141 | CgAgGgCgAgGgKPe&&Pe[Z] | SEQ ID NO: 65 | |
| 142 | YgYgYgYgYgYgYgPe&&Pe[Z] | SEQ ID NO: 65 | |
| 143 | K*[Z]K* | SEQ ID NO: 67 | |
| 144 | K*[Z]K* | SEQ ID NO: 68 | |
| 145 | K*[Z]K* | SEQ ID NO: 69 | |
| 146 | K*[Z]K* | SEQ ID NO: 70 | |
| 147 | K*[Z]K* | SEQ ID NO: 71 | |
| 148 | K*[Z]K* | SEQ ID NO: 72 | |
| 149 | K*[Z]K* | SEQ ID NO: 73 | |
| 150 | K*[Z]K* | SEQ ID NO: 74 | |
| 151 | K*[Z]K* | SEQ ID NO: 75 | |
| 152 | K*[Z]K* | SEQ ID NO: 76 | |
| 153 | [Z] | SEQ ID NO: 77 | |
| 154 | CgAgGgCgAgGgKPe&Pe[Z] | SEQ ID NO: 55 | |
| 155 | [Z] | SEQ ID NO: 78 | |
| 156 | [Z] | SEQ ID NO: 79 | |
| 157 | YgYgYgYgYgYgYgQxQx[Z] | SEQ ID NO: 80 | |
| 158 | {C(&)LSSRLDAC(&)}[Z]{R*R*R*R*} | SEQ ID NO: 81 | 316, |
| | | | 317 |
| 159 | {C(&)AGALC(&)Y}[Z]{R*R*R*R*} | SEQ ID NO: 81 | 318, |
| | | | 317 |
| 160 | {C(&)LEVSRKNC(&)}[Z]{R*R*R *R*} | SEQ ID NO: 81 | 319, |
| | | | 317 |
| 161 | {C(&)RTIGPSVC(&)}[Z]{R*R*R*R*} | SEQ ID NO: 81 | 301, |
| | | | 317 |
| 162 | {C(&)TSTSAPYC(&)}[Z]{R*R*R*R*} | SEQ ID NO: 81 | 320, |
| | | | 317 |
| 163 | {C(&)*MPRLRGC(&)}[Z]{R*R*R*R*} | SEQ ID NO: 81 | 321, |
| | | | 317 |
| 164 | {TGNYKALHPHNG}[Z]{R*R *R *R*} | SEQ ID NO: 81 | 322, |
| | | | 317 |
| 165 | [Z] | SEQ ID NO: 82 | |
| 166 | YgYgYgYgYgYgYgQxQx[Z] | SEQ ID NO: 81 | |
| 167 | Ac[Z] | SEQ ID NO: 27 | |

ªUnless otherwise noted, all C-termini are amidated.
bThe structure code corresponding to each SEQ ID NO: is provided in TABLE 3. For example, the structure code of Compound 94 is K*GpBxTpBxBx[Z]BxBxGpK*, which is K*GpBxTpBxBxTpGnCnTpGnCpTnGpCnTnGpBxBxGpK*.
^{c}Subsequences(s)of structure code within braces (e.g., “{TGNYKALHPHNG}”), when present, correspond to SEQ ID NOs provided in this column, listed from first to last occurrence in N to C terminal direction. When more than one subsequence is encompassed within braces in a single compound, corresponding SEQ ID NOs are listed from first to last occurrence in N to C terminal direction.

TABLE 2

| Cpd # | Structure Code (N to C terminus)^{a} | SEQ ID NO^{b} |
|---|---|---|
| 168 | CsCsTsAsCsGsCsCsAsTsCsAsGsCsTsCsCs | 84 |
| 169 | TsAnCsGnCsCnAsTnCsAnGsCnTsCnCsAnAs | 85 |
| 170 | TsAsCsGsCsCsAsTsCsAsGsCsTsCsCsAsAs | 86 |
| 171 | TsGsCsCsTsAsCsGsCsCsAsTsCsAsGsCsTs | 87 |
| 172 | GsCnCsTnAsCnGsCnCsAnTsCnAsGnCsTnCsCnAsAn | 88 |
| 173 | GsCsCsTsAsCsGsCsCsAsTsCsAsGsCsTsCsCsAsAs | 89 |
| 174 | TsGnCsCnTsAnCsGnCsCnAsAnCsAnGsCnTsCnCsAn | 90 |
| 175 | CsTsAsCsGsCsCsAsAsCsAsGsCsTsCsCsAs | 91 |
| 176 | CsCnTsAnCsGnCsCnAsAnCsAnGsCnTsCnCs | 92 |
| 177 | CsTnAsCnGsCnCsAnTsCnAsGnCsTnCsCnAs | 93 |
| 178 | CsCnTsAnCsGnCsCnAsTnCsAnGsCnTsCnCs | 94 |
| 179 | TsGnCsCnTsAnCsGnCsCnAsTnCsAnGsCnTs | 95 |
| 180 | TsAnCsGnCsCnAsTnCsAnGsCnTsCnCs | 96 |
| 181 | TsAsCsGsCsCsAsTsCsAsGsCsTsCsCs | 97 |
| 182 | TnAnCnGnCsCsAsTsCsAsGsCnTnCnCn | 98 |
| 183 | AsCsGsCsCsAsTsCsAsGsCsTs | 99 |
| 184 | CsCnTsAnCsGnCsCnAsAnCsAnGsCnTsCnCsAnAsCn | 100 |
| 185 | TsTnGsCnCsTnAsCnGsCnCsAnAsCnAsGnCsTnCsCn | 101 |
| 186 | TsAnCsGnCsCnAsAnCsAnGsCnTsCnCsAnAs | 102 |
| 187 | GsCnCsTnAsCnGsCnCsAnAsCnAsGnCsTnCs | 103 |
| 188 | TsGnCsCnTsAnCsGnCsCnAsAnCsAnGsCnTs | 104 |
| 189 | TsGsCsCsTsAsCsGsCsCsAsAsCsAsGsCsTs | 105 |
| 190 | AsCsGsCsCsAsAsCsAsGsCsTsCsCsAs | 106 |
| 191 | TsAnCsGnCsCnAsAnCsAnGsCnTsCnCs | 107 |
| 192 | CsTsAsCsGsCsCsAsAsCsAsGsCsTsCs | 108 |
| 193 | CsCsTsAsCsGsCsCsAsAsCsAsGsCsTs | 109 |
| 194 | CnTnAnCnGnCsCsAsAsCsAsGsCnTnCnCnAn | 110 |
| 195 | AsCsGsCsCsAsAsCsAsGsCsTs | 111 |
| 196 | AsCnGsCnCsAnCsAnAsGnCsTnCsCnAsAnCs | 112 |
| 197 | AsCsGsCsCsAsCsAsAsGsCsTsCsCsAsAsCs | 113 |
| 198 | TsAnCsGnCsCnAsCnAsAnGsCnTsCnCsAnAs | 114 |
| 199 | TsAsCsGsCsCsAsCsAsAsGsCsTsCsCsAsAs | 115 |
| 200 | CsTnAsCnGsCnCsAnCsAnAsGnCsTnCsCnAs | 116 |
| 201 | CsTsAsCsGsCsCsAsCsAsAsGsCsTsCsCsAs | 117 |
| 202 | CsCnTsAnCsGnCsCnAsCnAsAnGsCnTsCnCs | 118 |
| 203 | GsCnCsTnAsCnGsCnCsAnCsAnAsGnCsTnCs | 119 |
| 204 | GsCsCsAsCsAsAsGsCsTsCsCsAsAsCs | 120 |
| 205 | AsCnGsCnCsAnCsAnAsGnCsTnCsCnAs | 121 |
| 206 | AsCsGsCsCsAsCsAsAsGsCsTsCsCsAs | 122 |
| 207 | CsTsAsCsGsCsCsAsCsAsAsGsCsTsCs | 123 |
| 208 | YgYgYgYgYgYgYgTsGnGsAnGsCnTsGnAsTnGsGnCsGnTsAnGs{PKKKRKV} | 124, {300} |
| 209 | YgYgYgYgYgYgYgTsGnGsAnGsCnTsGnAsTnGsGnCsGnTsAnGs | 125 |
| 210 | YgYgYgYgYgYgYgTsZnZsAnZsCnTsZnAsTnZsZnCsZnTsAnZs | 126 |
| 211 | YgYgYgYgYgYgYgTsZnZsAnZsCnTsGnAsTnZsZnCsZnTsAnGs | 127 |
| 212 | YgYgYgYgYgYgYgTsYnGsAnYsCnTsGnAsTnYsGnCsYnTsAnGs{PKKKRKV} | 128, {300} |
| 213 | YgYgYgYgYgYgYgTsYnGsAnYsCnTsGnAsTnYsGnCsYnTsAnGs | 129 |
| 214 | YgYgYgYgYgYgYgTsYnGsDnYsCnTsGnDsTnYsGnCsYnTsDnGs{PKKKRKV} | 130, {300} |
| 215 | YgYgYgYgYgYgYgTsYnGsDnYsCnTsGnDsTnYsGnCsYnTsDnGs | 131 |
| 216 | YgYgYgYgYgYgYgGsDnGsCnTsGnDsTnGsGnCsGnTsDn{PKKKRKV} | 132, {300} |
| 217 | YgYgYgYgYgYgYgGsAnGsCnSsGnAsSnGsGnCsGnSsAn{PKKKRKV} | 133, {300} |
| 218 | YgYgYgYgYgYgYgGsAnGsCnTsGnAsTnGsGnCsGnTsAn{PKKKRKV} | 134, {300} |
| 219 | GgAnGgCnTgGnAgTnGgGnCgGnTgAn | 135 |
| 220 | GgAnGgCnTgGnAgTnGgGnCgGnTg | 136 |
| 221 | AnGgCnTgGnAgTnGgGnCgGnTg | 137 |
| 222 | AnGgCnTgGnAgTnGgGnCgGn | 138 |
| 223 | GgCnTgGnAgTnGgGnCgGn | 139 |
| 224 | TgGnGgAnGgCnTgGnAgTnGgGnCgGnTgAnGg | 140 |
| 225 | TgGnGgAnGgCnTgGnAgTnGgGnCgGnTgAn | 141 |
| 226 | GnGgAnGgCnTgGnAgTnGgGnCgGnTgAn | 142 |
| 227 | YgYgYgYgYgYgYgGsAnGsCnTsGnAsTnGsGnCsGnTsAn | 143 |
| 228 | YgYgYgYgYgYgYgZsAnZsCnTsZnAsTnZsZnCsZnTsAn | 144 |
| 229 | YgYgYgYgYgYgYgZsDnZsCnTsZnDsTnZsZnCsZnTsDn | 145 |
| 230 | YgYgYgYgYgYgYgGsAnYsCnTsGnAsTnYsGnCsYnTsAn{PKKKRKV} | 146, {300} |
| 231 | YgYgYgYgYgYgYgGsAnYsCnTsGnAsTnYsGnCsYnTsAn | 147 |
| 232 | YgYgYgYgYgYgYgGsDnYsCnTsGnDsTnYsGnCsYnTsDn{PKKKRKV} | 148, {300} |
| 233 | YgYgYgYgYgYgYgGsDnYsCnTsGnDsTnYsGnCsYnTsDn | 149 |
| 234 | YgYgYgYgYgYgYgTsGnGsAnGsCnTsGnTsTnGsGnCsGnTsAnGs{PKKKRKV} | 150, {300} |
| 235 | YgYgYgYgYgYgYgTsGnGsAnGsCnTsGnTsTnGsGnCsGnTsAnGs | 151 |
| 236 | YgYgYgYgYgYgYgTsYnGsAnYsCnTsGnTsTnYsGnCsYnTsAnGs{PKKKRKV} | 152, {300} |
| 237 | YgYgYgYgYgYgYgTsYnGsAnYsCnTsGnTsTnYsGnCsYnTsAnGs | 153 |
| 238 | YgYgYgYgYgYgYgTsYnGsDnYsCnTsGnTsTnYsGnCsYnTsDnGs{PKKKRKV} | 154, {300} |
| 239 | YgYgYgYgYgYgYgTsYnGsDnYsCnTsGnTsTnYsGnCsYnTsDnGs | 155 |
| 240 | YgYgYgYgYgYgYgGsDnGsCnTsGnTsTnGsGnCsGnTsDn{PKKKRKV} | 156, {300} |
| 241 | YgYgYgYgYgYgYgGsDnGsCnTsGnTsTnGsGnCsGnTsDn | 157 |
| 242 | YgYgYgYgYgYgYgGsAnGsCnSsGnSsSnGsGnCsGnSsAn{PKKKRKV} | 158, {300} |
| 243 | YgYgYgYgYgYgYgGsAnYsCnSsGnSsSnYsGnCsYnTsAn{PKKKRKV} | 159, {300} |
| 244 | YgYgYgYgYgYgYgGsAnGsCnTsGnTsTnGsGnCsGnTsAn{PKKKRKV} | 160, {300} |
| 245 | GgAnGgCnTgGnTgTnGgGnCgGnTgAn | 161 |
| 246 | GgAnGgCnTgGnTgTnGgGnCgGnTg | 162 |
| 247 | AnGgCnTgGnTgTnGgGnCgGnTg | 163 |
| 248 | AnGgCnTgGnTgTnGgGnCgGn | 164 |
| 249 | GgCnTgGnTgTnGgGnCgGn | 165 |
| 250 | TgGnGgAnGgCnTgGnTgTnGgGnCgGnTgAnGg | 166 |
| 251 | YgYgYgYgYgYgYgTgGnGgAnGgCnTgGnTgTnGgGnCgGnTgAn{PKKKRKV} | 167, {300} |
| 252 | YgYgYgYgYgYgYgGnGgAnGgCnTgGnTgTnGgGnCgGnTgAn{PKKKRKV} | 168, {300} |
| 253 | YgYgYgYgYgYgYgGsAnGsCnTsGnTsTnGsGnCsGnTsAn | 169 |
| 254 | YgYgYgYgYgYgYgZsAnZsCnTsZnTsTnZsZnCsZnTsAn | 170 |
| 255 | YgYgYgYgYgYgYgGsAnYsCnTsGnTsTnYsGnCsYnTsAn{PKKKRKV} | 171, {300} |
| 256 | YgYgYgYgYgYgYgGsAnYsCnTsGnTsTnYsGnCsYnTsAn | 172 |
| 257 | YgYgYgYgYgYgYgGsDnYsCnTsGnTsTnYsGnCsYnTsDn{PKKKRKV} | 173, {300} |
| 258 | YgYgYgYgYgYgYgGsDnYsCnTsGnTsTnYsGnCsYnTsDn | 174 |
| 259 | YgYgYgYgYgYgYgGsGnDsGnCsTnTsGnTsGnGsCnGsTn{PKKKRKV} | 175, {300} |
| 260 | YgYgYgYgYgYgYgGsGnAsGnCsSnSsGnSsGnGsCnGsSn{PKKKRKV} | 176, {300} |
| 261 | YgYgYgYgYgYgYgGsGnAsGnCsTnTsGnTsGnGsCnGsTn{PKKKRKV} | 177, {300} |
| 262 | YgYgYgYgYgYgYgTsGnGsAnGsCnTsTnGsTnGsGnCsGnTsAnGs{PKKKRKV} | 178, {300} |
| 263 | YgYgYgYgYgYgYgTsGnGsAnGsCnTsTnGsTnGsGnCsGnTsAnGs | 179 |
| 264 | YgYgYgYgYgYgYgTsYnGsAnYsCnTsTnGsTnYsGnCsYnTsAnGs{PKKKRKV} | 180, {300} |
| 265 | YgYgYgYgYgYgYgTsYnGsAnYsCnTsTnGsTnYsGnCsYnTsAnGs | 181 |
| 266 | YgYgYgYgYgYgYgTsYnGsDnYsCnTsTnGsTnYsGnCsYnTsDnGs{PKKKRKV} | 182, {300} |
| 267 | YgYgYgYgYgYgYgTsYnGsDnYsCnTsTnGsTnYsGnCsYnTsDnGs | 183 |
| 268 | YgYgYgYgYgYgYgGsAnGsCnTsTnGsTnGsGnCsGnTsAn{PKKKRKV} | 184, {300} |
| 269 | YgYgYgYgYgYgYgGsAnGsCnTsTnGsTnGsGnCsGnTsAn | 185 |
| 270 | YgYgYgYgYgYgYgGsAnYsCnTsTnGsTnYsGnCsYnTsAn{PKKKRKV} | 186, {300} |
| 271 | YgYgYgYgYgYgYgGsAnYsCnTsTnGsTnYsGnCsYnTsAn | 187 |
| 272 | YgYgYgYgYgYgYgGsDnYsCnTsTnGsTnYsGnCsYnTsDn{PKKKRKV} | 188, {300} |
| 273 | YgYgYgYgYgYgYgGsDnYsCnTsTnGsTnYsGnCsYnTsDn | 189 |
| 274 | YgYgYgYgYgYgYgTsZnZsAnZsCnTsZnTsTnZsZnCsZnTsAnZs | 190 |
| 275 | YgYgYgYgYgYgYgZsAnZsCnTsTnZsTnZsZnCsZnTsAn | 191 |
| 276 | YgYgYgYgYgYgYgZsAnZsCnTsTnGsTnZsZnCsZnTsAn | 192 |
| 277 | TnGgGnAgGnCgTnTgGnTgGnGgCnGgTnAgGn | 193 |
| 278 | TnGgGnAgGnCgTnTgGnTgGnGgCnGgTnAgGn{PKKKRKV] | 194, {300} |
| 279 | TnGgGnAgGnCgTnTgGnTgGnGgCnGgTnAgGn{PAAKRVKLD} | 195, {323} |
| 280 | TnYgGnAgGnCgTnTgGnTgGnYgCnYgTnAgGn | 196 |
| 281 | TnYgGnAgGnCgTnTgGnTgGnYgCnYgTnAgGn{PKKKRKV} | 197, {300} |
| 282 | TnYgGnAgGnCgTnTgGnTgGnYgCnYgTnAgGn{PAAKRVKLD} | 198, {323} |
| 283 | TnGgYnAgYnCgTnTgYnTgYnGgCnGgTnAgGn | 199 |
| 284 | TnGgYnAgYnCgTnTgYnTgYnGgCnGgTnAgGn{PKKKRKV} | 200, {300} |
| 285 | TnGgYnAgYnCgTnTgYnTgYnGgCnGgTnAgGn{PAAKRVKLD} | 201, {323} |
| 286 | TnGgGnAgGnCgTnGgAnTgGnGgCnGgTnAgGn | 202 |
| 287 | TnGgGnAgGnCgTnGgAnTgGnGgCnGgTnAgGn{PKKKRKV} | 203, {300} |
| 288 | TnGgGnAgGnCgTnGgAnTgGnGgCnGgTnAgGn{PAAKRVKLD} | 204, {323} |
| 289 | TnYgGnAgGnCgTnYgAnTgGnYgCnYgTnAgGn | 205 |
| 290 | TnYgGnAgGnCgTnYgAnTgGnYgCnYgTnAgGn{PKKKRKV} | 206, {300} |
| 291 | TnYgGnAgGnCgTnYgAnTgGnYgCnYgTnAgGn{PAAKRVKLD} | 207, {323} |
| 292 | TnGgYnAgYnCgTnGgAnTgYnGgCnGgTnAgGn | 208 |
| 293 | TnGgYnAgYnCgTnGgAnTgYnGgCnGgTnAgGn{PKKKRKV} | 209, {300} |
| 294 | TnGgYnAgYnCgTnGgAnTgYnGgCnGgTnAgGn{PAAKRVKLD} | 210, {323} |
| 295 | TnGgGnAgGnCgTnGgTnTgGnGgCnGgTnAgGn | 211 |
| 296 | TnGgGnAgGnCgTnGgTnTgGnGgCnGgTnAgGn{PKKKRKV} | 212, {300} |
| 297 | TnGgGnAgGnCgTnGgTnTgGnGgCnGgTnAgGn{PAAKRVKLD} | 213, {323} |
| 298 | TnYgGnAgGnCgTnYgTnTgGnYgCnGgTnAgGn | 214 |
| 299 | TnYgGnAgGnCgTnYgTnTgGnYgCnGgTnAgGn{PKKKRKV} | 215, {300} |
| 300 | TnYgGnAgGnCgTnYgTnTgGnYgCnGgTnAgGn{PAAKRVKLD} | 216, {323} |
| 301 | TnGgYnAgYnCgTnGgTnTgYnGgCnGgTnAgGn | 217 |
| 302 | TnGgYnAgYnCgTnGgTnTgYnGgCnGgTnAgGn{PKKKRKV} | 218, {300} |
| 303 | TnGgYnAgYnCgTnGgTnTgYnGgCnGgTnAgGn{PAAKRVKLD} | 219, {323} |
| 304 | YgYgYgYgYgYgYgGsCnTsGnAsTnGsGn | 220 |
| 305 | YgYgYgYgYgYgYgGsCnTsGnAsTnGsGnCs | 221 |
| 306 | YgYgYgYgYgYgYgGsCnTsGnAsTnGsGnCsGn | 222 |
| 307 | YgYgYgYgYgYgYgGsCnTsGnAsTnGsGnCsGnTs | 223 |
| 308 | YgYgYgYgYgYgYgGsAnGsCnTsGnAsTnGsGnCsGn | 224 |
| 309 | YgYgYgYgYgYgYgGsAnGsCnTsGnAsTnGsGnCsGnTs | 225 |
| 310 | YgYgYgYgYgYgYgTsGnGsAnGsCnTsGnAsTnGsGnCsGnTs | 226 |
| 311 | YgYgYgYgYgYgYgGsAnGsCnTsGnTsTnGsGnCsGn | 227 |
| 312 | YgYgYgYgYgYgYgGsAnGsCnTsGnTsTnGsGnCsGnTs | 228 |
| 313 | YgYgYgYgYgYgYgTsGnGsAnGsCnTsGnTsTnGsGnCsGnTs | 229 |
| 314 | YgYgYgYgYgYgYgTsGnGsAnGsCnTsGnTsTnGsGnCsGnTsAn | 230 |
| 315 | YgYgYgYgYgYgYgGsCnTsGnTsTnGsGn | 231 |
| 316 | YgYgYgYgYgYgYgGsCnTsGnTsTnGsGnCs | 232 |
| 317 | YgYgYgYgYgYgYgGsCnTsGnTsTnGsGnCsGn | 233 |
| 318 | YgYgYgYgYgYgYgGsCnTsGnTsTnGsGnCsGnTs | 234 |
| 319 | CsCsTsAsCsGsCsCsAsCsAsAsGsCsTsCsCsAsAsCs | 235 |
| 320 | GsCnCsTnAsCnGsCnCsAnCsAnAsGnCsTnCsCnAsAn | 236 |
| 321 | TnAnCnGnCnCsAsCsAsAsGsCsTnCnCnAnAn | 237 |
| 322 | CsGsCsCsAsCsAsAsGsCsTsCs | 238 |
| 323 | CsCsTsAsCsGsCsCsAsCsAsAsGsCsTsCsCs | 239 |
| 324 | CnCnTnAnCnGnCsCsAsTsCsAsGsCnTnCnCnAnAnCn | 240 |
| 325 | CnTnAnCnGnCsCsAsTsCsAsGsCnTnCnCnAn | 241 |
| 326 | CsTsAsCsGsCsCsAsTsCsAsGsCsTsCsCsAs | 242 |
| 327 | TsGnCsCnTsAnCsGnCsCnAsTnCsAnGsCnTsCnCsAnAsCn | 243 |
| 328 | TsGnCsCnTsAnCsGnCsCnAsTnCsAnGsCnTsCnCsAn | 244 |
| 329 | GsGnAsGnCsTnGsAnTsGnGsCnGsTnAsGnGsCnAsAn | 245 |
| 330 | CsTnTsGnCsCnTsAnCsGnCsCnAsTnCsAnGsCnTsCn | 246 |
| 331 | TsTnGsCnCsTnAsCnGsCnCsAnTsCnAsGnCsTnCsCnAsAn | 247 |
| 332 | TsCnTsTnGsCnCsTnAsCnGsCnCsAnTsCnAsGnCsTn | 248 |
| 333 | CsTnTsGnCsCnTsAnCsGnCsCnAsTnCsAnGsCnTsCnCsAn | 249 |
| 334 | TsCnTsTnGsCnCsTnAsCnGsCnCsAnTsCnAsGnCsTnCsCn | 250 |
| 335 | TsTnGsCnCsTnAsCnGsCnCsAnTsCnAsGnCsTnCsCnAsAnCsTn | 251 |
| 336 | CsTnTsGnCsCnTsAnCsGnCsCnAsTnCsAnGsCnTsCnCsAnAsCn | 252 |
| 337 | TsCnTsTnGsCnCsTnAsCnGsCnCsAnTsCnAsGnCsTnCsCnAsAn | 253 |
| 338 | CsTnCsTnTsGnCsCnTsAnCsGnCsCnAsTnCsAnGsCnTsCnCsAn | 254 |
| 339 | GvCnCvTnAvCnGvCnCvAnTvCnAvGnCvTnCvCnAvAn | 255 |
| 340 | AnCnGsCnCnAnSsCnAnGnCsTn | 256 |
| 341 | GsCsCsTsAsCsGsCsCsAsSnCsAsGsCsTsCsCsAsAs | 257 |
| 342 | GnCnCsTnAnCnGsCnCnAnSsCnAnGnCsTnCnCnAsAn | 258 |
| 343 | GnCsCnTnAnCsGnCnCnAsTnCnAnGnCsTnCnCnAsAn | 259 |
| 344 | AsCsGsCsCsAsSnCsAsGsCsTs | 260 |
| 345 | AnCsGnCnCnAsTnCnAnGsCnTn | 261 |
| 346 | GsCsCsTsAsCsGsCsCsAsAsCsAsGsCsTsCsCsAsAs | 262 |
| 347 | TsAsCsGsCsCsAsAsCsAsGsCsTsCsCs | 263 |
| 348 | TsAsCsGsCsCsAsAsCsAsGsCsTsCsCsAsAs | 264 |
| 349 | CsCsTsAsCsGsCsCsAsAsCsAsGsCsTsCsCs | 265 |
| 350 | CsTnAsCnGsCnCsAnAsCnAsGnCsTnCsCnAs | 266 |
| 351 | CsGsCsCsAsAsCsAsGsCsTsCs | 267 |
| 352 | GsCnCsTnAsCnGsCnCsAnAsCnAsGnCsTnCsCnAsAn | 268 |
| 353 | GsCsCsTsAsCsGsCsCsAsAsCsAsGsCsTsCs | 269 |
| 354 | CsTnTsGnCsCnTsAnCsGnCsCnAsAnCsAnGsCnTsCnCsAn | 270 |
| 355 | TsCnTsTnGsCnCsTnAsCnGsCnCsAnAsCnAsGnCsTnCsCn | 271 |
| 356 | CsTnTsGnCsCnTsAnCsGnCsCnAsAnCsAnGsCnTsCnCsAnAsCn | 272 |
| 357 | TsCnTsTnGsCnCsTnAsCnGsCnCsAnAsCnAsGnCsTnCsCnAsAn | 273 |
| 358 | CsTnCsTnTsGnCsCnTsAnCsGnCsCnAsAnCsAnGsCnTsCnCsAn | 274 |
| 359 | CsTnTsGnCsCnTsAnCsGnCsCnAsAnCsAnGsCnTsCn | 275 |
| 360 | TsCnTsTnGsCnCsTnAsCnGsCnCsAnAsCnAsGnCsTn | 276 |
| 361 | TsTnGsCnCsTnAsCnGsCnCsAnAsCnAsGnCsTnCsCnAsAn | 277 |
| 362 | YgYgYgYgYgYgYgTsZnZsAnZsCnTsTnZsTnZsZnCsZnTsAnZs | 278 |
| 363 | YgYgYgYgYgYgYgTsZnZsAnZsCnTsTnGsTnZsZnCsZnTsAnGs | 279 |
| 364 | YgYgYgYgYgYgYgGsCnCsAnTsCnAsGnCsTn | 280 |
| 365 | YgYgYgYgYgYgYgCnGsCnCsAnTsCnAsGnCsTnCs | 281 |
| 366 | YgYgYgYgYgYgYgAsCnGsCnCsAnTsCnAsGnCsTnCsCn | 282 |
| 367 | YgYgYgYgYgYgYgTnAsCnGsCnCsAnTsCnAsGnCsTnCsCnAs | 283 |
| 368 | YgYgYgYgYgYgYgGsCnCsTnAsCnGsCnCsAnTsCnAsGnCsTnCsCnAsAn | 284 |
| 369 | YgYgYgYgYgYgYgCnGsCnCsYnTsYnAsGnCsTnCs | 285 |
| 370 | YgYgYgYgYgYgYgTnAsCnGsCnCsYnTsYnAsGnCsTnCsCnAs | 286 |
| 371 | YgYgYgYgYgYgYgGsCnCsTnAsCnGsCnCsYnTsYnAsGnCsTnCsCnAsAn | 287 |
| 372 | YgYgYgYgYgYgYgCnGsCnCnAnTsCnAnGnCsTnCn | 288 |
| 373 | YgYgYgYgYgYgYgTnAnCnGsCnCnAnTsCsAnGnCsTnCnCnAs | 289 |
| 374 | YgYgYgYgYgYgYgGnCnCsTnAnCnGsCnCnAnTsCnAnGnCsTnCnCnAsAn | 290 |
| 375 | YgYgYgYgYgYgYgCnGsCnCnYnTsYnAnGnCsTnCn | 291 |
| 376 | YgYgYgYgYgYgYgTnAnCnGsCnCnYnTsYnAnGnCsTnCnCnAs | 292 |
| 377 | YgYgYgYgYgYgYgGnCnCsTnAnCnGsCnCnYnTsYnAnGnCsTnCnCnAsAn | 293 |
| 378 | YgYgYgYgYgYgYgTsAnCsGsCsCnAsTsCsAnGsCsTsCnCsAs | 294 |
| 379 | YgYgYgYgYgYgYgTsAnCsGsCsCnYsTsYsAnGsCsTsCnCsAs | 295 |
| 380 | YgYgYgYgYgYgYgGsCsCsTsAsCsGsCsCsAsTsCsAsGsCsTsCsCsAsAs | 296 |

ªUnless otherwise noted, all C-termini are amidated.bSubsequence of structure code outside of braces corresponds to SEQ ID NOs provided in this column without braces. Subsequences of structure code within braces (e.g., “{PKKKRKV}”), when present, correspond to SEQ ID NOs provided in this column withinin braces (e.g. “{300}”). For example, in compound 296, TnGgGnAgGnCgTnGgTnTgGnGgCnGgTnAgGn corresponds to SEQ ID NO: 212, and {PKKKRKV}corresponds to SEQ ID NO: 300.

TABLE 3

| SEQ ID | |
|---|---|
| NO: | SEQ |
| 1 | CsAnGsCnAsGnCsAnGsCnAsGnCsAn |
| 2 | AsGnCsAnGsCnCsGnCsAnGsCnAsAn |
| 3 | CsAnGsCnAsGnCsAnGsCn |
| 4 | CsAsGnCsAsGnCsAsGnCsAsGnCsAs |
| 5 | CsAsGnCsAsGnCsAsGnCs |
| 6 | CsAnGsCnAsGnCsAnGsCnAsGn |
| 7 | CsAsGnCsAsGnCsAsGnCsAsGn |
| 8 | CsAnGnCsAnGnCsAnGnCsAnGnCsAn |
| 9 | CsAnGsCnAsGnCsAnGsCnAsGnCsAnGsCnAs |
| 10 | CsAnGsCnAsGnCsAnGsCnAsGnCsAnGs |
| 11 | GsCnAsGnCsAnGsCnAsGnCsAnGsCn |
| 12 | GsCnAsGnCsAnGsCnAsGnCsAnGsCnAs |
| 13 | GsCnAsGnCsAnGsCnAsGnCsAnGsCnAsGn |
| 14 | GsCnAsGnCsAnGsCnAsGnCsAnGsCnAsGnCs |
| 15 | CsAnGsTnCsGnAsGnGsCnCsAnAsAn |
| 16 | CsAnGsTnCsGnAsGnGsCnCsAnAsAnGs |
| 17 | CsAnGsTnCsGnAsGnGsCnCsAnAsAnGsAn |
| 18 | CsAnGsTnCsGnAsGnGsCnCsAnAsAnGsAnAs |
| 19 | CsAnGsCnAsGnCsAnGsCnAsGnCsAnGsCn |
| 20 | AnGsCnAsGnCsAnGsCnAsGnCsAn |
| 21 | AnGsCnAsGnCsAnGsCnAsGnCsAnGs |
| 22 | AnGsCnAsGnCsAnGsCnAsGnCsAnGsCn |
| 23 | AnGsCnAsGnCsAnGsCnAsGnCsAnGsCnAs |
| 24 | AnGsCnAsGnCsAnGsCnAsGnCsAnGsCnAsGn |
| 25 | GsCnAsGnCsAnGsCnAsGnCsAnGs |
| 26 | CsAnGsTnCsGnAsGnGsCnCsAnAsAnGsAnAsGn |
| 27 | YgYgYgYgYgYgYgCsAnGsCnAsGnCsAnGsCnAsGnCsAn |
| 28 | YgYgYgYgYgYgCsAnGsCnAsGnCsAnGsCnAsGnCsAn |
| 29 | YgYgYgYgYgCsAnGsCnAsGnCsAnGsCnAsGnCsAn |
| 30 | YgYgYgYgCsAnGsCnAsGnCsAnGsCnAsGnCsAn |
| 31 | YgYgYgCsAnGsCnAsGnCsAnGsCnAsGnCsAn |
| 32 | CsAnGsCnAsGnCsAnGsCnAsGnCsAnYgYgYgYgYgYgYg |
| 33 | CsAnGsCnAsGnCsAnGsCnAsGnCsAnYgYgYg |
| 34 | YgYgYgYgYgYgYgAsGnCsAnGsCnCsGnCsAnGsCnAsAn |
| 35 | YgYgYgYgYgYgYgCsAnGsCnAsGnCsAnGsCn |
| 36 | YgYgYgYgYgYgYgCsAsGnCsAsGnCsAsGnCsAsGnCsAs |
| 37 | YgYgYgYgYgYgYgCsAsGnCsAsGnCsAsGnCs |
| 38 | YgYgYgYgYgYgYgCsAnGsCnAsGnCsAnGsCnAsGn |
| 39 | YgYgYgYgYgYgYgCsAsGnCsAsGnCsAsGnCsAsGn |
| 40 | YgYgYgYgYgYgYgCsAnGnCsAnGnCsAnGnCsAnGnCsAn |
| 41 | YgYgYgYgYgYgYgCsAnGsCnAsGnCsAnGsCnAsGnCsAnGsCnAs |
| 42 | YgYgYgYgYgYgYgCsAnGsCnAsGnCsAnGsCnAsGnCsAnGs |
| 43 | YgYgYgYgYgYgYgCsAnGsCnAsGnCsAnGsCnAsGnCsAnGsCn |
| 44 | YgYgYgYgYgYgYgCsAnGsCnAsGnCsAnGsCnAsGnCsAnGsCnAsGn |
| 45 | GgCnTgGnCgTnGgCnTgGnCgTnGg |
| 46 | GgCnTgGnCnTgGnCnTgGnCgTnGgCnTnGgCnTnGg |
| 47 | ZpCnTpZnCnTpZnCnTpZnCpTnZpCnTnZpCnTnZp |
| 48 | TpGnCnTpGnCpTnGpCnTnGp |
| 49 | YgCnYgGnCnTpGnCnTpBxCpTnGpCnTnYgCnTnYg |
| 50 | GpCnTpGnCnTgGnCnTgGnCgTnGpCnTnGpCnTnGp |
| 51 | GgCnTgGnCnTpGnCnTpGnCpTnGpCnTnGgCnTnGg |
| 52 | GpCnTpGnCnTgGnCnTpGnCgTnGpCnTnGpCnTnGp |
| 53 | GpCnTpGsCnTpGnCnTpGnCpTnGpCsTnGpCnTnGp |
| 54 | GpTnCnGpTnCnGpTnCpGnTpCnGnTpCnGnTpCnGp |
| 55 | GsCnTsGnCnTsGnCnTsGnCsTnGsCnTnGsCnTnGs |
| 56 | YgYgYgYgYgYgYgGsCnTsGnCnTsGnCnTsGnCsTnGsCnTnGsCnTnGs |
| 57 | GsCnTsGnCsTnGsCnTsGnCsTnGsCnTsGnCsTnGs |
| 58 | YgYgYgYgYgYgYgGsCnTsGnCsTnGsCnTsGnCsTnGsCnTsGnCsTnGs |
| 59 | GsCnTsGnCnTsGnCnTsGnCsTnGsCnTnGsCnTnGsCn |
| 60 | YgYgYgYgYgYgYgGsCnTsGnCnTsGnCnTsGnCsTnGsCnTnGsCnTnGsCn |
| 61 | GsCnTsGnCsTnGsCnTsGnCsTnGsCnTsGnCsTnGsCn |
| 62 | YgYgYgYgYgYgYgGsCnTsGnCsTnGsCnTsGnCsTnGsCnTsGnCsTnGsCn |
| 63 | GsCnTsGnCnTsGnCnTsGnCsTnGsCnTnGsCnTnGsCnTn |
| 64 | YgYgYgYgYgYgYgGsCnTsGnCnTsGnCnTsGnCsTnGsCnTnGsCnTnGsCnTn |
| 65 | GsCnTsGnCsTnGsCnTsGnCsTnGsCnTsGnCsTnGsCnTs |
| 66 | YgYgYgYgYgYgYgGsCnTsGnCsTnGsCnTsGnCsTnGsCnTsGnCsTnGsCnTs |
| 67 | GpCnTpGnCnTpGnCnTpGnCpTnGpCnTnGp |
| 68 | GpCnTpGnCnTpGnCnTpGnCpTnGpCnTnGpCn |
| 69 | GpCnTpGnCnTpGnCnTpGnCpTnGpCnTnGpCnTn |
| 70 | GpCnTpGnCnTpGnCnTpGnCpTnGpCnTnGpCnTnGpCn |
| 71 | GpCnTpGnCnTpGnCnTpGnCpTnGpCnTnGpCnTnGpCnTn |
| 72 | GpCnTpGnCnTpGnCnTpGnCpTnGpCnTnGpCnInGpCnTnGp |
| 73 | GpCnTpGnCnTpGnCnTpGnCpTnGpCnTnGpCnTnGpCnTnGpCn |
| 74 | GpCnTpGnCnTpGnCnTpGnCpTnGpCnTnGpCnTnGpCnTnGpCnTn |
| 75 | GpCnTnGnCnTpGnCnTpGnCpTnGpCnTnGnCnTnGp |
| 76 | GpCnTpGnCpTnGpCnTpGnCpTnGpCnTpGnCpTnGp |
| 77 | GpCnTpGnCnTpGnCnTpGnCpTnGpCnTnGpCnTnGp |
| 78 | YgYgYgYgYgYgYgGvCnTvGnCnTvGnCnTvGnCvTnGvCnTnGvGnTnGv |
| 79 | YgYgYgYgYgYgYgGvCnTvGnCvTnGvCnTvGnCvTnGvCn |
| 80 | GvCnTvGnCvTnGvCnTvGnCvTnGvCn |
| 81 | GsCnTsGnCsTnGsCnTsGnCsTnGsCn |
| 82 | YgYgYgYgYgYgYgGsCnTsGnCsTnGsCnTsGnCsTnGsCn |
| 83 | YgYgYgYgYgYgYgCsAnGsCnAsGnCsAnGsCnAsGnCsAn |

TABLE 4

| | | | Monomer Chemical |
|---|---|---|---|
| Code^{a,b,c} | Residue Structure | Nucleobase | Name^{d} |
| An | 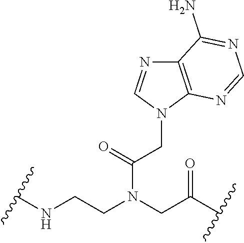 [SMILES: *NCCN(CC(*)=O)C(=O)Cn1cnc2c(N)ncnc21] | adenine | N-(2-(6-amino-9H-purin- 9-y1)acetyl)-N-(2- aminoethyl)glycine |
| Cn | 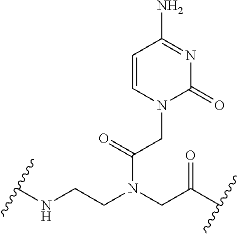 [SMILES: *NCCN(CC(*)=O)C(=O)Cn1ccc(N)nc1=O] | cytosine | N-(2-(4-amino-2- oxopyrimidin-1(2H)- yl)acetyl)-N-(2- aminoethyl)glycine |
| Gn | 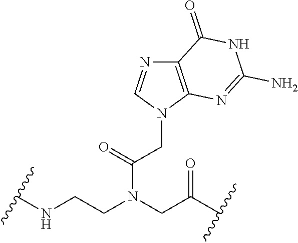 [SMILES: *NCCN(CC(*)=O)C(=O)Cn1cnc2c(=O)[nH]c(N)nc21] | guanine | N-(2-(2-amino-6-oxo-1,6- dihydro-9H-purin-9- yl)acetyl)-N-(2- aminoethyl)glycine |
| Tn | 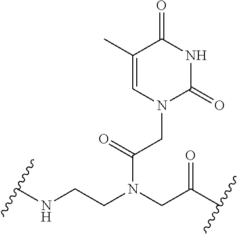 [SMILES: *NCCN(CC(*)=O)C(=O)Cn1cc(C)c(=O)[nH]c1=O] | thymine | N-(2-aminoethyl)-N-(2-(5- methyl-2,4-dioxo-3,4- dihydropyrimidin-1(2H)- yl)acetyl)glycine |
| Dn | 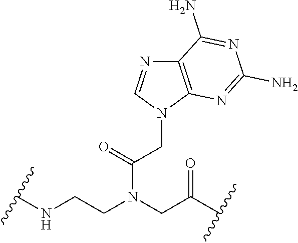 [SMILES: *NCCN(CC(*)=O)C(=O)Cn1cnc2c(N)nc(N)nc21] | 2,6- diaminopurine | N-(2-aminoethyl)-N-(2- (2,6-diamino-9H-purin-9- yl)acetyl)glycine |
| Zn | 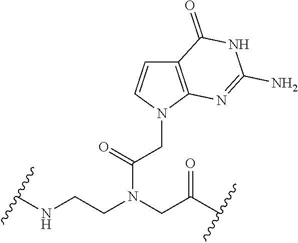 [SMILES: *NCCN(CC(*)=O)C(=O)Cn1ccc2c(=O)[nH]c(N)nc21] | 7- deazaguanine | N-(2-(2-amino-4-oxo-3,4- dihydro-7H-pyrrolo[2,3- d]pyrimidin-7-yl)acetyl)- N-(2-aminoethyl)glycine |
| Sn | 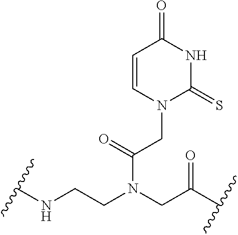 [SMILES: *NCCN(CC(*)=O)C(=O)Cn1ccc(=O)[nH]c1=S] | 2-thiouracil | N-(2-aminoethyl)-N-(2-(4- oxo-2-thioxo-3,4- dihydropyrimidin-1(2H)- yl)acetyl)glycine |
| Yn | 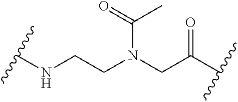 [SMILES: *NCCN(CC(*)=O)C(C)=O] | — | N-acetyl-N-(2- aminoethyl)glycine |
| As | 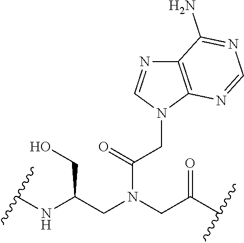 [SMILES: *N[C@@H](CO)CN(CC(*)=O)C(=O)Cn1cnc2c(N)ncnc21] | adenine | (R)-N-(2-amino-3- hydroxypropyl)-N-(2-(6- amino-9H-purin-9- yl)acetyl)glycine |
| Cs | 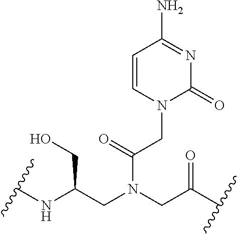 [SMILES: *N[C@@H](CO)CN(CC(*)=O)C(=O)Cn1ccc(N)nc1=O] | cytosine | (R)-N-(2-(4-amino-2- oxopyrimidin-1(2H)- yl)acetyl)-N-(2-amino-3- hydroxypropyl)glycine |
| Gs | 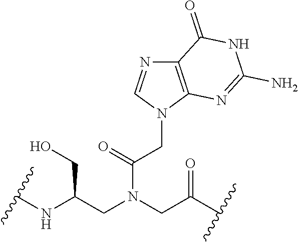 [SMILES: *N[C@@H](CO)CN(CC(*)=O)C(=O)Cn1cnc2c(=O)[nH]c(N)nc21] | guanine | (R)-N-(2-amino-3- hydroxypropyl)-N-(2-(2- amino-6-oxo-1,6-dihydro- 9H-purin-9- yl)acetyl)glycine |
| Ts | 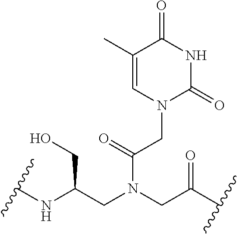 [SMILES: *N[C@@H](CO)CN(CC(*)=O)C(=O)Cn1cc(C)c(=O)[nH]c1=O] | thymine | (R)-N-(2-amino-3- hydroxypropyl)-N-(2-(5- methyl-2,4-dioxo-3,4- dihydropyrimidin-1(2H)- yl)acetyl)glycine |
| Ds | 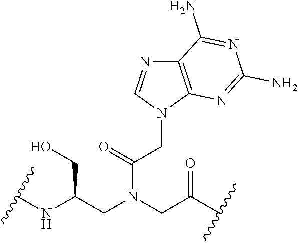 [SMILES: *N[C@@H](CO)CN(CC(*)=O)C(=O)Cn1cnc2c(N)nc(N)nc21] | 2,6- diaminopurine | (R)-N-(2-amino-3- hydroxypropyl)-N-(2-(2,6- diamino-9H-purin-9- yl)acetyl)glycine |
| Zs | 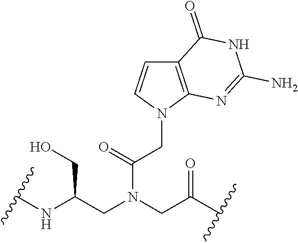 [SMILES: *N[C@@H](CO)CN(CC(*)=O)C(=O)Cn1ccc2c(=O)[nH]c(N)nc21] | 7- deazaguanine | (R)-N-(2-amino-3- hydroxypropyl)-N-(2-(2- amino-4-oxo-3,4-dihydro- 7H-pyrrolo[2,3- d]pyrimidin-7- yl)acetyl)glycine |
| Ss | 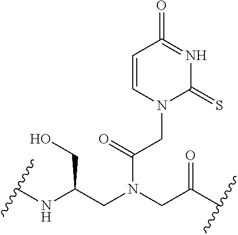 | 2-thiouracil | (R)-N-(2-amino-3- hydroxypropyl)-N-(2-(4- oxo-2-thioxo-3,4- dihydropyrimidin-1(2H)- yl)acetyl)glycine |
| Ys | 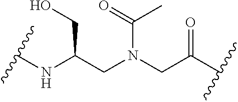 [SMILES: *N[C@@H](CO)CN(CC(*)=O)C(C)=O] | — | (R)-N-acetyl-N-(2-amino- 3-hydroxypropyl)glycine |
| Av | 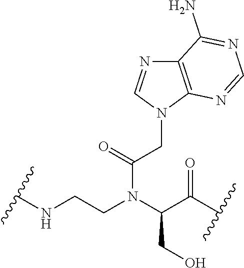 [SMILES: *NCCN(C(=O)Cn1cnc2c(N)ncnc21)[C@H](CO)C(*)=O] | adenine | N-(2-(6-amino-9H-purin- 9-yl)acetyl)-N-(2- aminoethyl)-D-serine |
| Cv | 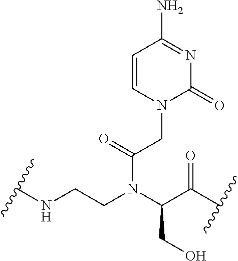 [SMILES: *NCCN(C(=O)Cn1ccc(N)nc1=O)[C@H](CO)C(*)=O] | cytosine | N-(2-(4-amino-2- oxopyrimidin-1(2H)- yl)acetyl)-N-(2- aminoethyl)-D-serine |
| Gv | 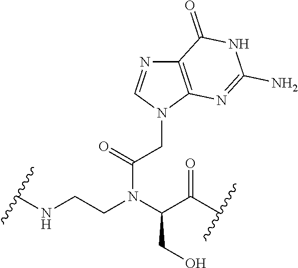 [SMILES: *NCCN(C(=O)Cn1cnc2c(=O)[nH]c(N)nc21)[C@H](CO)C(*)=O] | guanine | N-(2-(2-amino-6-oxo-1,6- dihydro-9H-purin-9- yl)acetyl)-N-(2- aminoethyl)-D-serine |
| Tv | 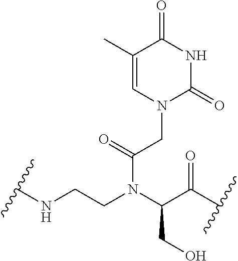 [SMILES: *NCCN(C(=O)Cn1cc(C)c(=O)[nH]c1=O)[C@H](CO)C(*)=O] | thymine | N-(2-aminoethyl)-N-(2-(5- methyl-2,4-dioxo-3,4- dihydropyrimidin-1(2H)- yl)acetyl)-D-serine |
| Dv | 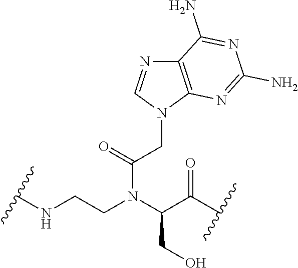 [SMILES: *NCCN(C(=O)Cn1cnc2c(N)nc(N)nc21)[C@H](CO)C(*)=O] | 2,6- diaminopurine | N-(2-aminoethyl)-N-(2- (2,6-diamino-9H-purin-9- yl)acetyl)-D-serine |
| Zv | 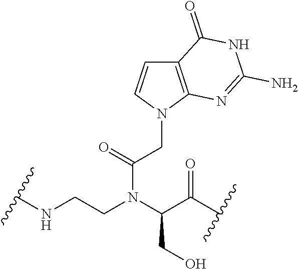 [SMILES: *NCCN(C(=O)Cn1ccc2c(=O)[nH]c(N)nc21)[C@H](CO)C(*)=O] | 7- deazaguanine | N-(2-(2-amino-4-oxo-3,4- dihydro-7H-pyrrolo[2,3- d]pyrimidin-7-yl)acetyl)- N-(2-aminoethyl)-D-serine |
| Sv | 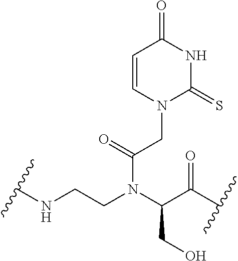 [SMILES: *NCCN(C(=O)Cn1ccc(=O)[nH]c1=S)[C@H](CO)C(*)=O] | 2-thiouracil | N-(2-aminoethyl)-N-(2-(4- oxo-2-thioxo-3,4- dihydropyrimidin-1(2H)- yl)acetyl)-D-serine |
| Yv | 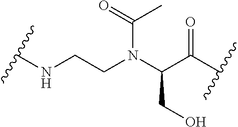 [SMILES: *NCCN(C(C)=O)[C@H](CO)C(*)=O] | — | N-acetyl-N-(2- aminoethyl)-D-serine |
| Ag | 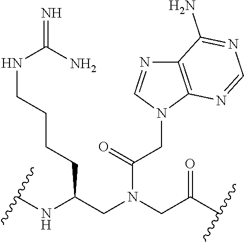 [SMILES: *N[C@@H](CCCCNC(=N)N)CN(CC(*)=O)C(=O)Cn1cnc2c(N)ncnc21] | adenine | (S)-N-(2-amino-6- guanidinohexyl)-N-(2-(6- amino-9H-purin-9- yl)acetyl)glycine |
| Cg | 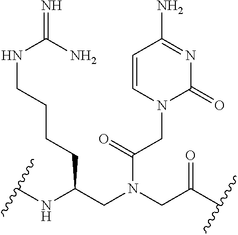 [SMILES: *N[C@@H](CCCCNC(=N)N)CN(CC(*)=O)C(=O)Cn1ccc(N)nc1=O] | cytosine | (S)-N-(2-(4-amino-2- oxopyrimidin-1(2H)- yl)acetyl)-N-(2-amino-6- guanidinohexyl)glycine |
| Gg | 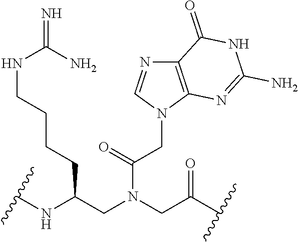 [SMILES: *N[C@@H](CCCCNC(=N)N)CN(CC(*)=O)C(=O)Cn1cnc2c(=O)[nH]c(N)nc21] | guanine | (S)-N-(2-amino-6- guanidinohexyl)-N-(2-(2- amino-6-oxo-1,6-dihydro- 9H-purin-9- yl)acetyl)glycine |
| Tg | 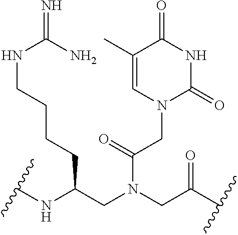 [SMILES: *N[C@@H](CCCCNC(=N)N)CN(CC(*)=O)C(=O)Cn1cc(C)c(=O)[nH]c1=O] | thymine | (S)-N-(2-amino-6- guanidinohexyl)-N-(2-(5- methyl-2,4-dioxo-3,4- dihydropyrimidin-1(2H)- yl)acetyl)glycine |
| Dg | 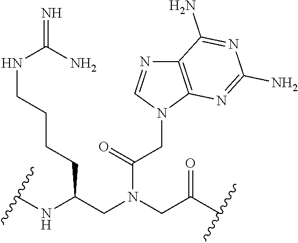 [SMILES: *N[C@@H](CCCCNC(=N)N)CN(CC(*)=O)C(=O)Cn1cnc2c(N)nc(N)nc21] | 2,6- diaminopurine | (S)-N-(2-amino-6- guanidinohexyl)-N-(2- (2,6-diamino-9H-purin-9- yl)acetyl)glycine |
| Zg | 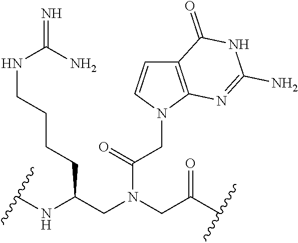 [SMILES: *N[C@@H](CCCCNC(=N)N)CN(CC(*)=O)C(=O)Cn1ccc2c(=O)[nH]c(N)nc21] | 7- deazaguanine | (S)-N-(2-(2-amino-4-oxo- 3,4-dihydro-7H- pyrrolo[2,3-d]pyrimidin-7- yl)acetyl)-N-(2-amino-6- guanidinohexyl)glycine |
| Sg | 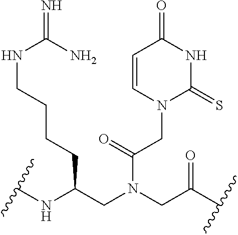 [SMILES: *N[C@@H](CCCCNC(=N)N)CN(CC(*)=O)C(=O)Cn1ccc(=O)[nH]c1=S] | 2-thiouracil | (S)-N-(2-amino-6- guanidinohexyl)-N-(2-(4- oxo-2-thioxo-3,4- dihydropyrimidin-1(2H)- yl)acetyl)glycine |
| Yg | 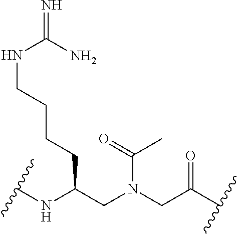 [SMILES: *N[C@@H](CCCCNC(=N)N)CN(CC(*)=O)C(C)=O] | — | (S)-N-acetyl-N-(2-amino- 6-guanidinohexyl)glycine |
| Ap | 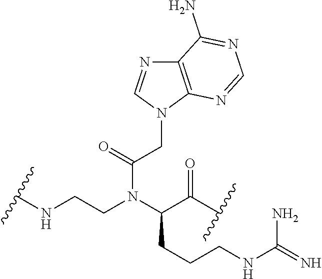 [SMILES: *NCCN(C(=O)Cn1cnc2c(N)ncnc21)[C@H](CCCNC(=N)N)C(*)=O] | adenine | N²-(2-(6-amino-9H-purin- 9-yl)acetyl)-N²-(2- aminoethyl)-D-arginine |
| Cp | 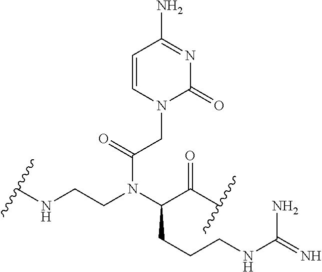 [SMILES: *NCCN(C(=O)Cn1ccc(N)nc1=O)[C@H](CCCNC(=N)N)C(*)=O] | cytosine | N²-(2-(4-amino-2- oxopyrimidin-1(2H)- yl)acetyl)-N²-(2- aminoethyl)-D-arginine |
| Gp | 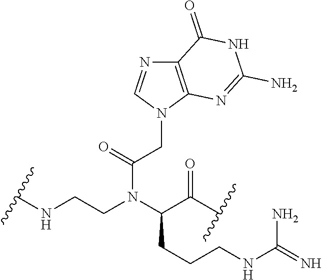 [SMILES: *NCCN(C(=O)Cn1cnc2c(=O)[nH]c(N)nc21)[C@H](CCCNC(=N)N)C(*)=O] | guanine | N²-(2-(2-amino-6-oxo-1,6- dihydro-9H-purin-9- yl)acetyl)-N²-(2- aminoethyl)-D-arginine |
| Tp | 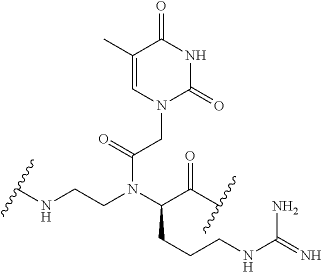 [SMILES: *NCCN(C(=O)Cn1cc(C)c(=O)[nH]c1=O)[C@H](CCCNC(=N)N)C(*)=O] | thymine | N²-(2-aminoethyl)-N²-(2- (5-methyl-2,4-dioxo-3,4- dihydropyrimidin-1(2H)- yl)acetyl)-D-arginine |
| Dp | 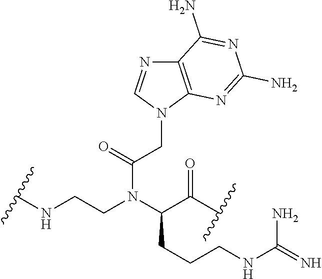 [SMILES: *NCCN(C(=O)Cn1cnc2c(N)nc(N)nc21)[C@H](CCCNC(=N)N)C(*)=O] | 2,6- diaminopurine | N²-(2-aminoethyl)-N2-(2- (2,6-diamino-9H-purin-9- yl)acetyl)-D-arginine |
| Zp | 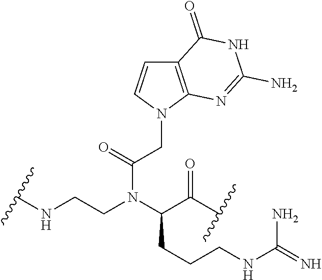 [SMILES: *NCCN(C(=O)Cn1ccc2c(=O)[nH]c(N)nc21)[C@H](CCCNC(=N)N)C(*)=O] | 7- deazaguanine | N²-(2-(2-amino-4-oxo-3,4- dihydro-7H-pyrrolo[2,3- d]pyrimidin-7-yl)acetyl)- N²-(2-aminoethyl)-D- arginine |
| Sp | 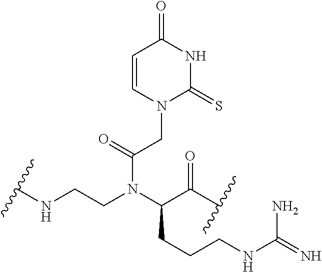 [SMILES: *NCCN(C(=O)Cn1ccc(=O)[nH]c1=S)[C@H](CCCNC(=N)N)C(*)=O] | 2-thiouracil | N²-(2-aminoethyl)-N²-(2- (4-oxo-2-thioxo-3,4- dihydropyrimidin-1(2H)- yl)acetyl)-D-arginine |
| Yp | 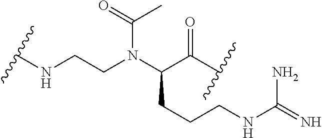 [SMILES: *NCCN(C(C)=O)[C@H](CCCNC(=N)N)C(*)=O] | — | N²-acetyl-N²-(2- aminoethyl)-D-arginine |
| Ct | 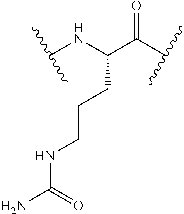 [SMILES: *N[C@@H](CCCNC(N)=O)C(*)=O] | — | L-citrulline |
| Qx | 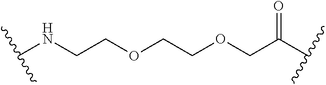 [SMILES: *NCCOCCOCC(*)=O] | — | 2-(2-(2- aminoethoxy)ethoxy)acetic acid |
| Pe&&Pe | 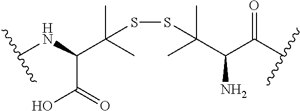 [SMILES: *N[C@H](C(=O)O)C(C)(C)SSC(C)(C)[C@H](N)C(*)=O] | — | (2R,2′R)-3,3′- disulfanediylbis(2-amino- 3-methylbutanoic acid) |
| Hx | 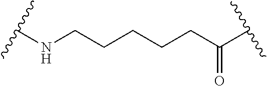 [SMILES: *NCCCCCC(*)=O] | − | 6-aminohexanoic acid |
| Bx | 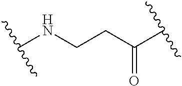 [SMILES: *NCCC(*)=O] | − | 3-aminopropanoic acid |
| Sx | 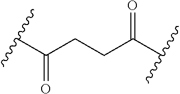 [SMILES: *C(=O)CCC(*)=O] | − | propanedioic acid |
| Px | 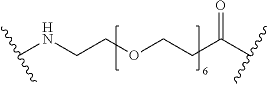 | − | Amino-PEG6-acid |
| Ve | 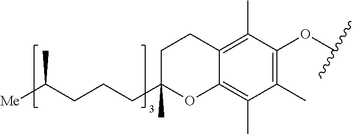 | − | Vitamin E |
| Lc | 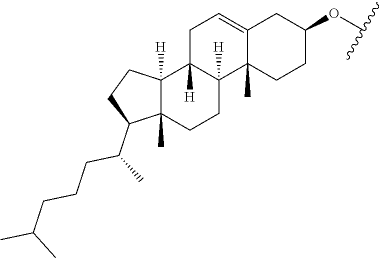 | − | Cholesterol |
| Lh | 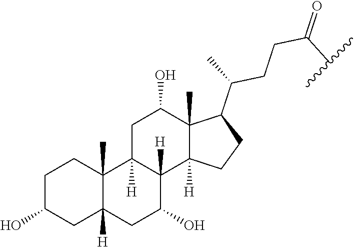 | − | Cholic acid |
| Hd | 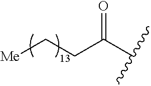 | − | Palmitic acid |
| Ac | 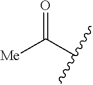 [SMILES: *C(C)=O] | − | Acetic acid |

ªProteinogenic amino acid residues in compounds provided in TABLE 1 and TABLE 2 are represented by the following one-letter codes: A: L-alanine, R: L-arginine, N: L-asparagine, D: L-aspartic acid, C: L-cysteine, E: L-glutamic acid, Q: L-glutamine, G: glycine, H: L-histidine, I: L-isoleucine, L: L-leucine , K: L-lysine, M: L-methionine, F: L-phenylalanine, P: L-proline, S: L-serine, T: L-threonine, W: L-tryptophan, Y: L-tyrosine, V: L-valine.
^{b}An antipode of an L-amino acid or chiral residue provided in this table is represented in TABLE 1 by the code of the chiral residue followed by an asterisk (*). For example, R* represents D-arginine.
^{c}In TABLE 1, C(&) represents an L-cysteine residue that is covalently bound via the sulfur atom of its side chain to a sulfur atom of another L-cysteine residue represented by C(&). For example, a compound having the sequence code GC(&)GGGGGC(&)G comprises two cysteine residues that are bound to each other via an intrachain disulfide bond.
^{d}For each residue, a chemical name is provided for the corresponding unincorporated monomer.

### Pharmaceutically-Acceptable Salts of Compounds Herein.

The disclosure provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, trifluoroacetic acid, mandelic acid, cinnamic acid, aspartic acid, stearic acid, palmitic acid, glycolic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a trifluoroacetate salt, a mandelate salt, a cinnamate salt, an aspartate salt, a stearate salt, a palmitate salt, a glycolate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

A pharmaceutically acceptable salt of a compound herein (e.g., Compound 1) can be prepared by a counterion exchange process. The counterion exchange process can be mediated by a strong anion exchange resin. The counterion exchange process can involve exposure of the compound to the strong anion exchange resin in the presence of excess acid of the desired salt form.

A compound herein (e.g., Compound 1) can comprise one or more ionizable sites, such as, for example, one or more amine moieties. In cases where a compound herein is provided as a pharmaceutically-acceptable salt, the pharmaceutically-acceptable salt can comprise one or more counterions. In some embodiments, the pharmaceutically-acceptable salt comprises a plurality of counterions. In some embodiments, the pharmaceutically-acceptable salt comprises a plurality of counterions, wherein the plurality of counterions is two counterions, three counterions, four counterions, five counterions, six counterions, seven counterions, eight counterions, nine counterions, ten counterions, eleven counterions, twelve counterions, thirteen counterions, fourteen counterion, fifteen counterions, sixteen counterions, seventeen counterions, eighteen counterions, nineteen counterions, or twenty counterions. In some embodiments, the pharmaceutically-acceptable salt comprises a plurality of counterions, wherein the plurality of counterions is two to twenty counterions. In some embodiments, at least one counterion of the plurality of counterions is independently selected from fluoride, chloride, bromide, iodide, acetate, trifluoroacetate, propionate, phosphate, nitrate, thiocyanate, and perchlorate. In some embodiments, at least one counterion of the plurality of counterions is independently selected from chloride, acetate, trifluoroacetate, and pamoate.

An amount of counterion in a pharmaceutically-acceptable salt of a compound herein (e.g., Compound 1) can be articulated in terms of a percent of compound mass that is counterion. In some embodiments, a pharmaceutically-acceptable salt of a compound herein (e.g., Compound 1), or an ionized form thereof, comprises about 1% to about 25%, about 1% to about 20%, about 1% to about 15%, about 1% to about 10%, about 1% to about 5%, about 5% to about 25%, about 5% to about 20%, about 5% to about 15%, or about 5% to about 10% (w/w) acetate. In some embodiments, a pharmaceutically-acceptable salt of a compound herein (e.g., Compound 1), or an ionized form thereof, comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30% (w/w) acetate.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutical Compositions.

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism.

In some embodiments, the present disclosure provides a pharmaceutical composition comprising:
(a) a compound comprising an oligomeric structure, wherein the oligomeric structure comprises a repeating unit of formula:

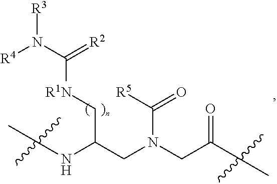

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
      or a pharmaceutically-acceptable salt or ionized form thereof; and
(b) an amino acid, or a pharmaceutically acceptable salt or ionized form thereof.

In some embodiments, the amino acid comprises a side chain that is positively charged at physiological pH. In some embodiments, the amino acid is histidine, lysine, ornithine, or arginine. In some embodiments, the amino acid is histidine. In some embodiments, the amino acid is arginine. In some embodiments, the amino acid is lysine. In some embodiments, the amino acid is ornithine. In some embodiments, the pharmaceutical composition further comprises a second amino acid or a pharmaceutically-acceptable salt or ionized form thereof. In some embodiments, the second amino acid comprises a side chain that is positively charged at physiological pH. In some embodiments, the second amino acid is arginine.

In some embodiments, the present disclosure provides a pharmaceutical composition comprising:
(a) a compound comprising an oligomeric structure, wherein the oligomeric structure comprises a repeating unit of formula (I):

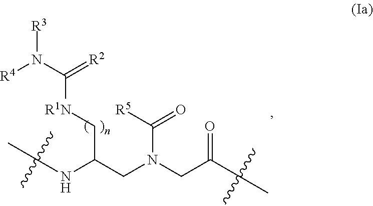

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
      or a pharmaceutically-acceptable salt or ionized form thereof; and
(b) an amino acid or a pharmaceutically-acceptable salt or ionized form thereof, wherein the amino acid comprises a side chain that is positively charged at physiological pH.

In some embodiments, the amino acid is a monomeric amino acid. In some embodiments, the amino acid is histidine, lysine, ornithine, or arginine. In some embodiments, the amino acid is histidine. In some embodiments, the amino acid is arginine. In some embodiments, the amino acid is lysine. In some embodiments, the amino acid is ornithine. In some embodiments, the pharmaceutical composition further comprises a second amino acid or a pharmaceutically-acceptable salt or ionized form thereof. In some embodiments, the second amino acid comprises a side chain that is positively charged at physiological pH. In some embodiments, the second amino acid is arginine.

In some embodiments, the pharmaceutical composition further comprises an anti-aggregation agent. In some embodiments, the anti-aggregation agent is a saccharide. In some embodiments, the anti-aggregation agent is a sugar or sugar alcohol. In some embodiments, the anti-aggregation agent is a sugar. In some embodiments, the anti-aggregation agent is a polyol.

In some embodiments, the anti-aggregation agent is a monosaccharide. In some embodiments, the anti-aggregation agent is a sugar alcohol. In some embodiments, the anti-aggregation agent is maltitol, mannitol, isomalt, sorbitol, xylitol, or erythritol. In some embodiments, the anti-aggregation agent is dextrose. In some embodiments, the anti-aggregation agent is mannitol.

In some embodiments, the present disclosure provides a pharmaceutical composition comprising:
(a) a compound comprising an oligomeric structure, wherein the oligomeric structure comprises a repeating unit of formula (I):

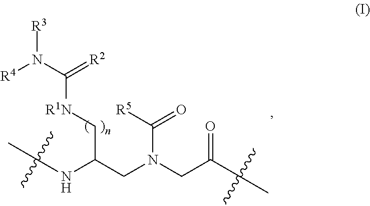

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
      or a pharmaceutically-acceptable salt or ionized form thereof; and
(b) mannitol at a concentration of about 5 mM to about 500 mM.

In some embodiments, mannitol is present in the composition at a concentration from about 5 mM to about 200 mM. In some embodiments, mannitol is present in the composition at a concentration from about 5 mM to about 25 mM, about 5 mM to about 50 mM, about 5 mM to about 75 mM, about 5 mM to about 100 mM, about 5 mM to about 150 mM, about 5 mM to about 200 mM, about 25 mM to about 75 mM, about 25 mM to about 100 mM, about 25 mM to about 150 mM, about 25 mM to about 200 mM, about 50 mM to about 75 mM, about 50 mM to about 100 mM, about 50 mM to about 150 mM, about 50 mM to about 200 mM, about 75 mM to about 100 mM, about 75 mM to about 150 mM, or about 75 mM to about 200 mM.

In some embodiments, the pharmaceutical composition further comprises a buffering agent. In some embodiments, the buffering agent is an amino acid or a pharmaceutically acceptable salt or ionized form thereof. In some embodiments, the amino acid comprises a side chain that is positively charged at physiological pH. In some embodiments, the amino acid is selected from histidine, lysine, ornithine, and arginine. In some embodiments, the amino acid is histidine. In some embodiments, the amino acid is arginine. In some embodiments, the amino acid is lysine. In some embodiments, the amino acid is ornithine.

In some embodiments, the buffering agent is a dicarboxylic acid or a pharmaceutically acceptable salt or ionized form thereof. In some embodiments, the dicarboxylic acid is malonic acid, succinic acid, glutaric acid, adipic acid, pimelic acid, or a pharmaceutically-acceptable salt thereof. In some embodiments, the buffering agent is succinic acid.

In some embodiments, the buffering agent is citric acid, acetic acid, boric acid, or phosphoric acid.

In some embodiments, the buffering agent comprises a 1,2-aminoalcohol moiety. In some embodiments, the buffering agent is TAPS ([tris(hydroxymethyl)methylamino]propanesulfonic acid), Bicine (2-(bis(2-hydroxyethyl)amino)acetic acid), Tris (tris(hydroxymethyl)aminomethane), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), or a pharmaceutically-acceptable salt thereof.

In some embodiments, the present disclosure provides a pharmaceutical composition comprising:
(a) a compound comprising an oligomeric structure, wherein the oligomeric structure comprises a repeating unit of formula:

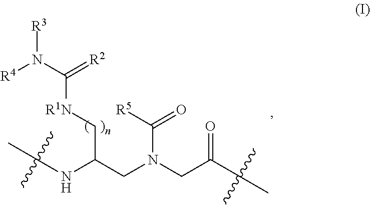

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
   or a pharmaceutically-acceptable salt or ionized form thereof, and
(b) a pharmaceutically acceptable excipient, wherein the pharmaceutical composition has an Apparent Molecular Weight (App MW) that is about 5 kDa to about 50 kDa.

In some embodiments, the pharmaceutical composition further comprises an anti-aggregation agent. In some embodiments, the anti-aggregation agent is a saccharide. In some embodiments, the anti-aggregation agent is a sugar or sugar alcohol. In some embodiments, the anti-aggregation agent is a sugar. In some embodiments, the anti-aggregation agent is a polyol. In some embodiments, the anti-aggregation agent is a monosaccharide. In some embodiments, the anti-aggregation agent is a sugar alcohol. In some embodiments, the anti-aggregation agent is maltitol, mannitol, isomalt, sorbitol, xylitol, or erythritol. In some embodiments, the anti-aggregation agent is dextrose. In some embodiments, the anti-aggregation agent is mannitol.

In some embodiments, the apparent molecular weight is from about 5 kDa to about 40 kDa, 5 kDa to about 35 kDa, about 5 kDa to about 30 kDa, about 5 kDa to about 25 kDa, about 5 kDa to about 20 kDa, about 5 kDa to about 15 kDa, about 5 kDa to about 10 kDa, 10 kDa to about 40 kDa, about 10 kDa to about 35 kDa, about 10 kDa to about 30 kDa, about 10 kDa to about 25 kDa, about 10 kDa to about 20 kDa, or about 10 kDa to about 15 kDa. In some embodiments, the apparent molecular weight is about 10 kDa to about 40 kDa.

In some embodiments, the apparent molecular weight is measured by light scattering. In some embodiments, the light scattering is static light scattering (SLS). In some embodiments, the light scattering is dynamic light scattering (DLS).

In some embodiments, each of R¹, R³, and R⁴ is hydrogen; and R² is NH or N(Pg^{N}). In some embodiments, R⁵ is linear alkyl. In some embodiments, R⁵ is methyl. In some embodiments, n is 3. In some embodiments, n is 4.

In some embodiments, the compound further comprises a first chemical moiety attached to the oligomeric structure, and a second chemical moiety attached to the oligomeric structure, wherein the oligomeric structure, wherein the first chemical moiety, and the second chemical moiety form:

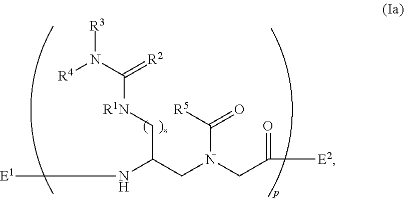

wherein:
E¹ is the first chemical moiety;
E² is the second chemical moiety; and
p is an integer that is 1-100.

In some embodiments, p is 6. In some embodiments, p is 7. In some embodiments, p is 8.

In some embodiments, E¹ is hydrogen, acyl, a group that together with the nitrogen atom to which E¹ is bound forms a carbamate, a probe, a metal chelator, an imaging agent, or a biologically-active agent; and E² is OH, OMe, NH₂, a probe, a metal chelator, an imaging agent, or a biologically-active agent. In some embodiments, E¹ is hydrogen and E² is the biologically-active agent.

In some embodiments, the biologically-active agent comprises a structure that interferes with expression of a gene associated with a neuromuscular disease phenotype. In some embodiments, the neuromuscular disease phenotype is a DM1 disease phenotype. In some embodiments, the DM1 disease phenotype is associated with a non-wild-type DM1 gene that differs from a wild type DM1 gene in a repeat expansion mutation. In some embodiments, the biologically-active agent binds to a mRNA sequence at a region that is (CUG)_{z}, wherein z is an integer from 1-100 (SEQ ID NO: 324). In some embodiments, the biologically active agent is a peptide nucleic acid according to any one of SEQ ID NOs: 1-14 and 19-25. In some embodiments, the biologically active agent and the oligomeric structure form a sequence according to any one of SEQ ID NOs: 27-44 and 83. In some embodiments, the compound is a sequence according to any one of SEQ ID NOs: 27-44 and 83.

In some embodiments, the biologically-active agent comprises a structure that interferes with expression of a gene associated with a neurodegenerative disease phenotype. In some embodiments, the neurodegenerative disease phenotype is a Huntington's disease phenotype. In some embodiments, the Huntington's disease phenotype is associated with a non-wild-type HTT gene that differs from a wild type HTT gene in a repeat expansion mutation. In some embodiments, the biologically-active agent binds to a mRNA sequence at a region that is (CAG)_{z}, wherein z is an integer from 1-100 (SEQ ID NO: 325). In some embodiments, the biologically active agent is a peptide nucleic acid according to any one of SEQ ID NOs: 15-18, 26, 45, 26, 45-47, 55, 57, 59, 61, 63, 65, 67-77, 80, and 81. In some embodiments, the biologically active agent and the oligomeric structure form a sequence according to any one of SEQ ID NOs: 27-44 and 83. In some embodiments, the compound is a sequence according to any one of SEQ ID NOs: 27-44 and 83.

In some embodiments, the biologically-active agent comprises a structure that interferes with expression of a cancer-causing protein. In some embodiments, the cancer-causing protein is mutant K-ras. In some embodiments, the cancer-causing protein is G12D K-ras. In some embodiments, the cancer-causing protein is G12C K-ras. In some embodiments, the cancer-causing protein is G12V K-ras.

In some embodiments, the biologically-active agent binds to a nucleic acid sequence encoding a cancer gene. In some embodiments, the biologically-active agent binds to a mRNA sequence transcripted from a cancer gene. In some embodiments, the biologically-active agent binds to a DNA sequence encoding a cancer gene. In some embodiments, the cancer gene is non-wild type KRAS. In some embodiments, the cancer gene is G12D KRAS. In some embodiments, the cancer gene is G12C KRAS. In some embodiments, the cancer gene is G12V KRAS.

In some embodiments, the biologically-active agent binds to the nucleic acid sequence encoding the mutant K-ras by interactions between the heterocycles of the R² groups and nucleobases of the nucleic acid sequence. In some embodiments, the nucleic acid sequence is a mRNA sequence. In some embodiments, the nucleic acid sequence is a DNA sequence. In some embodiments, the biologically-active agent is a peptide nucleic acid according to any one of SEQ ID NOs: 84-123, 135-142, 163-166, 193-219, and 235-277. In some embodiments, the biologically-active agent and the water solubilizing group form a sequence according to any one of SEQ ID NOs: 124-134, 143-160, 167-192, 220-234, and 278-296. In some embodiments, the compound is a sequence according to any one of SEQ ID NOs: 124-134, 143-160, 167-192, 220-234, and 278-296.

In some embodiments, the biologically-active agent is an oligonucleotide or oligonucleotide analogue. In some embodiments, the biologically-active agent is a peptide nucleic acid.

In some embodiments, the compound is:

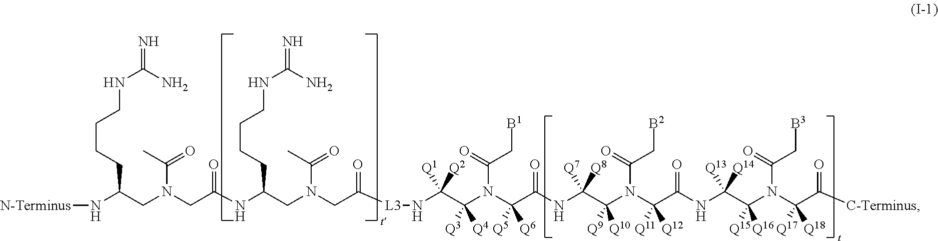

wherein:
each instance of B¹, B² and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L3 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

In some embodiments, L3 is absent. In some embodiments, each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl that is substituted or unsubstituted, or hydrogen.

In some embodiments, the compound is:

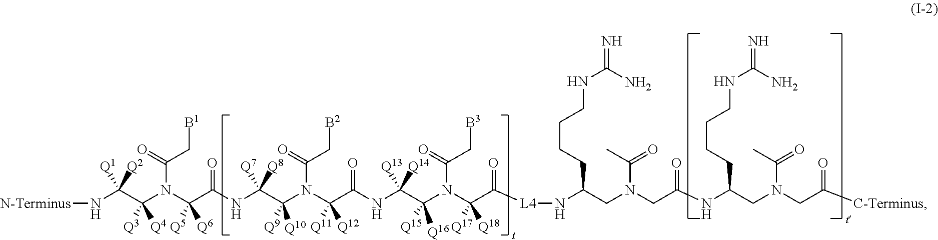

wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L4 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

In some embodiments, L4 is absent. In some embodiments, N-Terminus is H. In some embodiments, C-Terminus is NH₂.

In some embodiments, each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl that is substituted or unsubstituted, or hydrogen.

In some embodiments, the present disclosure provides a pharmaceutical composition comprising:
(a) oligonucleotide analogue comprising a structure according to formula (II):

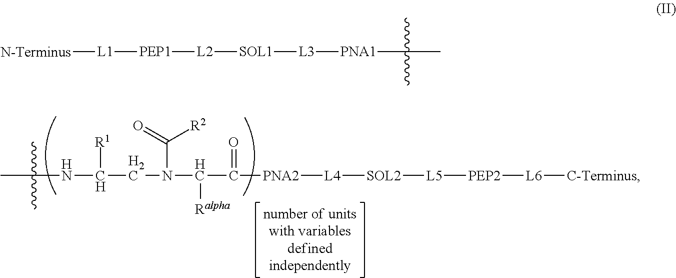

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof; and
(b) an amino acid or a pharmaceutically-acceptable salt or ionized form thereof.

In some embodiments, the amino acid comprises a side chain that is positively charged at physiological pH. In some embodiments, the amino acid comprises a side chain that is positively charged at physiological pH. In some embodiments, the amino acid is selected from histidine, lysine, ornithine, and arginine. In some embodiments, the amino acid is histidine. In some embodiments, the amino acid is arginine. In some embodiments, the amino acid is lysine. In some embodiments, the amino acid is ornithine. In some embodiments, the pharmaceutical composition further comprises a second amino acid or a pharmaceutically-acceptable salt or ionized form thereof. In some embodiments, the second amino acid comprises a side chain that is positively charged at physiological pH. In some embodiments, the second amino acid is arginine.

In some embodiments, the present disclosure provides a pharmaceutical composition comprising:
(a) an oligonucleotide analogue comprising a structure according to formula (II):

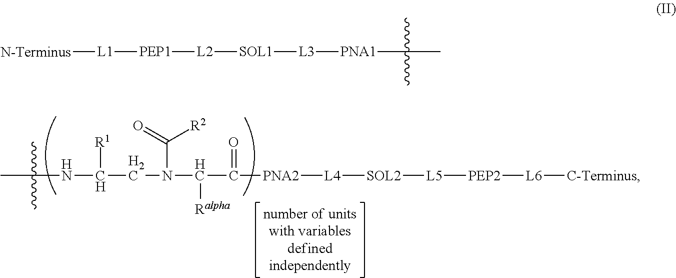

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof; and
(b) an amino acid or a pharmaceutically-acceptable salt or ionized form thereof thereof, wherein the amino acid comprises a side chain that is positively charged at physiological pH.

In some embodiments, the amino acid is histidine, lysine, ornithine, or arginine. In some embodiments, the amino acid is histidine. In some embodiments, the amino acid is arginine. In some embodiments, the amino acid is lysine. In some embodiments, the amino acid is ornithine.

In some embodiments, the pharmaceutical composition further comprises a second amino acid or a pharmaceutically-acceptable salt or ionized form thereof. In some embodiments, the second amino acid comprises a side chain that is positively charged at physiological pH. In some embodiments, the second amino acid is arginine.

In some embodiments, the pharmaceutical composition further comprises an anti-aggregation agent. In some embodiments, the anti-aggregation agent is a saccharide. In some embodiments, the anti-aggregation agent is a sugar or sugar alcohol. In some embodiments, the anti-aggregation agent is a sugar. In some embodiments, the anti-aggregation agent is a polyol.

In some embodiments, the anti-aggregation agent is a monosaccharide. In some embodiments, the anti-aggregation agent is a sugar alcohol. In some embodiments, the anti-aggregation agent is maltitol, mannitol, isomalt, sorbitol, xylitol, or erythritol. In some embodiments, the anti-aggregation agent is dextrose. In some embodiments, the anti-aggregation agent is mannitol.

In some embodiments, the present disclosure provides a pharmaceutical composition comprising:
(a) an oligonucleotide analogue comprising a structure according to formula (II):

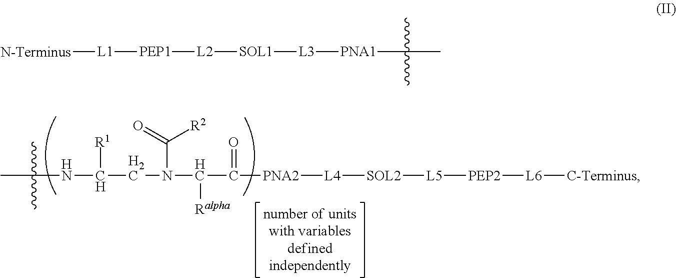

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof; and
(b) mannitol at a concentration of 5 mM to 500 mM.

In some embodiments, mannitol is present in the composition at a concentration from 5 mM to 200 mM. In some embodiments, mannitol is present in the composition at a concentration from about 5 mM to about 25 mM, about 5 mM to about 50 mM, about 5 mM to about 75 mM, about 5 mM to about 100 mM, about 5 mM to about 150 mM, about 5 mM to about 200 mM, about 25 mM to about 75 mM, about 25 mM to about 100 mM, about 25 mM to about 150 mM, about 25 mM to about 200 mM, about 50 mM to about 75 mM, about 50 mM to about 100 mM, about 50 mM to about 150 mM, about 50 mM to about 200 mM, about 75 mM to about 100 mM, about 75 mM to about 150 mM, or about 75 mM to about 200 mM.

In some embodiments, the pharmaceutical composition further comprises a buffering agent. In some embodiments, the buffering agent is an amino acid or a pharmaceutically acceptable salt or ionized form thereof. In some embodiments, the amino acid comprises a side chain that is positively charged at physiological pH. In some embodiments, the amino acid is histidine, lysine, ornithine, or arginine. In some embodiments, the amino acid is histidine. In some embodiments, the amino acid is arginine. In some embodiments, the amino acid is lysine. In some embodiments, the amino acid is ornithine.

In some embodiments, the buffering agent is a dicarboxylic acid or a pharmaceutically acceptable salt or ionized form thereof. In some embodiments, the dicarboxylic acid is malonic acid, succinic acid, glutaric acid, adipic acid, pimelic acid, or a pharmaceutically-acceptable salt thereof. In some embodiments, the buffer is succinic acid.

In some embodiments, the buffering agent is citric acid, acetic acid, boric acid, or phosphoric acid.

In some embodiments, the buffering agent comprises a 1,2-aminoalcohol moiety. In some embodiments, the buffering agent is TAPS ([tris(hydroxymethyl)methylamino]propanesulfonic acid), Bicine (2-(bis(2-hydroxyethyl)amino)acetic acid), Tris (tris(hydroxymethyl)aminomethane), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), or a pharmaceutically-acceptable salt or ionized form thereof.

In some embodiments, the present disclosure provides a pharmaceutical composition comprising:
(a) oligonucleotide analogue comprising a structure according to formula (II):

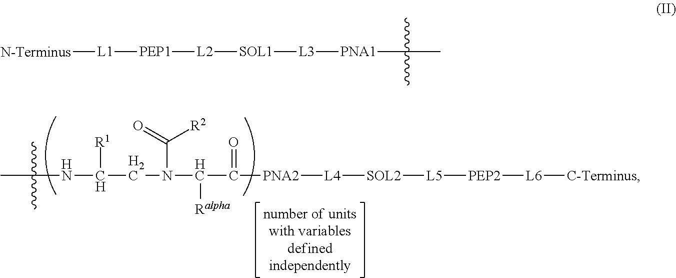

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof; and
(b) a pharmaceutically acceptable excipient.
   wherein the pharmaceutical composition has an apparent molecular weight that is about 5 kDa to about 50 kDa.

In some embodiments, the pharmaceutical composition further comprises an anti-aggregation agent. In some embodiments, the anti-aggregation agent is a saccharide. In some embodiments, the anti-aggregation agent is a sugar or sugar alcohol. In some embodiments, the anti-aggregation agent is a sugar. In some embodiments, the anti-aggregation agent is a polyol.

In some embodiments, the anti-aggregation agent is a monosaccharide. In some embodiments, the anti-aggregation agent is a sugar alcohol. In some embodiments, the anti-aggregation agent is maltitol, mannitol, isomalt, sorbitol, xylitol, or erythritol. In some embodiments, the anti-aggregation agent is dextrose. In some embodiments, the anti-aggregation agent is mannitol.

In some embodiments, the apparent molecular weight is from about 5 kDa to about 40 kDa, 5 kDa to about 35 kDa, about 5 kDa to about 30 kDa, about 5 kDa to about 25 kDa, about 5 kDa to about 20 kDa, about 5 kDa to about 15 kDa, about 5 kDa to about 10 kDa, 10 kDa to about 40 kDa, about 10 kDa to about 35 kDa, about 10 kDa to about 30 kDa, about 10 kDa to about 25 kDa, about 10 kDa to about 20 kDa, or about 10 kDa to about 15 kDa. In some embodiments, the apparent molecular weight is about 10 kDa to about 40 kDa.

In some embodiments, the apparent molecular weight is measured by light scattering. In some embodiments, the light scattering is static light scattering (SLS). In some embodiments, the light scattering is dynamic light scattering (DLS).

In some embodiments, the number of units with variables defined independently is 11, 12, 13, 14, 15, 16, or 17. In some embodiments, the number of units with variables defined independently is 14.

In some embodiments, N-Terminus is H and C-Terminus is NH₂.

In some embodiments, each R¹ is independently H, hydroxylmethyl, or 4-guanidinobut-1-yl. In some embodiments, at least one iteration of R¹ is hydroxylmethyl. In some embodiments, at least half the iterations of R¹ are hydroxylmethyl and the other iterations of R¹ are H.

In some embodiments, each of L1, L2, L3, L4, L5, and L6 is absent. In some embodiments, PEP1 and PEP2 are absent. In some embodiments, one of PEP1 and PEP2 is a peptide sequence that is a nuclear localization sequence and the other is absent. In some embodiments, SOL1 is the water-solubilizing group and SOL2 is absent. In some embodiments, each of L1, L2, L3, L4, L5, L6, PEP1, PEP2, and SOL2 is absent, and SOL1 is the water-solubilizing group.

In some embodiments, the water-solubilizing group is a group that contains multiple positive charges at physiological pH.

In some embodiments, the water-solubilizing group of SOL1 is a group of formula:

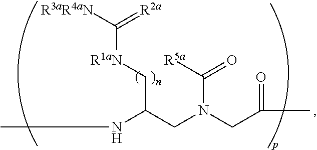

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is alkyl or O-alkyl, any of which is unsubstituted or substituted;
n is 1, 2, 3, 4, 5, 6, 7, 8, 9, or 10; and
p is an integer from 1-100.

In some embodiments, p is 5, 6, 7, or 8. In some embodiments, p is 7.

In some embodiments, the water-solubilizing group of SOL1 is a group of formula:

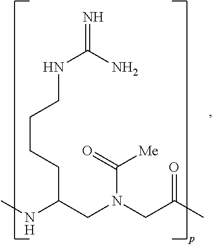

wherein p is an integer that is 5, 6, 7, or 8.

In some embodiments, the water-solubilizing group of SOL1 is a group of formula:

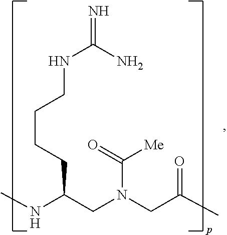

wherein p is an integer that is 5, 6, 7, or 8.

In some embodiments, p is 7.

In some embodiments, the heterocycles of the R² groups are each independently:

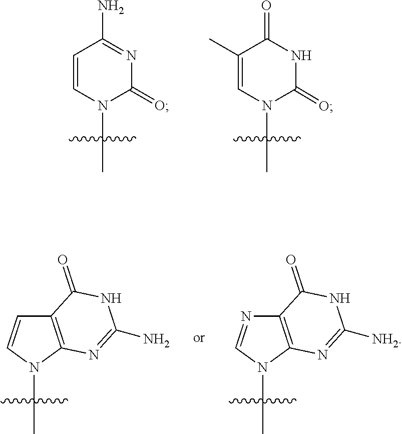

In some embodiments, each R² is independently: methyl,

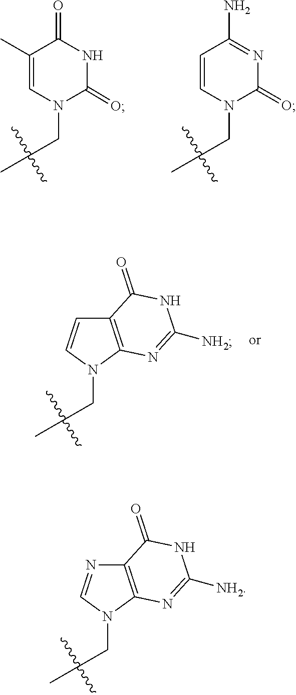 [SMILES: *Cn1cc(C)c(=O)[nH]c1=O ; *Cn1ccc(N)nc1=O ; *Cn1ccc2c(=O)[nH]c(N)nc21 ; "or" ; *Cn1cnc2c(=O)[nH]c(N)nc21]

In some embodiments, the heterocycles of the R² groups form a sequence that repeats at least twice, wherein the sequence is, from N-Terminus to C-Terminus:

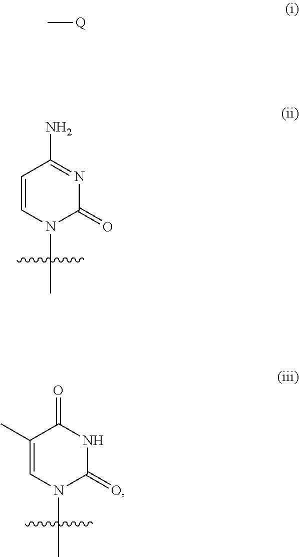

wherein Q is

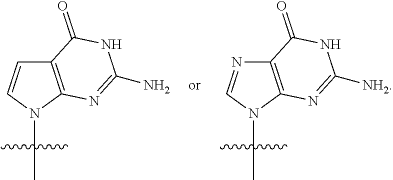 [SMILES: *n1ccc2c(=O)[nH]c(N)nc21 ; *n1cnc2c(=O)[nH]c(N)nc21]

In some embodiments, the heterocycles of the R² groups are each independently:

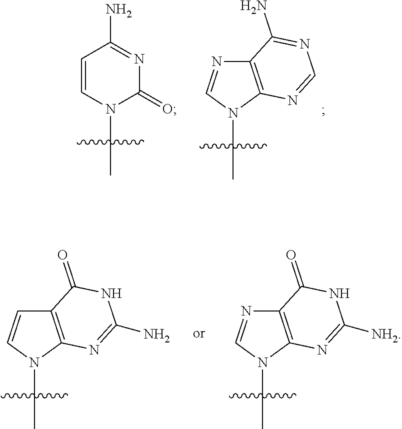

In some embodiments, each R² is independently: methyl,

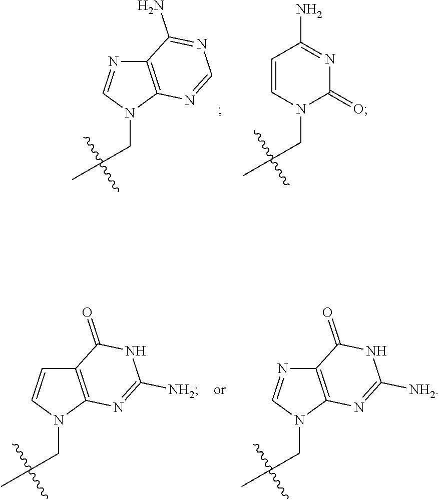 [SMILES: *Cn1cnc2c(N)ncnc21 ; *Cn1ccc(N)nc1=O ; *Cn1ccc2c(=O)[nH]c(N)nc21 ; *Cn1cnc2c(=O)[nH]c(N)nc21]

In some embodiments, the heterocycles of the R² groups form a sequence that repeats at least twice, wherein the sequence is, from N-Terminus to C-Terminus:

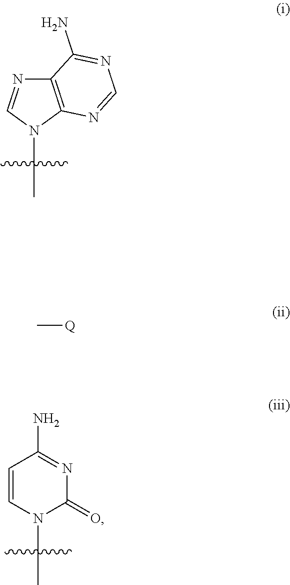

wherein Q is

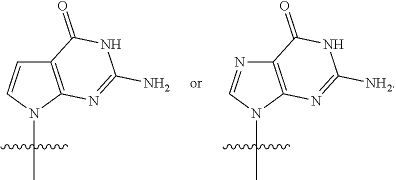 [SMILES: *n1ccc2c(=O)[nH]c(N)nc21 ; *n1cnc2c(=O)[nH]c(N)nc21]

In some embodiments, the heterocycles of the R² groups are each independently:

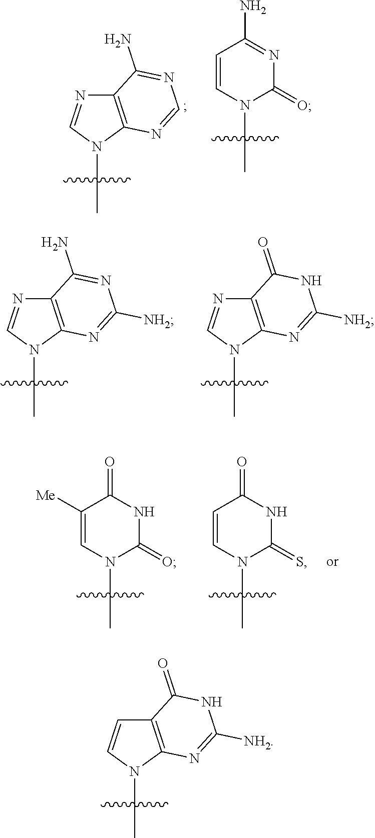

In some embodiments, each R² is independently: methyl,

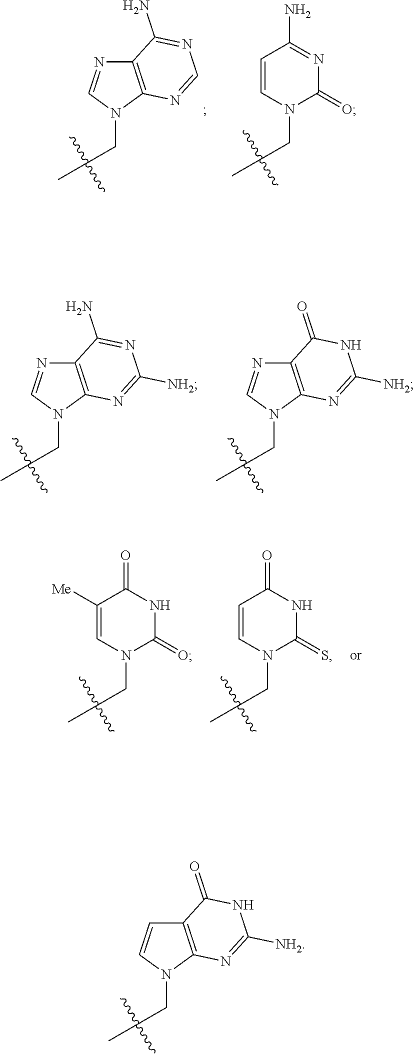

In some embodiments, the heterocycles of the R² groups form a sequence that repeats at least twice, wherein the sequence is, from N-Terminus to C-Terminus:

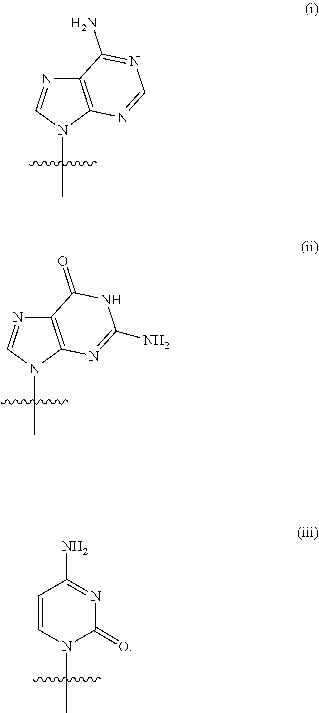 [SMILES: *n1cnc2c(N)ncnc21 ; *n1cnc2c(=O)[nH]c(N)nc21 ; *n1ccc(N)nc1=O]

In some embodiments, the compound binds to a nucleic acid sequence transcribed from a gene associated with a neuromuscular disease phenotype. In some embodiments, the compound binds to a nucleic acid sequence transcribed from a gene associated with a DM1 disease phenotype.

In some embodiments, the compound comprises a structure that interferes with expression of a gene associated with a neuromuscular disease phenotype. In some embodiments, the neuromuscular disease phenotype is a DM1 disease phenotype. In some embodiments, the DM1 disease phenotype is associated with a non-wild-type DM1 gene that differs from a wild type DM1 gene in a repeat expansion mutation. In some embodiments, the compound binds to a mRNA sequence at a region that is (CUG)_{z}, wherein z is an integer from 1-100 (SEQ ID NO: 324). In some embodiments, the units with variables defined independently form a sequence according to any one of SEQ ID NOs: 1-14 and 19-25. In some embodiments, the units with variables defined independently and the water solubilizing group form a sequence according to any one of SEQ ID NOs: 27-44 and 83. In some embodiments, the compound comprises a sequence according to any one of SEQ ID NOs: 1-14, 19-25, and 27-44 and 83. In some embodiments, the compound is a sequence according to any one of SEQ ID NOs: 1-14, 19-25, and 27-44 and 83.

In some embodiments, the compound binds to a nucleic acid sequence transcribed from a gene associated with a neurodegenerative disease phenotype. In some embodiments, the compound binds to a nucleic acid sequence transcribed from a gene associated with a Huntington's disease phenotype.

In some embodiments, the compound binds to a nucleic acid sequence transcribed from a gene associated with a Huntington's disease phenotype by interactions between the heterocycles of the R² groups and nucleobases of a HTT gene. In some embodiments, the HTT gene is a non-wild type HTT gene. In some embodiments, the non-wild type HTT gene differs from a wild type HTT gene in a repeat expansion mutation. In some embodiments, the compound binds to a mRNA sequence at a region that is (CAG)_{z}, wherein z is an integer from 1-100 (SEQ ID NO: 325). In some embodiments, the units with variables defined independently form a peptide nucleic acid according to any one of SEQ ID NOs: 15-18, 26, 45, 26, 45-47, 55, 57, 59, 61, 63, 65, 67-77, 80, and 81. In some embodiments, the units with variables defined independently and the water solubilizing group form a sequence according to any one of SEQ ID NOs: 56, 58, 60, 62, 64, 66, 78, 79, and 82. In some embodiments, the compound comprises a sequence according to any one of SEQ ID NOs: 15-18, 26, 45, 26, 45-47, and 55-82. In some embodiments, the compound is a sequence according to any one of SEQ ID NOs: 15-18, 26, 45, 26, 45-47, and 55-82.

In some embodiments, the compound binds to a nucleic acid sequence encoding a cancer-causing protein. In some embodiments, the cancer-causing protein is mutant K-ras. In some embodiments, the cancer-causing protein is G12D K-ras. In some embodiments, the cancer-causing protein is G12C K-ras. In some embodiments, the cancer-causing protein is G12V K-ras. In some embodiments, the compound binds to the nucleic acid sequence encoding the mutant K-ras by interactions between the heterocycles of the R² groups and nucleobases of the nucleic acid sequence. In some embodiments, the nucleic acid sequence is a mRNA sequence. In some embodiments, the nucleic acid sequence is a DNA sequence. In some embodiments, the units with variables defined independently form a peptide nucleic acid according to any one of SEQ ID NOs: 84-123, 135-142, 163-166, 193-219, and 235-277. In some embodiments, the units with variables defined independently and the water solubilizing group form a sequence according to any one of SEQ ID NOs: 124-134, 143-160, 167-192, 220-234, and 278-296. In some embodiments, the compound comprises a sequence according to any one of SEQ ID NOs: 84-296. In some embodiments, the compound is a sequence according to any one of SEQ ID NOs: 84-296.

In some embodiments, the compound is:

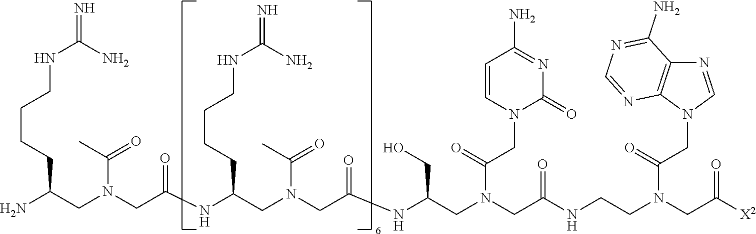

wherein X² is

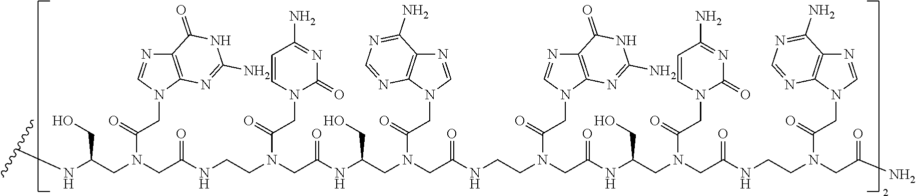

(SEQ ID NO: 27) or a pharmaceutically-acceptable salt or ionized form thereof.

In some embodiments, the compound is:

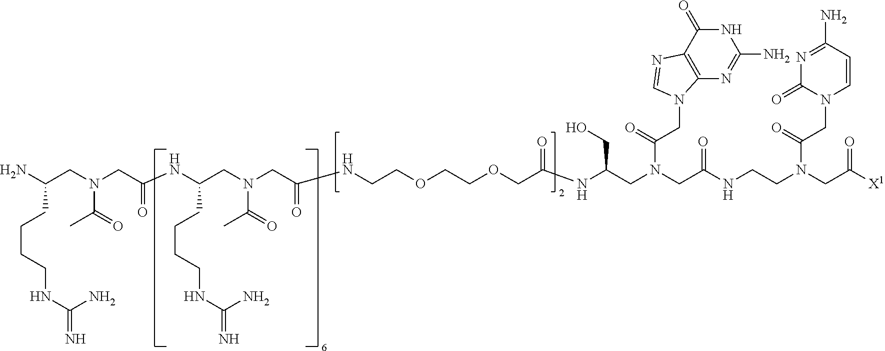

wherein X¹ is

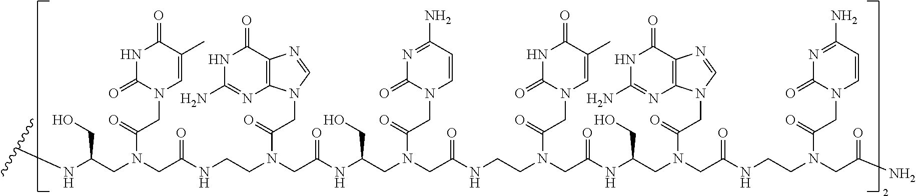

(Compound 166) (SEQ ID NO: 81), or a pharmaceutically-acceptable salt or ionized form thereof.

In some embodiments, the compound is:

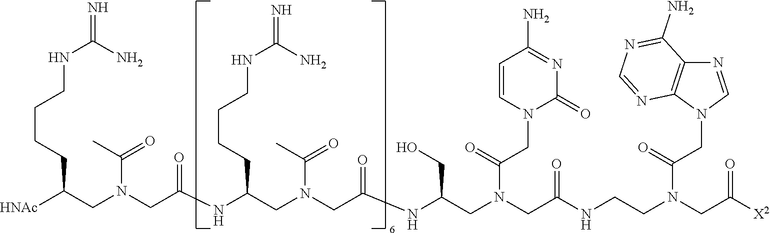

wherein X² is

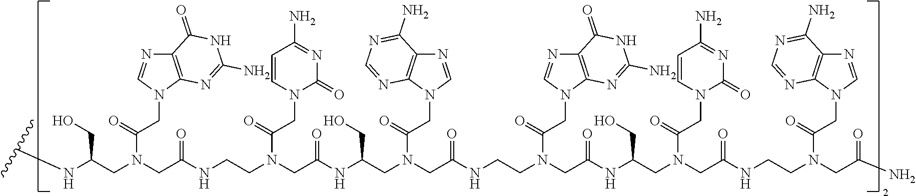

(Compound 167) (SEQ ID NO: 27), or a pharmaceutically-acceptable salt or ionized form thereof.

In some embodiments, the compound is present in the pharmaceutical composition at a concentration from about 1 mg/mL to about 300 mg/mL, about 5 mg/mL to about 300 mg/mL, about 10 mg/mL to about 300 mg/mL, about 20 mg/mL to about 300 mg/mL, about 30 mg/mL to about 300 mg/mL, about 40 mg/mL to about 300 mg/mL, about 50 mg/mL to about 300 mg/mL, about 60 mg/mL to about 300 mg/mL, about 70 mg/mL to about 300 mg/mL, about 80 mg/mL to about 300 mg/mL, about 90 mg/mL to about 300 mg/mL, about 100 mg/mL to about 300 mg/mL, about 1 mg/mL to about 200 mg/mL, about 5 mg/mL to about 200 mg/mL, about 10 mg/mL to about 200 mg/mL, about 20 mg/mL to about 200 mg/mL, about 30 mg/mL to about 200 mg/mL, about 40 mg/mL to about 200 mg/mL, about 50 mg/mL to about 200 mg/mL, about 60 mg/mL to about 200 mg/mL, about 70 mg/mL to about 200 mg/mL, about 80 mg/mL to about 200 mg/mL, about 90 mg/mL to about 200 mg/mL, about 100 mg/mL to about 200 mg/mL, about 20 mg/mL to about 100 mg/mL, about 30 mg/mL to about 100 mg/mL, about 40 mg/mL to about 100 mg/mL, about 50 mg/mL to about 100 mg/mL, about 60 mg/mL to about 100 mg/mL, about 70 mg/mL to about 100 mg/mL, about 80 mg/mL to about 100 mg/mL, or about 90 mg/mL to about 100 mg/mL.

A pharmaceutical composition of the disclosure can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

In some embodiments, a compound provided herein or a composition comprising a compound provided herein (e.g., a pharmaceutical composition) is administered orally. In some embodiments, a compound provided herein or a composition comprising a compound provided herein (e.g., a pharmaceutical composition) is administered by an intravenous, intratumoral, subcutaneous, intramuscular, intracerebral, intracerebroventricular, intra-articular, intraperitoneal, intracranial, intrathecal, intranasal, buccal, sublingual, oral, or rectal administration route. In some embodiments, a compound provided herein or a composition comprising a compound provided herein (e.g., a pharmaceutical composition) is administered by intravenous administration. In some embodiments, a compound provided herein or a composition comprising a compound provided herein (e.g., a pharmaceutical composition) is administered by subcutaneous administration. In some embodiments, a compound provided herein or a composition comprising a compound provided herein (e.g., a pharmaceutical composition) is administered by intramuscular administration. In some embodiments, a compound provided herein or a composition comprising a compound provided herein (e.g., a pharmaceutical composition) is administered by intracerebroventricular administration. In some embodiments, a compound provided herein or a composition comprising a compound provided herein (e.g., a pharmaceutical composition) is administered by oral administration. In some embodiments, a compound provided herein or a composition comprising a compound provided herein (e.g., a pharmaceutical composition) is administered by intrathecal administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended-release formulation, or in the form of an intermediate release formulation. A rapid release form can provide an immediate release. An extended-release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water soluble form. Suspensions of the active compounds can be prepared as oily injection suspensions.

Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives.

The compounds of the disclosure can be applied topically to the skin, or a body cavity, for example, oral, vaginal, bladder, cranial, spinal, thoracic, or pelvic cavity of a subject. The compounds of the disclosure can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, and synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers, and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Solid composition can include lyophilized formulations. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the disclosure is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the disclosure can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Sugars.

A compound described herein can be present in a composition with one or more sugars. Sugars can decrease aggregation in the pharmaceutical composition via formation of hydrogen bonds with polar groups of an active ingredient. In some embodiments, the sugar is a monosaccharide. Non-limiting examples of monosaccharides include glyceraldehyde, dihydroxyacetone, erythrose, threose, erythrulose, arabinose, lyxose, ribose, xylose, ribulose, xylulose, allose, altrose, galactose, glucose, idose, mannose, talose, fructose, psicose, sorbose, tagatose, mannoheptulose, sedoheptulose, octolose, 2-keto-3-deoxy-manno-octonate, sialose, dextrose, and mannitol. In some embodiments, the monosaccharide is mannitol. In some embodiments, the monosaccharide is dextrose. In some embodiments, the pharmaceutical composition comprises dextrose and mannitol. In some embodiments, the sugar is a disaccharide. Non-limiting examples of disaccharides include sucrose, maltose, lactose, lactulose, trehalose, cellbiose, or chitobiose. In some embodiments, the disaccharide is sucrose. In some embodiments, the pharmaceutical composition comprises one or more monosaccharides. In some embodiments, the pharmaceutical composition comprises one or more disaccharides. In some embodiments, the pharmaceutical composition comprises one or more monosaccharides and one or more disaccharides.

The concentration of the sugar (e.g., mannitol, dextrose, or sucrose) present in a pharmaceutical composition of the disclosure, for example can be from about 1 mM to about 1 M, from about 1 mM to about 1 mM, from about 1 mM to about 5 mM, from about 1 mM to about 10 mM, from about 5 mM to about 10 mM, from about 10 mM to about 15 mM, from about 15 mM to about 20 mM, from about 20 mM to about 25 mM, from about 25 mM to about 30 mM, from about 30 mM to about 35 mM, from about 35 mM to about 40 mM, from about 40 mM to about 45 mM, about 45 mM to about 50 mM, from about 50 mM to about 55 mM, from about 55 mM to about 60 mM, from about 60 mM to about 65 mM, from about 65 mM to about 70 mM, from about 70 mM to about 75 mM, about 75 mM to about 80 mM, from about 80 mM to about 85 mM, from about 85 mM to about 90 mM, from about 90 mM to about 95 mM, from about 95 mM to about 100 mM, from about 100 mM to about 110 mM, from about 110 mM to about 120 mM, from about 120 mM to about 130 mM, from about 130 mM to about 140 mM, from about 140 mM to about 150 mM, from about 150 mM to about 160 mM, from about 160 mM to about 170 mM, from about 170 mM to about 180 mM, from about 180 mM to about 190 mM, from about 190 mM to about 200 mM, from about 200 mM to about 210 mM, from about 210 mM to about 220 mM, from about 220 mM to about 230 mM, from about 230 mM to about 240 mM, from about 240 mM to about 250 mM, from about 250 mM to about 260 mM, from about 260 mM to about 270 mM, from about 270 mM to about 280 mM, from about 280 mM to about 290 mM, from about 290 mM to about 300 mM, from about 300 mM to about 310 mM, from about 320 mM to about 330 mM, from about 330 mM to about 340 mM, from about 340 mM to about 350 mM, from about 350 mM to about 360 mM, from about 360 mM to about 370 mM, from about 370 mM to about 380 mM, from about 380 mM to about 390 mM, from about 390 mM to about 400 mM, from about 400 mM to about 410 mM, from about 410 mM to about 420 mM, from about 420 mM to about 430 mM, from about 430 mM to about 440 mM, from about 440 mM to about 450 mM, from about 450 mM to about 460 mM, from about 460 mM to about 470 mM, from about 470 mM to about 480 mM, from about 480 mM to about 490 mM, from about 490 mM to about 500 mM, from about 500 mM to about 550 mM, from about 550 mM to about 600 mM, from about 600 mM to about 650 mM, from about 650 mM to about 700 mM, from about 700 mM to about 750 mM, from about 750 mM to about 800 mM, from about 850 mM to about 900 mM, from about 950 mM to about 1 M. The concentration of the sugar (e.g., mannitol, dextrose, or sucrose) present in a pharmaceutical composition of the disclosure can be at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 90 mM, at least about 95 mM, at least about 100 mM, at least about 110 mM, at least about 120 mM, at least about 130 mM, at least about 140 mM, at least about 150 mM, at least about 160 mM, at least about 170 mM, at least about 180 mM, at least about 190 mM, at least about 200 mM, at least about 210 mM, at least about 220 mM, at least about 230 mM, at least about 240 mM, at least about 250 mM, at least about 260 mM, at least about 270 mM, at least about 280 mM, at least about 290 mM, at least about 300 mM, at least about 310 mM, at least about 320 mM, at least about 330 mM, at least about 330 mM, at least about 340 mM, at least about 350 mM, at least about 360 mM, at least about 370 mM, at least about 380 mM, at least about 390 mM, at least about 400 mM, at least about 410 mM, at least about 420 mM, at least about 430 mM, at least about 440 mM, at least about 450 mM, at least about 460 mM, at least about 470 mM, at least about 480 mM, at least about 490 mM, at least about 500 mM, at least about 550 mM, at least about 600 mM, at least about 650 mM, at least about 700 mM, at least about 750 mM, at least about 800 mM, at least about 850 mM, at least about 900 mM, at least about 950 mM, at least about 1 M, or more. The concentration of the sugar (e.g., mannitol, dextrose, or sucrose) present in a pharmaceutical composition of the disclosure can be at most about 1 M, at most about 950 mM, at most about 900 mM, at most about 850 mM, at most about 800 mM, at most about 750 mM, at most about 700 mM, at most about 650 mM, at most about 550 mM, at most about 500 mM, at most about 490 mM, at most about 480 mM, at most about 470 mM, at most about 460 mM, at most about 450 mM, at most about 440 mM, at most about 430 mM, at most about 420 mM, at most about 410 mM, at most about 400 mM, at most about 390 mM, at most about 380 mM, at most about 370 mM, at most about 360 mM, at most about 350 mM, at most about 340 mM, at most about 330 mM, at most about 320 mM, at most about 310 mM, at most about 300 mM, at most about 290 mM, at most about 280 mM, at most about 270 mM, at most about 260 mM, at most about 250 mM, at most about 240 mM, at most about 230 mM, at most about 220 mM, at most about 210 mM, at most about 200 mM, at most about 190 mM, at most about 180 mM, at most about 170 mM, at most about 160 mM, at most about 150 mM, at most about 140 mM, at most about 130 mM, at most about 120 mM, at most about 110 mM, at most about 100, at most about 95 mM, at most about 90 mM, at most about 85 mM, at most about 80 mM, at most about 75 mM, at most about 70 mM, at most about 65 mM, at most about 60 mM, at most about 55 mM, at most about 50 mM, at most about 45 mM, at most about 40 mM, at most about 35 mM, at most about 30 mM, at most about 25 mM, at most about 20 mM, at most about 15 mM, at most about 10 mM, at most about 9 mM, at most about 8 mM, at most about 7 mM, at most about 6 mM, at most about 5 mM, at most about 4 mM, at most about 3 mM, at most about 2 mM, at most about 1 mM, or less. The concentration of the sugar (e.g., mannitol, dextrose, or sucrose) present in a pharmaceutical composition of the disclosure can be about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 90 mM, about 95 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, about 500 mM, about 550 mM, about 600 mM, about 650 mM, about 700 mM, about 750 mM, about 800 mM, about 850 mM, about 900 mM, about 950 mM, or about 1 M.

### Amino Acid Buffering Agents.

A compound described herein can be present in a composition with one or more buffering agents, such as an amino acid or pharmaceutically acceptable salt thereof or ionized form thereof. In some embodiments, the amino acid maintains a pH of the composition of below about 7. In some embodiments, a composition of the present disclosure exhibits decreased aggregation in solutions with a pH of below about 7. In some embodiments, the amino acid is positively-charged at physiological pH. In some embodiments, the amino acid is lysine. In some embodiments, the amino acid is arginine. In some embodiments, a hydrophilic amino acid buffering agent is histidine. In some embodiments, the amino acid buffering agent is negatively-charged at physiological pH. In some embodiments, the negatively-charged amino acid buffer is an aspartic acid buffer. In some embodiments, the negatively-charged amino acid buffer is a glutamic acid buffer. In some embodiments, the amino acid buffer is a hydrophilic amino acid buffer. In some embodiments, the hydrophilic amino acid buffer is a serine buffer. In some embodiments, the hydrophilic amino acid buffer is a threonine buffer. In some embodiments, the hydrophilic amino acid buffer is a tyrosine buffer. In some embodiments, the hydrophilic amino acid buffer is an asparagine amino acid buffer. In some embodiments, the hydrophilic amino acid buffer is a glutamine buffer.

In some embodiments, the amino acid is a hydrophobic amino acid. In some embodiments, the hydrophobic amino acid is valine. In some embodiments, the hydrophobic amino acid buffer is a leucine buffer. In some embodiments, the hydrophobic amino acid buffer is an isoleucine buffer. In some embodiments, the hydrophobic amino acid buffer is a methionine buffer. In some embodiments, the hydrophobic amino acid buffer is a phenylalanine buffer. In some embodiments, the hydrophobic amino acid buffer is a glycine buffer. In some embodiments, the hydrophobic amino acid buffer is an alanine buffer. In some embodiments, the hydrophobic amino acid buffer is a cysteine buffer. In some embodiments, the hydrophobic amino acid buffer is a proline buffer. In some embodiments, the hydrophobic amino acid buffer is a tryptophan buffer.

In some embodiments, a buffer is prepared by mixing a base form of a buffering agent (e.g., an amino acid) with an acid form of the buffering agent. In some embodiments, the buffering agent is histidine. In some embodiments, the acid form of histidine is histidine monohydrochloride monohydrate. In some examples, the base form of histidine is histidine free base. In some embodiments, the amino acid buffer is prepared from equal parts of the acid form and the base form of the amino acid. In some embodiments, an amino acid buffer is prepared from 25 nM of the acidic form of the amino acid and 25 mM of the basic form of the amino acid. In some embodiments, the amino acid buffer comprises different proportions of the acid form and the base form of the amino acid. In some embodiments, the ratio of the acidic form of the amino acid to the basic form of the amino acid or a non-amino acid buffering agent is at least about 1:1, at least about 2:1, at least about 3:1, at least about 4:1, at least about 5:1, at least about 6:1, at least about 8:1, at least about 10:1, at least about 20:1, at least about 40:1, at least about 60:1, at least about 80:1, at least about 100:1, at least about 120:1, at least about 125:1, at least about 150:1, or more. In some embodiments, the ratio of the acidic form of the amino acid to the basic form of the amino acid is at most about 150:1, at most about 125:1, at most about 120:1, at most about 100:1, at most about 80:1, at most about 60:1, at most about 40:1, at most about 20:1, at most about 10:1, at most about 8:1, at most about 6:1, at most about 5:1, at most about 4:1, at most about 3:1, at most about 2:1, at most about 1:1, or less. In some embodiments, the ratio of the acidic form of the amino acid to the basic form of the amino acid is about 1:1, about 2:1, about 3:1, about 4:1, about 5:1, about 6:1, about 8:1, about 10:1, about 20:1, about 40:1, about 60:1, about 80:1, about 100:1, about 120:1, about 125:1, about 150:1, or more. In some embodiments, the ratio of the acidic form of the amino acid buffer to the basic form of the amino acid buffer can comprise from about 1:1 to about 150:1, from about 1:1 to about 2:1, from about 1:1 to about 10:1, from about 10:1 to about 20:1, from about 20:1 to about 60:1, from about 60:1 to about 80:1, from about 80:1 to about 100:1, from about 100:1 to about 120:1, and from about 120:1 to about 150:1.

Alternatively, a compound described herein can be present in a composition with one or more non-amino acid buffering agents. Non-limiting examples of non-amino acid buffering agents include phosphate buffered saline (PBS), PBS without calcium or magnesium, HEPES, Tris EDTA, sodium phosphate buffer, TRIS buffer, Tris HCL, succinate buffer, disodium succinate, succinic acid, ammonium phosphate buffer, sodium citrate/citric acid, sodium acetate/acetic acid, sodium tartrate/tartaric acid, MES, Tricine, Bis-tris methane, ADA, ACES, PIPES, MOPSO, cholamine chloride, MOPS, TES, BES, DIPSO, MOBS, Acetamidoglycine, TAPSO, TEA, POPSO, HEPPSO, EPS, HEPPS, HEPES, Glycinamide, Glycylglycine, HEPBS, Bicine, TAPS, AMIPB, CHES, CAPSO, AMP, CAPS, CABS, sodium lactate/lactic acid, sodium malate/malic acid, ammonium chloride, ammonium acetate, ammonium hydroxide, ammonium bicarbonate, ammonium phosphate, and sodium bicarbonate. In some embodiments, the non-amino acid buffering agent is a PBS without calcium or magnesium. In some embodiments, the non-amino acid buffering agent is ammonium phosphate. In some embodiments, the non-amino acid buffering agent is sodium phosphate. In some embodiments, the non-amino acid buffering agent is Tris HCl. In some embodiments, the non-amino acid buffering agent can be a succinate buffer.

In some embodiments, the buffer is a mixture of two or more buffering agents, such as alanine, arginine, asparagine, aspartic acid, cysteine, glutamic acid, glutamine, glycine, histidine, isoleucine buffer, leucine buffer, lysine buffer, methionine buffer, phenylalanine buffer, proline buffer, serine buffer, threonine buffer, tryptophan buffer, tyrosine buffer, valine buffer, phosphate buffered saline (PBS), PBS without calcium or magnesium, HEPES, Tris EDTA, sodium phosphate buffer, TRIS buffer, Tris HCL, succinate buffer, disodium succinate, succinic acid, ammonium phosphate buffer, sodium citrate/citric acid, sodium acetate/acetic acid, sodium tartrate/tartaric acid, MES, Tricine, Bis-tris methane, ADA, ACES, PIPES, MOPSO, cholamine chloride, MOPS, TES, BES, DIPSO, MOBS, Acetamidoglycine, TAPSO, TEA, POPSO, HEPPSO, EPS, HEPPS, HEPES, Glycinamide, Glycylglycine, HEPBS, Bicine, TAPS, AMPB, CHES, CAPSO, AMP, CAPS, CABS, sodium lactate/lactic acid, sodium malate/malic acid, ammonium chloride, ammonium acetate, ammonium hydroxide, ammonium bicarbonate, ammonium phosphate, and sodium bicarbonate.

In some embodiments, the non-amino acid buffer is made by mixing a base form of the amino acid with an acid form of a buffering agent. In some embodiments, the non-amino acid buffer is made using the acid form of the buffering agent. In some embodiments, the non-amino acid buffer is made using the base form of the buffering agent. In some embodiments, the non-amino acid buffering agent is sodium phosphate. In some embodiments, the base form of sodium phosphate is sodium phosphate dibasic dihydrate. In some examples, the acid form of sodium phosphate is sodium phosphate monobasic. In some embodiments, the non-amino acid buffer is succinic acid or a salt thereof. In some examples, the acid form of succinate is succinic acid. In some examples, the base form of succinate is disodium succinate. In some embodiments, the non-amino acid buffer comprises equal parts of the acid form and the base form of the buffer compound. In some examples, a non-amino acid buffer is prepared from 25 nM of the acidic form of the buffering agent and 25 mM of the basic form of the buffering agent. In some embodiments, the non-amino acid buffer comprises proportions of the acid form and the base form of the buffering agent that are different.

In some embodiments, the concentration of the amino acid buffering agent (e.g., histidine or histidine monohydrochloride monohydrate) or non-amino acid buffering agent (e.g., TRIS, TRIS HCl, sodium phosphate dibasic, sodium phosphate monobasic dihydrate, ammonium phosphate dibasic, Succinate, or phosphate buffered saline) present in a pharmaceutical composition of the disclosure, for example, a histidine buffer, is from about 1 mM to about 750 mM, from about 1 mM to about 2 mM, from about 1 mM to about 5 mM, from about 1 mM to about 10 mM, from about 5 mM to about 10 mM, from about 10 mM to about 15 mM, from about 15 mM to about 20 mM, from about 20 mM to about 25 mM, from about 25 mM to about 30 mM, from about 30 mM to about 35 mM, from about 35 mM to about 40 mM, from about 40 mM to about 45 mM, about 45 mM to about 50 mM, from about 50 mM to about 55 mM, from about 55 mM to about 60 mM, from about 60 mM to about 65 mM, from about 65 mM to about 70 mM, from about 70 mM to about 75 mM, about 75 mM to about 80 mM, from about 80 mM to about 85 mM, from about 85 mM to about 90 mM, from about 90 mM to about 95 mM, from about 95 mM to about 100 mM, from about 100 mM to about 110 mM, from about 110 mM to about 120 mM, from about 120 mM to about 130 mM, from about 130 mM to about 140 mM, from about 140 mM to about 150 mM, from about 150 mM to about 160 mM, from about 160 mM to about 170 mM, from about 170 mM to about 180 mM, from about 180 mM to about 190 mM, from about 190 mM to about 200 mM, from about 200 mM to about 225 mM, from about 225 mM to about 250 mM, from about 250 mM to about 275 mM, from about 275 mM to about 300 mM, from about 300 mM to about 325 mM, from about 325 mM to about 350 mM, from about 350 mM to about 375 mM, from about 375 mM to about 400 mM, from about 400 mM to about 425 mM, from about 425 mM to about 450 mM, from about 450 mM to about 475 mM, from about 475 mM to about 500 mM, from about 500 mM to about 525 mM, from about 525 mM to about 550 mM, from about 550 mM to about 575 mM, from about 575 mM to about 600 mM, from about 600 mM to about 625 mM, from about 625 mM to about 650 mM, from about 650 mM to about 675 mM, from about 675 mM to about 700 mM, from about 700 mM to about 725 mM, from about 725 mM to about 750 mM. In some embodiments, the concentration of the amino acid buffering agent or non-amino acid buffering agent present in a pharmaceutical composition of the disclosure is at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 90 mM, at least about 95 mM, at least about 100 mM, at least about 110 mM, at least about 120 mM, at least about 130 mM, at least about 140 mM, at least about 150 mM, at least about 160 mM, at least about 170 mM, at least about 180 mM, at least about 190 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, or more. In some embodiments, the concentration of the amino acid buffering agent or non-amino acid buffering agent present in a pharmaceutical composition of the disclosure is at most about 750 mM, at most about 725 mM, at most about 700 mM, at most about 675 mM, at most about 650 mM, at most about 625 mM, at most about 600 mM, at most about 575 mM, at most about 550 mM, at most about 525 mM, at most about 500 mM, at most about 475 mM, at most about 450 mM, at most about 425 mM, at most about 400 mM, at most about 375 mM, at most about 350 mM, at most about 325 mM, at most about 300 mM, at most about 275 mM, at most about 250 mM, at most about 225 mM, at most about 200 mM, at most about 190 mM, at most about 180 mM, at most about 170 mM, at most about 160 mM, at most about 150 mM, at most about 140 mM, at most about 130 mM, at most about 120 mM, at most about 110 mM, at most about 100, at most about 95 mM, at most about 90 mM, at most about 85 mM, at most about 80 mM, at most about 75 mM, at most about 70 mM, at most about 65 mM, at most about 60 mM, at most about 55 mM, at most about 50 mM, at most about 45 mM, at most about 40 mM, at most about 35 mM, at most about 30 mM, at most about 25 mM, at most about 20 mM, at most about 15 mM, at most about 10 mM, at most about 9 mM, at most about 8 mM, at most about 7 mM, at most about 6 mM, at most about 5 mM, at most about 4 mM, at most about 3 mM, at most about 2 mM, at most about 1 mM, or less. In some embodiments, the concentration of the amino acid buffering agent or non-amino acid buffering agent present in a pharmaceutical composition of the disclosure is about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 90 mM, about 95 mM, about 100 mM, about 110 mM, about 120 mM, about 130 mM, about 140 mM, about 150 mM, about 160 mM, about 170 mM, about 180 mM, about 190 mM, about 200 mM, about 225 mM, about 250 mM, about 275 mM, about 300 mM, about 325 mM, about 350 mM, about 375 mM, about 400 mM, about 425 mM, about 450 mM, about 475 mM, about 500 mM, about 525 mM, about 550 mM, about 575 mM, about 600 mM, about 625 mM, about 650 mM, about 675 mM, about 700 mM, about 725 mM, or about 750 mM.

### Salts.

A compound described herein can be present in a composition with one or more salts. A salt (e.g., sodium chloride) can be used to raise the osmolality of a pharmaceutical composition. In some embodiments, a salt is added to a pharmaceutical composition to increase the osmolality of the pharmaceutical composition. In some embodiments, the salt is a sodium-based salt. Non-limiting examples of sodium-based salts include sodium chloride (NaCl), disodium succinate, sodium fluoride, sodium bromide, sodium iodine, sodium sulfate, sodium bicarbonate, and sodium carbonate.

In some embodiments, the salt is a calcium-based salt. Non-limiting examples of calcium-base salts include calcium acetate, calcium carbonate, calcium chloride, calcium citrate, calcium glubionate, calcium gluconate, and calcium lactate. In some embodiments, the salt is a magnesium-based salt. Non-limiting examples of magnesium-based salts include magnesium citrate, magnesium glycinate, magnesium chloride, magnesium lactate, magnesium malate, magnesium taurate, magnesium sulfate, and magnesium oxide. In some embodiments, the salt is a potassium-based salts. Non-limiting examples of potassium-based salts include potassium chloride, potassium citrate, potassium bicarbonate, potassium acetate, potassium iodide, monopotassium phosphate, dipotassium phosphate, potassium lactate, and potassium nitrate.

In some embodiments, the concentration of a salt (e.g., ammonium chloride, disodium succinate, succinic acid, or sodium chloride) present in a pharmaceutical composition of the disclosure is from about 0 mM to about 500 mM, from about 0 mM to about 1 mM, from about 1 mM to about 5 mM, from about 1 mM to about 10 mM, from about 5 mM to about 10 mM, from about 10 mM to about 15 mM, from about 15 mM to about 20 mM, from about 20 mM to about 25 mM, from about 25 mM to about 30 mM, from about 30 mM to about 35 mM, from about 35 mM to about 40 mM, from about 40 mM to about 45 mM, about 45 mM to about 50 mM, from about 50 mM to about 55 mM, from about 55 mM to about 60 mM, from about 60 mM to about 65 mM, from about 65 mM to about 70 mM, from about 70 mM to about 75 mM, about 75 mM to about 80 mM, from about 80 mM to about 85 mM, from about 85 mM to about 90 mM, from about 90 mM to about 95 mM, from about 95 mM to about 100 mM, from about 100 mM to about 110 mM, from about 110 mM to about 120 mM, from about 120 mM to about 130 mM, from about 130 mM to about 140 mM, from about 140 mM to about 150 mM, from about 150 mM to about 160 mM, from about 160 mM to about 170 mM, from about 170 mM to about 180 mM, from about 180 mM to about 190 mM, from about 190 mM to about 200 mM, from about 200 mM to about 220 mM, from about 220 mM to about 240 mM, from about 240 mM to about 260 mM, from about 260 mM to about 280 mM, from about 280 mM to about 300 mM, from about 300 mM to about 320 mM, from about 320 mM to about 340 mM, from about 340 mM to about 360 mM, from about 360 mM to about 380 mM, from about 380 mM to about 400 mM, from about 400 mM to about 420 mM, from about 420 mM to about 440 mM, from about 440 mM to about 460 mM, from about 460 mM to about 480 mM, from about 480 mM to about 500 mM. In some embodiments, the concentration of the one or more salts present in a pharmaceutical composition at least about 0 mM, at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 90 mM, at least about 95 mM, at least about 100 mM, at least about 110 mM, at least about 120 mM, at least about 130 mM, at least about 140 mM, at least about 150 mM, at least about 160 mM, at least about 170 mM, at least about 180 mM, at least about 190 mM, at least about 200 mM, at least about 220 mM, at least about 240 mM, at least about 260 mM, at least about 280 mM, at least about 300 mM, at least about 320 mM, at least about 340 mM, at least about 360 mM, at least about 380 mM, at least about 400 mM, at least about 420 mM, at least about 440 mM, at least about 460 mM, at least about 480 mM, at least about 500 mM, or more. In some embodiments, the concentration of the one or more salts present in the a pharmaceutical composition is at most about at most about 500 mM, at most about 480 mM, at most about 460 mM, at most about 440 mM, at most about 420 mM, at most about 400 mM, at most about 380 mM, at most about 360 mM, at most about 340 mM, at most about 320 mM, at most about 300 mM, at most about 280 mM, at most about 260 mM, at most about 240 mM, at most about 220 mM, at most about 200 mM, at most about 190 mM, at most about 180 mM, at most about 170 mM, at most about 160 mM, at most about 150 mM, at most about 140 mM, at most about 130 mM, at most about 120 mM, at most about 110 mM, at most about 100, at most about 95 mM, at most about 90 mM, at most about 85 mM, at most about 80 mM, at most about 75 mM, at most about 70 mM, at most about 65 mM, at most about 60 mM, at most about 55 mM, at most about 50 mM, at most about 45 mM, at most about 40 mM, at most about 35 mM, at most about 30 mM, at most about 25 mM, at most about 20 mM, at most about 15 mM, at most about 10 mM, at most about 9 mM, at most about 8 mM, at most about 7 mM, at most about 6 mM, at most about 5 mM, at most about 4 mM, at most about 3 mM, at most about 2 mM, at most about 1 mM, or at most about 0 mM. In some embodiments, the concentration of the salts present in a pharmaceutical composition is 0 mM, about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 90 mM, about 95 mM, about 100 mM, about 110 mM, about 120 mM, about 130 mM, about 140 mM, about 150 mM, about 160 mM, about 170 mM, about 180 mM, about 190 mM, about 200 mM, about 220 mM, about 240 mM, about 260 mM, about 280 mM, about 300 mM, about 320 mM, about 340 mM, about 360 mM, about 380 mM, about 400 mM, about 420 mM, about 440 mM, about 460 mM, about 480 mM, or about 500 mM.

### Excipients.

A compound described herein can be present in a composition with one or more excipients. In some embodiments, the excipient is a solubilizing agent. Solubilizing agents can prevent aggregation of the pharmaceutical composition, which, in some cases, can induce an immune response or reduce the bioavailability of the pharmaceutical composition. In some embodiments, a solubilizing agent increases the solubility of the compound in pharmaceutical composition. Non-limiting examples of solubilizing agents include mannose, ribose, erythrose, threose, arabinose, arabinose, xylose, lyxose, allose, altrose, gulose, glucose, idose, galactose, talose, erythrylose, ribulose, xylulose, psicose, sorbose, tagatose, dihydroxyacetone, trehalose, sorbitol, lactose, fructose, maltose, dextran, starch, cellulose, amylose, agarose, glycerol, polyethylene glycol, polypropylene, glycol, dimethylsulfoxide, guanidine hydrochloride, tween 20, adenosine, cytidine, guanosine, thymidine, uridine, inosine, adenosine monophosphate, cytidine monophosphate, guanosine monophosphate, thymidine monophosphate, uridine monophosphate, and inosine monophosphate.

In some embodiments, the excipient is a viscosity modifier. The viscosity modifier can reduce the viscosity of the pharmaceutical composition. In some embodiments, the viscosity of a pharmaceutical composition is adjusted for different modes of administration. For example, subcutaneous injection can require a low viscosity formulation. In some embodiments, the viscosity modifier is a arginine or glutamate salt. In some embodiments, the one or more excipients can be one or more tonicity modifiers. Non-limiting examples of tonicity modifiers include mannose, ribose, erythrose, threose, arabinose, arabinose, xylose, lyxose, allose, altrose, gulose, idose, galactose, talose, erythrylose, ribulose, xylulose, psicose, sorbose, tagatose, dihydroxyacetone, trehalose, sorbitol, lactose, fructose, maltose, dextran, starch, cellulose, amylose, agarose, glycerol, polyethylene glycol, polypropylene, glycol, dimethylsulfoxide, guanidine hydrochloride, tween 20, adenosine, cytidine, guanosine, thymidine, uridine, inosine, adenosine monophosphate, cytidine monophosphate, guanosine monophosphate, thymidine monophosphate, uridine monophosphate, and inosine monophosphate.

In some embodiments, the excipient is a surfactant. Surfactants can reduce the surface tension of the pharmaceutical composition, prevent the compound from sticking to equipment (e.g., tubing, pumps, containers) during the processing/manufacturing stage, and stabilize the compound inside a micelle-based formulation. Non-limiting examples of surfactants include polyoxyethylene compounds, ethoxylated alcohols, ethoxylated esters, ethoxylated amides, polyoxypropylene compounds, propoxylated alcohols, ethoxylated/propoxylated block polymers, and propoxylated esters, alkanolamides, amine oxides, fatty acid esters of polyhydric alcohols, ethylene glycol esters, diethylene glycol esters, propylene glycol esters, glyceryl esters, polyglyceryl fatty acid esters, sorbitan esters, sucrose esters, and glucose esters, reaction products of a natural or polyethoxylated castor oil and ethylene oxide alkylgluceosides, alkylmaltosides, alkylthioglucosides, lauryl macrogolglycenides, polyoxyethylene alkyl ethers, polyoxyethylene alkylphenols, polyethylene glycol fatty (mono- and di-) acid esters, polyethylene glycol glycerol fatty acid esters, polyoxyethylene sorbitan fatty acid esters, polyoxyethylene-polyoxypropylene block copolymers, polyglyceryl fatty acid esters, polyoxyethylene glycerides, polyoxyethylene sterols and analogues thereof, polyoxyethylene vegetable oils, polyoxyethylene hydrogenated vegetable oils, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS), PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS), propylene glycol mono- and di-fatty acid esters, such as propylene glycol laurate, propylene glycol caprylate/caprate, Brij® 700, acetic acid, ascorbyl-6-palmitate, stearylamine, sodium lauryl sulfate, polyoxethyleneglycerol triiricinoleate, lauric acid, oleic acid, stearic acid, PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate, PEG-20 dilaurate, PEG-20 dioleate, PEG-20 distearate, PEG-32 dilaurate, PEG-32 dioleate, PEG-20 glyceryl laurate, PEG-30 glyceryl laurate, PEG-40 glyceryl laurate, PEG-20 glyceryl oleate, PEG-30 glyceryl oleate, and any combination thereof.

In some embodiments, the surfactant is a mixture of polyols and at least one of fatty acids, glycerides, vegetable oils, hydrogenated vegetable oils, sterols, sugar esters, sugar ethers, sucroglycerides, fatty acid salts, bile salts, phospholipids, phosphoric acid esters, carboxylates, sulfates, and sulfonates.

In some embodiments, the excipient is a pH modifier. In some embodiments, the pH is modified to be near physiological pH. Non-limiting examples of pH modifiers include hydrochloric acid, perchloric acid, hydrobromic acid, hydroiodic acid, nitric acid, sulfuric acid, lithium hydroxide, sodium hydroxide, potassium hydroxide, calcium hydroxide, strontium hydroxide, and barium hydroxide. In some embodiments, the pH modifier is hydrochloric acid (HCl). In some embodiments, the pH modifier is sodium hydroxide (NaOH).

In some embodiments, the excipient is arginine. In some embodiments, the excipient is L-arginine. In some embodiments, the excipient is arginine monohydrochloride monohydrate. In some embodiments, the excipient is L-arginine monohydrochloride monohydrate. In some embodiments, the excipient is methionine. In some embodiments, the excipient is L-methionine.

In some embodiments, the methionine prevents oxidation of the formulation. In some embodiments, the excipient is sodium glutamate. In some embodiments, the excipient is Tween 80. In some embodiments, the excipient is imidazole. In some embodiments, the excipient is pyridoxine.

In some embodiments, the concentration of the excipient (e.g., imidazole, pyridoxine, arginine, sodium glutamate, methionine, Tween 80, hydrochloric acid, or sodium hydroxide) present in a pharmaceutical composition is from about 0 mM to about 500 mM, from about 0 mM to about 1 mM, from about 1 mM to about 5 mM, from about 1 mM to about 10 mM, from about 5 mM to about 10 mM, from about 10 mM to about 15 mM, from about 15 mM to about 20 mM, from about 20 mM to about 25 mM, from about 25 mM to about 30 mM, from about 30 mM to about 35 mM, from about 35 mM to about 40 mM, from about 40 mM to about 45 mM, about 45 mM to about 50 mM, from about 50 mM to about 55 mM, from about 55 mM to about 60 mM, from about 60 mM to about 65 mM, from about 65 mM to about 70 mM, from about 70 mM to about 75 mM, about 75 mM to about 80 mM, from about 80 mM to about 85 mM, from about 85 mM to about 90 mM, from about 90 mM to about 95 mM, from about 95 mM to about 100 mM, from about 100 mM to about 110 mM, from about 110 mM to about 120 mM, from about 120 mM to about 130 mM, from about 130 mM to about 140 mM, from about 140 mM to about 150 mM, from about 150 mM to about 160 mM, from about 160 mM to about 170 mM, from about 170 mM to about 180 mM, from about 180 mM to about 190 mM, from about 190 mM to about 200 mM, from about 200 mM to about 210 mM, from about 210 mM to about 220 mM, from about 220 mM to about 230 mM, from about 230 mM to about 240 mM, from about 240 mM to about 250 mM, from about 250 mM to about 260 mM, from about 260 mM to about 270 mM, from about 270 mM to about 280 mM, from about 280 mM to about 290 mM, from about 290 mM to about 300 mM, from about 300 mM to about 310 mM, from about 320 mM to about 330 mM, from about 330 mM to about 340 mM, from about 340 mM to about 350 mM, from about 350 mM to about 360 mM, from about 360 mM to about 370 mM, from about 370 mM to about 380 mM, from about 380 mM to about 390 mM, from about 390 mM to about 400 mM, from about 400 mM to about 410 mM, from about 410 mM to about 420 mM, from about 420 mM to about 430 mM, from about 430 mM to about 440 mM, from about 440 mM to about 450 mM, from about 450 mM to about 460 mM, from about 460 mM to about 470 mM, from about 470 mM to about 480 mM, from about 480 mM to about 490 mM, or from about 490 mM to about 500 mM. In some embodiments, the concentration of the excipient present in a pharmaceutical composition is at least about 0 mM, at least about 1 mM, at least about 2 mM, at least about 3 mM, at least about 4 mM, at least about 5 mM, at least about 6 mM, at least about 7 mM, at least about 8 mM, at least about 9 mM, at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 90 mM, at least about 95 mM, at least about 100 mM, at least about 110 mM, at least about 120 mM, at least about 130 mM, at least about 140 mM, at least about 150 mM, at least about 160 mM, at least about 170 mM, at least about 180 mM, at least about 190 mM, at least about 200 mM, at least about 210 mM, at least about 220 mM, at least about 230 mM, at least about 240 mM, at least about 250 mM, at least about 260 mM, at least about 270 mM, at least about 280 mM, at least about 290 mM, at least about 300 mM, at least about 310 mM, at least about 320 mM, at least about 330 mM, at least about 340 mM, at least about 350 mM, at least about 360 mM, at least about 370 mM, at least about 380 mM, at least about 390 mM, at least about 400 mM, at least about 410 mM, at least about 420 mM, at least about 430 mM, at least about 440 mM, at least about 450 mM, at least about 460 mM, at least about 470 mM, at least about 480 mM, at least about 490 mM, at least about 500 mM, or more. In some embodiments, the concentration of the excipient present in the a pharmaceutical composition is at most about 500 mM, at most about 490 mM, at most about 480 mM, at most about 470 mM, at most about 460 mM, at most about 450 mM, at most about 440 mM, at most about 430 mM, at most about 420 mM, at most about 410 mM, at most about 400 mM, at most about 390 mM, at most about 380 mM, at most about 370 mM, at most about 360 mM, at most about 350 mM, at most about 340 mM, at most about 330 mM, at most about 320 mM, at most about 310 mM, at most about 300 mM, at most about 290 mM, at most about 280 mM, at most about 270 mM, at most about 260 mM, at most about 250 mM, at most about 240 mM, at most about 230 mM, at most about 220 mM, at most about 210 mM, at most about 200 mM, at most about 190 mM, at most about 180 mM, at most about 170 mM, at most about 160 mM, at most about 150 mM, at most about 140 mM, at most about 130 mM, at most about 120 mM, at most about 110 mM, at most about 100, at most about 95 mM, at most about 90 mM, at most about 85 mM, at most about 80 mM, at most about 75 mM, at most about 70 mM, at most about 65 mM, at most about 60 mM, at most about 55 mM, at most about 50 mM, at most about 45 mM, at most about 40 mM, at most about 35 mM, at most about 30 mM, at most about 25 mM, at most about 20 mM, at most about 15 mM, at most about 10 mM, at most about 9 mM, at most about 8 mM, at most about 7 mM, at most about 6 mM, at most about 5 mM, at most about 4 mM, at most about 3 mM, at most about 2 mM, at most about 1 mM, or at most about 0 mM. In some embodiments, the concentration of the excipient present in a pharmaceutical composition is about 0 mM, about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 90 mM, about 95 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM.

### pH of the Pharmaceutical Composition.

A compound as described herein can be present in a composition with a defined pH range. The pH of the pharmaceutical compositions can be adjusted to a value near physiological pH. In some embodiments, the physiological pH is about 7 to about 7.8. In some embodiments, the pH range of the pharmaceutical composition is from about 1 to about 9, from about 1 to about 2, from about 2 to about 8, from about 2 to about 3, from about 3 to about 8, from about 3 to about 4, from about 4 to about 8, from about 4 to about 5, from about 5 to about 7.5, from about 5 to about 8, from about 5 to about 5.5, from about 5.5 to about 8, from about 5.5 to about 7.5, from about 5.5 to about 6, from about 6 to about 8, from about 6 to about 7.5, from about 6 to about 7, from about 7 to about 8, from about 7 to 7.5, or from about 7 to about 7.8. In some embodiments, the pH range of the pharmaceutical composition is from about 5 to about 7.5. In some embodiments, the pH of the pharmaceutical composition is at least about 1, at least about 1.5, at least about 2, at least about 2.5, at least about 3, at least about 3.5, at least about 3.5, at least about 4, at least about 4.5, at least about 5, at least about 5.5, at least about 6, at least about 6.5, at least about 7, at least about 7.5, at least about 7.8, or at least about 8, or more. In some embodiments, the pH of the pharmaceutical composition is a pH of at most about 8, at most about 7.8, at most about 7.5, at most about 7, at most about 6.5, at most about 6, at most about 5.5, at most about 5, at most about 4.5, at most about 4, at most about 3.5, at most about 3, at most about 2.5, at most about 2, at most about 1.5, at most about 1, or less. In some embodiments, the pH of the pharmaceutical composition is about 1, about 1.5, about 2, about 2.5, about 3, about 3.5, about 4, about 4.5, about 5, about 5.5, about 6, about 6.5, about 7, about 7.5, about 7.8, or about 8. In some embodiments, the pH of the pharmaceutical composition is about 5. In some embodiments, the pH of the pharmaceutical composition is about 5.5. In some embodiments, the pH of the pharmaceutical composition is about 6. In some embodiments, the pH of the pharmaceutical composition is about 6.5. In some embodiments, the pH of the pharmaceutical composition is about 7. In some embodiments, the pH of the pharmaceutical composition is about 7.5. In some embodiments, the pH of the pharmaceutical composition is about 7.8.

In some embodiments, the pH of the pharmaceutical composition is measured by a commercially available pH meter.

### Osmolality of the Pharmaceutical Composition.

A pharmaceutical composition of the disclosure can exhibit an osmolality that falls within a defined range. Osmolality can be a measurement of the concentration of particles found in a fluid. For dosage forms tailored for specific routes of administration, the osmolality of the pharmaceutical composition can be modulated to be near physiological osmolality. In some embodiments, the physiological osmolality is about 270 mOsm/kg to about 350 mOsm/kg. In some embodiments, the osmolality of the pharmaceutical composition is from about 200 milliosmoles per kilogram (mOsm/kg) to about 2,000 mOsm/kg, from about 200 mOsm/kg to about 250 mOsm/kg, from about 250 mOsm/kg to about 270 mOsm/kg, from about 270 mOsm/kg to about 300 mOsm/kg, from about 250 mOsm/kg to about 300 mOsm/kg, from about 300 mOsm/kg to about 350 mOsm/kg, from about 350 mOsm/kg to about 400 mOsm/kg, from about 400 mOsm/kg to about 450 mOsm/kg, from about 450 mOsm/kg to about 500 mOsm/kg, from about 500 mOsm/kg to about 550 mOsm/kg, from about 550 mOsm/kg to about 600 mOsm/kg, from about 600 mOsm/kg to about 650 mOsm/kg, from about 650 mOsm/kg to about 700 mOsm/kg, from about 700 mOsm/kg to about 750 mOsm/kg, from about 750 mOsm/kg to about 800 mOsm/kg, from about 800 mOsm/kg to about 850 mOsm/kg, from about 850 mOsm/kg to about 900 mOsm/kg, from about 900 mOsm/kg to about 950 mOsm/kg, from about 950 mOsm/kg to about 1,000 mOsm/kg, from about 1,000 mOsm/kg to about 1,100 mOsm/kg, from about 1,100 mOsm/kg to about 1,200 mOsm/kg, from about 1,200 mOsm/kg to about 1,300 mOsm/kg, from about 1,300 mOsm/kg to about 1,400 mOsm/kg, from about 1,400 mOsm/kg to about 1,500 mOsm/kg, from about 1,500 mOsm/kg to about 1,600 mOsm/kg, from about 1,600 mOsm/kg to about 1,700 mOsm/kg, from about 1,700 mOsm/kg to about 1,800 mOsm/kg, from about 1,800 mOsm/kg to about 1,900 mOsm/kg, from about 1,900 mOsm/kg to about 2,000. In some embodiments, the osmolality of the pharmaceutical composition is from about 250 mOsm/kg to about 350 mOsm/kg. In some embodiments, the osmolality of the pharmaceutical composition can comprise at least about 200 mOsm/kg, at least about 250 mOsm/kg, at least about 270 mOsm/kg, at least about 300 mOsm/kg, at least about 350 mOsm/kg, at least about 400 mOsm/kg, at least about 450 mOsm/kg, at least about 500 mOsm/kg, at least about 550 mOsm/kg, at least about 600 mOsm/kg, at least about 650 mOsm/kg, at least about 700 mOsm/kg, at least about 750 mOsm/kg, at least about 800 mOsm/kg, at least about 850 mOsm/kg, at least about 900 mOsm/kg, at least about 950 mOsm/kg, at least about 1,000 mOsm/kg, at least about 1,100 mOsm/kg, at least about 1,200 mOsm/kg, at least about 1,300 mOsm/kg, at least about 1,400 mOsm/kg, at least about 1,500 mOsm/kg, at least about 1,600 mOsm/kg, at least about 1,700 mOsm/kg, at least about 1,800 mOsm/kg, at least about 1,900 mOsm/kg, at least about 2,000 mOsm/kg, or more. In some embodiments, the osmolality of the pharmaceutical composition is at least about 250 mOsm/kg. In some embodiments, the osmolality of the pharmaceutical composition can be at least about 300 mOsm/kg. In some embodiments, the osmolality of the pharmaceutical composition is at least about 350 mOsm/kg. In some embodiments, the osmolality of the pharmaceutical composition is at most about 2,000 mOsm/kg, at most about 1,900 mOsm/kg, at most about 1,800 mOsm/kg, at most about 1,700 mOsm/kg, at most about 1,600 mOsm/kg, at most about 1,500 mOsm/kg, at most about 1,400 mOsm/kg, at most about 1,300 mOsm/kg, at most about 1,200 mOsm/kg, at most about 1,100 mOsm/kg, at most about 1,000 mOsm/kg, at most about 950 mOsm/kg, at most about 900 mOsm/kg, at most about 850 mOsm/kg, at most about 800 mOsm/kg, at most about 750 mOsm/kg, at most about 700 mOsm/kg, at most about 650 mOsm/kg, at most about 600 mOsm/kg, at most about 550 mOsm/kg, at most about 500 mOsm/kg, at most about 450 mOsm/kg, at most about 400 mOsm/kg, at most about 350 mOsm/kg, at most about 300 mOsm/kg, at most about 270 mOsm/kg, at most about 250 mOsm/kg, at most about 200 mOsm/kg, or less. In some embodiments, the osmolality of the pharmaceutical composition is at most about 350 mOsm/kg. In some embodiments, the osmolality of the pharmaceutical composition is at most about 300 mOsm/kg. In some embodiments, the osmolality of the pharmaceutical composition is at most about 250 mOsm/kg. In some embodiments, the osmolality of the pharmaceutical composition is about 200 mOsm/kg, about 250 mOsm/kg, about 270 mOsm/kg, about 300 mOsm/kg, about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, about 800 mOsm/kg, about 850 mOsm/kg, about 900 mOsm/kg, about 950 mOsm/kg, about 1,000 mOsm/kg, about 1,100 mOsm/kg, about 1,200 mOsm/kg, about 1,300 mOsm/kg, about 1,400 mOsm/kg, about 1,500 mOsm/kg, about 1,600 mOsm/kg, about 1,700 mOsm/kg, about 1,800 mOsm/kg, about 1,900 mOsm/kg, or about 2,000 mOsm/kg. In some embodiments, the osmolality of the pharmaceutical composition is about 250 mOsm/kg. In some embodiments, the osmolality of the pharmaceutical composition is about 300 mOsm/kg. In some embodiments, the osmolality of the pharmaceutical composition is about 350 mOsm/kg.

In some embodiments, the osmolality of the pharmaceutical composition is measured by a vapor pressure osmometer.

### Light Scattering Properties of Pharmaceutical Compositions.

A pharmaceutical composition of the disclosure can exhibit an apparent molecular weight that falls within a defined range. In some embodiments, the apparent molecular weight of the pharmaceutical composition is measured by light scattering. In some embodiments, the light scattering is dynamic light scattering (DLS). Dynamic light scattering can measure the size and size distribution of molecules. The size and size distribution of molecules can be determined by measuring random changes in the intensity of light scattering from a formulation. DLS can be used to obtain the average translational diffusion coefficient of molecules in the solution. The average translational diffusion coefficient of the molecules in the solution can be used to look for heterogenicity in a solution. In some embodiments, the light scattering is static light scattering (SLS). In some embodiments, the static light scattering measures the molecular weight of the compound in the formulation. The molecular weight can be determined using the relationship between the intensity of light scattered by a molecule and its molecular weight and size. In some embodiments, the light scattering measurement is recorded as an apparent molecular weight (App MW) value. The apparent molecule weight of the compound particle in a formulation can be measured by normalizing the intensity of the SLS to a blank formulation. In some cases, a blank formulation is a formulation without the compound. In some embodiments, the App MW value is from about 5 kilodaltons (kDa) to about 80 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kD to about 35 kDa, from about 35 kDa to about 40 kDa, from about 40 kDa to about 45 kDa, from about 45 kDa to about 50 kDa, from about 50 kDa to about 55 kDa, from about 55 kDa to about 60 kDa, from about 60 kDa to about 65 kDa, from about 65 kDa to about 70 kDa, from about 70 kDa to about 75 kDa, or from about 75 kDa to about 80 kDa. In some embodiments, the App MW value of the pharmaceutical composition is at least about 5 kDa, at least about 10 kDa, at least about 15 kDa, at least about 20 kDa, at least about 25 kDa, at least about 30 kDa, at least about 35 kDa, at least about 40 kDa, at least about 45 kDa, at least about 50 kDa, at least about 55 kDa, at least about 60 kDa, at least about 65 kDa, at least about 70 kDa, at least about 75 kDa, at least about 80 kDa, or more. In some embodiments, the App MW value of the pharmaceutical composition is at most about 80 kDa, at most about 75 kDa, at most about 70 kDa, at most about 65 kDa, at most about 60 kDa, at most about 55 kDa, at most about 50 kDa, at most about 45 kDa, at most about 40 kDa, at most about 35 kDa, at most about 30 kDa, at most about 25 kDa, at most about 20 kDa, at most about 15 kDa, at most about 10 kDa, at most about 5 kDa, or less.

In some embodiments, the App MW value of the pharmaceutical composition is about 5 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, or about 80 kDa.

### Dialysis of Pharmaceutical Compositions.

A compound as described herein can be present in a pharmaceutical composition prepared via dialysis. Dialysis can be used to control the levels of excipients, counterion content, and pH in the pharmaceutical composition. In some embodiments, the dialysis is standard static dialysis. In some embodiments, the dialysis is dynamic dialysis. In some embodiments, dialysis of the pharmaceutical composition is performed by dialyzing a solution of the compound against a formulation. In some embodiments, a molecular weight cut off (MWCO) membrane is used in dialysis. In some embodiments, the MWCO membrane is a 2 kDa MWCO membrane. In some embodiments, the MWCO membrane is a regenerated cellulose membrane.

Alternatively, the pharmaceutical composition can be prepared without dialysis. In some embodiments, the pharmaceutical composition is prepared by direct formulation. In some embodiments, the pharmaceutical composition is prepared by transflow filtration.

### NUMBERED EMBODIMENTS

Embodiment 1. A pharmaceutical composition comprising:
(a) a compound comprising an oligomeric structure, wherein the oligomeric structure comprises a repeating unit of formula (I):

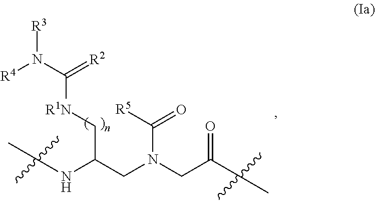

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
or a pharmaceutically-acceptable salt or ionized form thereof, and
(b) an amino acid or a pharmaceutically-acceptable salt or ionized form thereof, wherein the amino acid comprises a side chain that is positively charged at physiological pH.

Embodiment 2. The composition of embodiment 1, wherein the amino acid is arginine.

Embodiment 3. The composition of embodiment 1, wherein the amino acid is lysine.

Embodiment 4. The composition of embodiment 1, wherein the amino acid is ornithine.

Embodiment 5. The composition of embodiment 1, wherein the amino acid is histidine.

Embodiment 6. The composition of embodiment 1 or embodiment 5, wherein histidine or an ionized form thereof is present in the composition at a concentration of about 0.5 mM to about 100 mM.

Embodiment 7. The composition of embodiment 1 or embodiment 5, wherein histidine or an ionized form thereof is present in the composition at a concentration of about 25 mM to about 100 mM.

Embodiment 8. The composition of embodiment 1 or embodiment 5, wherein histidine or an ionized form thereof is present in the composition at a concentration of about 25 mM to about 75 mM.

Embodiment 9. The composition of embodiment 1 or embodiment 5, wherein histidine or an ionized form thereof is present in the composition at a concentration of about 40 mM.

Embodiment 10. The composition of embodiment 1 or embodiment 5, wherein histidine or an ionized form thereof is present in the composition at a concentration of about 50 mM.

Embodiment 11. The composition of any one of embodiments 5-10, wherein the histidine is L-histidine.

Embodiment 12. The composition of any one of embodiments 1-11, wherein the pharmaceutical composition further comprises a second amino acid or a pharmaceutically-acceptable salt or ionized form thereof.

Embodiment 13. The composition of embodiment 12, wherein the second amino acid comprises a side chain that is positively charged at physiological pH.

Embodiment 14. The composition of embodiment 12 or 13, wherein the second amino acid is arginine.

Embodiment 15. The composition of any one of embodiments 1-14, wherein the pharmaceutical composition further comprises an anti-aggregation agent.

Embodiment 16. The composition of embodiment 15, wherein the anti-aggregation agent is a polyol.

Embodiment 17. The composition of embodiment 15 or 16, wherein the anti-aggregation agent is a saccharide.

Embodiment 18. The composition of any one of embodiments 15-17, wherein the anti-aggregation agent is a sugar.

Embodiment 19. The composition of any one of embodiments 15-17, wherein the anti-aggregation agent is a sugar alcohol.

Embodiment 20. The composition of any one of embodiments 15-19, wherein the anti-aggregation agent is maltitol, mannitol, isomalt, sorbitol, xylitol, or erythritol.

Embodiment 21. The composition of any one of embodiments 15-19, wherein the anti-aggregation agent is mannitol.

Embodiment 22. The composition of embodiment 21, wherein mannitol is present in the composition at a concentration from about 5 mM to about 200 mM.

Embodiment 23. The composition of embodiment 21, wherein mannitol is present in the composition at a concentration from about 50 mM to about 150 mM.

Embodiment 24. The composition of embodiment 21, wherein mannitol is present in the composition at a concentration from about 5 mM to about 30 mM.

Embodiment 25. The composition of embodiment 21, wherein mannitol is present in the composition at a concentration of 15 mM.

Embodiment 26. The composition of embodiment 21, wherein mannitol is present in the composition at a concentration of 100 mM.

Embodiment 27. The composition of any one of embodiments 15-18, wherein the anti-aggregation agent is a sugar.

Embodiment 28. The composition of any one of embodiments 15-18 and 27, wherein anti-aggregation agent is a monosaccharide.

Embodiment 29. The composition of any one of embodiments 15-18, 27, and 28, wherein the anti-aggregation agent is dextrose.

Embodiment 30. The composition of embodiment 29, wherein dextrose is present in the composition at a concentration of about 50 mM to about 400 mM.

Embodiment 31. The composition of embodiment 29, wherein dextrose is present in the composition at a concentration of about 50 mM to about 300 mM.

Embodiment 32. The composition of embodiment 29, wherein dextrose is present in the composition at a concentration of about 200 mM to about 250 mM.

Embodiment 33. The composition of embodiment 29, wherein dextrose is present in the composition at a concentration of about 100 mM.

Embodiment 34. The composition of embodiment 29, wherein dextrose is present in the composition at a concentration of about 230 mM.

Embodiment 35. A pharmaceutical composition comprising:
(a) a compound comprising an oligomeric structure, wherein the oligomeric structure comprises a repeating unit of formula (I):

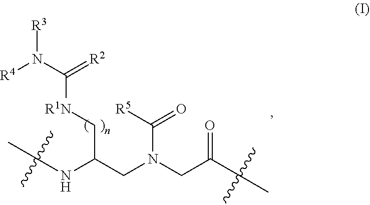

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
      or a pharmaceutically-acceptable salt or ionized form thereof; and
(b) mannitol at a concentration of about 5 mM to about 500 mM.

Embodiment 36. The composition of embodiment 35, wherein mannitol is present in the composition at a concentration from about 5 mM to about 200 mM.

Embodiment 37. The composition of embodiment 35, wherein mannitol is present in the composition at a concentration from about 50 mM to about 150 mM.

Embodiment 38. The composition of embodiment 35, wherein mannitol is present in the composition at a concentration from about 5 mM to about 30 mM.

Embodiment 39. The composition of embodiment 35, wherein mannitol is present in the composition at a concentration of 15 mM.

Embodiment 40. The composition of embodiment 35, wherein mannitol is present in the composition at a concentration of 100 mM.

Embodiment 41. The composition of any one of embodiments 35-40, wherein the pharmaceutical composition further comprises a buffering agent.

Embodiment 42. The composition of embodiment 41, wherein the buffering agent is an amino acid or a pharmaceutically acceptable salt or ionized form thereof.

Embodiment 43. The composition of embodiment 42, wherein the amino acid comprises a side chain that is positively charged at physiological pH.

Embodiment 44. The composition of any one of embodiments 41-43, wherein the amino acid is selected from histidine, lysine, ornithine, and arginine.

Embodiment 45. The composition of any one of embodiments 41-43, wherein the amino acid is arginine.

Embodiment 46. The composition of any one of embodiments 41-43, wherein the amino acid is lysine.

Embodiment 47. The composition of any one of embodiments 41-43, wherein the amino acid is ornithine.

Embodiment 48. The composition of any one of embodiments 41-43, wherein the amino acid is histidine.

Embodiment 49. The composition of embodiment 48, wherein histidine or an ionized form thereof is present in the composition at a concentration of about 0.5 mM to about 100 mM.

Embodiment 50. The composition of embodiment 48, wherein histidine or an ionized form thereof is present in the composition at a concentration of about 25 mM to about 100 mM.

Embodiment 51. The composition of embodiment 48, wherein histidine or an ionized form thereof is present in the composition at a concentration of about 25 mM to about 75 mM.

Embodiment 52. The composition of embodiment 48, wherein histidine or an ionized form thereof is present in the composition at a concentration of about 40 mM.

Embodiment 53. The composition of embodiment 48, wherein histidine or an ionized form thereof is present in the composition at a concentration of about 50 mM.

Embodiment 54. The composition of any one of embodiments 48-53, wherein the histidine is L-histidine.

Embodiment 55. The composition of embodiment 41, wherein the buffering agent is a dicarboxylic acid or a pharmaceutically acceptable salt or ionized form thereof.

Embodiment 56. The composition of embodiment 55, wherein the dicarboxylic acid is malonic acid, succinic acid, glutaric acid, adipic acid, pimelic acid, or a pharmaceutically-acceptable salt thereof.

Embodiment 57. The composition of embodiment 55, wherein the buffering agent is succinic acid.

Embodiment 58. The composition of embodiment 41, wherein the buffering agent is citric acid, acetic acid, boric acid, or phosphoric acid.

Embodiment 59. The composition of embodiment 41, wherein the buffering agent comprises a 1,2-aminoalcohol moiety.

Embodiment 60. The composition of embodiment 41 or 59, wherein the buffering agent is TAPS ([tris(hydroxymethyl)methylamino]propanesulfonic acid), Bicine (2-(bis(2-hydroxyethyl)amino)acetic acid), Tris (tris(hydroxymethyl)aminomethane), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), or a pharmaceutically-acceptable salt thereof.

Embodiment 61. A pharmaceutical composition comprising:
(a) a compound comprising an oligomeric structure, wherein the oligomeric structure comprises a repeating unit of formula:

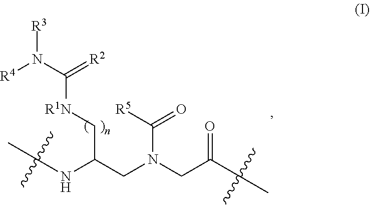

or an ionized form thereof, wherein:
   R¹ is H, alkyl, or a nitrogen atom protecting group;
   R² is O, NH, N(alkyl), or N(Pg^{N}), wherein Pg^{N} is a nitrogen atom protecting group;
   R³ is H, alkyl, or a nitrogen atom protecting group;
   R⁴ is H, alkyl, or a nitrogen atom protecting group;
   R⁵ is linear alkyl, branched alkyl, cyclic alkyl, linear alkenyl, branched alkenyl, cyclic alkenyl, linear alkynyl, branched alkynyl, cyclic alkynyl, aryl, heteroaryl, heterocyclyl, linear O-alkyl, branched O-alkyl, cyclic O-alkyl, linear O-alkenyl, branched O-alkenyl, cyclic O-alkenyl, linear O-alkynyl, branched O-alkynyl, cyclic O-alkynyl, O-aryl, O-heteroaryl, or O-heterocyclyl any of which is unsubstituted or substituted; and
   n is 1, 2, 3, 4, 5, 6, 7, 8, 9, or 10,
   or a pharmaceutically-acceptable salt or ionized form thereof, and
(b) a pharmaceutically acceptable excipient,
   wherein the pharmaceutical composition has an Apparent Molecular Weight (App MW) that is about 5 kDa to about 50 kDa.

Embodiment 62. The composition of embodiment 61, wherein the pharmaceutical composition further comprises an anti-aggregation agent.

Embodiment 63. The composition of embodiment 62, wherein the anti-aggregation agent is a polyol.

Embodiment 64. The composition of embodiment 62 or 63, wherein the anti-aggregation agent is a saccharide.

Embodiment 65. The composition of any one of embodiments 62-64, wherein the anti-aggregation agent is a sugar.

Embodiment 66. The composition of any one of embodiments 62-64, wherein the anti-aggregation agent is a sugar alcohol.

Embodiment 67. The composition of any one of embodiments 62-66, wherein the anti-aggregation agent is maltitol, mannitol, isomalt, sorbitol, xylitol, or erythritol.

Embodiment 68. The composition of any one of embodiments 62-64, wherein the anti-aggregation agent is mannitol.

Embodiment 69. The composition of embodiment 68, wherein mannitol is present in the composition at a concentration from about 5 mM to about 200 mM.

Embodiment 70. The composition of embodiment 68, wherein mannitol is present in the composition at a concentration from about 50 mM to about 150 mM.

Embodiment 71. The composition of embodiment 68, wherein mannitol is present in the composition at a concentration from about 5 mM to about 30 mM.

Embodiment 72. The composition of embodiment 68, wherein mannitol is present in the composition at a concentration of 15 mM.

Embodiment 73. The composition of embodiment 68, wherein mannitol is present in the composition at a concentration of 100 mM.

Embodiment 74. The composition of any one of embodiments 62-64, wherein the anti-aggregation agent is a sugar.

Embodiment 75. The composition of any one of embodiments 62-64 and 74, wherein the anti-aggregation agent is a monosaccharide.

Embodiment 76. The composition of any one of embodiments 62-64, 74, and 75, wherein the anti-aggregation agent is dextrose.

Embodiment 77. The composition of embodiment 76, wherein dextrose is present in the composition at a concentration of about 50 mM to about 400 mM.

Embodiment 78. The composition of embodiment 76, wherein dextrose is present in the composition at a concentration of about 50 mM to about 300 mM.

Embodiment 79. The composition of embodiment 76, wherein dextrose is present in the composition at a concentration of about 200 mM to about 250 mM.

Embodiment 80. The composition of embodiment 76, wherein dextrose is present in the composition at a concentration of about 100 mM.

Embodiment 81. The composition of embodiment 76, wherein dextrose is present in the composition at a concentration of about 230 mM.

Embodiment 82. The composition of any one of embodiments 61-81, wherein the pharmaceutical composition further comprises a buffering agent.

Embodiment 83. The composition of embodiment 82, wherein the buffering agent is an amino acid or a pharmaceutically acceptable salt or ionized form thereof.

Embodiment 84. The composition of embodiment 83, wherein the amino acid comprises a side chain that is positively charged at physiological pH.

Embodiment 85. The composition of any one of embodiments 82-84, wherein the amino acid is selected from histidine, lysine, ornithine, and arginine.

Embodiment 86. The composition of any one of embodiments 82-84, wherein the amino acid is arginine.

Embodiment 87. The composition of any one of embodiments 82-84, wherein the amino acid is lysine.

Embodiment 88. The composition of any one of embodiments 82-84, wherein the amino acid is ornithine.

Embodiment 89. The composition of any one of embodiments 82-84, wherein the amino acid is histidine.

Embodiment 90. The composition of embodiment 89, wherein histidine or an ionized form thereof is present in the composition at a concentration of about 0.5 mM to about 100 mM.

Embodiment 91. The composition of embodiment 89, wherein histidine or an ionized form thereof is present in the composition at a concentration of about 25 mM to about 100 mM.

Embodiment 92. The composition of embodiment 89, wherein histidine or an ionized form thereof is present in the composition at a concentration of about 25 mM to about 75 mM.

Embodiment 93. The composition of embodiment 89, wherein histidine or an ionized form thereof is present in the composition at a concentration of about 40 mM.

Embodiment 94. The composition of embodiment 89, wherein histidine or an ionized form thereof is present in the composition at a concentration of about 50 mM.

Embodiment 95. The composition of any one of embodiments 89-94, wherein the histidine is L-histidine.

Embodiment 96. The composition of embodiment 82, wherein the buffering agent is a dicarboxylic acid or a pharmaceutically acceptable salt or ionized form thereof.

Embodiment 97. The composition of embodiment 96, wherein the dicarboxylic acid is malonic acid, succinic acid, glutaric acid, adipic acid, pimelic acid, or a pharmaceutically-acceptable salt thereof.

Embodiment 98. The composition of embodiment 96, wherein the buffering agent is succinic acid.

Embodiment 99. The composition of embodiment 82, wherein the buffering agent is citric acid, acetic acid, boric acid, or phosphoric acid.

Embodiment 100. The composition of embodiment 82, wherein the buffering agent comprises a 1,2-aminoalcohol moiety.

Embodiment 101. The composition of embodiment 82 or 100, wherein the buffering agent is TAPS ([tris(hydroxymethyl)methylamino]propanesulfonic acid), Bicine (2-(bis(2-hydroxyethyl)amino)acetic acid), Tris (tris(hydroxymethyl)aminomethane), Tricine (N-[tris(hydroxymethyl)methyl]glycine), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), or a pharmaceutically-acceptable salt thereof.

Embodiment 102. The composition of any one of embodiments 61-101, wherein the apparent molecular weight is about 10 kDa to about 40 kDa.

Embodiment 103. The composition of embodiment 102, wherein the apparent molecular weight is measured by light scattering.

Embodiment 104. The composition of embodiment 102 or 103, wherein the light scattering is static light scattering (SLS).

Embodiment 105. The composition of embodiment 102 or 103, wherein the light scattering is dynamic light scattering (DLS).

Embodiment 106. The composition of any one of embodiments 1-105, wherein the compound further comprises a first chemical moiety attached to the oligomeric structure, and a second chemical moiety attached to the oligomeric structure, wherein the oligomeric structure, wherein the first chemical moiety, and the second chemical moiety form:

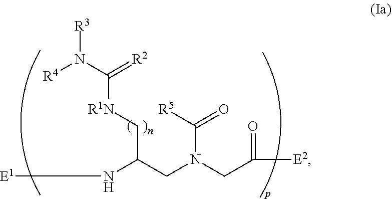

wherein:
E¹ is the first chemical moiety;
E² is the second chemical moiety; and
p is an integer that is 1-100.

Embodiment 107. The composition of embodiment 106, wherein p is 6.

Embodiment 108. The composition of embodiment 106, wherein p is 7.

Embodiment 109. The composition of embodiment 106, wherein p is 8.

Embodiment 110. The composition of any one of embodiments 1-109, wherein each of R¹, R³, and R⁴ is hydrogen; and R² is NH or N(Pg^{N}).

Embodiment 111. The composition of any one of embodiments 1-110, wherein R⁵ is linear alkyl.

Embodiment 112. The composition of any one of embodiments 1-110, wherein R⁵ is methyl.

Embodiment 113. The composition of any one of embodiments 1-112, wherein n is 3.

Embodiment 114. The composition of any one of embodiments 1-112, wherein n is 4.

Embodiment 115. The composition of embodiment 106, wherein E¹ is hydrogen, acyl, a group that together with the nitrogen atom to which E¹ is bound forms a carbamate, a probe, a metal chelator, an imaging agent, or a biologically-active agent; and E² is OH, OMe, NH₂, a probe, a metal chelator, an imaging agent, or a biologically-active agent.

Embodiment 116. The composition of embodiment 115, wherein E¹ is hydrogen and E² is the biologically-active agent.

Embodiment 117. The composition of embodiment 115 or 116, wherein the biologically-active agent comprises a structure that interferes with expression of a gene associated with a neuromuscular disease phenotype.

Embodiment 118. The composition of embodiment 117, wherein the neuromuscular disease phenotype is a DM1 disease phenotype.

Embodiment 119. The composition of embodiment 118, wherein the DM1 disease phenotype is associated with a non-wild-type DM1 gene that differs from a wild type DM1 gene in a repeat expansion mutation.

Embodiment 120. The composition of any one of embodiments 115-118, wherein the biologically-active agent comprises a structure that interferes with expression of a gene associated with a neurodegenerative disease phenotype.

Embodiment 121. The composition of embodiment 120, wherein the neurodegenerative disease phenotype is a Huntington's disease phenotype.

Embodiment 122. The composition of embodiment 121, wherein the Huntington's disease phenotype is associated with a non-wild-type HTT gene that differs from a wild type HTT gene in a repeat expansion mutation.

Embodiment 123. The composition of any one of embodiments 115-122, wherein the biologically-active agent binds to a mRNA sequence at a region that is (CUG)_{z}, wherein z is an integer from 1-100 (SEQ ID NO: 324).

Embodiment 124. The composition of embodiment 123, wherein the biologically-active agent binds to a mRNA sequence at a region that is (CAG)_{z}, wherein z is an integer from 1-100 (SEQ ID NO: 325).

Embodiment 125. The composition of any one of embodiments 115-124, wherein the biologically-active agent comprises a structure that interferes with expression of a cancer-causing protein.

Embodiment 126. The composition of embodiment 125, wherein the cancer-causing protein is mutant K-ras.

Embodiment 127. The composition of embodiment 125 or 126, wherein the cancer-causing protein is G12D K-ras.

Embodiment 128. The composition of embodiment 125 or 126, wherein the cancer-causing protein is G12C K-ras.

Embodiment 129. The composition of embodiment 125 or 126, wherein the cancer-causing protein is G12V K-ras.

Embodiment 130. The composition of any one of embodiments 115-124, wherein the biologically-active agent binds to a nucleic acid sequence encoding a cancer gene.

Embodiment 131. The composition of any one of embodiments 115-124, wherein the biologically-active agent binds to a mRNA sequence transcripted from a cancer gene.

Embodiment 132. The composition of any one of embodiments 115-124, wherein the biologically-active agent binds to a DNA sequence encoding a cancer gene.

Embodiment 133. The composition of any one of embodiments 130-132, wherein the cancer gene is non-wild type KRAS.

Embodiment 134. The composition of any one of embodiments 130-133, wherein the cancer gene is G12D KRAS.

Embodiment 135. The composition of any one of embodiments 130-133, wherein the cancer gene is G12C KRAS.

Embodiment 136. The composition of any one of embodiments 130-133, wherein the cancer gene is G12V KRAS.

Embodiment 137. The composition of any one of embodiments 115-136, wherein the biologically-active agent is an oligonucleotide or oligonucleotide analogue.

Embodiment 138. The composition of any one of embodiments 115-137, wherein the biologically-active agent is a peptide nucleic acid.

Embodiment 139. The composition of any one of embodiments 1-106, wherein the compound is:

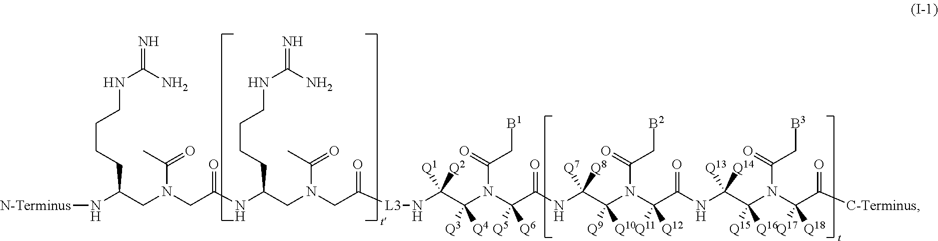

wherein:
each instance of B¹, B² and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L3 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

Embodiment 140. The composition of embodiment 139, wherein L3 is absent. In some embodiments, each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl that is substituted or unsubstituted, or hydrogen.

Embodiment 141. The composition of any one of embodiments 1-106, wherein the compound is:

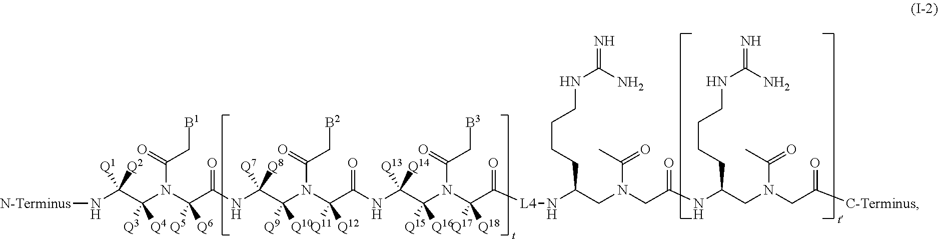

wherein:
each instance of B¹, B², and B³ is independently a heterocycle;
each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl, heteroalkyl, aryl, or heteroaryl, each of which is independently substituted or unsubstituted, or hydrogen;
L4 is a linker group or absent;
N-Terminus is H, acyl, a group that together with the nitrogen atom to which the N-Terminus is bound forms a carbamate, a probe, a fluorophore, a lipid, a metal chelator, or a biological agent;
C-Terminus is —N(H)-J, wherein J is H, acyl, a group that together with the nitrogen atom to which J is bound forms a carbamate, a probe, a fluorophore, a lipid, a metal chelator, or a biological agent;
t is an integer that is from 1 to 30; and
t′ is an integer that is from 2 to 9,
   or a pharmaceutically acceptable salt or ionized form thereof.

Embodiment 142. The composition of embodiment 141, wherein L4 is absent.

Embodiment 143. The composition of any one of embodiments 139-142, wherein N-Terminus is H.

Embodiment 144. The composition of any one of embodiments 139-142, wherein N-Terminus is acyl.

Embodiment 145. The composition of any one of embodiments 139-142, wherein N-Terminus is acetyl.

Embodiment 146. The composition of any one of embodiments 139-145, wherein C-Terminus is NH₂.

Embodiment 147. The composition of any one of embodiments 139-146, wherein each instance of Q¹, Q², Q³, Q⁴, Q⁵, Q⁶, Q⁷, Q⁸, Q⁹, Q¹⁰, Q¹¹, Q¹², Q¹³, Q¹⁴, Q¹⁵, Q¹⁶, Q¹⁷, and Q¹⁸ is independently an amino acid side chain, alkyl that is substituted or unsubstituted, or hydrogen.

Embodiment 148. The composition of any one of embodiments 1-147, wherein the composition comprises the pharmaceutically-acceptable salt of the compound or an ionized form thereof, wherein the pharmaceutically-acceptable salt of the compound is a chloride salt.

Embodiment 149. The composition of any one of embodiments 1-147, wherein the composition comprises the pharmaceutically-acceptable salt of the compound or an ionized form thereof, wherein the pharmaceutically-acceptable salt of the compound is a trifluoroacetate salt.

Embodiment 150. The composition of any one of embodiments 1-147, wherein the composition comprises the pharmaceutically-acceptable salt of the compound or an ionized form thereof, wherein the pharmaceutically-acceptable salt of the compound is an acetate salt.

Embodiment 151. The composition of embodiment 150, wherein the pharmaceutically-acceptable salt comprises about 5% to about 15% (w/w) acetate.

Embodiment 152. The composition of embodiment 150, wherein the pharmaceutically-acceptable salt comprises about 10% (w/w) acetate.

Embodiment 153. The composition of any one of embodiments 1-152, wherein the pharmaceutical composition is in the form of an aqueous solution.

Embodiment 154. A pharmaceutical composition comprising:
(a) an oligonucleotide analogue comprising a structure according to formula (II):

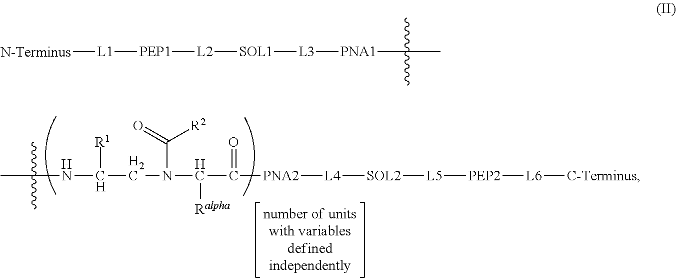

wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
   or a pharmaceutically-acceptable salt or ionized form thereof; and
(b) an amino acid or a pharmaceutically-acceptable salt or ionized form thereof thereof, wherein the amino acid comprises a side chain that is positively charged at physiological pH.

Embodiment 155. The composition of embodiment 154, wherein the amino acid is arginine.

Embodiment 156. The composition of embodiment 154, wherein the amino acid is lysine.

Embodiment 157. The composition of embodiment 154, wherein the amino acid is ornithine.

Embodiment 158. The composition of embodiment 154, wherein the amino acid is histidine.

Embodiment 159. The composition of embodiment 158, wherein histidine or an ionized form thereof is present in the composition at a concentration of about 0.5 mM to about 100 mM.

Embodiment 160. The composition of embodiment 158, wherein histidine or an ionized form thereof is present in the composition at a concentration of about 25 mM to about 100 mM.

Embodiment 161. The composition of embodiment 158, wherein histidine or an ionized form thereof is present in the composition at a concentration of about 25 mM to about 75 mM.

Embodiment 162. The composition of embodiment 158, wherein histidine or an ionized form thereof is present in the composition at a concentration of about 40 mM.

Embodiment 163. The composition of embodiment 158, wherein histidine or an ionized form thereof is present in the composition at a concentration of about 50 mM.

Embodiment 164. The composition of any one of embodiments 158-163, wherein the histidine is L-histidine.

Embodiment 165. The composition of any one of embodiments 154-164, wherein the pharmaceutical composition further comprises a second amino acid or a pharmaceutically-acceptable salt or ionized form thereof.

Embodiment 166. The composition of embodiment 165, wherein the second amino acid comprises a side chain that is positively charged at physiological pH.

Embodiment 167. The composition of embodiment 165 or 166, wherein the second amino acid is arginine.

Embodiment 168. The composition of any one of embodiments 154-167, wherein the pharmaceutical composition further comprises an anti-aggregation agent.

Embodiment 169. The composition of embodiment 168, wherein the anti-aggregation agent is a polyol.

Embodiment 170. The composition of embodiment 168 or 169, wherein the anti-aggregation agent is a saccharide.

Embodiment 171. The composition of any one of embodiments 168-170, wherein the anti-aggregation agent is a sugar.

Embodiment 172. The composition of any one of embodiments 168-170, wherein the anti-aggregation agent is a sugar alcohol.

Embodiment 173. The composition of any one of embodiments 168-172, wherein the anti-aggregation agent is maltitol, mannitol, isomalt, sorbitol, xylitol, or erythritol.

Embodiment 174. The composition of any one of embodiments 168-172, wherein the anti-aggregation agent is mannitol.

Embodiment 175. The composition of embodiment 174, wherein mannitol is present in the composition at a concentration from about 5 mM to about 200 mM.

Embodiment 176. The composition of embodiment 174, wherein mannitol is present in the composition at a concentration from about 50 mM to about 150 mM.

Embodiment 177. The composition of embodiment 174, wherein mannitol is present in the composition at a concentration from about 5 mM to about 30 mM.

Embodiment 178. The composition of embodiment 174, wherein mannitol is present in the composition at a concentration of 15 mM.

Embodiment 179. The composition of embodiment 174, wherein mannitol is present in the composition at a concentration of 100 mM.

Embodiment 180. The composition of any one of embodiments 168-170, wherein the anti-aggregation agent is a sugar.

Embodiment 181. The composition of any one of embodiments 168-170 and 180, wherein anti-aggregation agent is a monosaccharide.

Embodiment 182. The composition of any one of embodiments 168-170, 180, and 181, wherein the anti-aggregation agent is dextrose.

Embodiment 183. The composition of embodiment 182, wherein dextrose is present in the composition at a concentration of about 50 mM to about 400 mM.

Embodiment 184. The composition of embodiment 182, wherein dextrose is present in the composition at a concentration of about 50 mM to about 300 mM.

Embodiment 185. The composition of embodiment 182, wherein dextrose is present in the composition at a concentration of about 200 mM to about 250 mM.

Embodiment 186. The composition of embodiment 182, wherein dextrose is present in the composition at a concentration of about 100 mM.

Embodiment 187. The composition of embodiment 182, wherein dextrose is present in the composition at a concentration of about 230 mM.

Embodiment 188. The composition of any one of embodiments 154-187, wherein the number of units with variables defined independently is 11, 12, 13, 14, 15, 16, or 17.

Embodiment 189. The composition of any one of embodiments 154-187, wherein the number of units with variables defined independently is 14.

Embodiment 190. The composition of any one of embodiments 154-189, wherein N-Terminus is H and C-Terminus is NH₂.

Embodiment 191. The composition of any one of embodiments 154-190, wherein each R^{alpha} is independently H, hydroxylmethyl, or 4-guanidinobut-1-yl.

Embodiment 192. The composition of any one of embodiments 154-190, wherein each R^{alpha} is H.

Embodiment 193. The composition of any one of embodiments 154-190, wherein at least one iteration of R^{alpha} is hydroxylmethyl.

Embodiment 194. The composition of any one of embodiments 154-190, wherein at least one iteration of R^{alpha} is 4-guanidinobut-1-yl.

Embodiment 195. The composition of any one of embodiments 154-190, wherein at least half the iterations of R^{alpha} are hydroxylmethyl and the other iterations of R^{alpha} are H.

Embodiment 196. The composition of any one of embodiments 154-190, wherein at least half the iterations of R^{alpha} are 4-guanidinobut-1-yl and the other iterations of R^{alpha} are H.

Embodiment 197. The composition of any one of embodiments 154-196, wherein each R¹ is independently H, hydroxylmethyl, or 4-guanidinobut-1-yl.

Embodiment 198. The composition of any one of embodiments 154-196, wherein each R¹ is H.

Embodiment 199. The composition of any one of embodiments 154-196, wherein at least one iteration of R¹ is hydroxylmethyl.

Embodiment 200. The composition of any one of embodiments 154-196, wherein at least one iteration of R¹ is 4-guanidinobut-1-yl.

Embodiment 201. The composition of any one of embodiments 154-196, wherein at least half the iterations of R¹ are hydroxylmethyl and the other iterations of R¹ are H.

Embodiment 202. The composition of any one of embodiments 154-196, wherein at least half the iterations of R¹ are 4-guanidinobut-1-yl and the other iterations of R¹ are H.

Embodiment 203. The composition of any one of embodiments 154-202, wherein each of L1, L2, L3, L4, L5, and L6 is absent.

Embodiment 204. The composition of any one of embodiments 154-203, wherein PEP1 and PEP2 are absent.

Embodiment 205. The composition of any one of embodiments 154-203, wherein one of PEP1 and PEP2 is a peptide sequence that is a nuclear localization sequence and the other is absent.

Embodiment 206. The composition of any one of embodiments 154-205, wherein SOL1 is the water-solubilizing group and SOL2 is absent.

Embodiment 207. The composition of any one of embodiments 154-202, wherein each of L1, L2, L3, L4, L5, L6, PEP1, PEP2, and SOL2 is absent, and SOL1 is the water-solubilizing group.

Embodiment 208. The composition of any one of embodiments 154-202, 206, and 207, wherein the water-solubilizing group is a group that contains multiple positive charges at physiological pH.

Embodiment 209. The composition of any one of embodiments 154-208, wherein the water-solubilizing group of SOL1 is a group of formula:

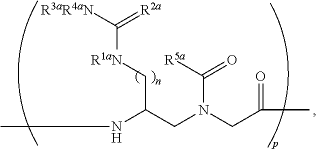

wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is alkyl or O-alkyl, any of which is unsubstituted or substituted;
n is 1, 2, 3, 4, 5, 6, 7, 8, 9, or 10; and
p is an integer from 1-100.

Embodiment 210. The composition of embodiment 209, wherein p is 5, 6, 7, or 8.

Embodiment 211. The composition of any one of embodiments 154-209, wherein the water-solubilizing group of SOL1 is a group of formula:

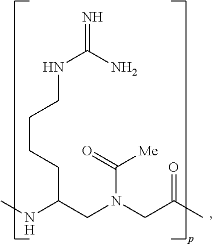

wherein p is an integer that is 5, 6, 7, or 8.

Embodiment 212. The composition of any one of embodiments 154-209, wherein the water-solubilizing group of SOL1 is a group of formula:

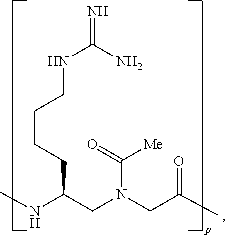

wherein p is an integer that is 5, 6, 7, or 8.

Embodiment 213. The composition of any one of embodiments 209-212, wherein p is 7.

Embodiment 214. The composition of any one of embodiments 154-212, wherein the heterocycles of the R² groups are each independently:

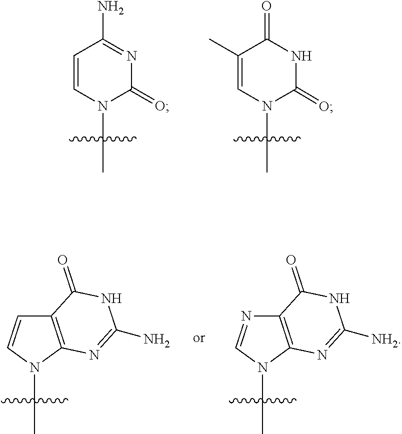

Embodiment 215. The composition of any one of embodiments 154-212, wherein each R² is independently: methyl,

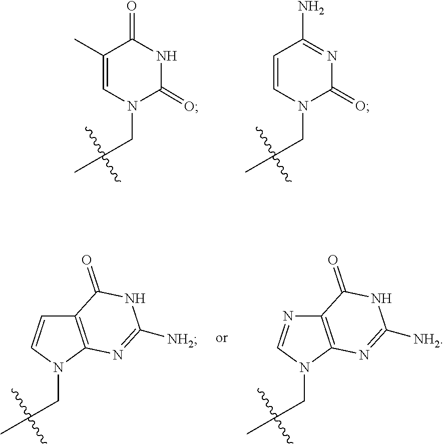 [SMILES: *Cn1cc(C)c(=O)[nH]c1=O ; *Cn1ccc(N)nc1=O ; *Cn1ccc2c(=O)[nH]c(N)nc21 ; "or" ; *Cn1cnc2c(=O)[nH]c(N)nc21]

Embodiment 216. The composition of any one of embodiments 154-212, wherein the heterocycles of the R² groups form a sequence that repeats at least twice, wherein the sequence is, from N-Terminus to C-Terminus:

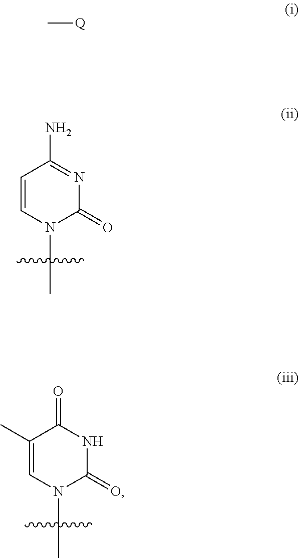

wherein Q is

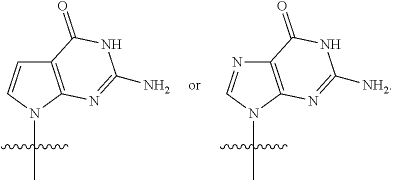 [SMILES: *n1ccc2c(=O)[nH]c(N)nc21 ; *n1cnc2c(=O)[nH]c(N)nc21]

Embodiment 217. The composition of any one of embodiments 154-212, wherein the heterocycles of the R² groups are each independently:

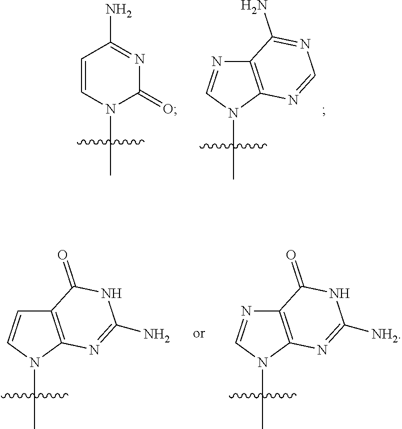

Embodiment 218. The composition of any one of embodiments 154-212, wherein each R² is independently: methyl,

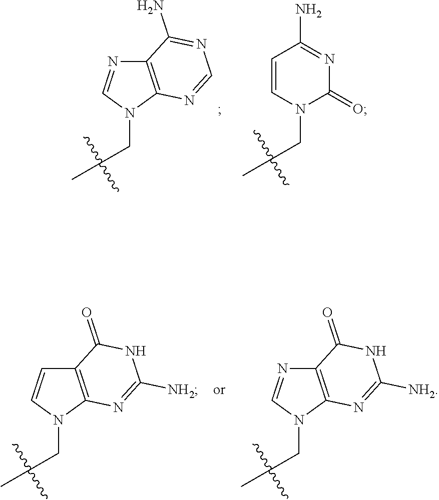 [SMILES: *Cn1cnc2c(N)ncnc21 ; *Cn1ccc(N)nc1=O ; *Cn1ccc2c(=O)[nH]c(N)nc21 ; *Cn1cnc2c(=O)[nH]c(N)nc21]

Embodiment 219. The composition of any one of embodiments 154-212, wherein the heterocycles of the R² groups form a sequence that repeats at least twice, wherein the sequence is, from N-Terminus to C-Terminus:

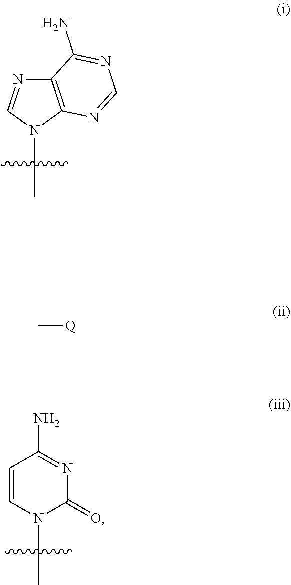

wherein Q is

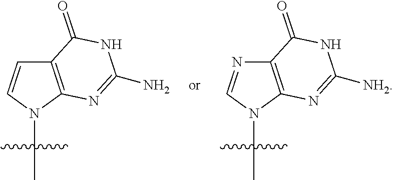 [SMILES: *n1ccc2c(=O)[nH]c(N)nc21 ; *n1cnc2c(=O)[nH]c(N)nc21]

Embodiment 220. The composition of any one of embodiments 154-212, wherein the heterocycles of the R² groups are each independently:

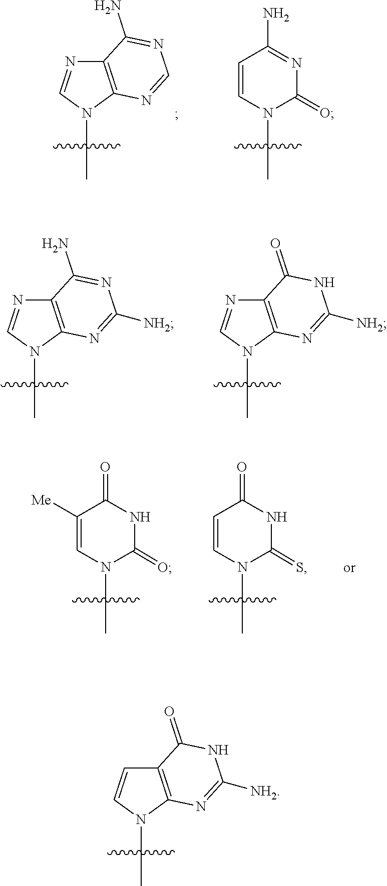

Embodiment 221. The composition of any one of embodiments 154-212, wherein each R² is independently: methyl,

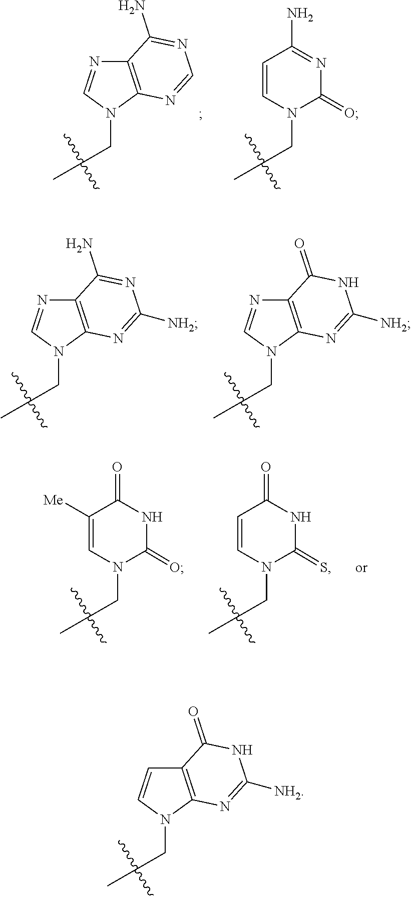

Embodiment 222. The composition of any one of embodiments 154-212, wherein the heterocycles of the R² groups form a sequence that repeats at least twice, wherein the sequence is, from N-Terminus to C-Terminus:

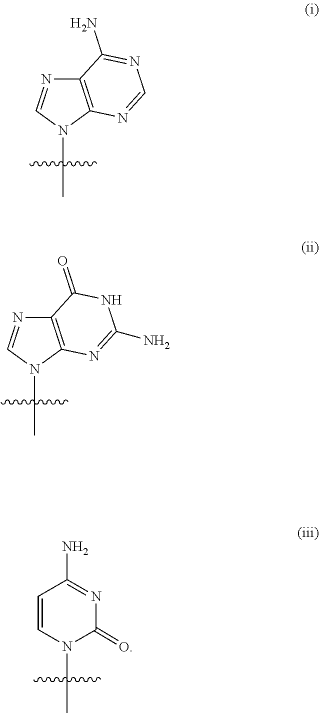 [SMILES: *n1cnc2c(N)ncnc21 ; *n1cnc2c(=O)[nH]c(N)nc21 ; *n1ccc(N)nc1=O]

Embodiment 223. The composition of any one of embodiments 154-222, wherein the compound binds to a nucleic acid sequence transcribed from a gene associated with a neuromuscular disease phenotype.

Embodiment 224. The composition of any one of embodiments 154-222, wherein the compound binds to a nucleic acid sequence transcribed from a gene associated with a DM1 disease phenotype.

Embodiment 225. The composition of any one of embodiments 154-224, wherein the compound binds to a nucleic acid sequence transcribed from a gene associated with a DM1 disease phenotype by interactions between the heterocycles of the R² groups and nucleobases of a DM1 gene.

Embodiment 226. The composition of embodiment 225, wherein the DM1 gene is a non-wild type DM1 gene. In some embodiments, the non-wild type DM1 gene differs from a wild type DM1 gene in a repeat expansion mutation.

Embodiment 227. The composition of any one of embodiments 154-223, wherein the compound binds to a nucleic acid sequence transcribed from a gene associated with a neurodegenerative disease phenotype.

Embodiment 228. The composition of any one of embodiments 154-223 and 227, wherein the compound binds to a nucleic acid sequence transcribed from a gene associated with a Huntington's disease phenotype.

Embodiment 229. The composition of any one of embodiments 154-223 and 227, wherein the compound binds to a nucleic acid sequence transcribed from a gene associated with a Huntington's disease phenotype by interactions between the heterocycles of the R² groups and nucleobases of a HTT gene.

Embodiment 230. The composition of embodiment 229, wherein the HTT gene is a non-wild type HTT gene.

Embodiment 231. The composition of embodiment 230, wherein the non-wild type HTT gene differs from a wild type HTT gene in a repeat expansion mutation.

Embodiment 232. The composition of any one of embodiments 154-209, wherein the compound binds to a mRNA sequence of (CUG)_{z}, wherein z is an integer from 1-100 (SEQ ID NO: 324).

Embodiment 233. The composition of any one of embodiments 154-209, wherein the compound binds to a mRNA sequence of (CAG)_{z}, wherein z is an integer from 1-100 (SEQ ID NO: 325).

Embodiment 234. The composition of any one of embodiments 154-222, wherein the compound binds to a nucleic acid sequence encoding a cancer-causing protein.

Embodiment 235. The composition of embodiment 234, wherein the cancer-causing protein is mutant K-ras.

Embodiment 236. The composition of embodiment 234, wherein the cancer-causing protein is G12D K-ras.

Embodiment 237. The composition of embodiment 234, wherein the cancer-causing protein is G12C K-ras.

Embodiment 238. The composition of embodiment 234, wherein the cancer-causing protein is G12V K-ras.

Embodiment 239. The composition of any one of embodiments 154-238, wherein the compound binds to the nucleic acid sequence encoding the mutant K-ras by interactions between the heterocycles of the R² groups and nucleobases of the nucleic acid sequence.

Embodiment 240. The composition of any one of embodiments 154-239, wherein the nucleic acid sequence is a mRNA sequence.

Embodiment 241. The composition of any one of embodiments 154-239, wherein the nucleic acid sequence is a DNA sequence.

Embodiment 242. The composition of any one of embodiments 1, 35, 61, 106, 139, 154 and 209, wherein the compound is:

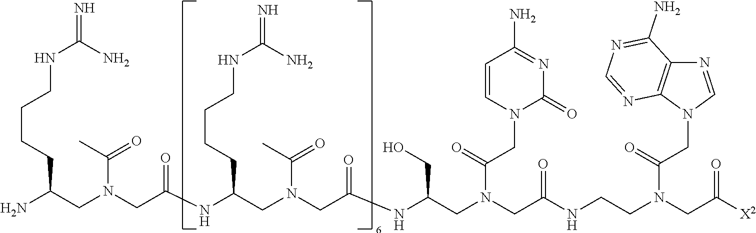

wherein X² is

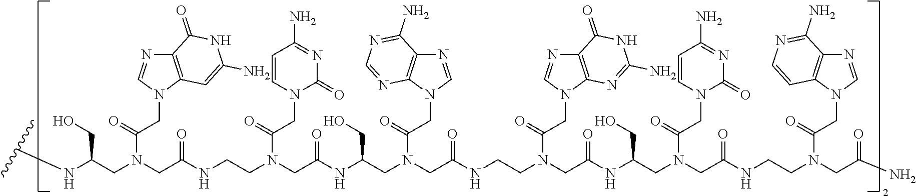

(Compound 1) (SEQ ID NO: 27) or a pharmaceutically-acceptable salt or ionized form thereof.

Embodiment 243. The composition of any one of embodiments 1, 35, 61, 106, 139, 154 and 209, wherein the compound is:

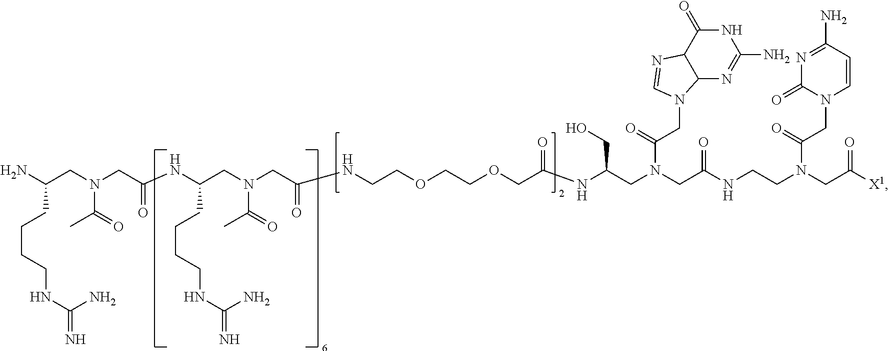

wherein X is

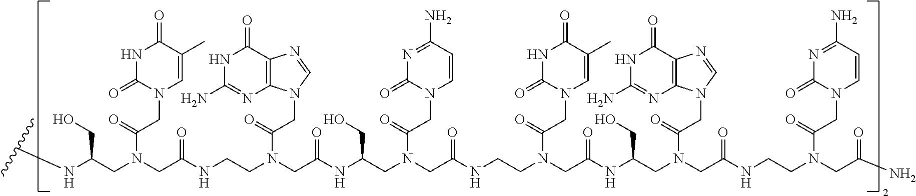

(Compound 166) (SEQ ID NO: 81), or a pharmaceutically-acceptable salt or ionized form thereof.

Embodiment 244. The composition of any one of embodiments 1, 35, 61, 106, 139, 154 and 209, wherein the compound is:

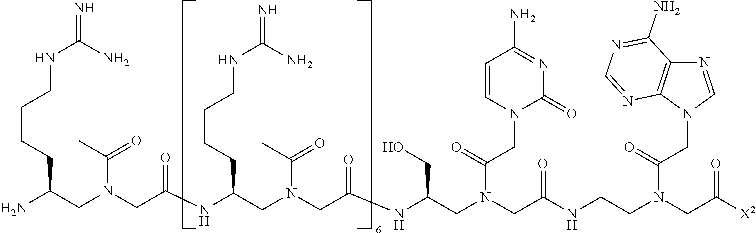

wherein X² is

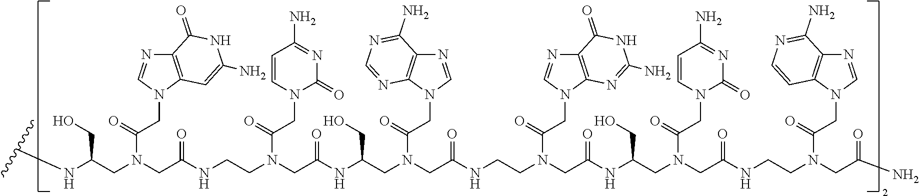

(Compound 167) (SEQ ID NO: 27), or a pharmaceutically-acceptable salt or ionized form thereof.

Embodiment 245. The composition of any one of embodiments 154-244, wherein the composition comprises the pharmaceutically-acceptable salt of the compound or an ionized form thereof, wherein the pharmaceutically-acceptable salt of the compound is a chloride salt.

Embodiment 246. The composition of any one of embodiments 154-244, wherein the composition comprises the pharmaceutically-acceptable salt of the compound or an ionized form thereof, wherein the pharmaceutically-acceptable salt of the compound is a trifluoroacetate salt.

Embodiment 247. The composition of any one of embodiments 154-244, wherein the composition comprises the pharmaceutically-acceptable salt of the compound or an ionized form thereof, wherein the pharmaceutically-acceptable salt of the compound is an acetate salt.

Embodiment 248. The composition of embodiment 247, wherein the pharmaceutically-acceptable salt comprises from about 5% to about 15% (w/w) acetate.

Embodiment 249. The composition of embodiment 247, wherein the pharmaceutically-acceptable salt comprises about 10% (w/w) acetate.

Embodiment 250. A method of treating a condition in a subject in need thereof, comprising administering to the subject a pharmaceutical composition of any one of embodiments 1-249.

Embodiment 251. The method of embodiment 250, wherein the administering is intravenous administration.

Embodiment 252. The method of embodiment 250, wherein the administering is subcutaneous administration.

Embodiment 253. The method of embodiment 250, wherein the administering is intramuscular administration.

Embodiment 254. The method of embodiment 250, wherein the administering is intracerebroventricular administration.

Embodiment 255. The method of embodiment 250, wherein the administering is intrathecal administration.

Embodiment 256. The method of any one of embodiments 250-255, wherein the condition is a neurodegenerative disease.

Embodiment 257. The method of any one of embodiments 250-256, wherein the condition is associated with an expanded trinucleotide repeat.

Embodiment 258. The method of any one of embodiments 250-257, wherein the condition is Huntington's disease.

Embodiment 259. The method of any one of embodiments 250-255, wherein the condition is a neuromuscular disease.

Embodiment 260. The method of any one of embodiments 250-255 and 259, wherein the condition is associated with an expanded trinucleotide repeat.

Embodiment 261. The method of any one of embodiments 250-255, 259, and 260, wherein the condition is myotonic dystrophy type 1.

Embodiment 262. The method of any one of embodiments 250-255, wherein the condition is cancer.

### EXAMPLES

### Example 1: Phosphate-Buffered Saline (PBS) Formulations

Formulations were made using a phosphate-buffered saline (PBS) solution combined with drug substance. Formulations made with lower concentrations of drug substance were made by dilution with PBS solution from formulations with a higher drug substance concentration.

Formulations listed in TABLE 5 were made using the trifluoroacetate salt of Compound 1 (as a lyophilized powder) and a commercially available PBS solution (1× without Ca²⁺ and Mg²⁺), having a pH of about 7.4. The commercially available 1×PBS solution without calcium and magnesium contained potassium dihydrogen phosphate at 0.144 g/L, sodium chloride at 9 g/L, and disodium phosphate at 0.795 g/L with a pH of about 7.4 (e.g., Corning Product Number 21-040-CM). Dilutions were made from the highest concentration of Compound 1 listed in Table 1 to the lowest listed concentrations. TABLE 5 shows the concentration in mg/mL of Compound 1 used and the apparent molecular weight derived from static light scattering (SLS) measurements.

TABLE 5
Formulations made with a Phosphate buffered saline (PBS) buffer.

| Formulation | Compound 1, | | App MW (kDa) |
|---|---|---|---|
| number | net mg/mL | Formulation | by SLS |
| 1 | 13.4 | 1X Phosphate | 33.6 |
| | | Buffered Saline | |
| | | (w/out Ca and Mg), | |
| | | pH 7.4 | |
| 2 | 10 | 1X Phosphate | 39 |
| | | Buffered Saline | |
| | | (w/out Ca and Mg), | |
| | | pH 7.4 | |
| 3 | 8 | 1X Phosphate | 42.7 |
| | | Buffered Saline | |
| | | (w/out Ca and Mg), | |
| | | pH 7.4 | |
| 4 | 6 | 1X Phosphate | 36.5 |
| | | Buffered Saline | |
| | | (w/out Ca and Mg), | |
| | | pH 7.4 | |
| 5 | 4 | 1X Phosphate | 38 |
| | | Buffered Saline | |
| | | (w/out Ca and Mg), | |
| | | pH 7.4 | |
| 6 | 3 | 1X Phosphate | 33.5 |
| | | Buffered Saline | |
| | | (w/out Ca and Mg), | |
| | | pH 7.4 | |
| 7 | 2 | 1X Phosphate | signal too low |
| | | Buffered Saline | |
| | | (w/out Ca and Mg), | |
| | | pH 7.4 | |
| 8 | 1 | 1X Phosphate | signal too low |
| | | Buffered Saline | |
| | | (w/out Ca and Mg), | |
| | | pH 7.4 | |

### Example 2: Ammonium Formulations Vs. Sodium Formulations

Formulations were made using solutions of ammonium or sodium phosphate combined with trifluoroacetate salt of Compound 1. Formulations with lower drug substance concentrations were prepared by diluting formulations having higher drug substance concentrations with blank formulations. Formulations were made by mixing concentrated formulation stock solutions with water and an aqueous solution of trifluoroacetate salt of Compound 1.

Formulation stock solutions were made with excipients at ten times the final formulated concentration listed in TABLE 6 and titrated to a pH of about 7.5 with 1 Molar (M) hydrochloric (HCl) acid. Blank formulations at the concentrations listed in TABLE 6 were made by mixing one part of stock solution with nine parts water. Formulations made with 12 mg/mL of Compound 1 were made by mixing one part of formulation stock solution with eight parts of a 2.67 mM (15 mg/mL) stock solution of Compound 1 in water and one part water. All formulations in TABLE 6 employed trifluoroacetate salt of Compound 1. Formulations with lower concentrations were made by diluting the 12 mg/mL formulation with formulation blanks to the concentrations of Compound 1 listed in TABLE 6. TABLE 6 shows the concentration of Compound 1 and the apparent molecular weight of each formulation derived from SLS measurements.

TABLE 6
Formulations made with Ammonium Phosphate
or Sodium Phosphate buffers

| Formu- | Compound | | 1M stock | |
|---|---|---|---|---|
| lation | 1, net | Formulation Final | solution | App SLS |
| Number | mg/mL | Concentrations | pH (pre-PNA) | MW, kDa |
| 9 | 12 | 100 mM | 7.58 | 30.4 |
| | | ammonium | | |
| | | phosphate dibasic | | |
| 10 | 10 | 100 mM | 7.58 | 16.3 |
| | | ammonium | | |
| | | phosphate dibasic | | |
| 11 | 8 | 100 mM | 7.58 | 17 |
| | | ammonium | | |
| | | phosphate dibasic | | |
| 12 | 6 | 100 mM | 7.58 | 11.4 |
| | | ammonium | | |
| | | phosphate dibasic | | |
| 13 | 4 | 100 mM | 7.58 | 17.3 |
| | | ammonium | | |
| | | phosphate dibasic | | |
| 14 | 12 | 100 mM | 7.53 | 37.3 |
| | | ammonium | | |
| | | phosphate dibasic, | | |
| | | 50 mM | | |
| | | Ammonium | | |
| | | chloride | | |
| 15 | 10 | 100 mM | 7.53 | 28.3 |
| | | ammonium | | |
| | | phosphate dibasic, | | |
| | | 50 mM | | |
| | | Ammonium | | |
| | | chloride | | |
| 16 | 8 | 100 mM | 7.53 | 17 |
| | | ammonium | | |
| | | phosphate dibasic, | | |
| | | 50 mM | | |
| | | Ammonium | | |
| | | chloride | | |
| 17 | 6 | 100 mM | 7.53 | 13.2 |
| | | ammonium | | |
| | | phosphate dibasic, | | |
| | | 50 mM | | |
| | | Ammonium | | |
| | | chloride | | |
| 18 | 4 | 100 mM | 7.53 | 16.7 |
| | | ammonium | | |
| | | phosphate dibasic, | | |
| | | 50 mM | | |
| | | Ammonium | | |
| | | chloride | | |
| 19 | 12 | 100 mM Sodium | 7.52 | 42.9 |
| | | phosphate dibasic | | |
| | | dihydrate | | |
| 20 | 10 | 100 mM Sodium | 7.52 | 45.6 |
| | | phosphate dibasic | | |
| | | dihydrate | | |
| 21 | 8 | 100 mM Sodium | 7.52 | 37.9 |
| | | phosphate dibasic | | |
| | | dihydrate | | |
| 22 | 6 | 100 mM Sodium | 7.52 | 25 |
| | | phosphate dibasic | | |
| | | dihydrate | | |
| 23 | 4 | 100 mM Sodium | 7.52 | 18.7 |
| | | phosphate dibasic | | |
| | | dihydrate | | |
| 24 | 12 | 100 mM Sodium | 7.5 | 20.7 |
| | | phosphate dibasic | | |
| | | dihydrate, 100 mM | | |
| | | Sodium chloride | | |
| 25 | 10 | 100 mM Sodium | 7.5 | 19.2 |
| | | phosphate dibasic | | |
| | | dihydrate, 100 mM | | |
| | | Sodium chloride | | |
| 26 | 8 | 100 mM Sodium | 7.5 | 17 |
| | | phosphate dibasic | | |
| | | dihydrate, 100 mM | | |
| | | Sodium chloride | | |
| 27 | 6 | 100 mM Sodium | 7.5 | 13 |
| | | phosphate dibasic | | |
| | | dihydrate, 100 mM | | |
| | | Sodium chloride | | |
| 28 | 4 | 100 mM Sodium | 7.5 | 13.8 |
| | | phosphate dibasic | | |
| | | dihydrate, 100 mM | | |
| | | Sodium chloride | | |

### Example 3: Initial Design of Experiment (DOE) Screen Formulations

Formulations were made using buffered solutions with a physiological pH range and an additional excipient combined with drug substance. Formulations with lower drug substance concentrations were prepared by diluting formulations having higher drug substance concentrations with blank formulations. Formulations were made by mixing concentrated formulation stock solutions with water and chloride salt of Compound 1 dissolved in water.

Formulation stock solutions were made with excipients at 1.6 times their final formulated concentration listed in TABLE 7 and titrated to a desired pH of about 5.5, 6.1, 7.0, or 7.5 with 1 M HCl or 1 M sodium hydroxide (NaOH). Blank formulations at the concentrations listed in TABLE 7 were made by mixing five parts of stock solution with three parts water. Formulations made with 15 mg/mL of Compound 1 were made by mixing 250 parts of formulation stock solutions with 84 parts of a 71.4 mg/mL stock solution of Compound 1 in water and 66 parts water. All formulations in TABLE 7 used chloride salt of Compound 1. Formulations with lower concentrations were made by diluting the 15 mg/mL formulation with formulation blanks to the concentrations of Compound 1 listed in TABLE 7.

TABLE 7 shows the concentration of Compound 1, the pH of the blank formulations, pH of final formulations, and the presence of aggregates as determined by dynamic light scattering (DLS) in 15 mg/mL formulations. Aggregates were noted as present if aggregates larger than 20 nm contained greater than 1% of Compound 1 by mass.

Use of Tris HCl buffer resulted in the formulation of aggregates at a pH of 7.5. Formulations containing sodium phosphate-based buffers exhibited visible precipitation of Compound 1. Formulations containing histidine exhibited the least aggregation. Mannitol and dextrose more effectively suppressed aggregation than sucrose. Observations indicated that sodium chloride induced aggregation.

TABLE 7
Formulations with different combinations of buffers and sugar/salt

| | | | | | | Presence of |
|---|---|---|---|---|---|---|
| | | | | | | Aggregates > |
| | | | | | | 20 nm > |
| | | | | | | 1% by Mass |
| | | | | Formul. | Final | @ 15 |
| | Compound | | Final | Blank pH | Formul. pH, | mg/mL |
| Formul. | 1, net | Final Buffer | Sugar/Salt | ambient | ambient | (DLS at |
| Number | mg/mL | Comp. | and conc. | temperature | temperature | 25 C) |
| 29 | 3 | 100 mM | 15 mM | 7.34 | 3.29 | No |
| | | TRIS*HCl | Sucrose | | | |
| | | solution | | | | |
| | | adjusted to | | | | |
| | | pH 7.6 with | | | | |
| | | 1N HCl | | | | |
| 30 | 6 | 100 mM | 15 mM | 7.34 | 2.2 | No |
| | | TRIS*HCl | Sucrose | | | |
| | | solution | | | | |
| | | adjusted to | | | | |
| | | pH 7.6 with | | | | |
| | | 1N HCl | | | | |
| 31 | 9 | 100 mM | 15 mM | 7.34 | 1.84 | No |
| | | TRIS*HCl | Sucrose | | | |
| | | solution | | | | |
| | | adjusted to | | | | |
| | | pH 7.6 with | | | | |
| | | 1N HCl | | | | |
| 32 | 12 | 100 mM | 15 mM | 7.34 | 1.56 | No |
| | | TRIS*HCl | Sucrose | | | |
| | | solution | | | | |
| | | adjusted to | | | | |
| | | pH 7.6 with | | | | |
| | | 1N HCl | | | | |
| 33 | 15 | 100 mM | 15 mM | 7.34 | 1.44 | No |
| | | TRIS*HCl | Sucrose | | | |
| | | solution | | | | |
| | | adjusted to | | | | |
| | | pH 7.6 with | | | | |
| | | 1N HCl | | | | |
| 34 | 3 | 100 mM L- | 15 mM | 5.52 | 4.82 | No |
| | | Histidine, | Mannitol | | | |
| | | adjusted to | | | | |
| | | pH 5.5 with | | | | |
| | | 1N HCl | | | | |
| 35 | 6 | 100 mM L- | 15 mM | 5.52 | 3.13 | No |
| | | Histidine, | Mannitol | | | |
| | | adjusted to | | | | |
| | | pH 5.5 with | | | | |
| | | 1N HCl | | | | |
| 36 | 9 | 100 mM L- | 15 mM | 5.52 | 2.55 | No |
| | | Histidine, | Mannitol | | | |
| | | adjusted to | | | | |
| | | pH 5.5 with | | | | |
| | | 1N HCl | | | | |
| 37 | 12 | 100 mML- | 15 mM | 5.52 | 2.36 | No |
| | | Histidine, | Mannitol | | | |
| | | adjusted to | | | | |
| | | pH 5.5 with | | | | |
| | | 1N HCl | | | | |
| 38 | 15 | 100 mM L- | 15 mM | 5.52 | 2.17 | No |
| | | Histidine, | Mannitol | | | |
| | | adjusted to | | | | |
| | | pH 5.5 with | | | | |
| | | 1N HCl | | | | |
| 39 | 3 | 0.1M | 100 mM | 6.8 | 2.12 | No |
| | | TRIS*HCl | Dextrose | | | |
| | | solution | | | | |
| | | adjusted to | | | | |
| | | pH 7.0 with | | | | |
| | | 1N HCl | | | | |
| 40 | 6 | 0.1M | 100 mM | 6.8 | 1.76 | No |
| | | TRIS*HCl | Dextrose | | | |
| | | solution | | | | |
| | | adjusted to | | | | |
| | | pH 7.0 with | | | | |
| | | 1N HCl | | | | |
| 41 | 9 | 0.1M | 100 mM | 6.8 | 1.61 | No |
| | | TRIS*HCl | Dextrose | | | |
| | | solution | | | | |
| | | adjusted to | | | | |
| | | pH 7.0 with | | | | |
| | | 1N HCl | | | | |
| 42 | 12 | 0.1M | 100 mM | 6.8 | 1.52 | No |
| | | TRIS*HCl | Dextrose | | | |
| | | solution | | | | |
| | | adjusted to | | | | |
| | | pH 7.0 with | | | | |
| | | 1N HCl | | | | |
| 43 | 15 | 0.1M | 100 mM | 6.8 | 1.4 | No |
| | | TRIS*HCl | Dextrose | | | |
| | | solution | | | | |
| | | adjusted to | | | | |
| | | pH 7.0 with | | | | |
| | | 1N HCl | | | | |
| 44 | 3 | 79.5 mM | 100 mM | 6.14 | 5.07 | No |
| | | Sodium | Dextrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 45 | 6 | 79.5 mM | 100 mM | 6.14 | 3.2 | No |
| | | Sodium | Dextrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 46 | 9 | 79.5 mM | 100 mM | 6.14 | 2.73 | No |
| | | Sodium | Dextrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 47 | 12 | 79.5 mM | 100 mM | 6.14 | 2.47 | No |
| | | Sodium | Dextrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 48 | 15 | 79.5 mM | 100 mM | 6.14 | 2.25 | No |
| | | Sodium | Dextrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 49 | 3 | 100 mM L- | 100 mM | 6.85 | 6.36 | Yes |
| | | Histidine, | Sucrose | | | |
| | | adjusted to | | | | |
| | | pH 7.0 with | | | | |
| | | 1N HCl | | | | |
| 50 | 6 | 100 mM L- | 100 mM | 6.85 | 6.06 | Yes |
| | | Histidine, | Sucrose | | | |
| | | adjusted to | | | | |
| | | pH 7.0 with | | | | |
| | | 1N HCl | | | | |
| 51 | 9 | 100 mM L- | 100 mM | 6.85 | 5.77 | Yes |
| | | Histidine, | Sucrose | | | |
| | | adjusted to | | | | |
| | | pH 7.0 with | | | | |
| | | 1N HCl | | | | |
| 52 | 12 | 100 mM L- | 100 mM | 6.85 | 5.36 | Yes |
| | | Histidine, | Sucrose | | | |
| | | adjusted to | | | | |
| | | pH 7.0 with | | | | |
| | | 1N HCl | | | | |
| 53 | 15 | 100 mM L- | 100 mM | 6.85 | 4.53 | Yes |
| | | Histidine, | Sucrose | | | |
| | | adjusted to | | | | |
| | | pH 7.0 with | | | | |
| | | 1N HCl | | | | |
| 54 | 3 | 79.5 mM | 15 mM | 6.15 | 5.01 | No |
| | | Sodium | Sodium | | | |
| | | Phosphate | Chloride | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 55 | 6 | 79.5 mM | 15 mM | 6.15 | 3.11 | No |
| | | Sodium | Sodium | | | |
| | | Phosphate | Chloride | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 56 | 9 | 79.5 mM | 15 mM | 6.15 | 2.69 | No |
| | | Sodium | Sodium | | | |
| | | Phosphate | Chloride | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 57 | 12 | 79.5 mM | 15 mM | 6.15 | 2.44 | No |
| | | Sodium | Sodium | | | |
| | | Phosphate | Chloride | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 58 | 15 | 79.5 mM | 15 mM | 6.15 | 2.27 | No |
| | | Sodium | Sodium | | | |
| | | Phosphate | Chloride | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 59 | 3 | 79.5 mM | 15 mM | 6.16 | 5.15 | No |
| | | Sodium | Sucrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 60 | 6 | 79.5 mM | 15 mM | 6.16 | 3.1 | No |
| | | Sodium | Sucrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 61 | 9 | 79.5 mM | 15 mM | 6.16 | 2.69 | No |
| | | Sodium | Sucrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 62 | 12 | 79.5 mM | 15 mM | 6.16 | 2.44 | No |
| | | Sodium | Sucrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 63 | 15 | 79.5 mM | 15 mM | 6.16 | 2.26 | No |
| | | Sodium | Sucrose | | | |
| | | Phosphate | | | | |
| | | monobasic, | | | | |
| | | 20.5 mM | | | | |
| | | Sodium | | | | |
| | | phosphate | | | | |
| | | dibasic | | | | |
| | | dihydrate, | | | | |
| | | pH 6.1 | | | | |
| 64 | 3 | 100 mM | 15 mM | 7.35 | 3.07 | No |
| | | TRIS*HCl | Sodium | | | |
| | | solution | Chloride | | | |
| | | adjusted to | | | | |
| | | pH 7.6 with | | | | |
| | | 1N HCl | | | | |
| 65 | 6 | 100 mM | 15 mM | 7.35 | 2.05 | No |
| | | TRIS*HCl | Sodium | | | |
| | | solution | Chloride | | | |
| | | adjusted to | | | | |
| | | pH 7.6 with | | | | |
| | | 1N HCl | | | | |
| 66 | 9 | 100 mM | 15 mM | 7.35 | 1.92 | No |
| | | TRIS*HCl | Sodium | | | |
| | | solution | Chloride | | | |
| | | adjusted to | | | | |
| | | pH 7.6 with | | | | |
| | | 1N HCl | | | | |
| 67 | 12 | 100 mM | 15 mM | 7.35 | 1.62 | No |
| | | TRIS*HCl | Sodium | | | |
| | | solution | Chloride | | | |
| | | adjusted to | | | | |
| | | pH 7.6 with | | | | |
| | | 1N HCl | | | | |
| 68 | 15 | 100 mM | 15 mM | 7.35 | 1.51 | No |
| | | TRIS*HCl | Sodium | | | |
| | | solution | Chloride | | | |
| | | adjusted to | | | | |
| | | pH 7.6 with | | | | |
| | | 1N HCl | | | | |
| 69 | 3 | 100 mM L- | 15 mM | 5.53 | 4.75 | No |
| | | Histidine, | Dextrose | | | |
| | | adjusted to | | | | |
| | | pH 5.5 with | | | | |
| | | 1N HCl | | | | |
| 70 | 6 | 100 mM L- | 15 mM | 5.53 | 3.11 | No |
| | | Histidine, | Dextrose | | | |
| | | adjusted to | | | | |
| | | pH 5.5 with | | | | |
| | | 1N HCl | | | | |
| 71 | 9 | 100 mM L- | 15 mM | 5.53 | 2.57 | No |
| | | Histidine, | Dextrose | | | |
| | | adjusted to | | | | |
| | | pH 5.5 with | | | | |
| | | 1N HCl | | | | |
| 72 | 12 | 100 mM L- | 15 mM | 5.53 | 2.29 | No |
| | | Histidine, | Dextrose | | | |
| | | adjusted to | | | | |
| | | pH 5.5 with | | | | |
| | | 1N HCl | | | | |
| 73 | 15 | 100 mM L- | 15 mM | 5.53 | 2.18 | No |
| | | Histidine, | Dextrose | | | |
| | | adjusted to | | | | |
| | | pH 5.5 with | | | | |
| | | 1N HCl | | | | |
| 74 | 3 | 24.5 mM | 100 mM | 7.42 | 6.94 | Yes |
| | | Sodium | Sodium | | | |
| | | Phosphate | Chloride | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 75 | 6 | 24.5 mM | 100 mM | 7.42 | 6.59 | Yes |
| | | Sodium | Sodium | | | |
| | | Phosphate | Chloride | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 76 | 9 | 24.5 mM | 100 mM | 7.42 | 6.27 | Yes |
| | | Sodium | Sodium | | | |
| | | Phosphate | Chloride | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 77 | 12 | 24.5 mM | 100 mM | 7.42 | 5.84 | Yes |
| | | Sodium | Sodium | | | |
| | | Phosphate | Chloride | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 78 | 15 | 24.5 mM | 100 mM | 7.42 | 4.93 | Yes |
| | | Sodium | Sodium | | | |
| | | Phosphate | Chloride | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 79 | 3 | 100 mM L- | 100 mM | 5.55 | 4.76 | No |
| | | Histidine, | Sodium | | | |
| | | adjusted to | Chloride | | | |
| | | pH 5.5 with | | | | |
| | | 1N HCl | | | | |
| 80 | 6 | 100 mM L- | 100 mM | 5.55 | 3.1 | No |
| | | Histidine, | Sodium | | | |
| | | adjusted to | Chloride | | | |
| | | pH 5.5 with | | | | |
| | | 1N HCl | | | | |
| 81 | 9 | 100 mM L- | 100 mM | 5.55 | 2.62 | No |
| | | Histidine, | Sodium | | | |
| | | adjusted to | Chloride | | | |
| | | pH 5.5 with | | | | |
| | | 1N HCl | | | | |
| 82 | 12 | 100 mM L- | 100 mM | 5.55 | 2.37 | No |
| | | Histidine, | Sodium | | | |
| | | adjusted to | Chloride | | | |
| | | pH 5.5 with | | | | |
| | | 1N HCl | | | | |
| 83 | 15 | 100 mM L- | 100 mM | 5.55 | 2.32 | No |
| | | Histidine, | Sodium | | | |
| | | adjusted to | Chloride | | | |
| | | pH 5.5 with | | | | |
| | | 1N HCl | | | | |
| 84 | 3 | 100 mM L- | 100 mM | 5.52 | 4.63 | No |
| | | Histidine, | Sucrose | | | |
| | | adjusted to | | | | |
| | | pH 5.5 with | | | | |
| | | 1N HCl | | | | |
| 85 | 6 | 100 mM L- | 100 mM | 5.52 | 3.04 | No |
| | | Histidine, | Sucrose | | | |
| | | adjusted to | | | | |
| | | pH 5.5 with | | | | |
| | | 1N HCl | | | | |
| 86 | 9 | 100 mM L- | 100 mM | 5.52 | 2.57 | No |
| | | Histidine, | Sucrose | | | |
| | | adjusted to | | | | |
| | | pH 5.5 with | | | | |
| | | 1N HCl | | | | |
| 87 | 12 | 100 mM L- | 100 mM | 5.52 | 2.33 | No |
| | | Histidine, | Sucrose | | | |
| | | adjusted to | | | | |
| | | pH 5.5 with | | | | |
| | | 1N HCl | | | | |
| 88 | 15 | 100 mM L- | 100 mM | 5.52 | 2.09 | No |
| | | Histidine, | Sucrose | | | |
| | | adjusted to | | | | |
| | | pH 5.5 with | | | | |
| | | 1N HCl | | | | |
| 89 | 3 | 24.5 mM | 15 mM | 7.44 | 6.93 | Yes |
| | | Sodium | Dextrose | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 90 | 6 | 24.5 mM | 15 mM | 7.44 | 6.59 | Yes |
| | | Sodium | Dextrose | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 91 | 9 | 24.5 mM | 15 mM | 7.44 | 6.23 | Yes |
| | | Sodium | Dextrose | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 92 | 12 | 24.5 mM | 15 mM | 7.44 | 5.82 | Yes |
| | | Sodium | Dextrose | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 93 | 15 | 24.5 mM | 15 mM | 7.44 | 4.96 | Yes |
| | | Sodium | Dextrose | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 94 | 3 | 100 mM L- | 15 mM | 6.87 | 6.32 | No |
| | | Histidine, | Dextrose | | | |
| | | adjusted to | | | | |
| | | pH 7.0 with | | | | |
| | | 1N HCl | | | | |
| 95 | 6 | 100 mM L- | 15 mM | 6.87 | 6.06 | No |
| | | Histidine, | Dextrose | | | |
| | | adjusted to | | | | |
| | | pH 7.0 with | | | | |
| | | 1N HCl | | | | |
| 96 | 9 | 100 mM L- | 15 mM | 6.87 | 5.77 | No |
| | | Histidine, | Dextrose | | | |
| | | adjusted to | | | | |
| | | pH 7.0 with | | | | |
| | | 1N HCl | | | | |
| 97 | 12 | 100 mM L- | 15 mM | 6.87 | 5.42 | No |
| | | Histidine, | Dextrose | | | |
| | | adjusted to | | | | |
| | | pH 7.0 with | | | | |
| | | 1N HCl | | | | |
| 98 | 15 | 100 mM L- | 15 mM | 6.87 | 4.45 | No |
| | | Histidine, | Dextrose | | | |
| | | adjusted to | | | | |
| | | pH 7.0 with | | | | |
| | | 1N HCl | | | | |
| 99 | 3 | 100 mM L- | 15 mM | 6.88 | 6.39 | No |
| | | Histidine, | Mannitol | | | |
| | | adjusted to | | | | |
| | | pH 7.0 with | | | | |
| | | 1N HCl | | | | |
| 100 | 6 | 100 mM L- | 15 mM | 6.88 | 6.08 | No |
| | | Histidine, | Mannitol | | | |
| | | adjusted to | | | | |
| | | pH 7.0 with | | | | |
| | | 1N HCl | | | | |
| 101 | 9 | 100 mM L- | 15 mM | 6.88 | 5.78 | No |
| | | Histidine, | Mannitol | | | |
| | | adjusted to | | | | |
| | | pH 7.0 with | | | | |
| | | 1N HCl | | | | |
| 102 | 12 | 100 mM L- | 15 mM | 6.88 | 5.39 | No |
| | | Histidine, | Mannitol | | | |
| | | adjusted to | | | | |
| | | pH 7.0 with | | | | |
| | | 1N HCl | | | | |
| 103 | 15 | 100 mM L- | 15 mM | 6.88 | 4.61 | No |
| | | Histidine, | Mannitol | | | |
| | | adjusted to | | | | |
| | | pH 7.0 with | | | | |
| | | 1N HCl | | | | |
| 104 | 3 | 0.1M | 100 mM | 6.78 | 1.88 | No |
| | | TRIS*HCl | Mannitol | | | |
| | | solution | | | | |
| | | adjusted to | | | | |
| | | pH 7.0 with | | | | |
| | | 1N HCl | | | | |
| 105 | 6 | 0.1M | 100 mM | 6.78 | 1.63 | No |
| | | TRIS*HCl | Mannitol | | | |
| | | solution | | | | |
| | | adjusted to | | | | |
| | | pH 7.0 with | | | | |
| | | 1N HCl | | | | |
| 106 | 9 | 0.1M | 100 mM | 6.78 | 1.45 | No |
| | | TRIS*HCl | Mannitol | | | |
| | | solution | | | | |
| | | adjusted to | | | | |
| | | pH 7.0 with | | | | |
| | | 1N HCl | | | | |
| 107 | 12 | 0.1M | 100 mM | 6.78 | 1.32 | No |
| | | TRIS*HCl | Mannitol | | | |
| | | solution | | | | |
| | | adjusted to | | | | |
| | | pH 7.0 with | | | | |
| | | 1N HCl | | | | |
| 108 | 15 | 0.1M | 100 mM | 6.78 | 1.38 | No |
| | | TRIS*HCl | Mannitol | | | |
| | | solution | | | | |
| | | adjusted to | | | | |
| | | pH 7.0 with | | | | |
| | | 1N HCl | | | | |
| 109 | 3 | 24.5 mM | 100 mM | 7.43 | 6.94 | Yes |
| | | Sodium | Mannitol | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 110 | 6 | 24.5 mM | 100 mM | 7.43 | 6.6 | Yes |
| | | Sodium | Mannitol | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 111 | 9 | 24.5 mM | 100 mM | 7.43 | 6.25 | Yes |
| | | Sodium | Mannitol | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 112 | 12 | 24.5 mM | 100 mM | 7.43 | 5.83 | Yes |
| | | Sodium | Mannitol | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 113 | 15 | 24.5 mM | 100 mM | 7.43 | 4.87 | Yes |
| | | Sodium | Mannitol | | | |
| | | Phosphate | | | | |
| | | monobasic, | | | | |
| | | 75.5 Sodium | | | | |
| | | phosphate | | | | |
| | | dibasic | | | | |
| | | dihydrate, | | | | |
| | | adjusted to | | | | |
| | | pH 7.5 with | | | | |
| | | 1N NaOH | | | | |
| 114 | 3 | 100 mM L- | 100 mM | 6.9 | 6.4 | Yes |
| | | Histidine, | Sodium | | | |
| | | adjusted to | Chloride | | | |
| | | pH 7.0 with | | | | |
| | | 1N HCl | | | | |
| 115 | 6 | 100 mM L- | 100 mM | 6.9 | 6.11 | Yes |
| | | Histidine, | Sodium | | | |
| | | adjusted to | Chloride | | | |
| | | pH 7.0 with | | | | |
| | | 1N HCl | | | | |
| 116 | 9 | 100 mM L- | 100 mM | 6.9 | 5.71 | Yes |
| | | Histidine, | Sodium | | | |
| | | adjusted to | Chloride | | | |
| | | pH 7.0 with | | | | |
| | | 1N HCl | | | | |
| 117 | 12 | 100 mM L- | 100 mM | 6.9 | 5.28 | Yes |
| | | Histidine, | Sodium | | | |
| | | adjusted to | Chloride | | | |
| | | pH 7.0 with | | | | |
| | | 1N HCl | | | | |
| 118 | 15 | 100 mM L- | 100 mM | 6.9 | 4.69 | Yes |
| | | Histidine, | Sodium | | | |
| | | adjusted to | Chloride | | | |
| | | pH 7.0 with | | | | |
| | | 1N HCl | | | | |

### Example 4: Additional Buffer Screen

Additional formulations of the invention were made using solutions with one or more organic buffers. Selected formulations also contained a sugar as an additional excipient. Formulations with lower drug substance concentrations were prepared by diluting formulations having higher drug substance concentrations with blank formulations.

Formulations listed in TABLE 8 used trifluoroacetate salt of Compound 1 (formulations 119 through 124), chloride salt of Compound 1 (formulations 125 through 135), or acetate salt of Compound 1 (formulations 136-138). Compound 1 stock solution concentrations varied from 5 to 20 mM (29 to 116 mg/mL). Formulation stock solutions were made with excipients at 1.6 times (formulations 119 through 126) or 1.5 times (formulations 127 through 138) the final formulated concentration listed in TABLE 8 and adjusted to a pH of about 6.5, 7.0, or 7.5 with 1 M HCl or 1 M NaOH. Blank formulations in TABLE 8 were made by mixing five parts of stock solution with three parts water (formulations 119 through 126) or two parts of stock solution with one part water (formulations 127 through 138). Formulations of 15 mg/mL Compound 1 were made by mixing five parts of formulation stock solution with three parts (water plus drug substance stock solution) to the listed concentration (formulations 119 through 126) or two parts of formulation stock solution with one part (water plus drug substance stock solution) to the listed concentration (formulations 127 through 138). Formulations of 3 mg/mL Compound 1 (formulations 119, 121, 123 and 125) were made by diluting the 15 mg/mL Compound 1 formulation with formulation blanks. TABLE 8 shows the concentration of Compound 1, the pH of the blank formulations, pH of final formulations, and the apparent molecular weight in kilodaltons (kDa) as measured by SLS.

Formulations containing histidine-based buffers exhibited decreased aggregation of the compound and maintained a more stable pH range compared to succinate-based buffers. Higher concentrations of dextrose and lower final formulation pH decreased aggregation. In addition, formulations containing the acetate salt of Compound 1 were less acidic relative to formulations that contained the chloride salt of Compound 1.

TABLE 8
Formulations with different base buffers with dextrose or disodium succinate

| | | | | | Final | App |
|---|---|---|---|---|---|---|
| | | | Final | Cpd 1, | Formul. pH, | SLS |
| Formul. | | Final Buffer | Sugar/Salt | net | ambient | MW, |
| Number | Counter Ion | Composition | and concentr. | mg/mL | temp. | kDa |
| 119 | trifluoroacetate | 100 mM | 100 mmolal | 3 | 6.7 | 15.6 |
| | | TRIS*HCl solution | Dextrose | | | |
| | | adjusted to pH 7.5 | | | | |
| | | with 1N HCl | | | | |
| 120 | trifluoroacetate | 100 mM | 100 mmolal | 15 | 2.3 | 6.7 |
| | | TRIS*HCl solution | Dextrose | | | |
| | | adjusted to pH 7.5 | | | | |
| | | with 1N HCl | | | | |
| 121 | trifluoroacetate | 100 mM L- | 100 mmolal | 3 | 5.3 | 13.3 |
| | | Histidine, | Dextrose | | | |
| | | adjusted to pH 5.5 | | | | |
| | | with 1N HCl | | | | |
| 122 | trifluoroacetate | 100 mM L- | 100 mmolal | 15 | 3.05 | 8.3 |
| | | Histidine, | Dextrose | | | |
| | | adjusted to pH 5.5 | | | | |
| | | with 1N HCl | | | | |
| 123 | trifluoroacetate | 100 mM Histidine | 100 mmolal | 3 | 6.6 | 6.7 |
| | | solution | Dextrose | | | |
| | | adjusted to pH 7.0 | | | | |
| | | with 1N HCl | | | | |
| 124 | trifluoroacetate | 100 mM Histidine | 100 mmolal | 15 | 5.9 | 9.1 |
| | | solution | Dextrose | | | |
| | | adjusted to pH 7.0 | | | | |
| | | with 1N HCl | | | | |
| 125 | Chloride | 100 mM L- | 100 mmolal | 3 | 6.41 | 11.9 |
| | | Histidine, | Dextrose | | | |
| | | adjusted to pH 7.0 | | | | |
| | | with 1N HCl | | | | |
| 126 | Chloride | 100 mM L- | 100 mmolal | 15 | 5.6 | 9.8 |
| | | Histidine, | Dextrose | | | |
| | | adjusted to pH 7.0 | | | | |
| | | with 1N HCl | | | | |
| 127 | chloride | 100 mM L- | 130 mM | 15 | 5.79 | 22.3 |
| | | Histidine | Dextrose | | | |
| 128 | chloride | 150 mM L- | 50 mM | 15 | 6.19 | 21.7 |
| | | Histidine | Dextrose | | | |
| 129 | chloride | 100 mM Disodium | 50 mM | 15 | 5.09 | 22.6 |
| | | succinate | Dextrose | | | |
| 130 | chloride | 96.7 mM L- | 150 mM | 15 | 5.79 | 24.8 |
| | | Histidine, | Dextrose | | | |
| | | 53.3 mM L- | | | | |
| | | Histidine | | | | |
| | | monohydrochloride | | | | |
| | | monohydrate, | | | | |
| | | adjusted to pH 6.5 | | | | |
| | | with 1N NaOH | | | | |
| 131 | chloride | 8.9 mM Succinic | 50 mM | 15 | 5.31 | 15.2 |
| | | acid, | Dextrose | | | |
| | | 141.1 mM | | | | |
| | | disodium | | | | |
| | | succinate, | | | | |
| | | adjusted to pH 6.5 | | | | |
| | | with 1N NaOH | | | | |
| 132 | chloride | 100 mM Disodium | 250 mM | 15 | 5.12 | 40.1 |
| | | succinate | Dextrose | | | |
| 133 | chloride | 117 mM L- | 250 mM | 15 | 5.98 | 19 |
| | | Histidine | Dextrose | | | |
| 134 | chloride | 150 mM Disodium | 150 mM | 15 | 5.37 | 9.3 |
| | | succinate | Dextrose | | | |
| 135 | chloride | 65 mM L-Histidine | 250 mM | 15 | 5.3 | 19.5 |
| | | 35 mM L-Histidine | Dextrose | | | |
| | | monohydrochloride | | | | |
| | | monohydrate, | | | | |
| | | adjusted to pH 6.5 | | | | |
| | | with 1N NaOH | | | | |
| 136 | acetate | 113.6 mM L- | 206.7 mM | 8.2 | 6.88 | 8.2 |
| | | Histidine | Dextrose | | | |
| | | 6.4 mM L- | | | | |
| | | Histidine | | | | |
| | | monohydrochloride | | | | |
| | | monohydrate, | | | | |
| | | adjusted to pH 7 | | | | |
| | | with 1N HCl | | | | |
| 137 | acetate | 113.6 mM L- | 80 mM | 8.2 | 7.04 | 41.1 |
| | | Histidine | Disodium | | | |
| | | 6.4 mM L- | succinate | | | |
| | | Histidine | | | | |
| | | monohydrochloride | | | | |
| | | monohydrate, | | | | |
| | | adjusted to pH 7 | | | | |
| | | with 1N HCl | | | | |
| 138 | acetate | 113.6 mM L- | 40 mM | 8.2 | 7.01 | 32.8 |
| | | Histidine | Disodium | | | |
| | | 6.4 mM L- | succinate | | | |
| | | Histidine | | | | |
| | | monohydrochloride | | | | |
| | | monohydrate, | | | | |
| | | adjusted to pH 7 | | | | |
| | | with 1N HCl | | | | |

### Example 5: Dialysis Excipient Screen

Formulations were made using solutions containing an organic buffer and one or more additional excipients combined with drug substance. Several formulations were made by dialyzing formulation buffer across a membrane enclosing a solution of drug substance. The molecular weight cut off (MWCO) of this membrane was smaller than that of the molecular weight of the drug substance and larger than that of the largest molecular weight excipient. Formulations having lower drug substance concentrations were prepared by diluting formulations having higher drug substance concentrations with blank formulations.

Formulations listed in TABLE 9 and TABLE 10 were made by dialyzing a solution of Compound 1 drug substance (acetate salt) at 22.8 net mg/mL in water against a formulation buffer composed of the desired concentrations of excipients listed in TABLE 9 and TABLE 10. A 2 kDa MWCO regenerated cellulose membrane enclosed in a cassette was used to exchange the formulation buffers. Post-dialysis concentrations of Compound 1 were measured by ultraviolet-visible spectroscopy (UV-Vis) and used to calculate the amount of formulation buffer to dilute to a final concentration of 15 mg/mL. TABLE 10 shows the pH, osmolality, and the apparent molecular weight by SLS in kilodaltons (kDa) of the final formulations.

This screen determined that mannitol decreased aggregation of Compound 1 to a greater degree than all other excipients examined in this screen. Formulations containing Tween80 exhibited tween micelles, which were largest in blank formulations. The micelles decreased in size as the concentration of Compound 1 increased. Formulations containing Tween80 and higher NaCl concentrations exhibited visible precipitation. Formulations containing sodium ions, such as NaCl and sodium glutamate, exhibited increased aggregation. NaCl concentrations of less than 40 mM were found to inhibit aggregation of Compound 1 into trimers. Final formulations that had a pH of 6 exhibited decreased aggregation compared to final formulations at a pH of 7. The 50 mM Histidine and 100 mM dextrose formulation at pH of 6 was resistant to aggregation even at a Compound 1 concentration of 63 mg/ml.

TABLE 9
Formulations made with different excipients

| | | | L-Histidine | | | |
|---|---|---|---|---|---|---|
| | Compound | L- | monohydrochloride | | | L-arginine |
| Formulation | 1 net | Histidine | monohydrate | Mannitol | NaCl | monohydrochloride |
| Number | mg/mL | mmolal | mmolal | mmolal | mmolal | mmolal |
| 139 | 15 | 10 | 10 | 100 | 0 | 0 |
| 140 | 15 | 18.2 | 1.8 | 175 | 50 | 40 |
| 141 | 15 | 10 | 10 | 0 | 30 | 20 |
| 142 | 15 | 18.2 | 1.8 | 75 | 10 | 50 |
| 143 | 15 | 10 | 10 | 150 | 0 | 10 |
| 144 | 15 | 10 | 10 | 50 | 40 | 0 |
| 145 | 15 | 18.2 | 1.8 | 25 | 60 | 70 |
| 146 | 15 | 18.2 | 1.8 | 100 | 70 | 30 |
| 147 | 15 | 10 | 10 | 125 | 20 | 60 |

TABLE 10
Formulations made with different excipients

| | | | | Final | | |
|---|---|---|---|---|---|---|
| | | | | Formulation | | |
| | Sodium | | L- | pH, | | |
| Formulation | Glutamate | Tween 80, | Methionine | ambient | Osmo- | App SLS |
| Number | mmolal | wt %/vol | mmolal | temperature | lality | MW, kDa |
| 139 | 0 | 0 | 0 | 6.2 | 131 | 5.4 |
| 140 | 20 | 0.1 | 10 | 6.9 | 403 | 60.3 |
| 141 | 50 | 0.1 | 10 | 6.3 | 216 | 29.2 |
| 142 | 10 | 0.1 | 0 | 6.9 | 217 | 23.2 |
| 143 | 40 | 0 | 10 | 6.3 | 278 | 15.6 |
| 144 | 0 | 0 | 0 | 6.2 | 154 | 37.3 |
| 145 | 30 | 0 | 10 | 6.8 | 341 | 73.7 |
| 146 | 70 | 0 | 0 | 6.9 | 433 | 63.1 |
| 147 | 60 | 0.1 | 0 | 6.2 | 401 | 33.7 |

### Example 6: Additional Direct Formulations

Selected formulations were made using L-histidine, dextrose, or sucrose, and optionally contained imidazole or pyridoxine as an additional organic base. All formulations used a acetate salt of Compound 1. Formulation stock solutions were made with excipients at two times the final formulated concentration listed in TABLE 11. Blank formulations at concentrations listed in TABLE 11 were made by mixing one part of stock solution with one part water. Stock solutions of acetate salt of Compound 1 were made by dissolving acetate salt of Compound 1 in water to a concentration twice that of the desired final formulated concentration. Water-only Formulation 148 was made by re-constituting acetate salt of the drug substance at a concentration of 72 mg/mL. Formulation 157 was prepared by reconstituting acetate salt of Compound 1 with the stock solution used for making Formulation 156. Concentrations of Compound 1 were checked by UV absorbance. Final formulations were made by mixing one part formulation stock solutions with one part drug substance stock solution.

TABLE 11 shows the pH, osmolality and the UV measured concentration of the final formulations. This formulation screen determined that the addition of imidazole or pyridoxine to the formulation did not result in significantly different pH or osmolality values relative to formulation that only had dextrose or sucrose as excipients.

TABLE 11
Imidazole and Pyridoxine excipient formulation testing

| | | | | | | | Final | |
|---|---|---|---|---|---|---|---|---|
| | | | | | | | Formul. | |
| | | | | | | Cpd 1, | pH, | |
| Formul. | Reconstit. | L-Histidine | | | Addit. | net | ambient | |
| Number | Solvent | Final Conc. | | Sugar | Excipient | mg/mL | temp. | Osmolality |
| 148 | water | none | | none | none | 72 | 4.8 | 121 |
| 149 | water | 20 | mM | 100 mM | 50 mM | 78 | 6.3 | 293 |
| | | | | Dextrose | Imidazole | | | |
| 150 | water | 20 | mM | 170 mM | 10 mM | 76 | 5.3 | 309 |
| | | | | Dextrose | imidazole | | | |
| 151 | water | 20 | mM | 160 mM | 20 mM | 87 | 5.4 | 337 |
| | | | | Sucrose | imidazole | | | |
| 152 | water | 20 | mM | 180 mM | none | 88 | 5.3 | 305 |
| | | | | Dextrose | | | | |
| 153 | water | 20 | mM | 180 mM | none | 75 | 5.2 | 309 |
| | | | | Sucrose | | | | |
| 154 | water | 20 | mM | 170 mM | 10 mM | 75 | 5.1 | 304 |
| | | | | Dextrose | Pyridoxine | | | |
| 155 | water | 20 | mmolal | 175 | none | 76 | 5.4 | 310 |
| | | | | mmolal | | | | |
| | | | | Dextrose | | | | |
| 156 | water | 90 | mmolal | 75 | none | 73.8 | 5.7 | 320 |
| | | | | mmolal | | | | |
| | | | | Dextrose | | | | |
| 157 | Formul. | 180 | mmolal | 150 | none | 74.9 | 6.1 | 471 |

| | |
|---|---|
| media | mmolal |
| | Dextrose |

### Example 7: In Vivo Formulations

Formulations of the invention for in vivo studies were made using solution composed of a histidine buffer and dextrose combined with drug substance. Formulations were made either by combining concentrated excipient stock solutions with drug substance or dialyzing drug substance across a membrane against a reservoir of formulation solution.

Formulation 158 was prepared by mixing chloride salt of Compound 1 with a concentrated stock solution of formulation buffer and was not dialyzed. The remaining formulations from TABLE 12 were made from acetate salt of Compound 1. The remaining formulations were prepared by dialyzing a buffer solution across a membrane enclosing a solution of drug substance. The molecular weight cut off (MWCO) of the membrane was smaller than that of the molecular weight of the drug substance and larger than that of the largest molecular weight excipient. Formulations with lower drug substance concentrations were prepared by diluting formulations with higher drug substance concentrations with blank formulations.

Selected formulations were made by dialyzing a solution of acetate salt of Compound 1 dissolved in water against a formulation buffer having the desired concentrations of excipients listed in Table 7. A 2 kDa MWCO regenerated cellulose membrane enclosed in a cassette was used to exchange the formulation buffers. Dilutions with dialysis formulation buffer were made to obtain the final concentrations listed in TABLE 12, which also shows the pH, osmolality, and the UV measured concentration of the final formulations.

This screen determined that the final formulations tested resulted in decreased aggregation even if high concentrations of Compound 1 were used.

TABLE 12
High concentration compound formulations

| | | | | | Final | |
|---|---|---|---|---|---|---|
| | | | | | Formul. | |
| | | | Formulation | Cpd 1, | pH, | |
| Formul. | | Counter | Buffer | net | ambient | |
| Number | Preparation Method | Ion | Composition | mg/mL | temp. | Osmolality |
| 158 | Dilution from a | chloride | 90.9 mM L- | 2.6 | 6.6 | 231 |
| | concentrated | | Histidine, | | | |
| | stock solution | | 9.1 mM L- | | | |
| | | | Histidine | | | |
| | | | monohydrochloride | | | |
| | | | monohydrate, | | | |
| | | | 100 mM Dextrose | | | |
| 159 | Dialysis in formulation | acetate | 25 mM L- | 63.3 | 6.1 | 200 |
| | buffer with | | Histidine, | | | |
| | a 2 kDa MWCO | | 25 mM L-Histidine | | | |
| | regenerated cellulose | | monohydrochloride | | | |
| | membrane | | monohydrate, | | | |
| | | | 100 mM Dextrose | | | |
| 160 | Dialysis in formulation | acetate | 25 mM L- | 72 | 6.1 | 200 |
| | buffer with | | Histidine, | | | |
| | a 2 kDa MWCO | | 25 mM L-Histidine | | | |
| | regenerated cellulose | | monohydrochloride | | | |
| | membrane; diluted to | | monohydrate, | | | |
| | desired concentration | | 100 mM Dextrose | | | |
| 161 | Dialysis in formulation | acetate | 25 mM L- | 3.15 | 6.2 | 181 |
| | buffer with | | Histidine, | | | |
| | a 2 kDa MWCO | | 25 mM L-Histidine | | | |
| | regenerated cellulose | | monohydrochloride | | | |
| | membrane; diluted to | | monohydrate, | | | |
| | desired concentration | | 100 mM Dextrose | | | |
| 162 | Dialysis in formulation | acetate | 25 mM L- | 8.7 | 6.2 | 183 |
| | buffer with | | Histidine, | | | |
| | a 2 kDa MWCO | | 25 mM L-Histidine | | | |
| | regenerated cellulose | | monohydrochloride | | | |
| | membrane; diluted to | | monohydrate, | | | |
| | desired concentration | | 100 mM Dextrose | | | |
| 163 | Dialysis in formulation | acetate | 37.5 mM L- | 116 | 6 | 298 |
| | buffer with | | Histidine, | | | |
| | a 2 kDa MWCO | | 37.5 mM L- | | | |
| | regenerated cellulose | | Histidine | | | |
| | membrane; diluted to | | monohydrochloride | | | |
| | desired concentration | | monohydrate, | | | |
| | | | 165 mM Dextrose | | | |
| 164 | Dialysis in formulation | acetate | 37.5 mM L- | 54.8 | 6 | 281 |
| | buffer with | | Histidine, | | | |
| | a 2 kDa MWCO | | 37.5 mM L- | | | |
| | regenerated cellulose | | Histidine | | | |
| | membrane; diluted to | | monohydrochloride | | | |
| | desired concentration | | monohydrate, | | | |
| | | | 165 mM Dextrose | | | |
| 165 | Dialysis in formulation | acetate | 37.5 mM L- | 26.7 | 6.1 | 279 |
| | buffer with | | Histidine, | | | |
| | a 2 kDa MWCO | | 37.5 mM L- | | | |
| | regenerated cellulose | | Histidine | | | |
| | membrane; diluted to | | monohydrochloride | | | |
| | desired concentration | | monohydrate, | | | |
| | | | 165 mM Dextrose | | | |
| 166 | Dialysis in formulation | acetate | 37.5 mM L- | 43.4 | 6 | 268 |
| | buffer with | | Histidine, | | | |
| | a 2 kDa MWCO | | 37.5 mM L- | | | |
| | regenerated cellulose | | Histidine | | | |
| | membrane; diluted to | | monohydrochloride | | | |
| | desired concentration | | monohydrate, | | | |
| | | | 165 mM Dextrose | | | |

### Example 8: Low Histidine Formulations

Formulations that contained low concentrations of L-histidine were prepared using a solution of a histidine buffer and dextrose combined with drug substance. Formulations were made by dialyzing formulation buffer across a membrane enclosing a solution of drug substance. The molecular weight cut off (MWCO) of the membrane was smaller than that of the molecular weight of the drug substance and larger than that of the largest molecular weight excipient. Formulations with lower drug substance concentrations were prepared by diluting formulations having higher drug substance concentrations with blank formulations. Formulations were made by dialyzing a solution of Compound 1 drug substance (acetate salt) dissolved in water at a concentration of about 30 mg/mL against a formulation buffer having the target concentrations of excipients listed in TABLE 13. A 2 kDa MWCO regenerated cellulose membrane enclosed in a cassette was used to exchange the formulation buffers. Dilutions with dialysis buffer were made to obtain final concentrations of about 2.9 or 14.4 mg/mL of Compound 1 in the final formulations. TABLE 13 shows the pH, osmolality and the UV measured concentration of the final formulations.

TABLE 13
Low Histidine buffer concentration formulations

| | | | | | Final | |
|---|---|---|---|---|---|---|
| | | | | | Formul. | |
| | | | | Cpd 1, | pH, | |
| Formul. | Preparation | Counter | Formulation | net | ambient | Osmolality, |
| Number | Method | Ion | Composition | mg/mL | temp. | mmols/ kg |
| 167 | Dialysis in | Acetate | 1.375 mM L- | 2.6 | 6.1 | 294 |
| | formulation | | Histidine, | | | |
| | buffer with | | 1.375 mM L- | | | |
| | a 2 kDa MWCO | | Histidine | | | |
| | regenerated | | monohydrochloride | | | |
| | cellulose | | monohydrate, | | | |
| | membrane | | 280 mM | | | |
| | | | Dextrose | | | |
| 168 | Dialysis in | Acetate | 1.375 mM L- | 15 | 6.2 | 272 |
| | formulation | | Histidine, | | | |
| | buffer with | | 1.375 mM L- | | | |
| | a 2 kDa MWCO | | Histidine | | | |
| | regenerated | | monohydrochloride | | | |
| | cellulose | | monohydrate, | | | |
| | membrane | | 280 mM | | | |
| | | | Dextrose | | | |
| 169 | Dialysis in | Acetate | 2.75 mM L- | 2.8 | 6.1 | 298 |
| | formulation | | Histidine, | | | |
| | buffer with | | 2.75 mM L- | | | |
| | a 2 kDa MWCO | | Histidine | | | |
| | regenerated | | monohydrochloride | | | |
| | cellulose | | monohydrate, | | | |
| | membrane | | 280 mM | | | |
| | | | Dextrose | | | |
| 170 | Dialysis in | Acetate | 2.75 mM L- | 14.4 | 6.2 | 280 |
| | formulation | | Histidine, | | | |
| | buffer with | | 2.75 mM L- | | | |
| | a 2 kDa MWCO | | Histidine | | | |
| | regenerated | | monohydrochloride | | | |
| | cellulose | | monohydrate, | | | |
| | membrane | | 280 mM | | | |
| | | | Dextrose | | | |
| 171 | Dialysis in | Acetate | 5.0 mM L- | 2.8 | 6.1 | 306 |
| | formulation | | Histidine, | | | |
| | buffer with | | 5.0 mM L- | | | |
| | a 2 kDa MWCO | | Histidine | | | |
| | regenerated | | monohydrochloride | | | |
| | cellulose | | monohydrate, | | | |
| | membrane | | 280 mM | | | |
| | | | Dextrose | | | |
| 172 | Dialysis in | Acetate | 5.0 mM L- | 14.8 | 6.2 | 287 |
| | formulation | | Histidine, | | | |
| | buffer with | | 5.0 mM L- | | | |
| | a 2 kDa MWCO | | Histidine | | | |
| | regenerated | | monohydrochloride | | | |
| | cellulose | | monohydrate, | | | |
| | membrane | | 280 mM | | | |
| | | | Dextrose | | | |

### Example 9: Formulations for In Vivo Studies

TABLE 14 provides formulations that were prepared in vivo use. The pH and the osmolality of the blank formulations (formulations without Compound 1) were measured before Compound 1 was added to the formulation. Following addition of Compound 1, the pH and the osmolality were measured again.

TABLE 14
In vivo formulations

| | | | | Osmolality |
|---|---|---|---|---|
| | Blank | Blank | pH with | with |
| Histidine | Formulation | Formulation | 72 mg/mL | 72 mg/mL |
| Concentration | pH | Osmolality | Compound 1 | Compound 1 |
| 50 mM, | 6.0-6.2 | 150 | 6.1 at 1-6 | 150 at 1-6 |
| 7.8 mg/mL | | | mg/mL | mg/mL |
| 65 mM, | 6.0-6.2 | 262-267 | 6.1 | 298 |
| 10.1 mg/mL | | | | |
| Initial 2X: | 7.6-7.8 | 310 (2X) | 5.7-6.4 | 266-320 |
| 170 mM, | | | | |
| 26.4 mg/mL | | | | |
| Final 1X: | | | | |
| 85 mM, | | | | |
| 13.2 mg/mL | | | | |

### Example 10: Example Procedure for Preparation of PNA Salts

Synthesis of crude PNA: PNA synthesis was performed using Fmoc solid-phase peptide chemistry on an Intavis MultiPep RSi (25 μmol scale) at ambient temperature. TentaGel® R RAM resin (0.18 meq/g) was swelled in N,N-dimethylformamide (DMF) (1 mL, 3×10 min). Fmoc groups were removed with 20% piperidine (v/v) in DMF (0.8 mL, 2×10 minutes). Following deprotection, the resin was washed with DMF (6×1 mL) and the next PNA residue was coupled to the N-terminus of resin-bound PNA upon treatment with a mixture of Fmoc-protected monomer (85 μmol, 3.4×, 0.2 M in NMP), HATU (80 μmol, 3.2×, 0.5 M in DMF), and N,N-diisopropylethylamine (DIPEA, 83 μmol, 3.3×, 0.52 M in DMF) (double coupling: 2 min pre-activation and 30 min coupling). After coupling, the resin was acetyl capped with 5% acetic anhydride and 6% 2,6-lutidine (v/v) in DMF (5 min), and subsequently washed with DMF (6×1 mL). Upon completion of all synthetic cycles, the resin was washed with DMF (5×) and DCM (5×). PNA was simultaneously cleaved and deprotected upon treatment with a mixture of trifluoroacetic acid/triflic acid/thioanisol/m-cresol (5 mL, 6:2:1:1) under agitation for 90 minutes. PNA was precipitated in ice-cold diethyl ether (45 mL), spun by centrifuge, isolated from the supernatant, washed with ice-cold diethyl ether (50 mL), and dried under vacuum.

Purification of PNA: Crude PNA was dissolved in Milli-Q water (5 mL) and filtered through a nylon syringe-tip filter (0.45 μm pore size). Preparative reversed-phase high-pressure liquid chromatography (RP-HPLC) was performed on a Dionex Ultimate 3000 equipped with a Hypersil Gold C18aq 30×250 mm column, with a 5 μm particle size and 175 Å pore size. A 30-minute linear gradient of acetonitrile (5-25%) in water with 0.1% TFA was used as the mobile phase. HPLC eluate was fractionated based on UV absorbance (λ=254 nm) and fractional purity was assessed via MALDI-TOF in linear positive mode with a matrix of CHCA. Pure fractions were lyophilized, dissolved in water, combined, and re-lyophilized to obtain the trifluoroacetate salts.

Pure PNA was dissolved in PBS and the concentration of the PNA solution was determined via absorbance spectroscopy (ε(λ=260 nm)=148,300 cm⁻¹ M⁻¹). Selected compounds were characterized by matrix-assisted laser desorption/ionization mass spectroscopy (MALDI-MS) or ESI+MS. Observed masses are presented in TABLE 15.

TABLE 15

| Cpd | |
|---|---|
| # | Observed Mass [M + 1] |
| 1 | 5812.8 |
| 2 | 6912.4, 6919.6, 6915.7 |
| 3 | 5333.7 |
| 4 | 6340.5 |
| 5 | 7427.5 |
| 6 | 6955.6 |
| 7 | 6089.7 |
| 8 | 6190.3 |
| 11 | 6130.1 |

Measurement of TFA Content in Compound 1: To measure TFA content, 50 nanomoles of Compound 1 (quantity determined via A260 absorbance measurements) was lyophilized, dissolved in deuterium oxide, and spiked with an internal standard of 100 nmoles of 2,2-TFE. The ratio of TFA to PNA was quantified by ¹⁹F-NMR and was found to vary between 20-50 across individual samples.

Preparation of Compound 1 Chloride Salt: The chloride salt of Compound 1 was prepared by incubation of 300 mg Compound 1 TFA salt in an aqueous solution of hydrochloric acid (0.1 M, 300 mL) at 0° C. for 5 minutes, after which time the sample was lyophilized to afford the hydrochloric acid salt of Compound 1. The ratio of TFA to PNA was quantified by ¹⁹F-NMR and was found to be below the LOD (<0.05 TFA:PNA).

Preparation of Compound 1 Acetate Salt: The acetate salt of Compound 1 was prepared from the TFA salt of Compound 1 by treatment with strong anion exchange resin. A solution of Compound 1 TFA salt (4.2 g) was dissolved in aqueous 10% acetic acid at a concentration of 20 mM. The resulting solution was passed through a column of strong anion exchange resin prepared with acetate counterion (85 g) with a flow rate of 0.1 bed volumes per minute and eluted with a stream of 10% acetic acid in water. The eluent was lyophilized to afford 4.2 g Compound acetate salt. Residual TFA content was quantified by ¹⁹F-NMR and was found to be 0.042% (w/w %). Acetate content was measured by HPLC and found to be 10.0% (w/w %).

### Example 11: Example Procedure for Preparation of Compound 1 Formulation

Preparation of Media A. 6.206 g (40 mmol) of L-histidine was combined with 63.056 g (350 mmol) of dextrose and 1000 g of 18.2 MΩ sterile USP-grade water and then thoroughly mixed by sonication and filtered using a sterile 0.22 um vacuum filter device.

Preparation of Media B. 12.412 g (80 mmol) of L-histidine was combined with 55.850 g (310 mmol) of dextrose and 1000 g of 18.2 MΩ sterile USP-grade water and then thoroughly mixed by sonication and filtered using a sterile 0.22 um vacuum filter device.

Preparation of Formulation. 2000 mg of Compound 1 acetate salt was thoroughly dissolved in 12.89 mL of 18.2 MΩ sterile USP-grade water by agitating the solution by vortex and heating to 30° C. This solution was diluted with 12.90 mL of Media A or Media B. The resultant solution was tested for pH and osmolality and then adjusted to obtain desired properties.

### Example 12: Formulations for In Vivo Studies Using Compounds 148 and 167

### Preparation of Media A.

12.413 g (80 mmol) of L-histidine was combined with 82.835 g (460 mmol) of dextrose and 943.4 g of 18.2 MΩ water, thoroughly mixed by a magnetic stir bar, and then filtered using a sterile 0.22 um vacuum filter device. The measured pH was 7.6 and the measured osmolality was 568 mmol/kg.

### Preparation of Media B.

25.0 mL of Media A was diluted with 25.0 mL of sterile 18.2 MΩ water and mixed by vortex then filtered using a sterile 0.22 um vacuum filter device. The measured pH was 7.6 and the measured osmolality was 273 mmol/kg.

### Preparation of Formulations 175-177.

36.3 mg of Compound 148 was thoroughly dissolved in sterile 18.2 MΩ water by vortex and heating to 30° C. This solution was diluted with 3.0 mL of Media A and mixed by vortexer to achieve a Compound 148 concentration of 6.1 mg/mL (TABLE 16, Formulation 175). This solution was further diluted as desired using media B to provide Formulations 176-177. Formulations were sterile filtered through 0.22 um syringe filters prior to vialing for in vivo mouse studies.

### Preparation of Formulation 174.

Formulations of Compound 167 were made by dialyzing a buffer solution enclosing a solution of drug substance. The molecular weight cut off of the membrane was smaller than that of the drug substance and larger than that of the largest molecular weight excipient.

Formulation 174 (TABLE 16) was made by dialyzing a solution of the acetate salt Compound 167 against a buffer containing of 25.0 mM L-Histidine, 25.0 mM L-Histidine monohydrochloride monohydrate, and 100 mM dextrose. Approximately 500 mg of the lyophilized acetate salt of Compound 167 was dissolved in 14.5 mL of sterile water to provide a final volume of 15 mL. The concentration of Compound 167 in the resulting solution was measured to be 39.3 mg/mL by UV absorbance at 260 nm.

The dialysis buffer was made by dissolving 38.79 g (250 mmol) L-Histidine, 52.415 g (250 mmol) L-Histidine monohydrochloride monohydrate, and 180.2 g (1000 mmol) dextrose in 9.96 kg in 18.2 MΩ water to provide a total volume of 10 L. The dialysis buffer was sterile filtered through a 0.22 um vacuum filter device. The pH of the dialysis buffer was 6.1, and the osmolality was 173 mmols/kg. The 39.3 mg/mL solution of Compound 167 was loaded into a 2 kDa MWCO regenerated cellulose membrane enclosed in a cassette then dialyzed against ˜3.5 L of the dialysis buffer. The dialysis buffer was changed twice to reach full equilibrium. 14.5 mL of the dialyzed Compound 167 solution was recovered. The concentration of Compound 167 was 38.1 mg/mL post-dialysis.

A dilution buffer of the same composition as the dialysis buffer was used to dilute the dialyzed Compound 167 solution to the target concentration of 3 mg/mL. The final concentration of the formulation was 2.97 mg/mL, the pH was 6.1, and the osmolality was 174.

TABLE 16
In vivo formulations of Compounds 148 and 167

| | | | | Formulation | Cpd, | Final pH, | Final |
|---|---|---|---|---|---|---|---|
| Formul. | Cpd | Preparation | Counter | Buffer | net | ambient | Osmolality |
| Number | Number | Method | Ion | Composition | mg/mL | temp | (mmol/kg) |
| 174 | 167 | Dialysis in | Acetate | 25.0 mM L- | 2.97 | 6.1 | 174 |
| | | formulation | | Histidine, | | | |
| | | buffer with a | | 25.0 mM L- | | | |
| | | 2 kDa MWCO | | Histidine | | | |
| | | regenerated | | monohydrochloride | | | |
| | | cellulose | | monohydrate, | | | |
| | | membrane | | 100 mM | | | |
| | | | | Dextrose | | | |
| 175 | 148 | Dilution from | Acetate | 40.0 mM L- | 6.11 | 7.2 | 314 |
| | | a concentrated | | Histidine, | | | |
| | | stock | | 230 mM | | | |
| | | | | Dextrose | | | |
| 176 | 148 | Dilution from | Acetate | 40.0 mM L- | 3.00 | 6.9 | 286 |
| | | Formulation | | Histidine, | | | |
| | | 175 | | 230 mM | | | |
| | | | | Dextrose | | | |
| 177 | 148 | Dilution from | Acetate | 40.0 mM L- | 1.48 | 6.6 | 280 |
| | | Formulation | | Histidine, | | | |
| | | 175 | | 230 mM | | | |
| | | | | Dextrose | | | |

What is claimed is:

1. A pharmaceutical composition comprising:
(a) an oligonucleotide analogue comprising a structure according to formula (II): 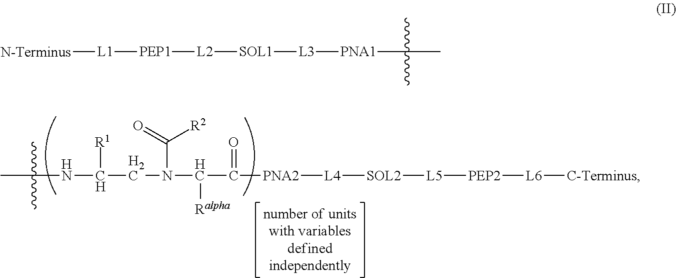
wherein:
the number of units with variables defined independently is at least 3;
N-Terminus is H, acyl, a group that together with the nitrogen atom to which N-Terminus is bound forms a carbamate, a probe, a metal chelator, or a biological agent;
each R¹ is independently alkyl that is unsubstituted or substituted or H;
each R^{alpha} is independently alkyl that is unsubstituted or substituted or H;
each R² is independently alkyl, O-alkyl, or methyl substituted with a heterocycle, wherein at least two R² groups in the structure are independently methyl substituted with a heterocycle;
C-Terminus is OH, O-alkyl, a peptide sequence, or NH₂;
PEP1 is a peptide sequence or absent;
PEP2 is a peptide sequence or absent;
SOL1 is a water-solubilizing group or absent;
SOL2 is a water-solubilizing group or absent;
PNA1 is a peptide nucleic acid sequence or absent;
PNA2 is a peptide nucleic acid sequence or absent;
L1 is a linker group or absent;
L2 is a linker group or absent;
L3 is a linker group or absent;
L4 is a linker group or absent;
L5 is a linker group or absent; and
L6 is a linker group or absent,
or a pharmaceutically-acceptable salt or ionized form thereof; and
(b) an amino acid or a pharmaceutically-acceptable salt or ionized form thereof thereof, wherein the amino acid comprises a side chain that is positively charged at physiological pH.

2. The composition of claim 1, wherein the amino acid is arginine.

3. The composition of claim 1, wherein the amino acid is lysine.

4. The composition of claim 1, wherein the amino acid is ornithine.

5. The composition of claim 1, wherein the amino acid is histidine.

6. The composition of claim 5, wherein histidine or an ionized form thereof is present in the composition at a concentration of about 0.5 mM to about 100 mM.

7. The composition of claim 5, wherein histidine or an ionized form thereof is present in the composition at a concentration of about 25 mM to about 100 mM.

8. The composition of claim 5, wherein histidine or an ionized form thereof is present in the composition at a concentration of about 25 mM to about 75 mM.

9. The composition of claim 5, wherein histidine or an ionized form thereof is present in the composition at a concentration of about 40 mM.

10. The composition of claim 5, wherein histidine or an ionized form thereof is present in the composition at a concentration of about 50 mM.

11. The composition of any one of claims 5-10, wherein the histidine is L-histidine.

12. The composition of any one of claims 1-11, wherein the pharmaceutical composition further comprises a second amino acid or a pharmaceutically-acceptable salt or ionized form thereof.

13. The composition of claim 12, wherein the second amino acid comprises a side chain that is positively charged at physiological pH.

14. The composition of claim 12 or 13, wherein the second amino acid is arginine.

15. The composition of any one of claims 1-14, wherein the pharmaceutical composition further comprises an anti-aggregation agent.

16. The composition of claim 15, wherein the anti-aggregation agent is a polyol.

17. The composition of claim 15 or 16, wherein the anti-aggregation agent is a saccharide.

18. The composition of any one of claims 15-17, wherein the anti-aggregation agent is a sugar.

19. The composition of any one of claims 15-17, wherein the anti-aggregation agent is a sugar alcohol.

20. The composition of any one of claims 15-19, wherein the anti-aggregation agent is maltitol, mannitol, isomalt, sorbitol, xylitol, or erythritol.

21. The composition of any one of claims 15-19, wherein the anti-aggregation agent is mannitol.

22. The composition of claim 21, wherein mannitol is present in the composition at a concentration from about 5 mM to about 200 mM.

23. The composition of claim 21, wherein mannitol is present in the composition at a concentration from about 50 mM to about 150 mM.

24. The composition of claim 21, wherein mannitol is present in the composition at a concentration from about 5 mM to about 30 mM.

25. The composition of claim 21, wherein mannitol is present in the composition at a concentration of 15 mM.

26. The composition of claim 21, wherein mannitol is present in the composition at a concentration of 100 mM.

27. The composition of any one of claims 15-17, wherein the anti-aggregation agent is a sugar.

28. The composition of any one of claims 15-17 and 27, wherein anti-aggregation agent is a monosaccharide.

29. The composition of any one of claims 15-17, 27, and 28, wherein the anti-aggregation agent is dextrose.

30. The composition of claim 29, wherein dextrose is present in the composition at a concentration of about 50 mM to about 400 mM.

31. The composition of claim 29, wherein dextrose is present in the composition at a concentration of about 50 mM to about 300 mM.

32. The composition of claim 29, wherein dextrose is present in the composition at a concentration of about 200 mM to about 250 mM.

33. The composition of claim 29, wherein dextrose is present in the composition at a concentration of about 100 mM.

34. The composition of claim 29, wherein dextrose is present in the composition at a concentration of about 230 mM.

35. The composition of any one of claims 1-34, wherein the number of units with variables defined independently is 11, 12, 13, 14, 15, 16, or 17.

36. The composition of any one of claims 1-34, wherein the number of units with variables defined independently is 14.

37. The composition of any one of claims 1-36, wherein N-Terminus is H and C-Terminus is NH₂.

38. The composition of any one of claims 1-37, wherein each R^{alpha} is independently H, hydroxylmethyl, or 4-guanidinobut-1-yl.

39. The composition of any one of claims 1-37, wherein each R^{alpha} is H.

40. The composition of any one of claims 1-37, wherein at least one iteration of R^{alpha} is hydroxylmethyl.

41. The composition of any one of claims 1-37, wherein at least one iteration of R^{alpha} is 4-guanidinobut-1-yl.

42. The composition of any one of claims 1-37, wherein at least half the iterations of R^{alpha} are hydroxylmethyl and the other iterations of R^{alpha} are H.

43. The composition of any one of claims 1-37, wherein at least half the iterations of R^{alpha} are 4-guanidinobut-1-yl and the other iterations of R^{alpha} are H.

44. The composition of any one of claims 1-43, wherein each R¹ is independently H, hydroxylmethyl, or 4-guanidinobut-1-yl.

45. The composition of any one of claims 1-43, wherein each R¹ is H.

46. The composition of any one of claims 1-43, wherein at least one iteration of R¹ is hydroxylmethyl.

47. The composition of any one of claims 1-43, wherein at least one iteration of R¹ is 4-guanidinobut-1-yl.

48. The composition of any one of claims 1-43, wherein at least half the iterations of R¹ are hydroxylmethyl and the other iterations of R¹ are H.

49. The composition of any one of claims 1-43, wherein at least half the iterations of R¹ are 4-guanidinobut-1-yl and the other iterations of R¹ are H.

50. The composition of any one of claims 1-49, wherein each of L1, L2, L3, L4, L5, and L6 is absent.

51. The composition of any one of claims 1-50, wherein PEP1 and PEP2 are absent.

52. The composition of any one of claims 1-50, wherein one of PEP1 and PEP2 is a peptide sequence that is a nuclear localization sequence and the other is absent.

53. The composition of any one of claims 1-52, wherein SOL1 is the water-solubilizing group and SOL2 is absent.

54. The composition of any one of claims 1-49, wherein each of L1, L2, L3, L4, L5, L6, PEP1, PEP2, and SOL2 is absent, and SOL1 is the water-solubilizing group.

55. The composition of any one of claims 1-49, 53, and 54, wherein the water-solubilizing group is a group that contains multiple positive charges at physiological pH.

56. The composition of any one of claims 1-55, wherein the water-solubilizing group of SOL1 is a group of formula: 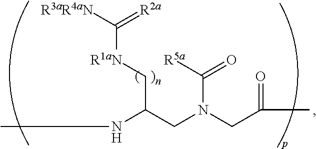
wherein
R^{1a} is H, alkyl, or a nitrogen atom protecting group;
R^{2a} is O, NH, N(alkyl), or N(Pg^{N}), wherein Pg^{N} is a nitrogen atom protecting group;
R^{3a} is H, alkyl, or a nitrogen atom protecting group;
R^{4a} is H, alkyl, or a nitrogen atom protecting group;
R^{5a} is alkyl or O-alkyl, any of which is unsubstituted or substituted;
n is 1, 2, 3, 4, 5, 6, 7, 8, 9, or 10; and
p is an integer from 1-100.

57. The composition of claim 56, wherein p is 5, 6, 7, or 8.

58. The composition of any one of claims 1-56, wherein the water-solubilizing group of SOL1 is a group of formula: 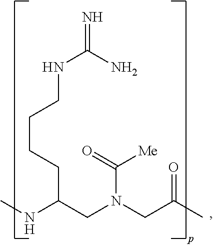
wherein p is an integer that is 5, 6, 7, or 8.

59. The composition of any one of claims 1-56, wherein the water-solubilizing group of SOL1 is a group of formula: 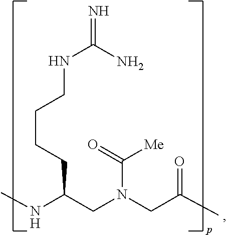
wherein p is an integer that is 5, 6, 7, or 8.

60. The composition of any one of claims 56-59, wherein p is 7.

61. The composition of any one of claims 1-59, wherein the heterocycles of the R² groups are each independently:

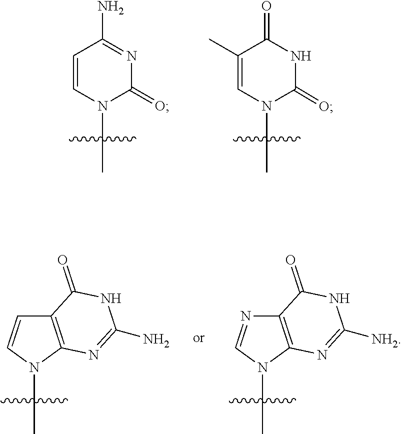

62. The composition of any one of claims 1-59, wherein each R² is independently: methyl,

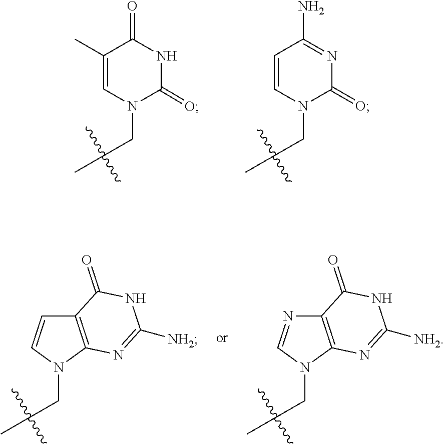 [SMILES: *Cn1cc(C)c(=O)[nH]c1=O ; *Cn1ccc(N)nc1=O ; *Cn1ccc2c(=O)[nH]c(N)nc21 ; "or" ; *Cn1cnc2c(=O)[nH]c(N)nc21]

63. The composition of any one of claims 1-59, wherein the heterocycles of the R² groups form a sequence that repeats at least twice, wherein the sequence is, from N-Terminus to C-Terminus: 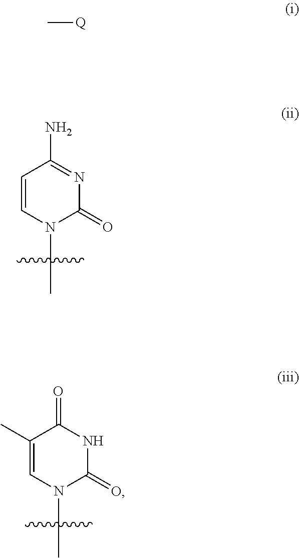
wherein Q is

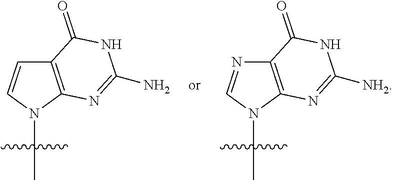 [SMILES: *n1ccc2c(=O)[nH]c(N)nc21 ; *n1cnc2c(=O)[nH]c(N)nc21]

64. The composition of any one of claims 1-59, wherein the heterocycles of the R² groups are each independently:

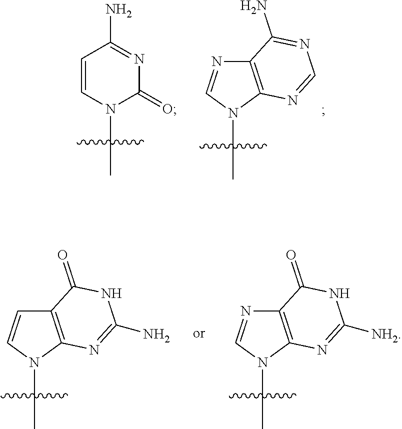

65. The composition of any one of claims 1-59, wherein each R² is independently: methyl,

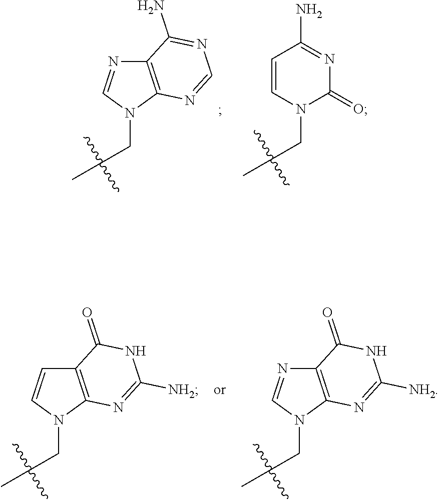 [SMILES: *Cn1cnc2c(N)ncnc21 ; *Cn1ccc(N)nc1=O ; *Cn1ccc2c(=O)[nH]c(N)nc21 ; *Cn1cnc2c(=O)[nH]c(N)nc21]

66. The composition of any one of claims 1-59, wherein the heterocycles of the R² groups form a sequence that repeats at least twice, wherein the sequence is, from N-Terminus to C-Terminus: 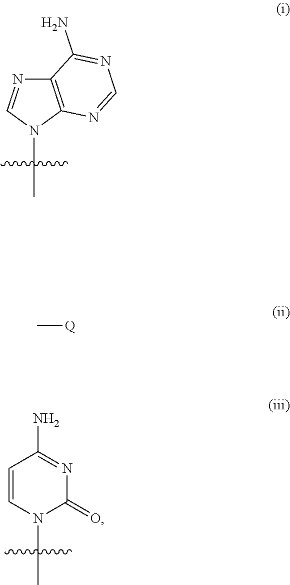
wherein Q is

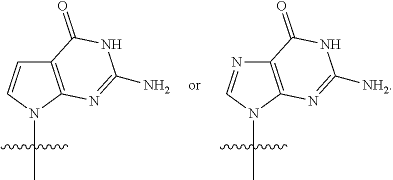 [SMILES: *n1ccc2c(=O)[nH]c(N)nc21 ; *n1cnc2c(=O)[nH]c(N)nc21]

67. The composition of any one of claims 1-59, wherein the heterocycles of the R² groups are each independently:

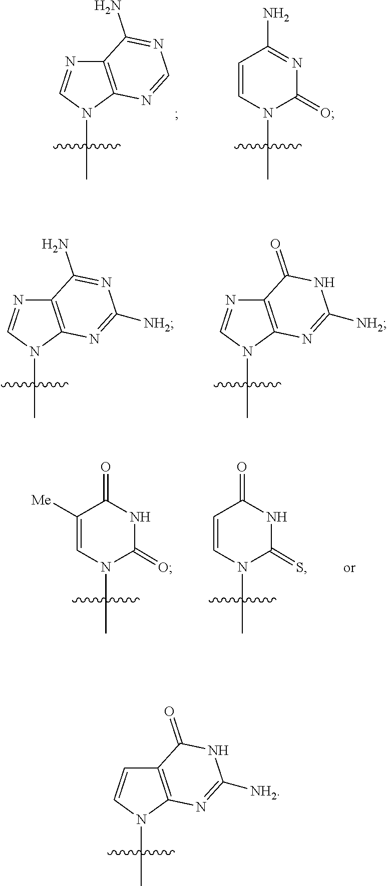

68. The composition of any one of claims 1-59, wherein each R² is independently: methyl,

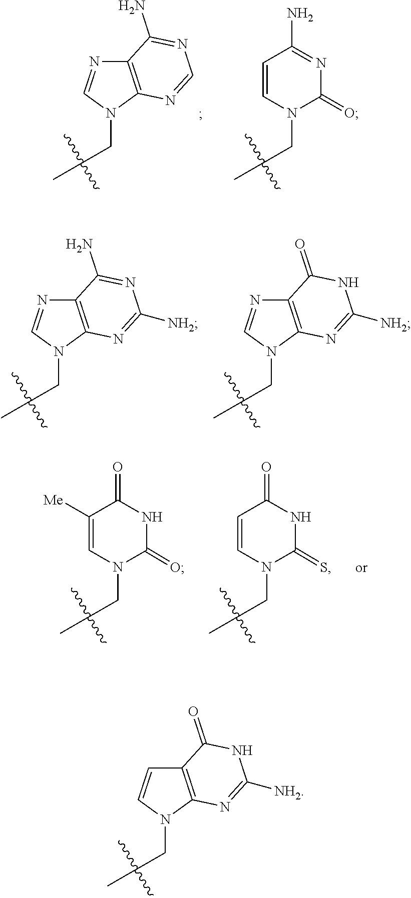

69. The composition of any one of claims 1-59, wherein the heterocycles of the R² groups form a sequence that repeats at least twice, wherein the sequence is, from N-Terminus to C-Terminus:

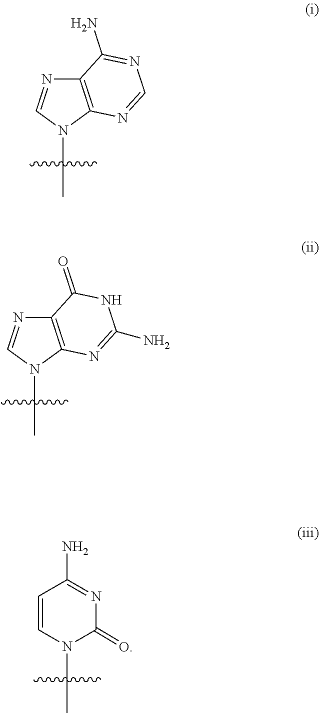 [SMILES: *n1cnc2c(N)ncnc21 ; *n1cnc2c(=O)[nH]c(N)nc21 ; *n1ccc(N)nc1=O]

70. The composition of any one of claims 1-69, wherein the compound binds to a nucleic acid sequence transcribed from a gene associated with a neuromuscular disease phenotype.

71. The composition of any one of claims 1-69, wherein the compound binds to a nucleic acid sequence transcribed from a gene associated with a DM1 disease phenotype.

72. The composition of any one of claims 1-71, wherein the compound binds to a nucleic acid sequence transcribed from a gene associated with a DM1 disease phenotype by interactions between the heterocycles of the R² groups and nucleobases of a DM1 gene.

73. The composition of claim 72, wherein the DM1 gene is a non-wild type DM1 gene. In some claims, the non-wild type DM1 gene differs from a wild type DM1 gene in a repeat expansion mutation.

74. The composition of any one of claims 1-70, wherein the compound binds to a nucleic acid sequence transcribed from a gene associated with a neurodegenerative disease phenotype.

75. The composition of any one of claims 1-70 and 74, wherein the compound binds to a nucleic acid sequence transcribed from a gene associated with a Huntington's disease phenotype.

76. The composition of any one of claims 1-70 and 74, wherein the compound binds to a nucleic acid sequence transcribed from a gene associated with a Huntington's disease phenotype by interactions between the heterocycles of the R² groups and nucleobases of a HTT gene.

77. The composition of claim 76, wherein the HTT gene is a non-wild type HTT gene.

78. The composition of claim 77, wherein the non-wild type HTT gene differs from a wild type HTT gene in a repeat expansion mutation.

79. The composition of any one of claims 1-56, wherein the compound binds to a mRNA sequence of (CUG)_{z}, wherein z is an integer from 1-100 (SEQ ID NO: 324).

80. The composition of any one of claims 1-56, wherein the compound binds to a mRNA sequence of (CAG)_{z}, wherein z is an integer from 1-100 (SEQ ID NO: 325).

81. The composition of any one of claims 1-69, wherein the compound binds to a nucleic acid sequence encoding a cancer-causing protein.

82. The composition of claim 81, wherein the cancer-causing protein is mutant K-ras.

83. The composition of claim 81, wherein the cancer-causing protein is G12D K-ras.

84. The composition of claim 81, wherein the cancer-causing protein is G12C K-ras.

85. The composition of claim 81, wherein the cancer-causing protein is G12V K-ras.

86. The composition of any one of claims 1-85, wherein the compound binds to the nucleic acid sequence encoding the mutant K-ras by interactions between the heterocycles of the R² groups and nucleobases of the nucleic acid sequence.

87. The composition of any one of claims 1-86, wherein the nucleic acid sequence is a mRNA sequence.

88. The composition of any one of claims 1-86, wherein the nucleic acid sequence is a DNA sequence.

89. The composition of claim 1 or 56, wherein the compound is: 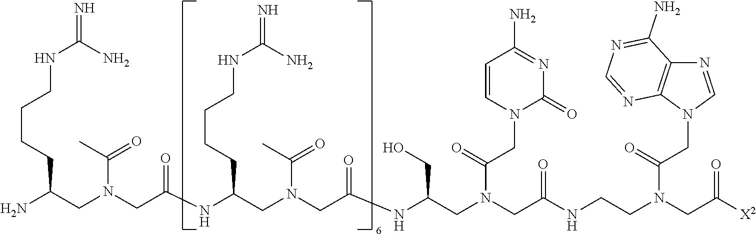
wherein X² is 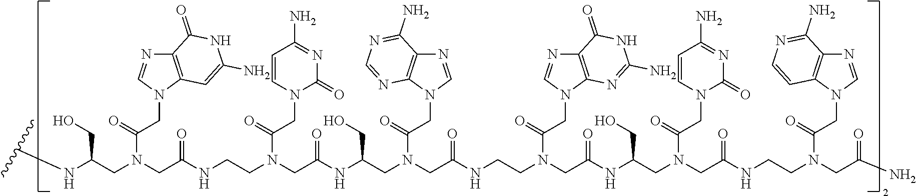
(Compound 1) (SEQ TD NO: 27) or a pharmaceutically-acceptable salt or ionized form thereof.

90. The composition of claim 1 or 56, wherein the compound is: 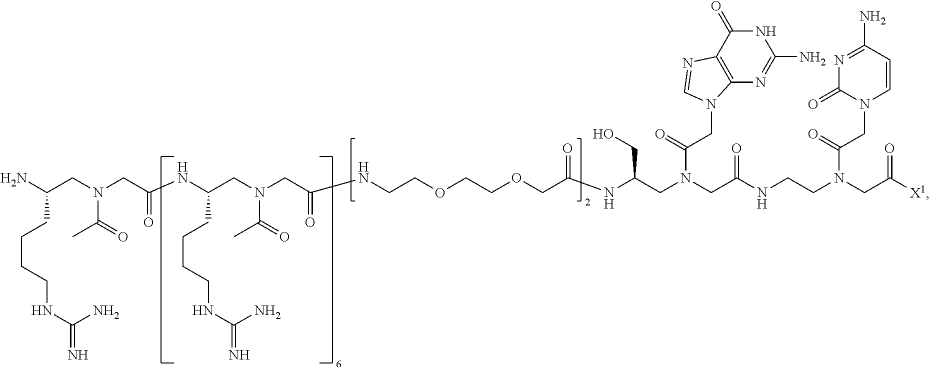
wherein X¹ is 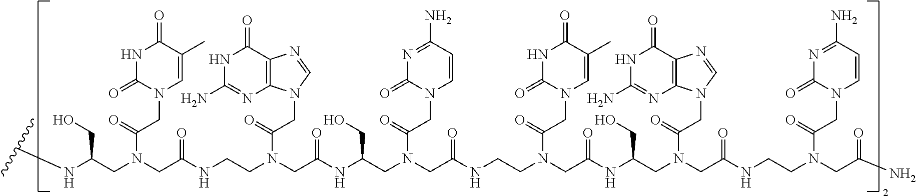
(Compound 166) (SEQ TD NO: 81), or a pharmaceutically-acceptable salt or ionized form thereof.

91. The composition claim 1 or 56, wherein the compound is: 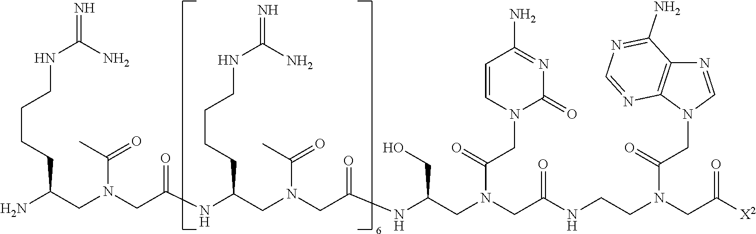
wherein X² is 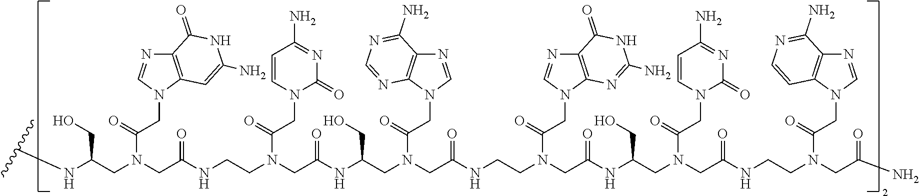
(Compound 167) (SEQ ID NO: 27), or a pharmaceutically-acceptable salt or ionized form thereof.

92. The composition of any one of claims 1-91, wherein the composition comprises the pharmaceutically-acceptable salt of the compound or an ionized form thereof, wherein the pharmaceutically-acceptable salt of the compound is a chloride salt.

93. The composition of any one of claims 1-91, wherein the composition comprises the pharmaceutically-acceptable salt of the compound or an ionized form thereof, wherein the pharmaceutically-acceptable salt of the compound is a trifluoroacetate salt.

94. The composition of any one of claims 1-91, wherein the composition comprises the pharmaceutically-acceptable salt of the compound or an ionized form thereof, wherein the pharmaceutically-acceptable salt of the compound is an acetate salt.

95. The composition of claim 94, wherein the pharmaceutically-acceptable salt comprises from about 5% to about 15% (w/w) acetate.

96. The composition of claim 94, wherein the pharmaceutically-acceptable salt comprises about 10% (w/w) acetate.

97. A method of treating a condition in a subject in need thereof, comprising administering to the subject a pharmaceutical composition of any one of claims 1-96.

98. The method of claim 97, wherein the administering is intravenous administration.

99. The method of claim 97, wherein the administering is subcutaneous administration.

100. The method of claim 97, wherein the administering is intramuscular administration.

101. The method of claim 97, wherein the administering is intracerebroventricular administration.

102. The method of claim 97, wherein the administering is intrathecal administration.

103. The method of any one of claims 97-102, wherein the condition is a neurodegenerative disease.

104. The method of any one of claims 97-103, wherein the condition is associated with an expanded trinucleotide repeat.

105. The method of any one of claims 97-104, wherein the condition is Huntington's disease.

106. The method of any one of claims 97-102, wherein the condition is a neuromuscular disease.

107. The method of any one of claims 97-102 and 106, wherein the condition is associated with an expanded trinucleotide repeat.

108. The method of any one of claims 97-102, 106, and 107, wherein the condition is myotonic dystrophy type 1.

109. The method of any one of claims 97-102, wherein the condition is cancer.
